(19) United States
(12) Patent Application Publication
(10) Pub. No.: US 2024/0150778 A1
(43) Pub. Date: May 9, 2024

(54) POLYPLOID HYBRID MAIZE BREEDING

(71) Applicant: Ohalo Genetics, Inc., Aptos, CA (US)

(72) Inventors: Judson Arthur WARD, Watsonville, CA (US); Jason A. PEIFFER, Hendersonville, NC (US); Lien Diana BERTIER, Santa Cruz, CA (US); Morgan Edward MCCAW, Aptos, CA (US); Xingang WANG, Aptos, CA (US); Benjamin Thomas BERUBE, Scotts Valley, CA (US)

(73) Assignee: Ohalo Genetics, Inc., Aptos, CA (US)

(21) Appl. No.: 18/504,917

(22) Filed: Nov. 8, 2023

Related U.S. Application Data
(60) Provisional application No. 63/461,170, filed on Apr. 21, 2023.
(60) Provisional application No. 63/497,670, filed on Apr. 21, 2023.
(60) Provisional application No. 63/461,174, filed on Apr. 21, 2023.
(60) Provisional application No. 63/423,768, filed on Nov. 8, 2022.
(60) Provisional application No. 63/423,765, filed on Nov. 8, 2022.

(51) Int. Cl. C12N 15/82

(52) U.S. Cl. CPC C12N 15/8242; C12N 15/8213

(57) ABSTRACT

The present inventions relate to a breeding system for the production of polyploid maize seeds, maize plants, or maize plant parts where cycles of meiosis, syngamy, and selection are used for interpopulation improvement of progenitor lines, and sexual polyploidization occurs during hybrid production by inducing clonal gamete formation in the parents that are to be crossed. Reciprocal recurrent selection can be used to inform selection of candidate maize lines that are either advanced to a gene editing or genetic modification system or crossed and selected to induce clonal gamete formation by arresting meiotic recombination and chromosome reduction. Crosses of parent maize plants bearing clonal gametes are planned and executed based upon predicted heterotic performance at the polyploid level. The final product is a homogeneous population of hybrid polyploid maize seed, or derivative thereof, bearing both parents' complete nuclear genomes.

### CROSS-REFERENCE TO RELATED APPLICATIONS

This application claims the benefit of priority to U.S. Provisional Application No. 63/423,765, filed on Nov. 8, 2022, to U.S. Provisional Application No. 63/423,768, filed on Nov. 8, 2022, to U.S. Provisional Application No. 63/497,670, filed Apr. 21, 2023, to U.S. Provisional Application No. 63/461,174, filed Apr. 21, 2023, and to U.S. Provisional Application No. 63/461,170, filed Apr. 21, 2023, each of which is incorporated by reference herein in its entirety.

### REFERENCE TO AN ELECTRONIC SEQUENCE LISTING

The content of the electronic sequence listing (197072000300seq1ist.xml; Size: 105,385 bytes; and Date of Creation: Nov. 3, 2023) is herein incorporated by reference in its entirety.

### FIELD

The inventions relate generally to the field of agricultural science, and specifically to crop improvement and systems of breeding novel hybrid polyploid maize cultivars. The inventions also relate to population improvement methods to desirably alter the genetic composition of diploid maize breeding populations for accelerated production of uniform hybrid polyploid maize seeds, maize plants, and maize plant parts suitable for cultivation. The inventions further relate to maize plant materials obtained by this process.

### BACKGROUND

The growing human population, a desire to reduce the environmental impact of agriculture, and consumer food preferences ensure a constant need for improved varieties of crops. Crop breeding has been used as a way to improve crop characteristics, yields, and robustness to environmental pressure for millennia. Despite growth in crop yields in the 20^{th} century, crop yields have begun to plateau in recent decades, signaling a need for improved breeding methods (e.g., Rizzo (2021). Climate and agronomy, not genetics, underpin recent maize yield gains in favorable environments. Proceedings of the National Academy of Sciences 119(4): e2113629119). In some staple and specialty crops, the most widely-grown cultivars are polyploid. This creates immense complexity in breeding such varieties due to the increased inefficiency of artificial selection in polyploid plant species, and, in some cases, the requirement for vegetative propagation of the varieties, which is likely not even feasible in maize. In other cases reduced genetic gain in a diploid could be resolved by moving from diploid to a hybrid polyploid system.

Hybrid crops are widely grown and preferred because they tend to exhibit more robust growth, higher yields, and resilience to environmental stressors compared to their inbred or open-pollinated counterparts. This phenomenon is known as heterosis, or “hybrid vigor”, and reflects the tendency of a cross-bred plant to show superior quality due to extensive heterozygosity in the plant's genome. Hybrid seed is typically produced by a single cross of fully inbred parent plants with different sets of alleles, resulting in a biallelic hybrid plant with two sets of alleles (also known as haplotypes) contributing to heterosis. In a further extension of the mechanism of heterosis, polyploid crops can exhibit progressive heterosis; for example, the additional hybrid vigor in a multiallelic double-cross tetraploid hybrid plant that is not found in its biallelic single-cross tetraploid parents or in its more inbred grandparents. Progressive heterosis has been documented in a number of tetraploid species including alfalfa, potato, and tetraploid maize (Gallais. (1984). An analysis of heterosis vs. inbreeding effects with an autotetraploid cross-fertilized plant: Medicago sativa L. Genetics 106, 123-137; Groose et al. 1989. Progressive heterosis in autotetraploid alfalfa: studies using two types of inbreds. Crop Sci. 29, 1173-1177; Mok and Peloquin. 1975. Breeding value of 2n pollen (diplandroids) in tetraploid×diploid crosses in potatoes. Theor. Appl. Genet. 46, 307-314; Washburn et al. 2013. Polyploids as a “model system” for the study of heterosis. Plant Reprod 27:1-5; Washburn et al. 2019. Progressive heterosis in genetically defined tetraploid maize (J Genet Genomics. 46(8):389-396) has resulted in increased above ground biomass and several other agronomically desirable traits. However, due to the need to cross heterozygous single-cross parents to generate double-cross polyploid hybrids, it is not feasible to generate a uniform population of true-breeding seed while taking advantage of progressive heterosis with current breeding techniques. Use of tetraploids for grain crops such as maize has also been considered undesirable due to meiotic associations that may result in non-viable gametes and reduce yield.

Prior research has established methods that allow plant geneticists to arrest meiosis in plants and replace it with a mitosis-like division in germline cells, resulting in formation of clonal gametes that contain the complete nonrecombinant genome of the parent. One such method, known as MiMe (Mitosis instead of Meiosis; d'Erfurth et al. 2009. Turning meiosis into mitosis. PLoS Biol 7, no. 6: e1000124.) is achieved through a triple knockout of three genes encoding gene products involved in meiosis, specifically, (1) a component of sister chromatid cohesion during the first division of meiosis, (2) a component of DNA double strand breakage during meiotic recombination, and (3) a component of progression through the second division of meiosis. This technology for MiMe is well-developed, but commercial development of MiMe has only begun in a select few applications (e.g., see US20190098858A1, US20120042408A1, and U.S. Ser. No. 10/883,112B2). These current applications of MiMe that are in development have only focused on generation of apomictic seed from diploid plants. However, the application of MiMe to develop novel and enhanced breeding techniques for plants at the polyploid level has not yet been realized.

### BRIEF SUMMARY

Provided herein are novel methods of breeding polyploid maize plants and genetically uniform maize populations that apply MiMe in innovative ways to accelerate breeding and produce maize plants or maize populations that are either very difficult to produce by traditional breeding or simply cannot be produced by traditional breeding.

In particular, certain methods leverage the production of clonal gametes to unlock the potential of progressive heterosis in breeding of polyploid maize. First, a diverse set of maize lines of a given ploidy is obtained, improved using traditional breeding methods, and organized into heterotic groups based on the predicted heterotic performance of their haplotypes when combined in maize plants of higher ploidy. Then, candidate lines together comprising three or more haplotypes are selected from the set of maize lines, and one or more candidate lines are induced to form clonal gametes by a method such as MiMe. The clonal gametes are then crossed (for example, with other gametes such as clonal gametes, haploid gametes derived from a fully inbred individual, or other types of unreduced gametes that would result in three or more haplotypes) to produce a homogenous population of multiallelic polyploid maize seed comprising the three or more haplotypes of the candidate lines. The polyploid maize seed is then grown, the maize plants are evaluated for the characteristics desired in the breeding program, and the heterotic performance of the haplotypes comprised by the maize plants is used to guide the breeding and selection of maize lines for further rounds of breeding. Exemplary embodiments of these methods are depicted in FIGS. 12-20C. For many species this method allows, for the first time, the production of genetically uniform polyploid maize seed comprising three or more haplotypes, thus allowing for practical application of progressive heterosis in polyploids.

In one aspect, the present disclosure provides a population of polyploid maize seed comprising three or more haplotypes of the same or related species of maize plant, wherein at least 50% of the population of polyploid maize seed are genetically uniform, and wherein the population was obtained from a single maize plant or a set of maize plants such as, for example, a set of F1 hybrids. In some embodiments, the present disclosure provides a population of polyploid maize seed comprising a subpopulation of genetically uniform polyploid maize seed in an amount of at least 50% of the total number of seeds, the genetically uniform polyploid maize seed comprising three or more haplotypes of the same or related species of maize plant, wherein the population was obtained from a single maize plant or a set of maize plants such as, for example, of F1 hybrids. The polyploid maize seed (e.g., the subpopulation of genetically uniform polyploid maize seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed are genetically uniform. In certain embodiments, the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid maize seed has a pairwise identity of at least 95%, at least 96%, at least 97%, at least 98%, at least 99%, or 100% as measured by the Jaccard similarity coefficient. In certain embodiments, the polyploid maize seed (e.g., the subpopulation of genetically uniform polyploid maize seed) comprises four or more haplotypes of the same or related species of maize plant.

In some embodiments, the polyploid maize seed (e.g., the subpopulation of genetically uniform polyploid maize seed) comprises one or more genetic modifications resulting in decreased expression of one or more, two or more, or three or more MiMe loci. The seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In some embodiments, the polyploid maize seed (e.g., the subpopulation of genetically uniform polyploid maize seed) comprises one or more genetic modifications resulting in altered activity of one or more, two or more, or three or more MiMe components. In some embodiments, the altered activity includes, for example, a dominant negative, constitutively active or null mutant of the one or more MiMe components. In one embodiment, the polyploid maize seed (e.g., the subpopulation of genetically uniform polyploid maize seed) comprises one or more genetic modifications resulting in decreased expression of REC8, SWITCH1/DYAD, or a combination thereof. In another embodiment, the polyploid maize seed (e.g., the subpopulation of genetically uniform polyploid maize seed) comprises one or more genetic modifications resulting in decreased expression of OSD1, CYCA1, TDM1, PC1, PC2, FC, or any combination thereof. In another embodiment, the polyploid maize seed (e.g., the subpopulation of genetically uniform polyploid maize seed) comprises one or more genetic modifications resulting in decreased expression of PAIR1, SPO11-1, SPO11-2, PRD1, PRD2, DFO, MTOPVIB, DSY1, SY1, SY2, SY3, SY4, or any combination thereof. In yet another embodiment, the polyploid maize seed (e.g., the subpopulation of genetically uniform polyploid maize seed) comprises one or more genetic modifications resulting in decreased expression of PS1, JASON, or a combination thereof. The polyploid maize seed may comprise genetic modifications in any combination of MiMe loci described herein. The one or more genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid maize seed or the subpopulation of genetically uniform polyploid maize seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid or the subpopulation of genetically uniform polyploid maize seed has a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid maize seed or the subpopulation of genetically uniform polyploid maize seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid maize seed or the subpopulation of genetically uniform polyploid maize seed has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments, the population of polyploid maize seed or the subpopulation of genetically uniform polyploid maize seed has (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC.

In certain embodiments, the population of polyploid maize seed or the subpopulation of genetically uniform polyploid maize seed has (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In still additional variations, the MiMe locus of the component of progression through the first division of meiosis of the third haplotype is PS1 or JASON. In yet additional variations, the MiMe locus of the component of progression through the second division of meiosis of the third haplotype is OSD1, CYCA1, TDM1, PC1, PC2 or FC.

In some embodiments, the population of polyploid maize seed or the subpopulation of genetically uniform polyploid maize seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the population of polyploid maize seed or the subpopulation of genetically uniform polyploid maize seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid maize seed or the subpopulation of genetically uniform polyploid maize seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid maize seed or the subpopulation of genetically uniform polyploid maize seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the population of polyploid maize seed or the subpopulation of genetically uniform polyploid maize seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid maize seed or the subpopulation of genetically uniform polyploid maize seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In another aspect, the present disclosure provides a population of polyploid maize seed having a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component, wherein at least 50% of the population of polyploid maize seed are genetically uniform, and wherein the population was obtained from a single maize plant or a set of maize plants such as, for example, a set of F1 hybrids. In some embodiments, the present disclosure provides a population of polyploid maize seed comprising a subpopulation of genetically uniform polyploid maize seed in an amount of at least 50% of the total number of seeds, the genetically uniform polyploid maize seed comprising a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component, and wherein the population was obtained from a single maize plant or a set of maize plants such as, for example, a set of F1 hybrids. The polyploid maize seed (e.g., the subpopulation of genetically uniform polyploid maize seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed are genetically uniform. In certain embodiments, the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid maize seed has a pairwise identity of at least 95%, at least 96%, at least 97%, at least 98%, at least 99%, or 100% as measured by the Jaccard similarity coefficient. In some embodiments, the partially-complemented genotype comprises only MiMe alleles at one or more MiMe loci of a third MiMe component. In other embodiments, the partially-complemented genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In certain embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In another aspect, the present disclosure provides a population of polyploid maize seed having a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein at least 50% of the population of polyploid maize seed are genetically uniform, and wherein the population was obtained from a single parent maize plant or a set of maize plants, such as, for example, a set of F1 hybrids. In some embodiments, the present disclosure provides a population of polyploid maize seed comprising a subpopulation of genetically uniform polyploid maize seed in an amount of at least 50% of the total number of seeds, the genetically uniform polyploid maize seed comprising a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, and wherein the population was obtained from a single parent maize plant or a set of maize plants, such as, for example, a set of F1 hybrids. The polyploid maize seed (e.g., the subpopulation of genetically uniform polyploid maize seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed are genetically uniform. In certain embodiments, the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid maize seed has a pairwise identity of at least 95%, at least 96%, at least 97%, at least 98%, at least 99%, or 100% as measured by the Jaccard similarity coefficient. In some embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In another aspect, the present disclosure provides a population of polyploid maize seed having a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein at least 50% of the population of polyploid maize seed are genetically uniform, and wherein the population was obtained from a single parent maize plant or a set of maize plants, such as, for example, a set of F1 hybrids. In some embodiments, the present disclosure provides a population of polyploid maize seed comprising a subpopulation of genetically uniform polyploid maize seed in an amount of at least 50% of the total number of seeds, the genetically uniform polyploid maize seed comprising a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, and wherein the population was obtained from a single parent maize plant or a set of maize plants, such as, for example, a set of F1 hybrids. The polyploid maize seed (e.g., the subpopulation of genetically uniform polyploid maize seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed are genetically uniform. In certain embodiments, the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid maize seed has a pairwise identity of at least 95%, at least 96%, at least 97%, at least 98%, at least 99%, or 100% as measured by the Jaccard similarity coefficient. In some embodiments, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In some embodiments of the foregoing aspects, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. In some embodiments, the polyploid maize seed (e.g., the subpopulation of genetically uniform polyploid maize seed) comprises one or more genetic modifications resulting in altered activity of one or more, two or more, or three or more MiMe components. In some embodiments, the altered activity includes, for example, a dominant negative, constitutively active or null mutant of the one or more MiMe components

In some embodiments of the foregoing aspects, the population of polyploid maize seed or the subpopulation of genetically uniform polyploid maize seed has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1.

In some embodiments of the foregoing aspects, the population of polyploid maize seed or the subpopulation of genetically uniform polyploid maize seed has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments of the foregoing aspects, the population of polyploid maize seed or the subpopulation of genetically uniform polyploid maize seed has a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In another aspect, the present disclosure provides a method of producing a population of polyploid maize seed described herein, the method comprising: (a) providing clonal gametes from a pair of parent MiMe maize plants that together comprise three or more haplotypes; and (b) crossing the clonal gametes to produce the population of polyploid maize seed. In some embodiments, at least 50% of the population of polyploid maize seed are genetically uniform and comprise three or more haplotypes. In some embodiments, at least 70%, at least 90%, at least 95%, at least 96%, at least 97%, at least 98%, or at least 99% of the population of polyploid maize seed produced by said method are genetically uniform. In some embodiments, the population of polyploid maize seed comprises a subpopulation of genetically uniform polyploid maize seed in an amount of at least 50% of the total number of seeds, the subpopulation of genetically uniform polyploid maize seed comprising three or more haplotypes. In certain embodiments, the population of polyploid maize seed comprises a subpopulation of genetically uniform polyploid maize seed in an amount of at least 70%, at least 90%, at least 95%, at least 96%, at least 97%, at least 98%, or at least 99% of the total number of seeds. In certain embodiments, the polyploid maize seed (e.g., the subpopulation of genetically uniform polyploid maize seed) produced by said method comprises four or more haplotypes of the same or related species of maize plant. The population of polyploid maize seed or the subpopulation of genetically uniform polyploid maize seed may be, for example, triploid, tetraploid, pentaploid, hexaploid, heptaploid, or octoploid.

In some embodiments, the method of producing the population of polyploid seeds comprises producing a population of polyploid maize seed comprising one or more genetic modifications resulting in decreased expression of one or more, two or more, or three or more MiMe loci. The population of polyploid maize seed or the subpopulation of genetically uniform polyploid maize seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the polyploid maize seed produced by said method, or the subpopulation of genetically uniform polyploid maize seed, comprises one or more genetic modifications resulting in decreased expression of REC8, SWITCH1/DYAD, or a combination thereof. In another embodiment, the polyploid maize seed produced by said method, or the subpopulation of genetically uniform polyploid maize seed, comprises one or more genetic modifications resulting in decreased expression of OSD1, CYCA1, TDM1, PC1, PC2, FC, or a combination thereof. In another embodiment, the polyploid maize seed produced by said method, or the subpopulation of genetically uniform polyploid maize seed, comprises one or more genetic modifications resulting in decreased expression of PAIR1, SPO11-1, SPO11-2, PRD1, PRD2, DFO, MTOPVIB, DSY1, SY1, SY2, SY3, SY4, or a combination thereof. In yet another embodiment, the polyploid maize seed produced by said method comprises one or more genetic modifications resulting in decreased expression of PS1, JASON, or a combination thereof. The polyploid maize seed produced by said method, or the subpopulation of genetically uniform polyploid maize seed, may comprise genetic modifications in any combination of MiMe loci described herein. The genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid maize seed produced by said method, or the subpopulation of genetically uniform polyploid maize seed, comprises a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid maize seed produced by said method, or the subpopulation of genetically uniform polyploid maize seed, comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid maize seed produced by said method, or the subpopulation of genetically uniform polyploid maize seed, has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid maize seed produced by said method, or the subpopulation of genetically uniform polyploid maize seed, has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In another aspect, the present disclosure provides a method of producing a population of polyploid maize seed described herein having a partially-complemented MiMe genotype, the method comprising (a) providing clonal gametes from a first parent MiMe maize plant, wherein the first parent MiMe maize plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) providing clonal gametes from a second parent MiMe maize plant, wherein the second parent MiMe maize plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (c) crossing the clonal gametes from the first and second parent MiMe maize plants to produce the population of polyploid maize seed having a partially-complemented MiMe genotype. In some embodiments, at least 50% of the population of polyploid maize seed are genetically uniform and comprise two, three, or more haplotypes. In some embodiments, the population of polyploid maize seed comprises a subpopulation of genetically uniform polyploid maize seed in an amount of at least 50% of the total number of seeds, the subpopulation of genetically uniform polyploid maize seed comprising the partially-complemented MiMe genotype. In certain embodiments, at least one of the MiMe loci having only MiMe alleles of the first MiMe component of the first parent MiMe maize plant is the same as at least one of the MiMe loci having only MiMe alleles of the first MiMe component of the second parent MiMe maize plant. In some embodiments, at least one of the MiMe loci having only MiMe alleles of the third MiMe component of the first parent MiMe maize plant is the same as at least one of the MiMe loci having only MiMe alleles of the third MiMe component of the second parent MiMe maize plant. In other embodiments, the one or more MiMe loci having only MiMe alleles of the third MiMe component of the first parent MiMe maize plant are distinct from the one or more MiMe loci having only MiMe alleles of the third MiMe component of the second parent MiMe maize plant. In some embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In another aspect, the present disclosure provides a method of producing a population of polyploid maize seed having a partially-complemented MiMe genotype described herein, the method comprising (a) providing clonal gametes from a first parent MiMe maize plant, wherein the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (b) providing clonal gametes from a second parent MiMe maize plant, wherein the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component; and (c) crossing the clonal gametes from the first and second parent MiMe maize plants to produce the population of polyploid maize seed having a partially-complemented MiMe genotype. In some embodiments, at least 50% of the population of polyploid maize seed are genetically uniform and comprise two, three, or more haplotypes. In some embodiments, the population of polyploid maize seed comprises a subpopulation of genetically uniform polyploid maize seed in an amount of at least 50% of the total number of seeds, the subpopulation of genetically uniform polyploid maize seed comprising the partially-complemented MiMe genotype. In some embodiments at least one of the MiMe loci having only MiMe alleles of the first MiMe component of the first parent MiMe maize plant is the same as at least one of the MiMe loci having only MiMe alleles of the first MiMe component of the second parent MiMe maize plant. In certain embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In yet another aspect, the present disclosure provides a method of producing a population of polyploid maize seed having a partially-complemented MiMe genotype described herein, the method comprising (a) providing clonal gametes from a first parent MiMe maize plant, wherein the first parent MiMe maize plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination; (b) providing clonal gametes from a second parent MiMe maize plant, wherein the second parent MiMe maize plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and (c) crossing the clonal gametes from the first and second parent MiMe maize plants to produce the population of polyploid maize seed having a partially-complemented MiMe genotype. In some embodiments, at least 50% of the population of polyploid maize seed are genetically uniform and comprise two, three, or more haplotypes. In some embodiments, the population of polyploid maize seed comprises a subpopulation of genetically uniform polyploid maize seed in an amount of at least 50% of the total number of seeds, the subpopulation of genetically uniform polyploid maize seed comprising the partially-complemented MiMe genotype. In some embodiments, at least one of the MiMe loci having only MiMe alleles of the first MiMe component of the first parent MiMe maize plant is the same as at least one of the MiMe loci having only MiMe alleles of the first MiMe component of the second parent MiMe maize plant. In certain embodiments, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In some embodiments of the foregoing methods, the first MiMe component is a component of sister chromatid cohesion during the first division of meiosis. In some variations of said method, the MiMe loci of the first MiMe component of both the first and second parent MiMe maize plants comprise REC8. In certain embodiments of said method, the second MiMe component is a component of DNA double strand breakage during meiotic recombination. In some variations of said method, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation of said method, the first MiMe locus of the second MiMe component is PAIR1 and the second MiMe locus of the second MiMe component is SPO11-1. In further embodiments of said method, the third MiMe component is a component of progression through the second division of meiosis. In some embodiments of said method, at least one of the MiMe loci having only MiMe alleles of the third MiMe component of the first parent MiMe maize plant is the same as at least one of the MiMe loci having only MiMe alleles of the third MiMe component of the second parent MiMe maize plant. In some variations, the MiMe loci having only MiMe alleles of the component of progression through the second division of meiosis comprise OSD1, CYCA1, TDM1, PC1, PC2, FC, or any combination thereof. In one variation, the MiMe locus having only MiMe alleles of the third MiMe component is OSD1. In other embodiments of said method, the one or more MiMe loci having only MiMe alleles of the third MiMe component of the first parent MiMe maize plant are distinct from the one or more MiMe loci having only MiMe alleles of the third MiMe component of the second parent MiMe maize plant. In some variations, the MiMe loci having only MiMe alleles of the third MiMe component comprise OSD1, CYCA1, TDM1, PC1, PC2, FC, or any combination thereof. In one variation of said method, the MiMe loci having only MiMe alleles of the first MiMe component comprise REC8, the first MiMe locus of the second MiMe component is PAIR1, the second MiMe locus of the second MiMe component is SPO11-1, and the MiMe loci having only MiMe alleles of the third MiMe component comprise OSD1.

In another aspect, the present disclosure provides a method of breeding a polyploid hybrid maize line, comprising: (a) obtaining a set of maize lines; (b) breeding the maize lines using traditional plant breeding methods to produce a set of candidate maize lines of the maize; (c) selecting two or more candidate maize lines together comprising three or more haplotypes; (d) generating two parent MiMe maize plants from the two or more candidate maize lines that together comprise the three or more haplotypes; (e) providing clonal gametes from each of the parent MiMe maize plants; (f) crossing the clonal gametes to produce a hybrid polyploid maize seed comprising the three or more haplotypes; (g) growing the hybrid polyploid maize seed to produce a hybrid polyploid maize plant comprising three or more haplotypes; and (h) evaluating one or more characteristics of the hybrid polyploid maize plant. The candidate maize lines and the parent MiMe maize plants may be any ploidy, including, but not limited to, haploid, monoploid, diploid, triploid, or tetraploid. In additional embodiments of said breeding method, the hybrid polyploid maize plant is tetraploid, pentaploid, hexaploid, heptaploid, or octoploid.

In some embodiments of said breeding method, step (d) comprises introducing a complete MiMe genotype directly into two candidate maize lines to produce the two parent MiMe maize plants. In other embodiments of said breeding method, step (d) comprises introducing a partial MiMe genotype into two candidate maize lines to produce two grandparent non-MiMe maize plants each having a partial MiMe genotype, crossing said grandparent non-MiMe maize plants each having a partial MiMe genotype to produce the first parent MiMe maize plant, and introducing a complete MiMe genotype directly into a third candidate maize line to produce the second parent MiMe maize plant. In yet other embodiments of said breeding method, step (d) comprises introducing a partial MiMe genotype into four candidate maize lines to produce four grandparent non-MiMe maize plants each having a partial MiMe genotype, and crossing pairs of said grandparent non-MiMe maize plants each having a partial MiMe genotype to produce the two parent MiMe maize plants. In certain embodiments, step (d) further comprises propagating parent MiMe maize plants to scale production of homogenous seed.

In some embodiments of said breeding method, the parent MiMe maize plants of step (d) each have a complete MiMe genotype comprising MiMe alleles that are naturally-occurring, introduced via genetic modification, or a combination thereof. In certain embodiments, the genetic modifications result in decreased expression of one or more, two or more, or three or more MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the genetic modifications result in decreased expression of REC8, SWITCH1/DYAD, or a combination thereof. In another embodiment, the genetic modifications result in decreased expression of OSD1, CYCA1, TDM1, PC1, PC2, FC, or any combination thereof. In another embodiment, the genetic modifications result in decreased expression of PAIR1, SPO11-1, SPO11-2, PRD1, PRD2, DFO, MTOPVIB, DSY1, SY1, SY2, SY3, SY4, or any combination thereof. In yet another embodiment, the genetic modifications result in decreased expression of PS1, JASON, or a combination thereof. In some embodiments, the one or more genetic modifications are introduced by gene editing, transgenesis, or a combination thereof. The genetic modifications may be achieved by any methods described herein, including, but not limited to, gene disruption, gene knockout, gene knockdown, gene silencing, RNA interference, induction of methylation, or any combination thereof.

In certain embodiments, the population of polyploid maize seed produced by said breeding method comprises a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid maize seed produced by said breeding method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid maize seed produced by said breeding method has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid maize seed produced by said breeding method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments of said breeding method, the method of breeding a population of polyploid maize seed further comprises (i) repeating steps (b)-(h) or steps (c)-(h) using the one or more characteristics of the hybrid polyploid maize plant evaluated in step (h) to guide the breeding of maize lines of step (b), the selecting of candidate maize lines of step (c), or both. In some variations, the one or more characteristics includes the heterotic performance of the three or more haplotypes of the polyploid hybrid maize plant evaluated in step (h).

In some embodiments of said breeding method, the set of maize lines in step (a) are obtained from one or more of natural diversity or existing breeding programs. In certain embodiments of said breeding method, step (a) further comprises organizing the set of maize lines into three or more heterotic groups, wherein each heterotic group comprises a haplotype, and wherein the haplotypes are grouped based on observed or predicted heterotic performance when combined in the hybrid polyploid maize plant of step (g). In one variation, step (a) comprises organizing the set of maize lines into four or more heterotic groups. In certain embodiments, heterotic performance is predicted via genome prediction modeling. In some embodiments of said breeding method, step (b) comprises reciprocal recurrent selection, inbreeding one or more of the maize lines to homozygosity, production of a doubled haploid maize line (e.g., a doubled monoploid maize line), backcrossing, or any other method known in the art for creating maize lines with high degrees of homozygosity, or a combination thereof. The candidate maize lines of step (c) may be inbred maize lines, hybrid maize lines, or a combination thereof.

In another aspect, the present disclosure provides a method of producing a population of polyploid maize seed comprising: (a) providing clonal gametes from a pair of parent MiMe maize plants that together comprise three or more haplotypes that were selected using the methods of breeding described herein based upon the polyploid maize plant comprising said three or more haplotypes having one or more desired characteristics; and (b) crossing the clonal gametes to produce the population of polyploid maize seed, wherein at least 50% of the population of polyploid maize seed are genetically uniform and comprise three or more haplotypes. The polyploid maize seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed produced by said method are genetically uniform. In certain embodiments, the polyploid maize seed produced by said method comprises four or more haplotypes of the same or related species of maize. In some variations of said method, maize lines are maintained via vegetative propagation, selfing, apomixis, cell culture, or any combination thereof. In some embodiments, said method further comprises maintaining an inventory of maize lines from which haplotypes may be selected for rapid deterministic stacking of the haplotypes. In some variations, the inventory of maize lines comprises one or more maize lines having a complete MiMe genotype that is maintained through vegetative propagation, hybridization with a haploid inducer, or a combination thereof. In additional variations, the inventory of maize lines comprises one or more maize lines having a partial MiMe genotype.

In another aspect, the present disclosure provides a method of producing a population of polyploid maize seed comprising: (a) providing clonal gametes from a parent MiMe maize plant; (b) providing haploid (e.g., monoploid) gametes from a homozygous parent non-MiMe maize plant; and (c) crossing the clonal gametes with the haploid (e.g., monoploid) gametes to produce the population of polyploid maize seed, wherein the clonal gametes and the haploid (e.g., monoploid) gametes together comprise three or more haplotypes, and wherein at least 50% of the population of polyploid maize seed are genetically uniform and comprise three or more haplotypes. The parent MiMe maize plant may be, for example, diploid, triploid, or tetraploid. The homozygous parent non-MiMe maize plant may be, for example, diploid or tetraploid. The polyploid maize seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed produced by said method are genetically uniform. In certain embodiments, the polyploid maize seed produced by said method comprises four or more haplotypes of the same or related species of maize plant.

In some embodiments, the method of producing a population of polyploid maize seed comprises producing a population of polyploid maize seed comprising one or more genetic modifications resulting in decreased expression of one or more, two or more, or three or more MiMe loci. The seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the polyploid maize seed produced by said method comprises one or more genetic modifications resulting in decreased expression of REC8, SWITCH1/DYAD, or a combination thereof. In another embodiment, the polyploid maize seed produced by said method comprises one or more genetic modifications resulting in decreased expression of OSD1, CYCA1, TDM1, PC1, PC2, FC, or a combination thereof. In another embodiment, the polyploid maize seed produced by said method comprises one or more genetic modifications resulting in decreased expression of PAIR1, SPO11-1, SPO11-2, PRD1, PRD2, DFO, MTOPVIB, DSY1, SY1, SY2, SY3, SY4, or a combination thereof. In yet another embodiment, the polyploid maize seed produced by said method comprises one or more genetic modifications resulting in decreased expression of PS1, JASON, or a combination thereof. The polyploid maize seed produced by said method may comprise genetic modifications in any combination of MiMe loci described herein. The genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid maize seed produced by said method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid maize seed produced by said method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In another aspect, the present disclosure provides a method of breeding a polyploid hybrid maize line, comprising: (a) obtaining a set of maize lines; (b) breeding the maize lines using traditional plant breeding methods to produce a set of candidate maize lines of maize; (c) selecting two or more candidate maize lines together comprising three or more haplotypes; (d) generating a parent MiMe maize plant from one of the two or more candidate maize lines; (e) providing clonal gametes from the parent MiMe maize plant; (f) providing haploid (e.g., monoploid) gametes from a homozygous parent non-MiMe maize plant of one of the two or more candidate maize lines; (g) crossing the clonal gametes with the haploid (e.g., monoploid) gametes to produce a hybrid polyploid maize seed; (h) growing the hybrid polyploid maize seed to produce a hybrid polyploid maize plant; and (i) evaluating one or more characteristics of the hybrid polyploid maize plant, wherein the parent MiMe maize plant and the homozygous parent non-MiMe maize plant together comprise three or more haplotypes, wherein the crossing of step (g) results in the hybrid polyploid maize seed comprising three or more haplotypes, and wherein the growing of step (h) results in the hybrid polyploid maize plant comprising three or more haplotypes. The candidate maize lines, parent MiMe maize plant, and the homozygous parent non-MiMe maize plant may be any ploidy, including, but not limited to, haploid, monoploid, diploid, triploid, or tetraploid. In some embodiments of said breeding method, the hybrid polyploid maize plant is tetraploid, pentaploid, hexaploid, heptaploid, or octoploid.

In some embodiments of said breeding method, step (d) comprises introducing a complete MiMe genotype directly into a candidate maize line to produce the parent MiMe maize plant. In further embodiments of said breeding method, step (d) comprises introducing a partial MiMe genotype into two candidate maize lines to produce two grandparent non-MiMe maize plants each having a partial MiMe genotype, crossing said grandparent non-MiMe maize plants each having a partial MiMe genotype to produce the parent MiMe maize plant. In certain embodiments, step (d) further comprises propagating the parent MiMe maize plant to scale production of homogenous seed.

In some embodiments of said breeding method, the parent MiMe maize plant of step (d) has a complete MiMe genotype comprising MiMe alleles that are naturally-occurring, introduced via genetic modification, or a combination thereof. In certain embodiments, the genetic modifications result in decreased expression of one or more, two or more, or three or more MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the genetic modifications result in decreased expression of REC8, SWITCH1/DYAD, or a combination thereof. In another embodiment, the genetic modifications result in decreased expression of OSD1, CYCA1, TDM1, PC1, PC2, FC, or any combination thereof. In another embodiment, the genetic modifications result in decreased expression of PAIR1, SPO11-1, SPO11-2, PRD1, PRD2, DFO, MTOPVIB, DSY1, SY1, SY2, SY3, SY4, or any combination thereof. In yet another embodiment, the genetic modifications result in decreased expression of PS1, JASON, or a combination thereof. In some embodiments, the one or more genetic modifications are introduced by gene editing, transgenesis, or a combination thereof. The genetic modifications may be achieved by any methods described herein, including, but not limited to, gene disruption, gene knockout, gene knockdown, gene silencing, RNA interference, induction of methylation, or any combination thereof.

In certain embodiments, the population of polyploid maize seed produced by said breeding method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid maize seed produced by said breeding method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments of said breeding method, the method of breeding a population of polyploid maize seed further comprises (j) repeating steps (b)-(i) or steps (c)-(i) using the one or more characteristics of the hybrid polyploid maize plant evaluated in step (i) to guide the breeding of maize lines of step (b), the selecting of candidate maize lines of step (c), or both. In some variations, the one or more characteristics includes the heterotic performance of the three or more haplotypes of the polyploid hybrid maize plant evaluated in step (i).

In some embodiments of said breeding method, the set of maize lines in step (a) are obtained from one or more of natural diversity or existing breeding programs. In certain embodiments of said breeding method, step (a) further comprises organizing the set of maize lines into three or more heterotic groups, wherein each heterotic group comprises a haplotype, and wherein the haplotypes are grouped based on observed or predicted heterotic performance when combined in the hybrid polyploid maize plant of step (h). In one variation, step (a) comprises organizing the set of maize lines into four or more heterotic groups. In certain embodiments, heterotic performance is predicted via genome prediction modeling. In some embodiments of said breeding method, step (b) comprises reciprocal recurrent selection, inbreeding one or more of the maize lines to homozygosity, production of a doubled haploid maize line (e.g., a doubled monoploid maize line), backcrossing, or any other method known in the art for creating maize lines with high degrees of homozygosity, or a combination thereof. The candidate maize lines of step (c) may be inbred maize lines, hybrid maize lines, or a combination thereof.

In another aspect, the present disclosure provides a method of producing a population of polyploid maize seed comprising: (a) selecting three or more haplotypes using the method of breeding described herein based upon the polyploid maize plant comprising said three or more haplotypes having one or more desired characteristics; (b) providing clonal gametes from a parent MiMe maize plant; (c) providing haploid (e.g., monoploid) gametes from a homozygous parent non-MiMe maize plant; (d) crossing the clonal gametes with the haploid (e.g., monoploid) gametes to produce the population of polyploid maize seed; wherein the parent MiMe maize plant and the homozygous parent non-MiMe maize plant together comprise the three or more haplotypes selected in step (a), wherein the crossing of step (d) results in a population of polyploid maize seed comprising the three or more haplotypes selected in step (a), and wherein at least 50% of the population of polyploid maize seed are genetically uniform and comprise three or more haplotypes. The polyploid maize seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid maize seed produced by said method are genetically uniform. In certain embodiments, the polyploid maize seed produced by said method comprises four or more haplotypes of the same or related species of maize. In some variations of said method, maize lines are maintained via vegetative propagation, selfing, apomixis, cell culture, or any combination thereof. In some embodiments, said method further comprises maintaining an inventory of maize lines of maize from which haplotypes may be selected for rapid deterministic stacking of the haplotypes. In some variations, the inventory of maize lines comprises one or more maize lines having a complete MiMe genotype that is maintained through vegetative propagation, hybridization with a haploid inducer, or a combination thereof. In additional variations, the inventory of maize lines comprises one or more maize lines having a partial MiMe genotype.

In another aspect, the present disclosure provides a method of breeding a polyploid maize plant, comprising (a) obtaining a set of maize lines; (b) breeding the maize lines using traditional plant breeding methods to produce a set of candidate maize lines of maize; (c) selecting one or more candidate maize lines; (d) generating a first parent MiMe maize plant from one of the candidate maize lines, wherein the first parent MiMe maize plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (e) generating a second parent MiMe maize plant from one of the candidate maize lines, wherein the second parent MiMe maize plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component, wherein at least one of the MiMe loci having only MiMe alleles of the first MiMe component of the first parent MiMe maize plant is the same as at least one of the MiMe loci having only MiMe alleles of the first MiMe component of the second parent MiMe maize plant; (f) providing clonal gametes from each of the parent MiMe maize plants; (g) crossing the clonal gametes to produce a polyploid maize seed; (h) growing the polyploid maize seed to produce a polyploid maize plant; and (i) evaluating one or more characteristics of the polyploid maize plant. In some embodiments, at least one of the MiMe loci having only MiMe alleles of the third MiMe component of the first parent MiMe maize plant is the same as at least one of the MiMe loci having only MiMe alleles of the third MiMe component of the second parent MiMe maize plant. In other embodiments, the one or more MiMe loci having only MiMe alleles of the third MiMe component of the first parent MiMe maize plant are distinct from the one or more MiMe loci having only MiMe alleles of the third MiMe component of the second parent MiMe maize plant. In certain embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In some embodiments of said breeding method, the first MiMe component is a component of sister chromatid cohesion during the first division of meiosis. In some variations of said breeding method, the MiMe loci of the first MiMe component of both the first and second parent MiMe maize plants comprise REC8. In certain embodiments of said breeding method, the second MiMe component is a component of DNA double strand breakage during meiotic recombination. In some variations of said breeding method, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation of said breeding method, the first MiMe locus of the second MiMe component is PAIR1 and the second MiMe locus of the second MiMe component is SPO11-1. In further embodiments of said breeding method, the third MiMe component is a component of progression through the second division of meiosis. In some embodiments of said breeding method, at least one of the MiMe loci having only MiMe alleles of the third MiMe component of the first parent MiMe maize plant is the same as at least one of the MiMe loci having only MiMe alleles of the third MiMe component of the second parent MiMe maize plant. In some variations, the MiMe loci having only MiMe alleles of the component of progression through the second division of meiosis comprise OSD1, CYCA1, TDM1, PC1, PC2, FC, or any combination thereof. In one variation, the MiMe locus having only MiMe alleles of the third MiMe component is OSD1. In other embodiments of said breeding method, the one or more MiMe loci having only MiMe alleles of the third MiMe component of the first parent MiMe maize plant are distinct from the one or more MiMe loci having only MiMe alleles of the third MiMe component of the second parent MiMe maize plant. In some variations, the MiMe loci having only MiMe alleles of the third MiMe component comprise OSD1, CYCA1, TDM1, PC1, PC2, FC, or any combination thereof. In one variation of said breeding method, the MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1.

In another aspect, the present disclosure provides a method of producing a population of polyploid maize seed comprising (a) providing clonal gametes from a pair of parent MiMe maize plants together comprising two or more haplotypes that were selected using the foregoing method of breeding based upon the polyploid maize plant comprising said two or more haplotypes having one or more desired characteristics, wherein: (i) the first parent MiMe maize plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (ii) the second parent MiMe maize plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (iii) at least one of the MiMe loci having only MiMe alleles of the first MiMe component of the first parent MiMe maize plant is the same as at least one of the MiMe loci having only MiMe alleles of the first MiMe component of the second parent MiMe maize plant; and (b) crossing the clonal gametes to produce the population of polyploid maize seed, wherein at least 50% of the population of polyploid maize seed are genetically uniform and comprise two or more haplotypes. In some embodiments, at least one of the MiMe loci having only MiMe alleles of the third MiMe component of the first parent MiMe maize plant is the same as at least one of the MiMe loci having only MiMe alleles of the third MiMe component of the second parent MiMe maize plant. In other embodiments, the one or more MiMe loci having only MiMe alleles of the third MiMe component of the first parent MiMe maize plant are distinct from the one or more MiMe loci having only MiMe alleles of the third MiMe component of the second parent MiMe maize plant.

In another aspect, the present disclosure provides a method of breeding a polyploid maize plant, comprising (a) obtaining a set of maize lines; (b) breeding the maize lines using traditional plant breeding methods to produce a set of candidate maize lines of maize; (c) selecting one or more candidate maize lines; (d) generating a first parent MiMe maize plant from one of the candidate maize lines, wherein the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (e) generating a second parent MiMe maize plant from one of the candidate maize lines, wherein the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component, and at least one of the MiMe loci having only MiMe alleles of the first MiMe component of the first parent MiMe maize plant is the same as at least one of the MiMe loci having only MiMe alleles of the first MiMe component of the second parent MiMe maize plant; (f) providing clonal gametes from each of the parent MiMe maize plants; (g) crossing the clonal gametes to produce a polyploid maize seed; (h) growing the polyploid maize seed to produce a polyploid maize plant; and (i) evaluating one or more characteristics of the polyploid maize plant. In some embodiments of said breeding method, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In another aspect, the present disclosure provides a method of producing a population of polyploid maize seed comprising (a) providing clonal gametes from a pair of parent MiMe maize plants together comprising two or more haplotypes that were selected using the foregoing method of breeding based upon the polyploid maize plant comprising said two or more haplotypes having one or more desired characteristics, wherein: (i) the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (ii) the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component; and (iii) at least one of the MiMe loci having only MiMe alleles of the first MiMe component of the first parent MiMe maize plant is the same as at least one of the MiMe loci having only MiMe alleles of the first MiMe component of the second parent MiMe maize plant; and (b) crossing the clonal gametes to produce the population of polyploid maize seed wherein at least 50% of the population of polyploid maize seed are genetically uniform and comprise two or more haplotypes.

In another aspect, the present disclosure provides a method of breeding a polyploid maize plant, comprising (a) obtaining a set of maize lines; (b) breeding the maize lines using traditional plant breeding methods to produce a set of candidate maize lines; (c) selecting one or more candidate maize lines; (d) generating a first parent MiMe maize plant from one of the candidate maize lines, wherein the first parent MiMe maize plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination; (e) generating a second parent MiMe maize plant from one of the candidate maize lines, wherein the second parent MiMe maize plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component, and at least one of the MiMe loci having only MiMe alleles of the first MiMe component of the first parent MiMe maize plant is the same as at least one of the MiMe loci having only MiMe alleles of the first MiMe component of the second parent MiMe maize plant; (f) providing clonal gametes from each of the parent MiMe maize plants; (g) crossing the clonal gametes to produce a polyploid maize seed; (h) growing the polyploid maize seed to produce a polyploid maize plant; and (i) evaluating one or more characteristics of the polyploid maize plant. In some embodiments of said breeding method, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In another aspect, the present disclosure provides a method of producing a population of polyploid maize seed comprising (a) providing clonal gametes from a pair of parent MiMe maize plants together comprising two or more haplotypes that were selected using the foregoing method of breeding based upon the polyploid maize plant comprising said two or more haplotypes having one or more desired characteristics, wherein: (i) the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (ii) the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component; and (iii) at least one of the MiMe loci having only MiMe alleles of the first MiMe component of the first parent MiMe maize plant is the same as at least one of the MiMe loci having only MiMe alleles of the first MiMe component of the second parent MiMe maize plant; and (b) crossing the clonal gametes to produce the population of polyploid maize seed, wherein at least 50% of the population of polyploid maize seed are genetically uniform and comprise two or more haplotypes.

In some embodiments of the foregoing breeding methods, steps (d) and (e) comprise introducing a complete MiMe genotype directly into two candidate maize lines to produce the two parent MiMe maize plants. In further embodiments of the foregoing breeding methods, steps (d) and (e) comprise introducing a partial MiMe genotype into two candidate maize lines to produce two grandparent non-MiMe maize plants each having a partial MiMe genotype, crossing said grandparent non-MiMe maize plants each having a partial MiMe genotype to produce the first parent MiMe maize plant, and introducing a complete MiMe genotype directly into a third candidate maize line to produce the second parent MiMe maize plant. In yet further embodiments of the foregoing breeding methods, steps (d) and (e) comprise introducing a partial MiMe genotype into four candidate maize lines to produce four grandparent non-MiMe maize plants each having a partial MiMe genotype, and crossing pairs of said grandparent non-MiMe maize plants each having a partial MiMe genotype to produce the two parent MiMe maize plants. In certain embodiments of the foregoing breeding methods, steps (d) and (e) further comprise propagating parent MiMe maize plants to scale production of homogenous seed.

In some embodiments of the foregoing breeding methods, the method further comprises (j) repeating steps (b)-(i) or steps (c)-(i) using the one or more characteristics of the polyploid maize plant evaluated in step (i) to guide the breeding of the maize lines of step (b), the selecting of candidate maize lines of step (c), or both. In certain embodiments of the foregoing breeding methods, the one or more characteristics includes the heterotic performance of the two, three, four, or more haplotypes of the polyploid maize plant evaluated in step (i). In some embodiments of the foregoing breeding methods, the set of maize lines in step (a) are obtained from one or more of: natural diversity or existing breeding programs. In certain embodiments of the foregoing breeding methods, step (a) further comprises organizing the set of maize lines into two, three, four, or more heterotic groups, wherein each heterotic group comprises a haplotype, and wherein the haplotypes are grouped based on observed or predicted heterotic performance when combined in the polyploid maize plant of step (i). In some variations, heterotic performance is predicted via genome prediction modeling. In some embodiments of the foregoing breeding methods, step (b) comprises reciprocal recurrent selection, inbreeding one or more of the maize lines to homozygosity, production of a doubled haploid maize line (e.g., a doubled monoploid maize line), backcrossing, or any other method known in the art for creating maize lines with high degrees of homozygosity, or a combination thereof. In some embodiments of the foregoing breeding methods, one or more of the candidate maize lines of step (c) are inbred maize lines. In further embodiments of the foregoing breeding methods, one or more of the candidate maize lines of step (c) are hybrid maize lines.

In some embodiments of any of the forgoing breeding methods, the first and second parent MiMe maize plants together comprise two, three, four, or more haplotypes, resulting in a polyploid maize plant comprising two, three, four, or more haplotypes. In some embodiments of the foregoing breeding methods, the maize lines are maintained via vegetative propagation, selfing, apomixis, cell culture, or any combination thereof. In further embodiments of the forgoing methods, the method further comprises maintaining an inventory of maize lines from which haplotypes may be selected for rapid deterministic stacking of the haplotypes. In some variations, the inventory of maize lines comprises one or more maize lines having a complete MiMe genotype that is maintained through vegetative propagation, hybridization with a haploid inducer, or a combination thereof. In additional variations, the inventory of maize lines comprises one or more maize lines having a partial MiMe genotype.

In some embodiments of the foregoing aspects and embodiments, the one or more MiMe loci of the component of sister chromatid cohesion during the first division of meiosis comprise REC8, SWITCH1/DYAD, or a combination thereof. In some embodiments of the foregoing aspects and embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In some embodiments of the foregoing aspects and embodiments, the one or more MiMe loci of the component of progression through the second division of meiosis comprise OSD1, CYCA1, TDM1, PC1, PC2, FC, or any combination thereof. In some embodiments of the foregoing aspects and embodiments, the one or more MiMe loci of the component of progression through the first division of meiosis comprise PS1, JASON, or a combination thereof.

In some embodiments of any of the foregoing aspects and embodiments, the MiMe alleles comprise alleles that are naturally-occurring, introduced via genetic modification, or a combination thereof. In certain embodiments of the foregoing aspects and embodiments, the MiMe alleles comprise one or more genetic modifications. In further embodiments of the foregoing aspects and embodiments, one or more of the genetic modifications are at MiMe loci encoding gene products of the MiMe components. In some variations, the genetic modifications comprise modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci, or any combination thereof. In some embodiments of the foregoing aspects and embodiments, one or more genetic modifications are introduced by gene editing, transgenesis, or a combination thereof. In further embodiments of the foregoing aspects and embodiments, the decreased expression of the one or more MiMe loci is achieved by gene disruption, gene knockout, gene knockdown, gene silencing, RNA interference, induction of methylation, or any combination thereof.

In any of the foregoing aspects and embodiments, the population of polyploid maize seed may be, for example, triploid, tetraploid, pentaploid, hexaploid, heptaploid, or octoploid. In some embodiments of the foregoing aspects and embodiments, at least 50%, at least 70%, at least 80%, at least 90%, at least 95%, at least 96%, at least 97%, at least 98%, or at least 99% of the population of polyploid maize seed are genetically uniform. In some embodiments of the foregoing aspects and embodiments, the population of polyploid maize seed comprises a subpopulation of genetically uniform polyploid maize seed in an amount of at least 50%, at least at least 70%, at least 75%, at least 80%, at least 90%, at least 95%, at least 96%, at least 97%, at least 98%, or at least 99% of the total number of seeds. In certain embodiments of the foregoing aspects and embodiments, the polyploid maize seed (e.g., the subpopulation of genetically uniform polyploid maize seed) comprises two, three, four, or more haplotypes of the same or related species of maize.

In another aspect, the present disclosure provides a genetically modified maize plant, plant part, or plant cell. In some embodiments, the genetically modified maize plant, plant part, or plant cell comprises: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the genetically modified maize plant, plant part, or plant cell comprises: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations of the foregoing embodiments, the one or more MiMe loci of the component of sister chromatid cohesion during the first division of meiosis comprise REC8, SWITCH1/DYAD, or a combination thereof. In additional variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations of the foregoing embodiments, the one or more MiMe loci of the component of progression through the second division of meiosis comprise OSD1, CYCA1, TDM1, PC1, PC2, FC, or any combination thereof.

In some embodiments of said genetically modified maize plant, plant part, or plant cell, the present disclosure provides a genetically modified maize plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments of this aspect, the genetically modified maize plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of progression through the first division of meiosis comprise PS1, JASON, or a combination thereof.

In some embodiments of said genetically modified maize plant, plant part, or plant cell, the genetically modified maize plant, plant part, or plant cell comprises: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations of the foregoing embodiments, the one or more MiMe loci of the component of sister chromatid cohesion during the first division of meiosis comprise REC8, SWITCH1/DYAD, or a combination thereof. In additional variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations of the foregoing embodiments, the MiMe locus of the component of progression through the first division of meiosis of the third haplotype is PS1 or JASON. In still additional variations of the foregoing embodiments, the one or more MiMe loci of the component of progression through the second division of meiosis of the first and second haplotype comprise OSD1, CYCA1, TDM1, PC1, PC2, FC, or any combination thereof. In yet additional variations of the foregoing embodiments, the locus of the component of progression through the second division of meiosis of the third haplotype is OSD1, CYCA1, TDM1, PC1, PC2, or FC.

In some embodiments of said genetically modified maize plant, plant part, or plant cell, the genetically modified maize plant, plant part, or plant cell comprises: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of progression through the first division of meiosis of the first and second haplotype comprise PS1, JASON, or a combination thereof. In yet additional variations of the foregoing embodiments, the MiMe locus of the component of progression through the first division of meiosis of the third haplotype is PS1 or JASON. In still additional variations of the foregoing embodiments, the MiMe locus of the component of progression through the second division of meiosis of the third haplotype is OSD1, CYCA1, TDM1, PC1, PC2 or FC.

In some embodiments, the genetically modified maize plant, plant part, or plant cell has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified maize plant, plant part, or plant cell has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the genetically modified maize plant, plant part, or plant cell has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified maize plant, plant part, or plant cell has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified maize plant, plant part, or plant cell has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1 In some embodiments, the genetically modified maize plant, plant part, or plant cell has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified maize plant, plant part, or plant cell has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified maize plant, plant part, or plant cell has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the genetically modified maize plant, plant part, or plant cell has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified maize plant, plant part, or plant cell has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified maize plant, plant part, or plant cell has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified maize plant, plant part, or plant cell has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some aspects, provided herein is a genetically modified maize plant, plant part, or plant cell having a partially-complemented MiMe genotype. In some embodiments, the genetically modified maize plant, plant part, or plant cell has a partially-complemented MiMe genotype comprising: (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1.

In some embodiments, the present disclosure provides a genetically modified maize plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof.

In some embodiments, the present disclosure provides a genetically modified maize plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof.

In some embodiments, the genetically modified maize plant, plant part, or plant cell has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1.

In some embodiments, the genetically modified maize plant, plant part, or plant cell has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments, the genetically modified maize plant, plant part, or plant cell has a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments of said genetically modified maize plant, plant part, or plant cell, which may be combined with any of the preceding embodiments, the genetically modified maize plant, plant part, or plant cell is diploid, triploid, tetraploid, pentaploid, hexaploid, heptaploid, or octaploid. In additional embodiments which may be combined with any of the preceding embodiments, the genetically modified maize plant, plant part, or plant cell comprises two, three, four, or more haplotypes of the same or related species of maize. In certain embodiments, which may be combined with any of the preceding embodiments, the genetically modified maize plant part is a non-regenerable plant part. In certain embodiments, which may be combined with any of the preceding embodiments, the genetically modified maize plant cell is a non-regenerable maize plant cell. In certain embodiments, which may be combined with any of the preceding embodiments, the maize plant part is a flower, a pistil, a stamen, a leaf, a stem, a cutting, a tissue, a seed coat, an ovule, a pollen, a root, a rootstock, a scion, a pericarp, a cotyledon, a hypocotyl, a protoplast, an embryo, an endosperm, an anther, a seed, a cob, a glume, a husk, a leaf sheath, a ligule, a trichome, or a portion thereof.

In another aspect, provided herein is a processed maize product derived from any of the foregoing embodiments of genetically modified maize plants, plant parts, or plant cells, wherein the processed maize product comprises a detectable amount of the one or more MiMe alleles of the genetically modified maize plant, plant part, or plant cell. In some embodiments, the maize product is selected from the group consisting of maize plant parts, fresh corn, canned corn, dehydrated corn, starch, hulls, hominy, popcorn, cereal, grain, margarine, fermented alcoholic beverage, biofuel, gluten, corn syrup, table syrup, candy, confections, soft drinks, ice cream, shoe polish, corn sugar, infant formulas, dietetic foods, caramel coloring, vinegar, lactic acid, tanning mixtures, brewing additive, artificial silk, edible starch, dextrin, mucilage, glue, textile sizing, food sauces, fireworks, industrial starch, laundry starch, filler in paper, cosmetics, explosives, germ, oil cake or meal, cattle feed, plastic resin, ribber substitutes, erasers, elastic heels, soap, glycerin, soluble corn oil, cloth coloring, salad oils, cooking oils, medicinal oils, animal feed, paper, wallboard, filling material, fuel, charcoal, indusial solvent, biomass, oil, meal, food starch, syrup, sugar, animal feed, flour, flakes, bran, processed seed, and seed. In certain embodiments, the processed maize product is non-regenerable.

### DESCRIPTION OF THE FIGURES

The present application can be understood by reference to the following description taken in conjunction with the accompanying figures.

FIG. 1 depicts a phylogenetic tree of REC8 protein sequences from monocotyledonous plants.

FIG. 2 depicts a phylogenetic tree of SPO11-1 protein sequences from monocotyledonous plants.

FIG. 3 depicts a phylogenetic tree of PAIR1 protein sequences from monocotyledonous plants.

FIG. 4 depicts a phylogenetic tree of OSD1 protein sequences from monocotyledonous plants.

FIG. 5 depicts a plasmid map of pLDB3, used to introduce MiMe gene edits in Zea mays.

FIG. 6 depicts a plasmid map of pLDB11B, used to introduce MiMe gene edits in Zea mays.

FIG. 7 depicts a plasmid map of pLDB12B, used to introduce MiMe gene edits in Zea mays.

FIG. 8 depicts a plasmid map of pMEM4, used to introduce MiMe gene edits in Zea mays.

FIG. 9 depicts a plasmid map of pMEM6b, used to introduce MiMe gene edits in Zea mays.

FIG. 10 depicts a plasmid map of pOGZ2, used to introduce MiMe gene edits in Zea mays.

FIG. 11 depicts a plasmid map of pOGZ4, used to introduce MiMe gene edits in Zea mays.

FIG. 12 illustrates an embodiment where homozygous gene editing occurs at the parent stage.

FIG. 13 illustrates an embodiment where grandparents are inbred and homozygous gene editing occurs at the parent stage.

FIG. 14 illustrates an embodiment where heterozygous gene editing occurs at the grandparent stage.

FIG. 15 illustrates an embodiment where grandparents are inbred and heterozygous gene editing at the grandparent stage.

FIG. 16 illustrates an embodiment where MiMe loci are propagated at the grandparent stage.

FIG. 17 illustrates an embodiment where grandparents are inbred and MiMe loci are propagated at the grandparent stage.

FIG. 18 illustrates an embodiment where MiMe loci are propagated at the great-grandparent stage.

FIG. 19 illustrates an embodiment where unilateral editing results in sexual tetraploidization.

FIGS. 20A-20C illustrate embodiments where sterility of the polyploid hybrid seed is induced via complementation of one of the MiMe loci. FIG. 20A illustrates generation of a plant having a first exemplary partially-complemented MiMe genotype where MiMe loci are propagated at the parent stage and each parent has a different set of edited MiMe loci. FIG. 20B illustrates generation of a plant having a second exemplary partially-complemented MiMe genotype where MiMe loci are propagated at the parent stage and each parent has a different set of edited MiMe loci. FIG. 20C illustrates generation of a plant having a third exemplary partially-complemented MiMe genotype where MiMe loci are propagated at the parent stage and each parent has a different set of edited MiMe loci.

FIG. 21 depicts a plasmid map of pOGZ1, used to introduce MiMe gene edits in Zea mays.

FIGS. 22A-22B show the screening results for guide RNA screening for edits in maize. FIG. 22A shows the screening results where editing efficiency is displayed as the proportion of sequence reads edited compared to wild-type (vertical axis, in percentage) across a range of selected protospacers (horizontal axis) across four different genes (horizontal axis, grey boxes). From left to right, the maize orthologs are listed for OSD1 on chromosome 2 (“ZmOsd1-chr2”), OSD1 on chromosome 5 (“ZmOsd1-chr5-1”) and OSD1 duplication on chromosome 5 (“ZmOsd1-chr5-2”), CYCA1 on chromosome 3 (“ZmTAM-chr3”), and CYCA1 on chromosome 8 (“ZmTAM-chr8”). FIG. 22B shows further results of screening, as in FIG. 22A, with the exception that the exhibited genes are the maize orthologs of PAIR1 (“ZmPair1-1” and “ZmPair1-9”), REC8 (“ZmRec8”), and SPOT 1-1 (“ZmSpo11-1”).

FIGS. 23A-23F show the resultant editing of MiMe alleles and disruption of MiMe components in edited maize lines. FIG. 23A shows that four conserved amino acids in the REC8 protein are deleted as a result of the edited rec8 allele. FIG. 23B shows the truncated, inactivated OSD1 proteins resulting from frameshift edits that result in premature stop codons in the edited osd1 alleles. FIG. 23C shows the truncated, inactivated SPO11-1 protein resulting from a frameshift edit that results in a premature stop codon in the edited spo11-1 allele. FIG. 23D shows an alignment of edited “scar” nucleotide sequence of the edited rec8 allele (bottom portion continues from the top portion). FIG. 23E shows an alignment of edited “scar” nucleotide sequences of edited osd1 alleles (bottom portion continues from the top portion). FIG. 23F shows an alignment of edited “scar” nucleotide sequences of edited spo11-1 alleles (bottom portion continues from the top portion).

FIG. 24 illustrates the expected pairwise identity (or percentage of shared alleles as estimated by the Jaccard Similarity among genotypes) of randomly selected individuals within each population (SAP—Standard Arabidopsis Population, BAP1—Boosted Arabidopsis Population 1. BMP—Boosted Maize Population). The diamonds denote the expected or mean pairwise identity between genotyped plants in each population. The black bars are the median or second quartile. The boxes each represent the interquartile range from the first to the third quartile, and the dashed whiskers display the minimum and maximum pairwise identity between genotyped individuals in each population.

FIG. 25A shows a matrix for pairwise identity as estimated by the Jaccard similarity coefficient of 44 molecular markers genotyped between 48 tetraploid maize plants in Boosted Maize Population (BMP).

FIG. 25B shows a summary of the genotyping results at 44 triallelic markers across 48 progeny comprising the Boosted Maize Population (BMP). The horizontal axis corresponds to individual tetraploid progeny as well as the two diploid parent plants for reference. The vertical axis corresponds to individual triallelic markers distributed across 8 separate chromosomes. The coloration of each cell denotes the specific configuration of haplotypes observed at that marker (where A=LH244, B=A188, and C=FFMM-AT6). Samples with markers that are “AABC” have three haplotypes. Marker design was limited to regions of the genome where heterozygous parental SNPs were present. No usable markers were found on chromosome 2 or chromosome 10. The parent genotypes are displayed along the left-most rows.

FIGS. 26A-26D show examples of BMP individuals. FIG. 26A shows multiple rows of BMP plants. FIG. 26B shows a close-up of the right-hand side of FIG. 26A, displaying a close-up view of three BMP plants. FIG. 26C shows healthy BMP plants readily producing ears. FIG. 26D shows a close-up view of the ears of BMP plants as shown in FIG. 26C.

FIG. 27 shows the results for guide RNA screening for edits in A. thaliana. Editing efficiency is displayed as the proportion of sequence reads edited compared to wild-type (vertical axis, in percentage) across a range of selected protospacers (horizontal axis) across four different genes (horizontal axis, grey boxes). From left to right, the A. thaliana orthologs are listed for OSD1 (“AtOsd1 (Col-0)”), PAIR1 (“AtPair1 (Col-0)”), REC8 (“AtRec8 (Col-0)”), and SPO11-1 (“AtSpo11-1 (Col-0)”). The black arrows indicate which sites were explored further, based on the guide RNA screening results.

FIGS. 28A-2811 show the resultant editing of MiMe alleles and disruption of MiMe components in edited Arabidopsis lines. FIG. 28A shows the truncated, inactivated proteins resulting from premature stop codons that result from the edited osd1 alleles. FIG. 28B shows the truncated, inactivated proteins resulting from insertion of a premature stop codon in edited spo11-1 alleles. FIG. 28C shows a truncated, inactivated protein resulting from a premature stop codon in one of the edited rec8 alleles, and the deletion of three conserved amino acids resulting from the other edited rec8 allele. FIG. 28D shows an alignment of edited “scar” nucleotide sequences of edited osd1 alleles targeted at a first target site (bottom portion continues from the top portion). FIG. 28E shows an alignment of edited “scar” nucleotide sequences of edited osd1 alleles targeted at a second target site (bottom portion continues from the top portion). FIG. 28F shows an alignment of edited “scar” nucleotide sequences of edited spo11-1 alleles (bottom portion continues from the top portion). FIG. 28G shows an alignment of edited “scar” nucleotide sequences of edited rec8 alleles. FIG. 28H shows an alignment of edited “scar” nucleotide sequences of edited pair1 alleles (bottom portion continues from the top portion).

FIGS. 29A-29D show heatmaps comparing genotype distributions and measures of uniformity in MiMe and control populations of A. thaliana. FIG. 29A illustrates a summary of the genotyping results at 58 triallelic markers across 99 progeny comprising the Boosted Arabidopsis Population 1 (BAP1). The horizontal-axis corresponds to individual tetraploid progeny as well as the two diploid parent plants for reference. The vertical-axis corresponds to individual triallelic markers distributed across 4 separate chromosomes. When referring to the axis, vertical and horizontal are in respect to the legend text for their respective figure. The coloration of each cell denotes the specific configuration of haplotypes observed at that marker (where A=Shandara, B=Col-0, and C=HR-10). Samples with markers that are “AABC” have three haplotypes. Marker design was limited to regions of the genome where heterozygous parental SNPs were present. No usable markers were found on chromosome 3. The parent genotypes are displayed in the two left-most columns. FIG. 29B illustrates a summary of the genotyping results at 57 triallelic markers across 9 progeny of the Standard Arabidopsis Population (SAP, top). The vertical-axis corresponds to individual tetraploid progeny as well as the two tetraploid parent plants for reference. The horizontal-axis corresponds to individual triallelic markers distributed across 5 separate chromosomes. The coloration of each cell denotes the specific configuration of haplotypes observed at that marker (where A=Shandara, B=Col-0, and C=HR-10). 9 representative individuals from BAP1 (FIG. 29A) are shown for comparison (bottom). FIG. 29C shows a matrix illustrating pairwise identity as estimated by the Jaccard similarity coefficient of 57 molecular markers genotyped between 9 tetraploid Arabidopsis plants in the Standard Arabidopsis Population (SAP). FIG. 29D shows a matrix for pairwise identity as estimated by the Jaccard similarity coefficient of 58 molecular markers genotyped between 99 tetraploid Arabidopsis plants in Boosted Arabidopsis Population 1 (BAP1).

FIGS. 30A-30B show BAP1 plants. FIG. 30A shows a top-down view of BAP1 plants at 48 days after planting. FIG. 30B shows a subset of FIG. 30A's BAP1 plants, at a closer proximity and an oblique view, also at 48 days after planting.

FIGS. 31A-31D show formation of parthenocarpic (seedless) fruits across MiMe Arabidopsis plants and controls. FIG. 31A shows seedless fruit from BAP1 plants whose pistils were treated, untreated, or mock-treated with gibberellic acid (“GA3”) to induce fruit development, compared to fruit resulting from pistils treated or mock-treated from fertile Arabidopsis control plants. FIG. 31B shows comparison of the average number of seeds per silique (vertical axis) between BAP1 plants, the doubled version of the parent MiMe plant PED-AR-BC (doubled as “PED-AR-BCBC”) plants, and the doubled version of the parent MiMe plant PED-AR-AA (doubled as “PED-AR-AAAA”) plants, all along the horizontal axis. The population means and standard deviations are represented in the graph by crosses and error bars, respectively FIG. 31C shows comparison of the silique lengths (vertical axis) between the same plant groups in FIG. 31B (horizontal axis). The length of fruit (siliques) resulting from GA3-treated pistils, mock-treated pistils (“mock”), and untreated pistils was measured across the respective plant groups at 8 days after GA3 application. The population means and standard deviations are represented in the graph by crosses and error bars, respectively. FIG. 31D illustrates a comparison of the average number of seeds per silique (vertical axis) between BAP2 plants, the doubled version of the parent MiMe plant PED-AR-BC (doubled as “PED-AR-BCBC”), and the doubled version of the parent MiMe plant PED-AR-DE (doubled as “PED-AR-DEDE”), all along the horizontal axis. The population means and standard deviations are represented in the graph by crosses and error bars, respectively.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Overview

In one aspect, described herein is a population of polyploid maize seed comprising three or more haplotypes of the same or related species of maize, wherein at least 50% of the population of polyploid maize seed is genetically uniform, and wherein the population was obtained from a single maize plant or a set of maize plants such as, for example, a set of genetically uniform F1 hybrids. In some embodiments, the population of polyploid maize seed comprises a subpopulation of genetically uniform polyploid maize seed in an amount of at least 50% of the total number of seed, the genetically uniform polyploid maize seed comprising three or more haplotypes of the same or related species of maize. The genetic uniformity of the seeds of the population addresses a long-felt need for genetically uniform populations of polyploid maize seed comprising three or more haplotypes with improved heterotic performance over the pairs of haplotypes present in existing hybrid maize plants. In some embodiments, the population of polyploid maize seed and/or the subpopulation of genetically uniform polyploid maize seed comprises one or more genetic modifications resulting in decreased expression of one or more MiMe loci. The population of polyploid maize seed and/or the subpopulation of genetically uniform polyploid maize seed may have a complete or partial MiMe genotype comprising MiMe alleles conferring decreased expression of MiMe loci of one or more MiMe components.

In another aspect, provided herein are methods of producing a population of polyploid maize seed comprising three or more haplotypes wherein at least 50% of the population of polyploid maize seed are genetically uniform. In some embodiments, the population of polyploid maize seed comprises a subpopulation of genetically uniform polyploid maize seed in an amount of at least 50% of the total number of seed, the genetically uniform polyploid maize seed comprising three or more haplotypes of the same or related species of maize. In some embodiments, the method comprises (a) providing clonal gametes from a pair of parent MiMe maize plants that together comprise three or more haplotypes; and (b) crossing the clonal gametes to produce the population of polyploid maize seed. In other embodiments, the method comprises (a) providing clonal gametes from a parent MiMe maize plant; (b) providing haploid (e.g., monoploid, dihaploid, or higher ploidy) gametes from a homozygous parent non-MiMe maize plant; and (c) crossing the clonal gametes with the haploid (e.g., monoploid) gametes to produce the population of polyploid maize seed.

In yet another aspect, provided herein are methods of breeding a polyploid hybrid maize line comprising three or more haplotypes, the methods comprising: obtaining a set of maize lines; breeding the maize lines using traditional plant breeding methods to produce a set of candidate maize lines; and selecting two or more candidate maize lines, together comprising three or more haplotypes, for crossing. In some embodiments, after the selection of candidate maize lines, the methods further comprise generating two parent MiMe maize plants from the two or more candidate maize lines; providing clonal gametes from each of the parent MiMe maize plants; and crossing the clonal gametes to produce a hybrid polyploid maize seed comprising the three or more haplotypes. In alternative embodiments, after the selection of candidate maize lines, the methods further comprise generating a single parent MiMe maize plant from one of the two or more candidate maize lines; providing clonal gametes from the parent MiMe maize plant; providing haploid (e.g., monoploid) gametes from a homozygous parent non-MiMe maize plant of one of the two or more candidate maize lines; and crossing the clonal gametes with the haploid (e.g., monoploid) gametes to produce a hybrid polyploid maize seed. In some embodiments, after the crossing of the clonal gametes or the crossing of the clonal gametes with the haploid (e.g., monoploid) gametes, the methods further comprise growing the hybrid polyploid maize seed to produce a hybrid polyploid maize plant and evaluating one or more characteristics of the hybrid polyploid maize plant.

In another aspect, described herein is a population of polyploid maize seed comprising a partially-complemented MiMe genotype, wherein at least 50% of the population of polyploid maize seed is genetically uniform, and wherein the population was obtained from a single maize plant or a set of maize plants such as, for example, a set of genetically uniform F1 hybrids. In some embodiments, the population of polyploid maize seed comprises a subpopulation of genetically uniform polyploid maize seed in an amount of at least 50% of the total number of seed, the genetically uniform polyploid maize seed comprising the partially-complemented MiMe genotype. The polyploid maize seed comprising the partially-complemented MiMe genotype may comprise one, two, three, or more haplotypes. The partially-complemented MiMe genotype of the population of polyploid maize seed results in a maize plant having neither a wild-type meiosis phenotype nor a MiMe phenotype. Thus, in some embodiments, germination of a seed of the population of polyploid maize seed or the subpopulation of genetically uniform polyploid maize seed results in a maize plant that produces inviable gametes. The genetic uniformity of the seeds of the population addresses a long-felt need for genetically uniform populations of polyploid maize seed of plants. In some embodiments, the partially-complemented MiMe genotype comprises (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component. In certain embodiments, the partially-complemented MiMe genotype further comprises (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component.

In another aspect, provided herein are methods of producing a population of polyploid maize seed comprising a partially-complemented MiMe genotype wherein at least 50% of the population of polyploid maize seed are genetically uniform. In some embodiments, the population of polyploid maize seed comprises a subpopulation of genetically uniform polyploid maize seed in an amount of at least 50% of the total number of seed, the genetically uniform polyploid maize seed comprising the partially-complemented MiMe genotype. The polyploid maize seed comprising the partially-complemented MiMe genotype may comprise one, two, three, or more haplotypes. In some embodiments, the method comprises: (a) providing clonal gametes from a first parent MiMe maize plant; (b) providing clonal gametes from a second parent MiMe maize plant; and (c) crossing the clonal gametes to produce the population of polyploid maize seed comprising a partially-complemented MiMe genotype. In some embodiments, the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; and the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe maize plant is the same as at least one of the MiMe loci having only MiMe alleles of the first MiMe component of the second parent MiMe maize plant. In some embodiments, the first and second parent MiMe maize plants further have only MiMe alleles at one or more MiMe loci of a third MiMe component, wherein the one or more MiMe loci having only MiMe alleles of the third MiMe component of the first and second parent MiMe maize plants are the same or different.

In yet another aspect, provided herein are methods of breeding a polyploid maize plant, the methods comprising: obtaining a set of maize lines; breeding the maize lines using traditional plant breeding methods to produce a set of candidate maize lines; and selecting two or more candidate maize lines for crossing. In some embodiments, after the selection of candidate maize lines, the methods further comprise generating two parent MiMe maize plants from the two or more candidate maize lines. In some embodiments, the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; and the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe maize plant is the same as at least one of the MiMe loci having only MiMe alleles of the first MiMe component of the second parent MiMe maize plant. In some embodiments, the first and second parent MiMe maize plants further have only MiMe alleles at one or more MiMe loci of a third MiMe component, wherein the one or more MiMe loci having only MiMe alleles of the third MiMe component of the first and second parent MiMe maize plants are the same or different. In some embodiments, the method further comprises providing clonal gametes from each of the parent MiMe maize plants, crossing the clonal gametes to produce a polyploid maize seed, growing the polyploid maize seed to produce a polyploid maize plant, and evaluating one or more characteristics of the polyploid maize plant.

In some variations of the methods of breeding, the methods may further comprise repeating the steps of the method, using the one or more characteristics of the hybrid polyploid maize plant evaluated to guide the breeding of maize lines, the selecting of candidate maize lines, or both. In additional variations, the methods may further comprise organizing the set of maize lines into three or more heterotic groups, wherein each heterotic group comprises a haplotype, and wherein the haplotypes are grouped based on observed or predicted heterotic performance when combined in the hybrid polyploid maize plant. This method allows for deterministic combination of three or more haplotypes in a polyploid maize plant, addressing the need for maize plant breeding methods that yield predictable results on time scales shorter than those required for traditional breeding methods.

### Definitions

As used herein, the terms “maize” and “maize plant” include the whole maize plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, clonally propagated plants, roots, stems, cotyledons, hypocotyls, root tips and the like. The plant parts or derivatives thereof can also include any of the aforementioned plant parts in an encapsulated form such as, for example, shoot meristems, nodes, stolon tips, and the like, encapsulated in alginate, e.g., in a synthetic seed. Any developmental stage is also included, such as seeds, seedlings, immature and mature, etc. As used herein, the term “non-regenerable” generally refers to a maize plant part, a plant cell, a processed maize product, or a portion of any of the foregoing, that cannot be induced to form a whole maize plant or that cannot be induced to form a whole maize plant that is capable of sexual and/or asexual reproduction.

As used herein, the term “seed” typically refers to a true seed rather than another plant part used for propagation.

As used herein, the term “non-regenerable” generally refers to a plant part, a plant cell, a processed plant product, or a portion of any of the foregoing, that cannot be induced to form a whole plant or that cannot be induced to form a whole plant that is capable of sexual and/or asexual reproduction.

As used herein, “ploidy” refers to the number of complete sets of chromosomes in a cell or organism. Ploidy may be annotated using “n” as the unit of complete sets of chromosomes. For example, a cell or organism with a single set of chromosomes may be referred to as “1n”, or the single set of chromosomes itself may be referred to as “1n”. A diploid cell or organism with two sets of chromosomes may be referred to as “2n”; a triploid cell or organism with three sets of chromosomes may be referred to as “3n”; and so on.

As used herein, “monoploid” refers to a cell or organism with a ploidy of 1n.

As used herein, “diploid” refers to a cell or organism with a ploidy of 2n.

As used herein, “polyploid” refers to a cell or organism with a ploidy of greater than 2n. “Polyploid” may refer to organisms which are triploid (3n), tetraploid (4n), pentaploid (5n), hexaploid (6n), heptaploid (7n), octoploid (8n), or higher ploidies (greater than 8n).

As used herein, “allele” refers to one of two or more alternative forms of a single gene or locus within the genome. As used herein, “monoallelic” typically describes the presence of a single allele at a given locus or set of loci within a cell or organism. As used herein, “biallelic” typically describes the presence of two different alleles at a given locus or set of loci within a cell or organism. As used herein, “multiallelic” typically describes the presence of three or more alleles at a given locus or set of loci within a cell or organism.

As used herein, “haplotype” refers to a distinct In set of chromosomes with a unique set of alleles. As used herein, each haplotype is distinct from other haplotypes in that it contains a set of alleles that confers a unique set of characteristics not conferred by other haplotypes. As used herein, as a feature of the present disclosure, each distinct haplotype need not be inherited from a different parent—a polyploid organism of the present disclosure may comprise three or more haplotypes inherited from two parents. As used herein, “monoallelic plant” typically refers to a plant line containing a single haplotype, “biallelic plant” typically refers to a plant line containing two haplotypes, and “multiallelic plant” typically refers to a plant line containing three or more haplotypes. In the case of allopolyploid maize plants that contain multiple subgenomes between which there is little to no recombination, as used herein, the term “three or more haplotypes” typically refers to three or more haplotypes of the same subgenome.

As used herein, “clonal” describes a body of DNA that is substantially identical to another body of DNA; or a set of cells or organisms that comprise such DNA. For example, mitosis results in two clonal genomes comprised by two clonal cells. Due to random errors in natural DNA replication, clonal bodies of DNA, clonal cells, or clonal organisms may not be completely identical. “Clonal” may describe two genomes that are not completely identical in sequence but that contain the same set of alleles.

As used herein, “genetically uniform” typically describes a set of individual plants, plant parts (e.g., seeds), or plant cells whose genomes are identical across at least 80% of loci, or are clonal. Genetic uniformity of a set of individual plants, plant parts (e.g., seeds), or plant cells may be measured using methods known in the art and described herein. For example, a set of genetic markers may be identified and used to determine the estimated pairwise identity of a pair of individuals, or to determine the average pairwise genetic uniformity of a population of individuals, using the Jaccard similarity coefficient. For example, a population of genetically uniform plants or seeds may consist of plants or seeds having genomes that are identical to one another across at least 80%, at least 85%, at least 90%, at least 95%, at least 96%, at least 97%, at least 98%, at least 99%, or 100% of genetic markers analyzed, or may consist of seeds having an average pairwise genetic uniformity of at least 80%, at least 85%, at least 90%, at least 95%, at least 96%, at least 97%, at least 98%, at least 99%, or 100% as measured by the Jaccard similarity coefficient. Additionally, each pair of seeds in a population of genetically uniform plants or seed may have genomes that have a pairwise identity of at least 95%, at least 96%, at least 97%, at least 98%, at least 99%, or 100% as measured by the Jaccard similarity coefficient.

As used herein, “expression” and “expression level” refer to the relative or absolute amount of a functional gene product present in a cell. As used herein, “gene products” include, but are not limited to, nucleic acids (e.g., RNA), post-transcriptionally modified nucleic acids (e.g., spliced RNA, poly-adenylated mRNA), proteins (e.g., enzymes, structural proteins, etc.), and post-translationally modified proteins (e.g., glycoproteins, lipoproteins, etc.). The function of the gene product refers to the wild-type, unmodified, uninhibited function of the gene product. As used herein, “decreased expression” refers to a relative decrease in the amount of a functional gene product of a gene or genetic locus, such as a MiMe locus, present in a cell. The decreased expression may refer to a decrease in the total amount of a gene product present in a cell (e.g., a decrease in the amount of a protein) or to a decrease in the amount of functional gene products present in a cell (e.g., a decrease in the percentage of proteins with wild-type function, e.g., an altered activity of the protein) or to a decrease in the function of gene products present in a cell (e.g., a decrease in the activity of proteins as compared to proteins with wild-type function, e.g., elimination of activity). The decreased expression may be of a gene product encoded at a certain genomic locus. Decreased expression also includes “non-expression” and “eliminated expression.” As used herein, “non-expression” or “eliminated expression” refers to the absence of a functional gene product present in a cell, or to an expression level insufficient for detection of the gene product in the cell, or to an expression level insufficient to result in the function of the gene product within the cell, or to an activity level insufficient to result in the detectable activity of the gene product within the cell.

As used herein, “homozygous” describes a cell or organism in which all sets of chromosomes encode the same allele or set of alleles at a certain chromosomal locus, a set of chromosomal loci, or at all chromosomal loci. For example, a triploid cell or organism with the same allele at a specific locus in all three sets of chromosomes is homozygous for that allele. An organism may be homozygous for a specific allele or set of alleles at a certain chromosomal locus or set of chromosomal loci, or an organism may be homozygous for a haplotype. For example, a triploid cell or organism containing three copies of the same haplotype is homozygous for that haplotype. As used herein, “homozygous plant” typically refers to an inbred plant or plant line, a monoallelic plant or plant line, or a plant or plant line which is homozygous at all loci within its genome.

As used herein, “heterozygous” describes a cell or organism in which at least one set of chromosomes encodes an allele or set of alleles at a certain chromosomal locus or set of chromosomal loci that is distinct from those of the other sets of chromosomes within the cell or organism. For example, a triploid cell or organism having allele a₁ at locus A in two sets of chromosomes and having allele a₂ at locus A in the third set of chromosomes is heterozygous for alleles a₁ and a₂. An organism may be heterozygous for a specific set of alleles at a certain chromosomal locus or set of chromosomal loci, or an organism may be heterozygous for a haplotype. For example, a triploid cell or organism containing two copies of one first haplotype and one copy of a second haplotype is heterozygous for the first and second haplotype. As used herein, “heterozygous plant” typically refers to a biallelic or multiallelic plant or plant line containing two or more haplotypes.

As used herein, “crossing” refers to the act of forming a zygote from gametes of two distinct plants or plant lines. Crossing may refer to pollinating a plant or plant line using the pollen of a different plant or plant line.

As used herein, “related species of maize” refers to two or more species of maize (e.g., two or more species of the genus Zea) that, when crossed, result in viable seed.

As used herein, “hybrid” describes a plant comprising two or more haplotypes from the same or related species of plant.

As used herein, “F1 hybrid” refers to the first filial generation of hybrid seeds or plants resulting from the cross of parents comprising two or more haplotypes. For clarity, this refers to the first filial generation of the cross and not the first filial generation of the hybrids of a cross.

As used herein, “heterotic performance” refers to the performance of a set of two or more haplotypes in conferring certain desirable characteristics when combined in a hybrid plant. The desired characteristics of heterotic performance may include characteristics of plant vigor including, but not limited to, plant size, hardiness, grain yield, and the like.

As used herein, “MiMe” typically refers to a phenotype of a plant wherein the wild-type meiosis phenotype of the plant is disrupted in such a way that results in the formation of clonal female gametes and/or clonal male gametes. “MiMe” may refer to any one of several known methods to promote the formation of clonal female gametes and/or clonal male gametes in plants including, but not limited to, Mitosis instead of Meiosis as disclosed in d'Erfurth et al. (2009. Turning meiosis into mitosis. PLoS Biol 7, e1000124) and first division restitution without crossing over (FDR-NCO) as disclosed in Peloquin et al. (1999. Meiotic mutants in potato: valuable variants. Genetics 153: 1493-1499), a prime example of FDR-NCO being spo11-1, ps1 mutants as disclosed in Brownfield and Kohler (2010. Unreduced gamete formation in plants: mechanisms and prospects. J Exp Bot 62:5, 1659-1668).

In organisms with a wild-type meiosis phenotype, meiosis in germline cells results in haploid gametes. As used herein, “haploid” typically refers to a cell or organism with a ploidy half that of the parent organism. As used herein, “haploid gametes” typically refers to gamete cells with a ploidy half that of the parent organism. For example, in a diploid (2n) organism with a wild-type meiosis phenotype, meiosis in germline cells results in In haploid gametes. In another example, in a tetraploid (4n) organism with a wild-type meiosis phenotype, meiosis in germline cells results in 2n haploid gametes. As used herein, “parent non-MiMe plant” typically refers to a plant with the wild-type meiosis phenotype wherein meiosis in germline cells results in haploid gametes (pollen and egg cells). As used herein, “homozygous parent non-MiMe plant” typically refers to an inbred parent non-MiMe parent, a monoallelic parent non-MiMe plant, or a parent non-MiMe plant which is homozygous at all loci within its genome. A homozygous parent non-MiMe plant may be produced through inbreeding, production of a doubled haploid line (e.g., a doubled monoploid line), or any other method known in the art for creating plant lines with high degrees of homozygosity.

In plants with the MiMe phenotype, meiosis is replaced by a mitosis-like process in male and/or female germline cells, resulting in clonal gametes. As used herein, “clonal gametes” typically refers to gametes which comprise unreduced, unrecombined copies of the parent plant's genome and, therefore, have the same ploidy as, and are typically genetically identical to, the parent plant. Clonal gametes are produced when germline cells in the parent plant do not undergo recombination as they would in a normal meiotic process, and also undergo a first division restitution or a second division restitution, resulting in unreduced gametes. As a result, clonal gametes are typically both unreduced and unrecombined and therefore typically genetically identical to the parent plant. For example, in a diploid (2n) plant with a MiMe phenotype, germline cells undergo mitosis instead of meiosis, typically resulting in 2n unrecombined gametes, i.e., clonal gametes. In another example, in a tetraploid (4n) plant with a MiMe phenotype, germline cells undergo mitosis instead of meiosis, typically resulting in 4n unrecombined gametes, i.e., clonal gametes. Clonal gametes may refer to female clonal gametes, male clonal gametes, or a combination thereof.

As used herein, “unreduced, non-clonal gametes” typically refers to gametes which comprise unreduced, yet recombined, copies of the parent plant's genome and, therefore, have the same ploidy as the parent plant, but are not genetically identical to the parent plant. Unreduced, non-clonal gametes are produced when germline cells in the parent plant undergo recombination as they would in a normal meiotic process, but undergo a first division restitution or a second division restitution, resulting in unreduced gametes. Therefore, even though unreduced, non-clonal gametes are unreduced, they are the result of a normal recombination process, and are therefore not genetically identical to the parent plant. For example, germline cells in a diploid (2n) plant that undergo a normal recombination process but undergo a first division restitution or a second division restitution result in 2n, recombined gametes, i.e., unreduced, non-clonal gametes. In another example, germline cells in a tetraploid (4n) plant that undergo a normal recombination process but undergo a first division restitution or a second division restitution result in 4n, recombined gametes, i.e., unreduced, non-clonal gametes. Unreduced, non-clonal gametes may refer to female clonal gametes, male clonal gametes, or a combination thereof.

As used herein, “MiMe component” typically refers to a gene function that contributes to a MiMe phenotype, including, but not limited to, genes and gene products involved in meiosis that may be modified or altered to disrupt a wild-type meiotic phenotype in a manner relevant to the formation of clonal female gametes and/or clonal male gametes via MiMe. MiMe components include (1) a component of sister chromatid cohesion during the first division of meiosis, (2) a component of DNA double strand breakage during meiotic recombination, (3) a component of progression through the second division of meiosis, and (4) a component of progression through the first division of meiosis. In general, each MiMe component includes one or more MiMe loci discussed further below. Further, generally, each MiMe locus may have MiMe alleles and non-MiMe alleles.

As used herein, “MiMe allele” typically refers to an allele at a MiMe locus that disrupts the normal meiotic function of a MiMe component (e.g., an allele that disrupts sister chromatid cohesion during the first division of meiosis). MiMe alleles may be naturally occurring MiMe alleles, or may be introduced into a plant line via genetic modification using the methods described herein.

As used herein, “non-MiMe allele” typically refers to any allele that contributes to wild-type function of a MiMe component that therefore does not contribute to conferring a MiMe phenotype in a plant. A non-MiMe allele typically refers to an allele at a MiMe locus that contributes to the wild-type meiotic function of a MiMe component (e.g., an allele that provides the wild-type function that contributes to sister chromatid cohesion during the first division of meiosis).

As used herein, “complete MiMe genotype” typically refers to any set of alleles that confers the MiMe phenotype in a plant. The individual alleles that make up the complete MiMe genotypes are referred to as MiMe alleles. The complete MiMe genotype may be naturally present in a plant or may be introduced via, for example, plant breeding, transgenic techniques, gene-editing techniques, or any combination thereof to introduce one or more naturally-occurring alleles, non-naturally-occurring alleles, or a combination thereof. A complete MiMe genotype may comprise any number of MiMe alleles that results in a MiMe phenotype, such as one, two, three, or more MiMe alleles. As used herein, “MiMe locus” and “MiMe loci” typically refers to any chromosomal locus or loci which may be the site of MiMe alleles, including genes and intergenic loci. A MiMe locus or MiMe loci may correspond to a specific MiMe component, for example, if the MiMe locus encodes a MiMe component gene product. A complete MiMe genotype may comprise MiMe alleles at any number of MiMe loci, such as one, two, three, or more MiMe loci. A complete MiMe genotype may comprise different alleles at the same MiMe locus on different sets of chromosomes and need not be homozygous to confer a MiMe phenotype. For example, a diploid plant with a complete MiMe genotype may have two different REC8 alleles each of which reduces or eliminates REC8 expression or activity such that the plant has two MiMe alleles for REC8 and thus exhibits disruption of sister chromatid cohesion during the first division of meiosis. Specific examples of complete MiMe genotypes are described in detail herein.

As used herein, “partial MiMe genotype” typically refers to a set of alleles that comprises both MiMe and non-MiMe alleles at one or more MiMe loci such that a plant with a partial MiMe genotype exhibits a wild-type meiosis phenotype. Two plants having compatible partial MiMe genotypes each exhibit a wild-type meiosis phenotype and may be crossed to produce F1 offspring having a complete MiMe genotype and, thus, a MiMe phenotype. As used herein, “compatible partial MiMe genotypes” typically refers to two or more partial MiMe genotypes that comprise sets of MiMe alleles at the same MiMe loci. For example, a partial MiMe genotype comprising MiMe alleles of REC8, SPO11-1, and OSD1 is compatible with another partial MiMe genotype that comprises the same or different MiMe alleles of REC8, SPO11-1, and OSD1. The MiMe alleles of a partial MiMe genotype may be combined with the MiMe alleles of the same or different partial MiMe genotype in a single cross to create a complete MiMe genotype and confer a MiMe phenotype in the F1 offspring. In general, where MiMe alleles and non-MiMe alleles are referred to together they are alleles of the same MiMe loci. The partial MiMe genotype may be naturally present in a plant or may be introduced via, for example, plant breeding, transgenic techniques, gene-editing techniques, or any combination thereof to introduce one or more naturally-occurring alleles, non-naturally-occurring alleles, or a combination thereof. A partial MiMe genotype may comprise any number of alleles, such as one, two, three, or more alleles. Further, a partial MiMe genotype may comprise MiMe alleles at any number of MiMe loci, such as one, two, three, or more MiMe loci. Specific examples of partial MiMe genotypes are described in detail herein.

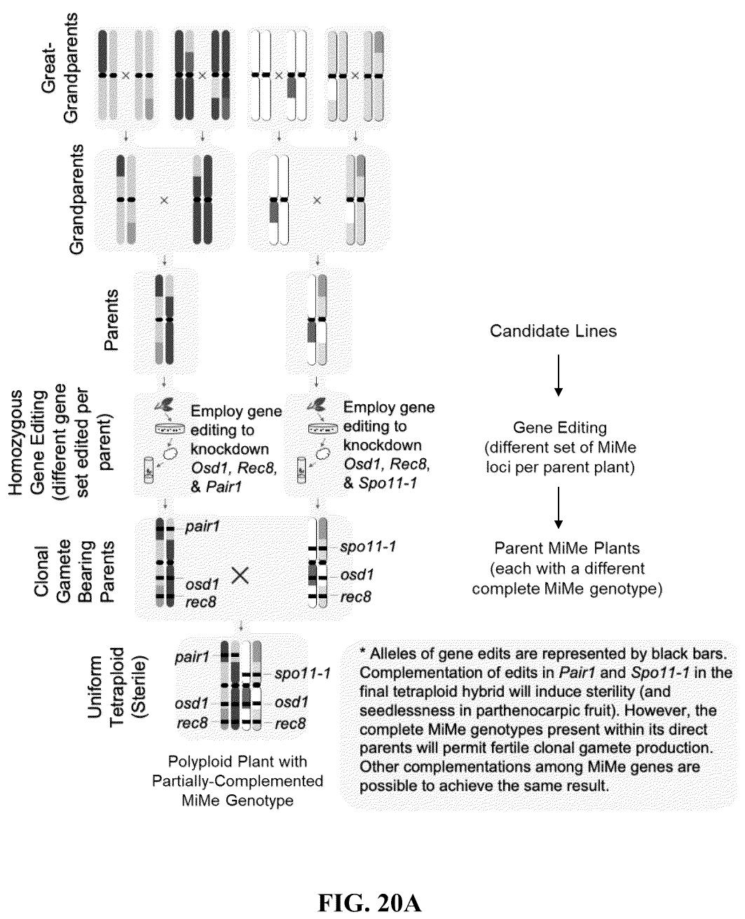

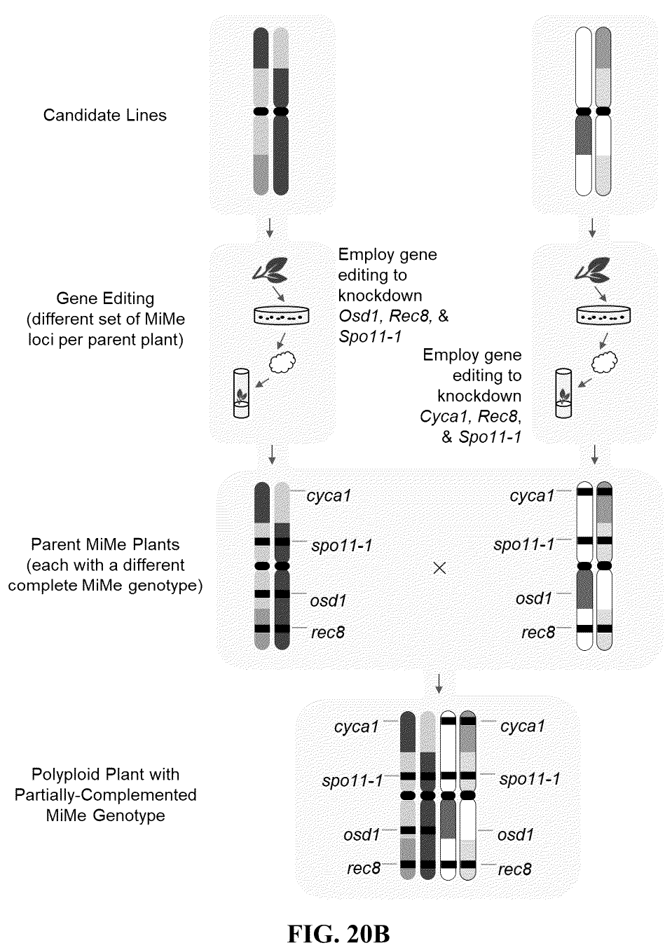

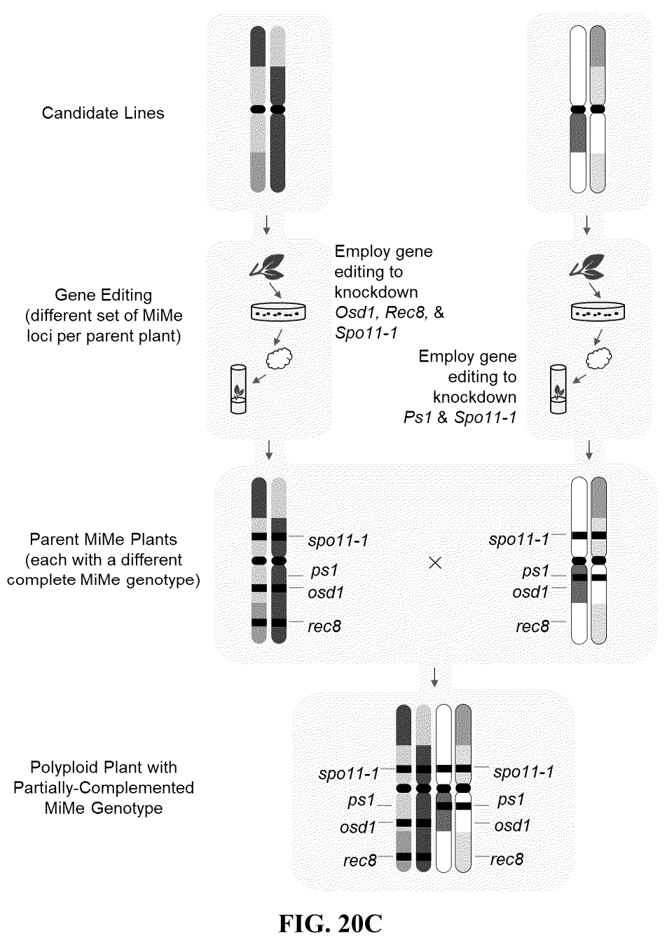

As used herein, “partially-complemented MiMe genotype” typically refers to a set of alleles that comprises only MiMe alleles at each of one or more MiMe loci of a first MiMe component, both MiMe and non-MiMe alleles at a first MiMe locus of a second MiMe component, and both MiMe and non-MiMe alleles at a second MiMe locus of the second MiMe component. A plant with a partially-complemented MiMe genotype typically does not exhibit a wild-type meiosis phenotype because the first MiMe component has only MiMe alleles at each of one or more MiMe loci, disrupting wild-type meiosis. The plant will also not exhibit a MiMe phenotype due to the complementation of the MiMe alleles by the non-MiMe alleles of each of the first and second MiMe loci of the second MiMe component. Therefore, a plant with a partially-complemented MiMe genotype exhibits neither a wild-type meiosis phenotype nor a MiMe phenotype. A partially-complemented MiMe genotype may also include MiMe alleles for a third MiMe component, e.g., only MiMe alleles at each of one or more MiMe loci of a third MiMe component, or both MiMe and non-MiMe alleles at a first MiMe locus of a third MiMe component and both MiMe and non-MiMe alleles at a second MiMe locus of the third MiMe component. As used herein, a plant or genotype “comprising only MiMe alleles at a MiMe locus” is a plant or genotype wherein each set of chromosomes has a MiMe allele at said locus, thus conferring decreased expression (including non-expression or altered activity) of the functional gene product of said locus. Exemplary partially-complemented MiMe genotypes are shown in FIGS. 20A-20C and described in detail herein. The MiMe alleles of each MiMe locus having only MiMe alleles of the first MiMe component may comprise any number of distinct MiMe alleles on different sets of chromosomes and need not be homozygous, as long as there are no non-MiMe alleles at that locus on any set of chromosomes. The partially-complemented MiMe genotype may comprise, for example, one, two, three, or more MiMe alleles and one, two, three, or more non-MiMe alleles at each of the first and second MiMe loci of the second MiMe component, as long as at least one MiMe allele and at least one non-MiMe allele are present at each of the first and second MiMe loci of the second MiMe component. Further, a partially-complemented MiMe genotype may comprise MiMe alleles at more than three MiMe loci, such as four, five, or more MiMe loci. Specific examples of partially-complemented MiMe genotypes are described in detail herein.

As used herein, “parent MiMe plant” typically refers to a plant which has a complete MiMe genotype and exhibits a MiMe phenotype, and which may be a source of clonal gametes (pollen and/or egg cells).

As used herein, “introducing a complete MiMe genotype directly” typically refers to introducing genetic modifications resulting in a complete MiMe genotype into a plant or plant cell including using the methods described herein, selecting a plant or plant cell that has a complete MiMe genotype, if needed, and regenerating the cell that has the complete MiMe genotype into a plant that exhibits a MiMe phenotype, if needed.

As used herein, “grandparent non-MiMe plant having a partial MiMe genotype” typically refers to a plant which has a partial MiMe genotype and exhibits a wild-type meiosis phenotype. A grandparent non-MiMe plant having a partial MiMe genotype produces haploid gametes that may be crossed with haploid gametes from the same or another grandparent non-MiMe plant having a partial MiMe genotype to produce one or more seeds that have a complete MiMe genotype and can be grown to produce one or more parent MiMe plants.

As used herein, “introducing a partial MiMe genotype” refers to introducing genetic modifications resulting in a partial MiMe genotype into a plant or plant cell including using the methods described herein, selecting a plant or plant cell that has a partial MiMe genotype, if needed, and regenerating the cell that has a partial MiMe genotype into a plant that exhibits a wild-type meiosis phenotype. For example, introducing a partial MiMe genotype could include crossing a plant with a MiMe and a non-MiMe allele for a component of sister chromatid cohesion during the first division of meiosis and a MiMe and a non-MiMe allele for a component of DNA double strand breakage during meiotic recombination with a plant that has a MiMe and a non-MiMe allele for a component of progression through the second division of meiosis, and then selecting for offspring that are heterozygous for all three of the parental MiMe alleles and therefore have a partial MiMe genotype.

As used herein, “genetic modification” typically refers to any sequence or portion thereof within a nucleic acid molecule that differs from the sequence of an ancestral nucleic acid molecule. For example, a seed that contains an inserted or deleted genomic sequence that is not present in one of its parent plants comprises a genetic modification. A genetic modification may be naturally occurring or introduced. A genetic modification may be introduced via, for example: plant breeding to introduce a naturally-occurring genetic modification of one plant line into another plant line; transgenic methods; gene editing; chemical mutagenesis; and the like.

As used herein, “transgenesis” refers to the insertion of an exogenous genetic element into the genome of an organism. Any exogenous genetic element may be inserted via transgenesis, including, but not limited to, genes, protein coding sequences, non-protein coding sequences, regulatory sequences, spacer DNA, and the like.

As used herein, “gene editing” refers to a type of genetic modification in which DNA is inserted, deleted or substituted in the genome of an organism using one or more natural or engineered nucleases. Gene editing may be carried out using site-specific nucleases, guided nucleases, or a combination thereof. The nuclease creates one or more site-specific breaks, such as double-strand breaks (DSBs) at target loci in the genome. Each site-specific break may be repaired, for example via non-homologous end joining (NHEJ), resulting in a genetic modification in the genome at the target locus; or via homologous recombination of the target locus with a provided repair nucleic acid molecule comprising homology to the target genomic sequence and the desired genetic modification.

Populations of Polyploid Seed

In one aspect, described herein is a population of polyploid maize seed comprising three or more haplotypes of the same or related species of maize wherein at least 50% of the population of polyploid seed are genetically uniform, and wherein the population was obtained from a single maize plant or a set of maize plants (e.g., a set of genetically uniform maize plants, e.g., a set of genetically uniform F1 maize hybrids). In some embodiments, the population of polyploid maize seed comprises a subpopulation of genetically uniform polyploid maize seed in an amount of at least 50% of the total number of maize seed, the genetically uniform polyploid maize seed comprising three or more haplotypes of the same or related species of maize. In some embodiments, the population of polyploid seed may have a complete MiMe genotype or a partial MiMe genotype.

In another aspect, described herein is a population of polyploid maize seed comprising a partially-complemented MiMe genotype, wherein at least 50% of the population of polyploid maize seed are genetically uniform, and wherein the population was obtained from a single maize plant or a set of maize plants (e.g., a set of genetically uniform maize plants, e.g., a set of genetically uniform F1 maize hybrids). In some embodiments, the population of polyploid maize seed comprises a subpopulation of genetically uniform polyploid maize seed in an amount of at least 50% of the total number of seed, the genetically uniform polyploid maize seed comprising the partially-complemented MiMe genotype. In some embodiments, the population of polyploid maize seed comprising the partially-complemented MiMe genotype may comprise one, two, three, or more haplotypes.

Haplotypes

In some embodiments, the population of polyploid maize seed or the subpopulation of genetically uniform polyploid maize seed comprises one, two, three, or more haplotypes. In some variations, the population of polyploid maize seed or the subpopulation of genetically uniform polyploid maize seed comprises two or more haplotypes, three or more haplotypes, four or more haplotypes, five or more haplotypes, six or more haplotypes, seven or more haplotypes, or eight or more haplotypes. In additional variations, the population of polyploid maize seed or the subpopulation of genetically uniform polyploid maize seed comprises two, three, four, five, six, seven, or eight haplotypes. In some embodiments wherein the polyploid maize seed is from an allopolyploid maize plant having multiple subgenomes, the two, three, or more haplotypes are two, three, or more haplotypes of the same subgenome.

Maize Plants

In some embodiments, the population of polyploid maize seed can be any species of maize plant including, but not limited to, Zea spp. including Z. diploperennis, Z. luxurians, Z. nicaraguensis, and Z perennis, Zea mays including Z. mays spp. mays (modern maize), Z. mays spp. Parviglumis, and Z mays spp. mexicana.

In some embodiments, the population of polyploid seed comprises two, three, or more haplotypes of the same or related species of maizeplant. In another embodiment, the population of polyploid maize seed comprises two, three, or more haplotypes of related species of maize plant. In some embodiments, related species of maize plant are species of plant within the Poaceae family. In other embodiments, related species of maize plant are species of plant within the genus Zea.

In some embodiments, the population of polyploid maize seed is a population of seed of the family Poaceae comprising two, three, or more haplotypes from one or more species within the family Poaceae. In some embodiments, the population of polyploid maize seed comprises two, three, or more haplotypes of maize. In some variations, the two, three, or more haplotypes may be from different subspecies of maize. In some embodiments, the population of polyploid seed is a population of seed of the genus Zea comprising two, three, or more haplotypes from one or more species within the genus Zea. The population of polyploid seed of the genus Zea may be a population of seed of any plant within the genus Zea including, but not limited to, maize and teosinte. In some variations, the two, three, or more haplotypes may be from any species or subspecies in the genus Zea including, but not limited to, Zea mays, Zea diploperennis, Zea nicaraguensis, Zea perennis, and Zea spp. In certain embodiments, the population of polyploid seed is a population of maize seed comprising two, three, or more haplotypes from the same or related species of maize or teosinte including, but not limited to, the species of Zea described herein. Genetic Uniformity, Ploidy, and Origin

In some embodiments, at least 50% of the population of polyploid maize seed comprising two, three, or more haplotypes are genetically uniform. In some variations, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid maize seed comprising two, three, or more haplotypes are genetically uniform. In some embodiments, at least 50% of the population of polyploid maize seed produced are genetically uniform, wherein the polyploid maize seed comprises three or more haplotypes. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid maize seed produced are genetically uniform, wherein the polyploid maize seed comprises three or more haplotypes. In some embodiments, the population of polyploid maize maize seed has an average pairwise genetic uniformity of at least 80% as measured by the Jaccard similarity coefficient. In some variations, the population of polyploid maize seed has an average pairwise genetic uniformity of at least 80%, at least 85%, at least 90%, at least 91%, at least 92%, at least 93%, at least 94%, at least 95%, at least 96%, at least 97%, at least 98%, at least 99%, or 100% as measured by the Jaccard similarity coefficient. In one variation, the population of polyploid maize seed has an average pairwise genetic uniformity of at least 85% as measured by the Jaccard similarity coefficient. In another variation, the population of polyploid maize seed has an average pairwise identity of at least 90% as measured by the Jaccard similarity coefficient.

In some embodiments, the population of polyploid maize seed comprises a subpopulation of genetically uniform polyploid maize seed in an amount of at least 50% of the total number of maize seeds. In some variations, the population of polyploid maize seed comprises a subpopulation of genetically uniform polyploid maize seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of maize seeds. In some embodiments, each pair of the subpopulation of genetically uniform polyploid maize maize seed has a pairwise identity of at least about 90% as measured by the Jaccard similarity coefficient. In some embodiments, each pair of the subpopulation of genetically uniform polyploid maize maize seed has a pairwise identity of at least about 90%, at least about 95%, at least about 96%, at least about 97%, at least about 98%, at least about 99%, or about 100% as measured by the Jaccard similarity coefficient.

In some embodiments, the population of polyploid maize seed or the subpopulation of genetically uniform polyploid maize seed comprising two, three, or more haplotypes is triploid, tetraploid, pentaploid, hexaploid, heptaploid, or octoploid. In other embodiments, the population of polyploid maize seed or the subpopulation of genetically uniform polyploid maize seed comprising two, three, or more haplotypes has a ploidy of 9n, 10n, 11n, 12n, 13n, 14n, 15n, 16n, or higher.

In some embodiments, the population of polyploid maize seed was obtained from a single maize plant or a set of maize plants such as, for example, a set of F1 hybrids. In some embodiments, the population of polyploid maize seed was obtained from a single maize plant. In other embodiments, the population of polyploid maize seed was obtained from a set of F1 hybrids. In some variations, the population of polyploid maize seed was obtained from a set of two, three, four, five, 10, 20, 50, 100, or more F1 hybrids. In some additional variations, the population of polyploid maize seed was obtained from a set of genetically uniform maize plants, e.g., a set of F1 hybrids derived from the same inbred parents. In yet additional variations, the population of polyploid maize seed was obtained from a set of two, three, four, five, 10, 20, 50, 100, or more genetically uniform maize plants, e.g., genetically uniform F1 hybrids. In certain embodiments, the genetically uniform set of maize plants (e.g., the genetically uniform set of F1 hybrids) has an average pairwise genetic uniformity of at least 80%, at least 85%, at least 90%, at least 91%, at least 92%, at least 93%, at least 94%, at least 95%, at least 96%, at least 97%, at least 98%, at least 99%, or 100% as measured by the Jaccard similarity coefficient. In certain embodiments, each pair of the genetically uniform set of maize plants (e.g., the genetically uniform set of F1 hybrids) has a pairwise identity of at least about 90%, at least about 95%, at least about 96%, at least about 97%, at least about 98%, at least about 99%, or about 100% as measured by the Jaccard similarity coefficient.

Methods of measuring genetic uniformity are known in the art. One exemplary method of measuring genetic uniformity is by using the Jaccard similarity coefficient, also known as the Jaccard index or Jacard similarity index. In the context of molecular plant genetics, the Jaccard index, Jaccard similarity index, or Jaccard similarity coefficient (Jaccard, P. (1908) Nouvelles Recherches sur la Distribution Florale. Bulletin de la Societe Vaudoise des Sciences Naturelles. Vol. 44), is commonly applied to quantify the pairwise genetic similarity or uniformity of plants based on the presence or absence of shared alleles at loci spread throughout the genome. Exemplary methods of using the Jaccard similarity coefficient to measure genetic uniformity between two plants are described in Example 1 below and, for example, in Paz and Veilleux (1997. Genetic diversity based on randomly amplified polymorphic DNA (RAPD) and its relationship with the performance of diploid potato hybrids. Journal of the American Society for Horticultural Sci. 122(6): 740-747), Vosman et al. (2004. The establishment of ‘essential derivation’ among rose varieties, using AFLP. Theoretical and Applied Genetics. 109: 1718-1725), Noli et al. (2013. Criteria for the definition of similarity thresholds for identifying essentially derived varieties. Plant Breeding. 132(6): 525-531), Vijayakumar et al. (2021. High temperature induced changes in quality and yield parameters of tomato (Solanum lycopersicum L.) and similarity coefficients among genotypes using SSR markers. Heliyon. 7(2)), and Dalamu et al. (2023. Genetic Diversity and Population Structure Analyses Using Simple Sequence Repeat Markers and Phenotypic Traits in Native Potato Collection in India. Potato Research: 1-25). The Jaccard similarity coefficient is defined as the ratio of the number of shared items to the total number of distinct items in the two sets. In the context of molecular plant genetics, it quantifies the proportion of shared alleles between two plants. The formula for calculating the Jaccard similarity coefficient is:

J(A,B)=|A∩B|/|A∪B|

Where A represents the unique set of alleles without duplicates in one plant, B the unique set of alleles without duplicates in the other plant, |A∩B| represents the number of shared alleles (the cardinality of the intersection) between the plants, and |A∪B| represents the number of distinct alleles (the cardinality of the union) between the plants. This formula computes the cardinality of the intersection (common elements) of two sets (the shared alleles) divided by the cardinality of the union (all alleles) of the two sets (all distinct alleles present). The resulting value of the Jaccard similarity coefficient ranges from 0 to 1, where 0 indicates no shared alleles, and 1 indicates complete uniformity. The average pairwise genetic uniformity of the populations was calculated as the average Jaccard similarity of all possible pairs of plants within the population. We note that in the context of genetic pairwise similarity estimations, the size of A should be the same as, or very close to the size of B to avoid misinterpretation.

Genetic Modifications

In some embodiments, the polyploid maize seed (e.g., the subpopulation of genetically uniform polyploid maize seed) comprises one or more genetic modifications. Genetic modifications may be generated by modification of any nucleic acid sequence or genetic element by insertion, deletion, or substitution of one or more nucleotides in a nucleic acid molecule. This can occur by a replacement of at least one nucleotide, a deletion of at least one nucleotide, an insertion of at least one nucleotide, a chemical alteration of at least one nucleotide, or a combination thereof as long as the result is a detectable (e.g., by PCR, DNA sequencing, chromatography, etc.) change of nucleotide sequence compared to the sequence of the nucleic acid molecule prior to modification. Such modifications can be achieved by any of several well-known methods known in the art including, but not limited to, random mutagenesis, genome editing, insertion of a recombinant nucleic acid, crossing of an unmodified maize plant with a modified maize plant to introduce the modification of the modified maize plant into the unmodified maize plant, and the like. A genetic modification may be naturally occurring or non-naturally occurring.

The genetic modifications described herein may be present in any known genetic element including, but not limited to, protein-coding sequences, non-protein-coding sequences, promoter regions, 5′ untranslated leaders, genes, exons, introns, poly-A signal sequences, 3′ untranslated regions, regions encoding small RNAs (such as microRNAs and small-interfering RNAs), and any other sequences that affect transcription or translation of one or more nucleic acid sequences. In some embodiments, genetic modifications may include, but are not limited to, modifying or replacing nucleotide sequences of interest (such as a regulatory elements), gene disruption, gene knockout, gene knockdown, gene knock-in, gene silencing (including, e.g., by expressing an inverted repeat into a gene of interest), RNA interference (including, e.g., by insertion and/or expression of an RNA interference construct), modification of methylation status, modification of splicing sites, introducing alternate splicing sites, or any combination thereof. As used herein, gene disruption refers to the alteration or insertion of a sequence into a gene or locus that results in decreased expression (including non-expression or altered activity) of a functional protein gene product. A gene disruption may be achieved by introduction of a genetic modification in a protein-coding sequence, including, but not limited to, as a mis-sense or non-sense mutation, or an insertion, deletion, or substitution. As used herein, a knockout is a genetic modification wherein a gene or gene product has been rendered completely inoperative. A knockout of a gene product may be achieved by introduction of a genetic modification in a protein-coding sequence of a gene or any non-protein-coding or regulatory sequence described herein. As used herein, a knockdown is a genetic modification wherein a gene or gene product has been rendered partially inoperative. A knockdown of a gene product may be achieved by introduction of a genetic modification in a protein-coding sequence of a gene or in a non-protein-coding or regulatory sequence, or insertion of a trans-acting element, such as a construct that expresses an inverted repeat of the gene product or a construct that expresses a DNA- or RNA-binding protein such as a transcriptional repressor which may include, for example, a deactivated targeted nuclease such as deactivated Cas9 (dCas9). As used herein, knock-in represents the replacement or insertion of a DNA sequence at a specific DNA locus in a cell. Knock-ins may include, but are not limited to, specific insertion of a heterologous amino acid coding sequence in a coding region of a gene, an insertion of a transcriptional regulatory element in a genetic locus, or any of several methods of inserting a DNA sequence into a cell that are known to one of ordinary skill in the art.

In certain embodiments, the polyploid maize seed (e.g., the subpopulation of genetically uniform polyploid maize seed) comprises one or more genetic modifications resulting in decreased expression (including non-expression or altered activity) of a gene product of a genomic locus. In some embodiments, genetic modifications resulting in decreased expression (including non-expression or altered activity) of a gene product or locus may include, but are not limited to, modification of an enhancer, modification of a promoter, modification of a 5′ untranslated leader, modification of a coding region, modification of a non-coding region, insertion and/or expression of an RNA interference construct that targets an mRNA, modification of a region encoding a small RNA, modification of methylation status of a genomic locus, expression of a repressor protein that targets a DNA or mRNA sequence, and any other sequences that affect transcription or translation of one or more nucleic acid sequences. In some embodiments, genetic modifications resulting in decreased expression (including non-expression or altered activity) of a gene product or locus may include, but are not limited to, modifying or replacing nucleotide sequences of interest (such as a regulatory elements), gene disruption, gene knockout, gene knockdown, gene knock-in, gene silencing (including, e.g., by inserting and/or expressing an inverted repeat into a gene of interest), RNA interference (including, e.g., by insertion and/or expression of an RNA interference construct), expression of a repressor protein (e.g. dCas9), modification of methylation status of gene loci, modification of splicing sites, introducing alternate splicing sites, or any combination thereof. In some variations, the genetic modification is positioned in the first 70%, the first 60%, the first 50%, the first 40%, the first 30%, the first 20%, or the first 10% of the nucleotides of the coding sequence of the genomic locus following the start codon in the 3′ direction. In certain variations, the genetic modification is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction.

In some embodiments, one or more genetic modifications each independently comprise an insertion, a deletion, one or more nucleotide changes, or an inversion that results in decreased expression of the one or more genomic loci (e.g., MiMe loci). In some variations, the insertion, the deletion, the one or more nucleotide changes, or the inversion eliminates expression (e.g., eliminates activity) of the genomic locus. In some variations, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the genomic locus following the start codon in the 3′ direction. In certain variations, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some embodiments, the insertion, the deletion, the one or more nucleotide changes, or the inversion eliminates expression (e.g., eliminates activity) of the genomic locus. In some variations, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the genomic locus. In some variations, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction, thereby eliminating expression (e.g., activity) of the genomic locus.

In some embodiments, the one or more genetic modifications comprise one or more polynucleotide sequences selected from the group consisting of SEQ ID NOs: 108-111. In certain embodiments, the one or more genetic modifications comprise one or more polynucleotide sequences each having at least 80%, at least 85%, at least 90%, at least 95%, at least 96%, at least 97%, at least 98%, or at least 99% identity or complementarity to a nucleotide sequence selected from the group consisting of SEQ ID NOs: 108-111.

MiMe Loci, MiMe Genotypes, and MiMe Components

In some embodiments, the polyploid maize seed (e.g., the subpopulation of genetically uniform polyploid maize seed) comprises one or more genetic modifications resulting in decreased expression (including non-expression or altered activity) of one or more MiMe loci. In some variations, the polyploid maize seed (e.g., the subpopulation of genetically uniform polyploid maize seed) comprises one or more genetic modifications resulting in a decreased amount of a functional gene product encoded at one or more MiMe loci. The gene products at the MiMe loci may include, but are not limited to, nucleic acids (e.g. RNA), post-transcriptionally modified nucleic acids (e.g. spliced RNA, poly-adenylated mRNA), proteins (e.g. enzymes, structural proteins, etc.), and post-translationally modified proteins (e.g. glycoproteins, lipoproteins, etc.). The function of the gene product at the MiMe locus refers to the wild-type, unmodified function of the gene product. The decreased expression of a MiMe locus may refer to a decrease in the total amount of a gene product encoded at a MiMe locus present in a cell (e.g. a decrease in the amount of a protein, including up to no detectable expression) or to a decrease in the amount of a functional gene product encoded at a MiMe locus present in a cell (e.g. a decrease in the percentage of proteins with wild-type function, or an increase in the percentage of proteins with altered activity). In some embodiments, the one or more genetic modifications resulting in decreased expression of one or more MiMe loci may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci. In some embodiments, the polyploid maize seed (e.g., the subpopulation of genetically uniform polyploid maize seed) may comprise one or more genetic modifications resulting in non-expression of one or more MiMe loci. In some embodiments, the polyploid maize seed (e.g., the subpopulation of genetically uniform polyploid maize seed) may comprise one or more genetic modifications resulting in decreased expression (including non-expression or altered activity) of a combination of two or more MiMe loci.

In some embodiments, the polyploid maize seed comprises one or more genetic modifications resulting in decreased expression of one or more MiMe loci. In other embodiments, the polyploid maize seed (e.g., the subpopulation of genetically uniform polyploid maize seed) comprises one or more genetic modifications resulting in decreased expression of two or more MiMe loci. In yet another embodiment, the polyploid maize seed (e.g., the subpopulation of genetically uniform polyploid maize seed) comprises one or more genetic modifications resulting in decreased expression of three or more MiMe loci. In some variations, the MiMe loci may include, but are not limited to, REC8, OSD1 (e.g., OSD1-1, OSD1-2, and/or OSD1-3 in maize), CYCA1, TDM1, PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, SWITCH1/DYAD, PS1, PS1-LIKE PROTEIN, JASON (e.g., JASON-1 and/or JASON-2 in maize), PC1, PC2, and FC. In one variation, the polyploid maize seed (e.g., the subpopulation of genetically uniform polyploid maize seed) comprises one or more genetic modifications resulting in decreased expression of REC8. In a second variation, the polyploid maize seed (e.g., the subpopulation of genetically uniform polyploid maize seed) comprises one or more genetic modifications resulting in decreased expression of OSD1 (e.g., OSD1-1, OSD1-2, and/or OSD1-3 in maize). In a third variation, the polyploid maize seed (e.g., the subpopulation of genetically uniform polyploid maize seed) comprises one or more genetic modifications resulting in decreased expression of PAIR1 (e.g., PAIR1-1 and/or PAIR1-2 in maize). In a fourth variation, the polyploid maize seed (e.g., the subpopulation of genetically uniform polyploid maize seed) comprises one or more genetic modifications resulting in decreased expression of SPO11-1, SPO11-2, or a combination thereof. In a fifth variation, the polyploid maize seed (e.g., the subpopulation of genetically uniform polyploid maize seed) comprises one or more genetic modifications resulting in decreased expression of REC8 and SPO11-1. In a sixth variation, the polyploid maize seed (e.g., the subpopulation of genetically uniform polyploid maize seed) comprises one or more genetic modifications resulting in decreased expression of REC8 and OSD1 (e.g., OSD1-1, OSD1-2, and/or OSD1-3 in maize). In a seventh variation, the polyploid maize seed (e.g., the subpopulation of genetically uniform polyploid maize seed) comprises one or more genetic modifications resulting in decreased expression of REC8 and PAIR1 (e.g., PAIR1-1 and/or PAIR1-2 in maize). In an eighth variation, the polyploid maize seed (e.g., the subpopulation of genetically uniform polyploid maize seed) comprises one or more genetic modifications resulting in decreased expression of OSD1 (e.g., OSD1-1, OSD1-2, and/or OSD1-3 in maize) and SPO11-1. In a ninth variation, the polyploid maize seed (e.g., the subpopulation of genetically uniform polyploid maize seed) comprises one or more genetic modifications resulting in decreased expression of OSD1 (e.g., OSD1-1, OSD1-2, and/or OSD1-3 in maize) and PAIR1 (e.g., PAIR1-1 and/or PAIR1-2 in maize). In a tenth variation, the polyploid maize seed (e.g., the subpopulation of genetically uniform polyploid maize seed) comprises one or more genetic modifications resulting in decreased expression of REC8, OSD1 (e.g., OSD1-1, OSD1-2, and/or OSD1-3 in maize), and PAIR1 (e.g., PAIR1-1 and/or PAIR1-2 in maize). In an eleventh variation, the polyploid maize seed (e.g., the subpopulation of genetically uniform polyploid maize seed) comprises one or more genetic modifications resulting in decreased expression of REC8, OSD1 (e.g., OSD1-1, OSD1-2, and/or OSD1-3 in maize), and SPO11-1. In a twelfth variation, the polyploid maize seed (e.g., the subpopulation of genetically uniform polyploid maize seed) comprises one or more genetic modifications resulting in decreased expression of PS1 and SPO11-1. In a thirteenth variation, the polyploid maize seed (e.g., the subpopulation of genetically uniform polyploid maize seed) comprises one or more genetic modifications resulting in decreased expression of PS1 and SY3. In a fourteenth variation, the polyploid maize seed (e.g., the subpopulation of genetically uniform polyploid maize seed) comprises one or more genetic modifications resulting in decreased expression of OSD1 (e.g., OSD1-1, OSD1-2, and/or OSD1-3 in maize), SPO11-1, and REC8. In a fifteenth variation, the polyploid maize seed (e.g., the subpopulation of genetically uniform polyploid maize seed) comprises one or more genetic modifications resulting in decreased expression of REC8, OSD1 (e.g., OSD1-1, OSD1-2, and/or OSD1-3 in maize), SPO11-1, and PAIR1 (e.g., PAIR1-1 and/or PAIR1-2 in maize). In a sixteenth variation, the polyploid maize seed (e.g., the subpopulation of genetically uniform polyploid maize seed) comprises one or more genetic modifications resulting in decreased expression of PS1 or PS1-LIKE PROTEIN, SY3, and SPO11-1. The polyploid maize seed (e.g., the subpopulation of genetically uniform polyploid maize seed) may comprise one or more genetic modifications resulting in decreased expression of any combination of MiMe loci described herein or known in the art. In some embodiments, the polyploid maize seed (e.g., the subpopulation of genetically uniform polyploid maize seed) may comprise one or more genetic modifications resulting in non-expression of any combination of MiMe loci described here or known in the art. In further embodiments, the polyploid maize seed (e.g., the subpopulation of genetically uniform polyploid maize seed) may comprise one or more genetic modifications resulting in decreased expression (including non-expression or altered activity) of a combination of two or more MiMe loci described here or known in the art.

In some embodiments, the polyploid maize seed (e.g., the subpopulation of genetically uniform polyploid maize seed) comprises one or more genetic modifications resulting in decreased expression of REC8, SWITCH1/DYAD, or a combination thereof. In some variations, the polyploid maize seed (e.g., the subpopulation of genetically uniform polyploid maize seed) comprises one or more genetic modifications resulting in decreased expression of REC8, SWITCH1/DYAD, or a combination thereof and one or more genetic modifications resulting in decreased expression of one or more additional MiMe loci. In additional variations, the polyploid maize seed (e.g., the subpopulation of genetically uniform polyploid maize seed) comprises one or more genetic modifications resulting in decreased expression of REC8, SWITCH1/DYAD, or a combination thereof and one or more genetic modifications resulting in decreased expression of one or more additional MiMe loci which may include, but are not limited to, OSD1 (e.g., OSD1-1, OSD1-2, and/or OSD1-3 in maize), CYCA1, TDM1, PC1, PC2, and FC. In yet additional variations, the polyploid maize seed (e.g., the subpopulation of genetically uniform polyploid maize seed) comprises one or more genetic modifications resulting in decreased expression of REC8, SWITCH1/DYAD, or a combination thereof; one or more genetic modifications resulting in decreased expression of OSD1 (e.g., OSD1-1, OSD1-2, and/or OSD1-3 in maize), CYCA1, TDM1, PC1, PC2, FC, or any combination thereof; and one or more genetic modifications resulting in decreased expression of one or more additional MiMe loci, which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the polyploid maize seed (e.g., the subpopulation of genetically uniform polyploid maize seed) comprises one or more genetic modifications resulting in decreased expression of one or more MiMe loci which may include, but are not limited to, OSD1 (e.g., OSD1-1, OSD1-2, and/or OSD1-3 in maize), CYCA1, TDM1, PC1, PC2, and FC. In some variations, the polyploid maize seed (e.g., the subpopulation of genetically uniform polyploid maize seed) comprises one or more genetic modifications resulting in decreased expression of OSD1 (e.g., OSD1-1, OSD1-2, and/or OSD1-3 in maize), CYCA1, TDM1, PC1, PC2, FC, or any combination thereof and further comprises one or more genetic modifications resulting in decreased expression of one or more additional MiMe loci, which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the polyploid maize seed (e.g., the subpopulation of genetically uniform polyploid maize seed) comprises one or more genetic modifications resulting in decreased expression of one or more MiMe loci which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4. In some variations, the polyploid maize seed (e.g., the subpopulation of genetically uniform polyploid maize seed) comprises one or more genetic modifications resulting in decreased expression of PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1, PS1-LIKE PROTEIN, and JASON (e.g., JASON-1 and/or JASON-2 in maize).

In some embodiments, each of the one or more MiMe loci encodes a protein of a MiMe component as described herein. In certain embodiments, each of the one or more MiMe loci encodes a protein having an amino acid sequence selected from the group consisting of SEQ ID NOs: 1-12. In some variations, each of the one or more MiMe loci encodes a protein having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to an amino acid sequence selected from the group consisting of SEQ ID NOs: 1-12.

In some embodiments, the population of polyploid maize seed or the subpopulation of genetically uniform polyploid maize seed has a complete MiMe genotype. In alternative embodiments, the population of polyploid maize seed or the subpopulation of genetically uniform polyploid maize seed has a partial MiMe genotype. In still other embodiments, the population of polyploid maize seed or the subpopulation of genetically uniform polyploid maize seed has a partially-complemented MiMe genotype. In certain embodiments, the complete, partial, or partially-complemented MiMe genotype comprises one or more genetic modifications resulting in decreased expression of one or more MiMe loci. In other embodiments, the complete, partial, or partially-complemented MiMe genotype comprises one or more genetic modifications resulting in decreased expression of two or more MiMe loci. In yet another embodiment, the complete, partial, or partially-complemented MiMe genotype comprises one or more genetic modifications resulting in decreased expression of three or more MiMe loci. In some variations, the MiMe loci may include, but are not limited to, REC8, OSD1 (e.g., OSD1-1, OSD1-2, and/or OSD1-3 in maize), CYCA1, TDM1, PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, SWITCH1/DYAD, PS1, PS1-LIKE PROTEIN, JASON (e.g., JASON-1 and/or JASON-2 in maize), PC1, PC2, and FC. In one variation, the complete, partial, or partially-complemented MiMe genotype comprises one or more genetic modifications resulting in decreased expression of REC8. In a second variation, the complete, partial, or partially-complemented MiMe genotype comprises one or more genetic modifications resulting in decreased expression of OSD1 (e.g., OSD1-1, OSD1-2, and/or OSD1-3 in maize). In a third variation, the complete, partial, or partially-complemented MiMe genotype comprises one or more genetic modifications resulting in decreased expression of PAIR1 (e.g., PAIR1-1 and/or PAIR1-2 in maize). In a fourth variation, the complete, partial, or partially-complemented MiMe genotype comprises one or more genetic modifications resulting in decreased expression of SPO11-1, SPO11-2, or a combination thereof. In a fifth variation, the complete, partial, or partially-complemented MiMe genotype comprises one or more genetic modifications resulting in decreased expression of REC8 and SPO11-1. In a sixth variation, the complete, partial, or partially-complemented MiMe genotype comprises one or more genetic modifications resulting in decreased expression of REC8 and OSD1 (e.g., OSD1-1, OSD1-2, and/or OSD1-3 in maize). In a seventh variation, the complete, partial, or partially-complemented MiMe genotype comprises one or more genetic modifications resulting in decreased expression of REC8 and PAIR1 (e.g., PAIR1-1 and/or PAIR1-2 in maize). In an eighth variation, the complete, partial, or partially-complemented MiMe genotype comprises one or more genetic modifications resulting in decreased expression of OSD1 (e.g., OSD1-1, OSD1-2, and/or OSD1-3 in maize) and SPO11-1. In a ninth variation, the complete, partial, or partially-complemented MiMe genotype comprises one or more genetic modifications resulting in decreased expression of OSD1 (e.g., OSD1-1, OSD1-2, and/or OSD1-3 in maize) and PAIR1 (e.g., PAIR1-1 and/or PAIR1-2 in maize). In a tenth variation, the complete, partial, or partially-complemented MiMe genotype comprises one or more genetic modifications resulting in decreased expression of REC8, OSD1 (e.g., OSD1-1, OSD1-2, and/or OSD1-3 in maize), and PAIR1 (e.g., PAIR1-1 and/or PAIR1-2 in maize). In an eleventh variation, the complete, partial, or partially-complemented MiMe genotype comprises one or more genetic modifications resulting in decreased expression of REC8, OSD1 (e.g., OSD1-1, OSD1-2, and/or OSD1-3 in maize), and SPO11-1. In a twelfth variation, the complete, partial, or partially-complemented MiMe genotype comprises one or more genetic modifications resulting in decreased expression of PS1 and SPO11-1. In a thirteenth variation, the complete, partial, or partially-complemented MiMe genotype comprises one or more genetic modifications resulting in decreased expression of PS1 and SY3. In a fourteenth variation, the complete, partial, or partially-complemented MiMe genotype comprises one or more genetic modifications resulting in decreased expression of OSD1 (e.g., OSD1-1, OSD1-2, and/or OSD1-3), SPO11-1, and REC8. In a fifteenth variation, the complete, partial, or partially-complemented MiMe genotype comprises one or more genetic modifications resulting in decreased expression of REC8, OSD1 (e.g., OSD1-1, OSD1-2, and/or OSD1-3 in maize), SPO11-1, and PAIR1 (e.g., PAIR1-1 and/or PAIR1-2 in maize). In a sixteenth variation, the complete, partial, or partially-complemented MiMe genotype comprises one or more genetic modifications resulting in decreased expression of PS1 or PS1-LIKE PROTEIN, SY3, and SPO11-1. The complete, partial, or partially-complemented MiMe genotype may comprise one or more genetic modifications resulting in decreased expression of any combination of MiMe loci described herein or known in the art, wherein a maize plant that has the complete MiMe genotype exhibits a MiMe phenotype. In some embodiments, the complete, partial, or partially-complemented MiMe genotype may comprise one or more genetic modifications resulting in non-expression of any combination of MiMe loci described here or known in the art wherein a maize plant that has the complete MiMe genotype exhibits a MiMe phenotype. In further embodiments, the complete, partial, or partially-complemented MiMe genotype may comprise one or more genetic modifications resulting in decreased expression (including non-expression or altered activity) of a combination of two or more MiMe loci described here or known in the art, wherein a maize plant that has the complete MiMe genotype exhibits a MiMe phenotype. Specific examples of complete MiMe genotypes are shown in Table 6.

In some embodiments, the complete, partial, or partially-complemented MiMe genotype comprises one or more genetic modifications resulting in decreased expression of REC8, SWITCH1/DYAD, or a combination thereof. In some variations, the complete, partial, or partially-complemented MiMe genotype comprises one or more genetic modifications resulting in decreased expression of REC8, SWITCH1/DYAD, or a combination thereof and one or more genetic modifications resulting in decreased expression of one or more additional MiMe loci. In additional variations, the complete, partial, or partially-complemented MiMe genotype comprises one or more genetic modifications resulting in decreased expression of REC8, SWITCH1/DYAD, or a combination thereof and one or more genetic modifications resulting in decreased expression of one or more additional MiMe loci, including, but not limited to, OSD1 (e.g., OSD1-1, OSD1-2, and/or OSD1-3 in maize), CYCA1, TDM1, PC1, PC2, and FC. In yet additional variations, the complete, partial, or partially-complemented MiMe genotype comprises one or more genetic modifications resulting in decreased expression of REC8, SWITCH1/DYAD, or a combination thereof; one or more genetic modifications resulting in decreased expression of OSD1 (e.g., OSD1-1, OSD1-2, and/or OSD1-3 in maize), CYCA1, TDM1, PC1, PC2, FC, or any combination thereof; and one or more genetic modifications resulting in decreased expression of one or more additional MiMe loci, including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the complete, partial, or partially-complemented MiMe genotype comprises one or more genetic modifications resulting in decreased expression of one or more MiMe loci including, but not limited to, OSD1 (e.g., OSD1-1, OSD1-2, and/or OSD1-3 in maize), CYCA1, TDM1, PC1, PC2, and FC. In some variations, the complete, partial, or partially-complemented MiMe genotype comprises one or more genetic modifications resulting in decreased expression of OSD1 (e.g., OSD1-1, OSD1-2, and/or OSD1-3 in maize), CYCA1, TDM1, PC1, PC2, FC, or any combination thereof and further comprises one or more genetic modifications resulting in decreased expression of one or more additional MiMe loci, including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the complete, partial, or partially-complemented MiMe genotype comprises one or more genetic modifications resulting in decreased expression of one or more MiMe loci including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4. In some variations, the complete, partial, or partially-complemented MiMe genotype comprises one or more genetic modifications resulting in decreased expression of PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1, PS1-LIKE PROTEIN, and JASON (e.g., JASON-1 and/or JASON-2 in maize).

In some embodiments, the population of polyploid maize seed or the subpopulation of genetically uniform polyploid maize seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In other embodiments, the population of polyploid maize seed or the subpopulation of genetically uniform polyploid maize seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe components wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the partial MiMe genotype comprises one or more non-MiMe alleles at the one or more MiMe loci of each of the first, second, and third MiMe components. In other variations, the partial MiMe genotype comprises two or more non-MiMe alleles at the one or more MiMe loci of each of the first, second, and third MiMe components.

In some embodiments, the population of polyploid maize seed or the subpopulation of genetically uniform polyploid maize seed has a complete MiMe genotype comprising MiMe alleles conferring decreased expression of one or more MiMe loci each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the population of polyploid maize seed or the subpopulation of genetically uniform polyploid maize seed having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes.

In other embodiments, the population of polyploid maize seed or the subpopulation of genetically uniform polyploid maize seed has a partial MiMe genotype comprising (a) one or more MiMe alleles conferring decreased expression of one or more MiMe loci of each of a first and second MiMe component; and (b) one or more non-MiMe alleles at the one or more MiMe loci of each of the first and second MiMe components wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the partial MiMe genotype comprises one or more non-MiMe alleles at the one or more MiMe loci of each of the first and second MiMe components. In other variations, the partial MiMe genotype comprises two or more non-MiMe alleles at the one or more MiMe loci of each of the first and second MiMe components.

In yet other embodiments, the population of polyploid maize seed or the subpopulation of genetically uniform polyploid maize seed has (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the third haplotype comprises a non-MiMe allele at the one or more MiMe loci of one or more of the first, second, and third MiMe components.

In still other embodiments, the population of polyploid maize seed or the subpopulation of genetically uniform polyploid maize seed has (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the third haplotype comprises a non-MiMe allele at the one or more MiMe loci of one or more of the first and second MiMe components.

In some embodiments, the population of polyploid maize seed or the subpopulation of genetically uniform polyploid maize seed has a partially-complemented MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the partially-complemented MiMe genotype comprises (a) only MiMe alleles at one or more MiMe loci of the first MiMe component; (b) one or more MiMe alleles and one or more non-MiMe alleles at a first MiMe locus of the second MiMe component, and one or more MiMe alleles and one or more non-MiMe alleles at a second MiMe locus of the second MiMe component; and (c) either (i) only MiMe alleles at one or more MiMe loci of the third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component. In some embodiments, the partially-complemented MiMe genotype comprises (a) only MiMe alleles at one or more MiMe loci of a component of sister chromatid cohesion during the first division of meiosis; (b) one or more MiMe alleles and one or more non-MiMe alleles at a first MiMe locus of a component of DNA double strand breakage during meiotic recombination, and one or more MiMe alleles and one or more non-MiMe alleles at a second MiMe locus of the component of DNA double strand breakage during meiotic recombination; and (c) either (i) only MiMe alleles at one or more MiMe loci of a component of progression through the second division of meiosis, or (ii) one or more MiMe alleles and one or more non-MiMe alleles at a first MiMe locus of a component of progression through the second division of meiosis, and one or more MiMe alleles and one or more non-MiMe alleles at a second MiMe locus of the component of progression through the second division of meiosis. Exemplary MiMe loci of each of said MiMe components are extensively described herein below. In one embodiment, the partially-complemented MiMe genotype comprises (a) only MiMe alleles of REC8; (b) one or more MiMe alleles and one or more non-MiMe alleles of SPO11-1, and one or more MiMe alleles and one or more non-MiMe alleles of PAIR1; and (c) only MiMe alleles of OSD1.

In other embodiments, the population of polyploid maize seed or the subpopulation of genetically uniform polyploid maize seed has a partially-complemented MiMe genotype comprising MiMe alleles conferring decreased expression of one or more MiMe loci each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the population of polyploid maize seed or the subpopulation of genetically uniform polyploid maize seed having a partially-complemented MiMe genotype comprising MiMe alleles conferring decreased expression of MiMe loci of (2) a component of DNA double strand breakage during meiotic recombination and (4) a component of progression through the first division of meiosis exhibits neither a MiMe phenotype nor a wild-type meiosis phenotype in male germline cells and does not produce viable male gametes, and exhibits a wild-type meiosis phenotype in female germline cells and/or produces viable haploid female gametes. In some embodiments, the partially-complemented MiMe genotype comprises (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component. In certain embodiments, the partially-complemented MiMe genotype comprises (a) only MiMe alleles at one or more MiMe loci of a component of DNA double strand breakage during meiotic recombination; and (b) one or more MiMe alleles and one or more non-MiMe alleles at a first MiMe locus of a component of progression through the first division of meiosis, and one or more MiMe alleles and one or more non-MiMe alleles at a second MiMe locus of the component of progression through the first division of meiosis. In other embodiments, the partially-complemented MiMe genotype comprises (a) only MiMe alleles at one or more MiMe loci of a component of progression through the first division of meiosis; and (b) one or more MiMe alleles and one or more non-MiMe alleles at a first MiMe locus of a component of DNA double strand breakage during meiotic recombination and one or more MiMe alleles and one or more non-MiMe alleles at a second MiMe locus of the component of DNA double strand breakage during meiotic recombination. Exemplary MiMe loci of each of said MiMe components are extensively described below.

In some embodiments, the population of polyploid maize seed or the subpopulation of genetically uniform polyploid maize seed has a partially-complemented MiMe genotype comprising MiMe alleles conferring decreased expression of one or more MiMe loci of a first, second, third, and fourth MiMe component are selected from the group consisting of (1) a component of sister chromatid cohesion during the first division of meiosis, (2) a component of DNA double strand breakage during meiotic recombination, (3) a component of progression through the second division of meiosis, and (4) a component of progression through the first division of meiosis. In certain embodiments, the partially-complemented genotype comprises (a) only MiMe alleles at one or more MiMe loci of the first MiMe component, wherein the first MiMe component is a component of DNA double strand breakage during meiotic recombination; (b) one or more MiMe alleles and one or more non-MiMe alleles at one or more MiMe loci of the second MiMe component; (c) one or more MiMe alleles and one or more non-MiMe alleles at one or more MiMe loci of the third MiMe component; (d) one or more MiMe alleles and one or more non-MiMe alleles at one or more MiMe loci of the fourth MiMe component. In some embodiments, the partially-complemented genotype comprises (a) only MiMe alleles at one or more MiMe loci of a component of DNA double strand breakage during meiotic recombination; (b) one or more MiMe alleles and one or more non-MiMe alleles at one or more MiMe loci of a component of sister chromatid cohesion during the first division of meiosis; (c) one or more MiMe alleles and one or more non-MiMe alleles at one or more MiMe loci of a component of progression through the second division of meiosis; (d) one or more MiMe alleles and one or more non-MiMe alleles at one or more MiMe loci of a component of progression through the first division of meiosis. Exemplary MiMe loci of each of said MiMe components are extensively described below. In one variation, the partially-complemented genotype comprises (a) only MiMe alleles at SPO11-1; (b) one or more MiMe alleles and one or more non-MiMe alleles at REC8; (c) one or more MiMe alleles and one or more non-MiMe alleles at OSD1; (d) one or more MiMe alleles and one or more non-MiMe alleles at PS1 or JASON.

In some embodiments, the population of polyploid maize seed or the subpopulation of genetically uniform polyploid maize seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more OSD1 loci comprise OSD1-1, OSD1-2, and/or OSD1-3, and/or the one or more PAIR1 loci comprise PAIR1-1 and/or PAIR1-2.

In some embodiments, the population of polyploid maize seed or the subpopulation of genetically uniform polyploid maize seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the one or more OSD1 loci comprise OSD1-1, OSD1-2, and/or OSD1-3.

In some embodiments, the population of polyploid maize seed or the subpopulation of genetically uniform polyploid maize seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid maize seed or the subpopulation of genetically uniform polyploid maize seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more PAIR1 loci comprise PAIR1-1 and/or PAIR1-2.

In some embodiments, the population of polyploid maize seed or the subpopulation of genetically uniform polyploid maize seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid maize seed or the subpopulation of genetically uniform polyploid maize seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid maize seed or the subpopulation of genetically uniform polyploid maize seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid maize seed or the subpopulation of genetically uniform polyploid maize seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more PAIR1 loci comprise PAIR1-1 and/or PAIR1-2.

In some embodiments, the population of polyploid maize seed or the subpopulation of genetically uniform polyploid maize seed has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1. In some variations, the one or more OSD1 loci comprise OSD1-1, OSD1-2, and/or OSD1-3, and/or the one or more PAIR1 loci comprise PAIR1-1 and/or PAIR1-2.

In some embodiments, the population of polyploid maize seed or the subpopulation of genetically uniform polyploid maize seed has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments, the population of polyploid maize seed or the subpopulation of genetically uniform polyploid maize seed has a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In some variations, the one or more PAIR1 loci comprise PAIR1-1 and/or PAIR1-2.

In some embodiments, the subpopulation of genetically uniform polyploid maize seed comprises one or more polynucleotide sequences selected from the group consisting of SEQ ID NOs: 108-111. In certain embodiments, the subpopulation of genetically uniform polyploid maize seed comprises a) a MiMe allele at one or more OSD1-2 loci, each independently comprising a polynucleotide sequence selected from the group consisting of SEQ ID NOs: 108 and 109; b) a MiMe allele at a REC8 locus comprising the polynucleotide, sequence SEQ ID NO: 110; and/or c) a MiMe allele at a SPO11-1 locus comprising the polynucleotide sequence of SEQ ID NO: 111 In some variations, each the one or more OSD1-2 loci, each of the REC8 or more REC8 loci, and/or each of the one or more SPO11-1 loci are present on a different homologous chromosome.

In some embodiments, the population of polyploid maize seed comprises one or more genetic modifications resulting in decreased expression of one or more MiMe loci. In certain embodiments, the genetic modification resulting in decreased expression of a MiMe locus is positioned in the first 70%, the first 60%, the first 50%, the first 40%, the first 30%, the first 20%, or the first 10% of the nucleotides of the coding sequence of the MiMe locus following the start codon in the 3′ direction. In some embodiments, the genetic modification resulting in decreased expression of a MiMe locus is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In some embodiments, the genetic modification resulting in decreased expression of a MiMe locus is an insertion, a deletion, one or more nucleotide changes, or an inversion. In some variations, the insertion, the deletion, the one or more nucleotide changes, or the inversion eliminates expression (e.g., eliminates activity) of the MiMe locus. In certain embodiments, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the MiMe locus following the start codon in the 3′ direction. In certain embodiments, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In certain embodiments, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the MiMe locus. In certain embodiments, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction, thereby eliminating expression (e.g., activity) of the MiMe locus.

In some embodiments, the one or more genetic modifications resulting in decreased expression of one or more MiMe loci comprise one or more sequences selected from the group consisting of SEQ ID NOs: 108-111.

Components of Sister Chromatid Cohesion during the First Division of Meiosis

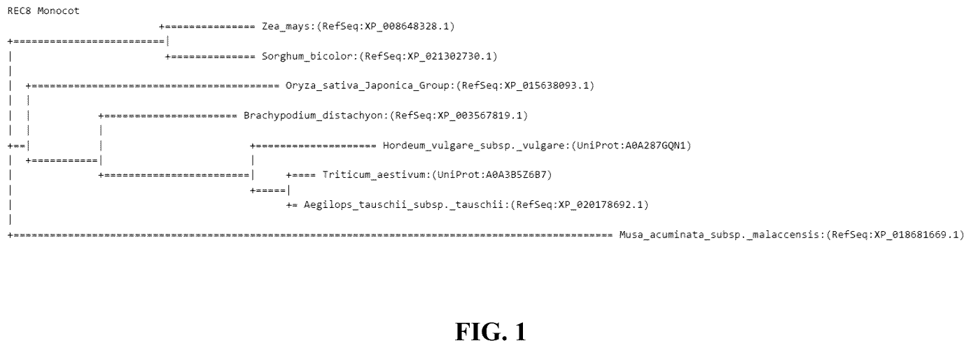

In some embodiments, the complete, partial, or partially-complemented MiMe genotype comprises one or more MiMe alleles conferring decreased expression of one or more MiMe loci of a component of sister chromatid cohesion during the first division of meiosis. In certain embodiments, the one or more MiMe loci of the component of sister chromatid cohesion during the first division of meiosis comprise REC8, SWITCH1/DYAD, or a combination thereof. One of skill in the art will understand that MiMe loci that are components of sister chromatid cohesion during the first division of meiosis are not limited to REC8 and SWITCH1/DYAD, and may include any loci encoding gene products required for sister chromatid cohesion during the first division of meiosis. By way of example only, a gene product of the component of sister chromatid cohesion during the first division of meiosis is exemplified by a REC8 protein and specifically by the REC8 protein sequences, sequence alignments, and percent identities described in “MiMe Gene Product Sequences” below. A representative REC8 protein sequence from maize (SEQ ID NO: 1) is provided in the sequence listing as outlined in Table 5, including eight native sequences and a consensus sequence identified by multiple sequence alignment of the eight native sequences (Sequence Alignment 1). Table 1 shows a matrix of percent identities of the REC8 protein sequences from monocotyledonous plants, and a phylogenetic tree showing the relationship between the sequences is shown in FIG. 1. The gene products of MiMe loci of the component of sister chromatid cohesion during the first division of meiosis include REC8 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the REC8 protein of SEQ ID NO: 1.

There is an abundance of known REC8 gene products frommaize including all of the following, which sequences are hereby incorporated by reference in their form as of the effective filing date. Exemplary REC8 gene products from maize include those of, by way of example, Zea mays (NP 001105829.1, XP 008648327.1, XP 008648329.1, and XP 008648328.1.

A gene product of the component of sister chromatid cohesion during the first division of meiosis is also exemplified by a SWITCH1 protein. There is an abundance of known SWITCH1 gene products from maize including all of the following, which sequences are hereby incorporated by reference in their form as of the effective filing date. Exemplary SWITCH1 gene products from maize include those of, by way of example, Zea mays (NP_001139538.1, XP 008662288.1 and CORWW9).

Components of DNA Double Strand Breakage During Meiotic Recombination

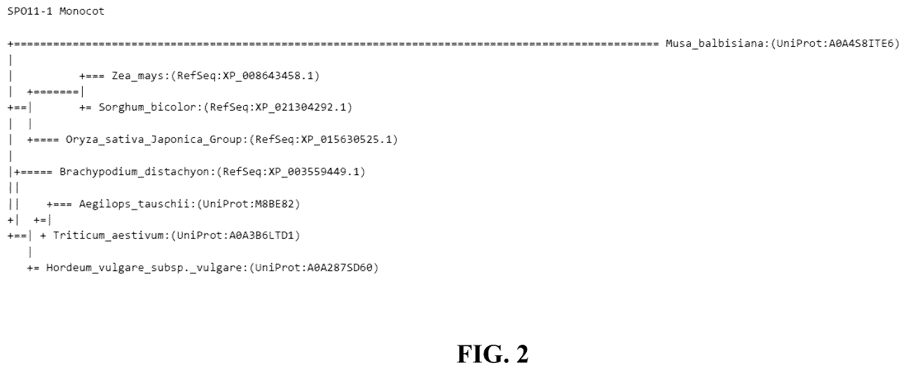

In some embodiments, the complete, partial, or partially-complemented MiMe genotype comprises one or more MiMe alleles conferring decreased expression of one or more MiMe loci of a component of DNA double strand breakage during meiotic recombination. In certain embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. One of skill in the art will understand that MiMe loci of the component of DNA double strand breakage during meiotic recombination are not limited to PAIR1, SPO11-1, SPO11-2, PRD1, PRD2, DFO, MTOPVIB, DSY1, SY1, SY2, SY3, and SY4 and may include any loci encoding gene products required for DNA double strand breakage during meiotic recombination. By way of example only, a gene product of the component of DNA double strand breakage during meiotic recombination is exemplified by a SPO11-1 protein and specifically by the SPO11-1 protein sequences, sequence alignments, and percent identities described in “MiMe Gene Product Sequences” below. A representative SPO11-1 protein sequences from maize (SEQ ID NO: 2) is provided in the sequence listing as outlined in Table 5, including eight native sequences and a consensus sequence identified by multiple sequence alignment of the eight native sequences (Sequence Alignment 2). Table 2 shows a matrix of percent identities of the SPO11-1 protein sequences from monocotyledonous plants, and a phylogenetic tree showing the relationship between the sequences is shown in FIG. 2. The gene products of MiMe loci of the component of DNA double strand breakage during meiotic recombination include SPO11-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the SPO11-1 proteins of SEQ ID NO: 2.

There is an abundance of known SPO11-1 gene products from maize including all of the following, which sequences are hereby incorporated by reference in their form as of the effective filing date. Exemplary SPO11-1 gene products from maize include those of, by way of example, Zea luxurians (A0A1P8W173), Zea mays (NP 001347894.1, XP 008643457.1, XP 008643458.1, XP 008643459.1, XP 020408860.1, and XP 008643458.1), Zea mays subsp. huehuetenangensis (A0A1P8W126), Zea mays subsp. mays (A0A1P8W137 and A0A1P8W169), Zea mays subsp. mexicana (A0A1P8W110), Zea mays subsp. parviglumis (A0A1P8W0Z9, A0A1P8W103, A0A1P8W125, and A0A1P8W189).

A gene product of the component of DNA double strand breakage during meiotic recombination is also exemplified by a SPO11-2 protein. There is an abundance of known SPO11-2 gene products from maize including all of the following, which sequences are hereby incorporated by reference in their form as of the effective filing date. Exemplary SPO11-2 gene products from maize include those of, by way of example, Zea mays (XP 020406911.1, NP_001298099.1 and NP_001141583.1), Zea mays (A0A1P8W150, A0A1P8W149 and A0A1P8W163), Zea mays (A0A1P8W133, A0A1P8W171 and A0A1P8W158), Zea diploperennis (A0A1P8W179), Zea mays (A0A1P8W114), Zea luxurians (A0A1P8W147), and Zea mays (A0A1P8W1E8).

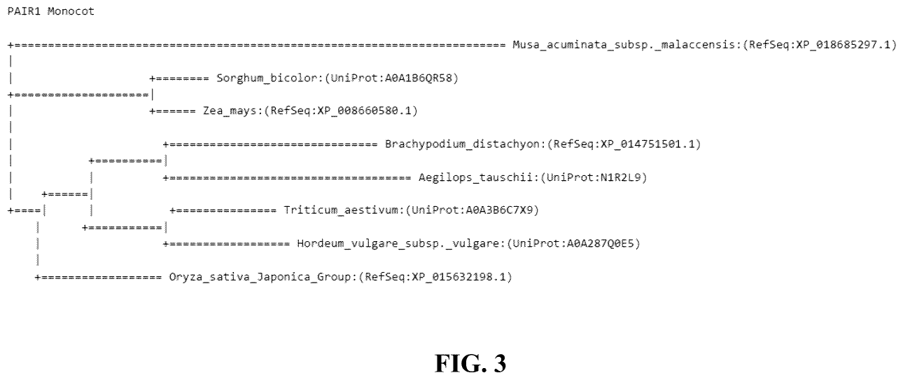

A gene product of the component of DNA double strand breakage during meiotic recombination is also exemplified by a PAIR1 protein (e.g., a PAIR1-1 protein or a PAIR1-2 protein) and specifically by the PAIR1 protein sequences, sequence alignments, and percent identities described in “MiMe Gene Product Sequences” below. Representative PAIR1 protein sequences from maize (SEQ ID NOs: 3 and 5), including a representative PAIR1-1 protein sequence from maize (SEQ ID NO: 3) and a representative PAIR1-2 protein sequence from maize (SEQ ID NO: 5), are provided in the sequence listing as outlined in Table 5, including eight native sequences and a consensus sequence identified by multiple sequence alignment of the eight native sequences (Sequence Alignment 3). Table 3 shows a matrix of percent identities of the PAIR1 protein sequences from monocotyledonous plants, and a phylogenetic tree showing the relationship between the sequences is shown in FIG. 3. The gene products of MiMe loci of the component of DNA double strand breakage during meiotic recombination includes PAIR1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the PAIR1 proteins of SEQ ID NOs: 3 and 5. The gene products of MiMe loci of the component of DNA double strand breakage during meiotic recombination also include: a) PAIR1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PAIR1-1 protein of SEQ ID NO: 3; and b) PAIR1-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PAIR1-2 protein of SEQ ID NO: 5.

There is an abundance of known PAIR1 gene products maize including all of the following, which sequences are hereby incorporated by reference in their form as of the effective filing date. Exemplary PAIR1 gene products from maize include those of, by way of example, Zea mays (XP 008660580.1, A0A1D6JK92, A0A1D6JK93, A0A1D6JK94, and A0A1D6PM18.

A gene product of the component of DNA double strand breakage during meiotic recombination is also exemplified by a PRD1 protein. There is an abundance of known PRD1 gene products from maize including all of the following, which sequences are hereby incorporated by reference in their form as of the effective filing date. Exemplary PRD1 gene products from maize include those of, by way of example, Zea mays (XP 020399554.1, XP 020399553.1 and A0A1D6P5Q4).

A gene product of the component of DNA double strand breakage during meiotic recombination is also exemplified by a PRD2 protein. There is an abundance of known PRD2 gene products from maize including all of the following, which sequences are hereby incorporated by reference in their form as of the effective filing date. Exemplary PRD2 gene products from maize include those of, by way of example, Zea mays (NP_001130070.1, XP 035822500.1 and A0A3L6F902), Setaria viridis (XP 034583479.1, XP 034583480.1 and XP 034600292.1).

A gene product of the component of DNA double strand breakage during meiotic recombination is also exemplified by a DFO protein. There is an abundance of known DFO gene products from maize including all of the following, which sequences are hereby incorporated by reference in their form as of the effective filing date. Exemplary DFO gene products from dicots include those of, by way of example, Zea mays (XP 020397706.1, A0A1D6QG87 and A0A3L6EXP8).

A gene product of the component of DNA double strand breakage during meiotic recombination is also exemplified by a MTOPVIB protein. There is an abundance of known MTOPVIB gene products from maize including all of the following, which sequences are hereby incorporated by reference. Exemplary MTOPVIB gene products from maize include those of, by way of example, Zea mays (XP 008645058.1, A0A3L6EMU7 and A0A1D6GVU9).

Components of Progression through the Second Division of Meiosis

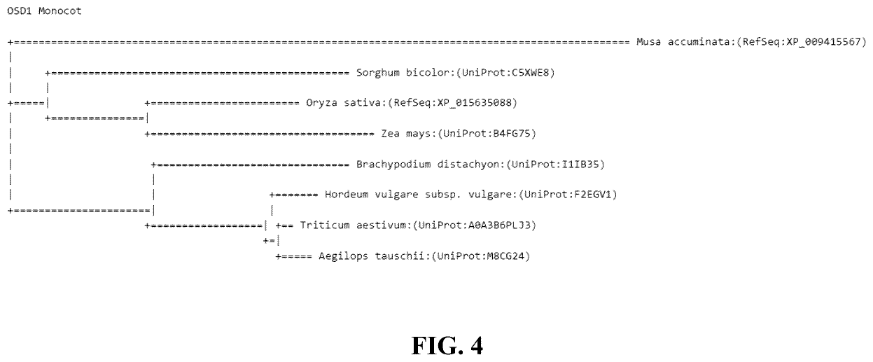

In some embodiments, the complete, partial, or partially-complemented MiMe genotype comprises one or more MiMe alleles conferring decreased expression of one or more MiMe loci of a component of progression through the second division of meiosis. Components of progression through the second division of meiosis include, for example, both (a) MiMe loci that encode gene products that are required for progression through the second division of meiosis and (b) MiMe loci that that are associated with second division restitution mechanisms, since the effective end result of both is as if the second division of meiosis did not occur, with the resulting gametes of each containing sister chromatids. Second division restitution (also known as nuclear restitution) mechanisms are known in the art and described in Brownfield and Kohler (2010. Unreduced gamete formation in plants: mechanisms and prospects. J Exp Bot 62:5, 1659-1668). In certain embodiments, the one or more MiMe loci of the component of progression through the second division of meiosis comprise OSD1, CYCA1, TDM1, PC1, PC2, FC, or any combination thereof. One of skill in the art will understand that MiMe loci of the component of progression through the second division of meiosis are not limited to OSD1, CYCA1, TDM1, PC1, PC2, and FC, and may include any loci encoding gene products required for progression through the second division of meiosis or associated with second division restitution mechanisms. By way of example only, a gene product of the component of progression through the second division of meiosis of the first division sister chromatid segregation is exemplified by an OSD1 protein (e.g., an OSD1-1 protein, an OSD1-2 protein, or an OSD1-3 protein) and specifically by the OSD1 protein sequences, sequence alignments, and percent identities described in “MiMe Gene Product Sequences” below. Representative OSD1 protein sequences from maize (SEQ ID NOs: 4, 6, and 7) are provided in the sequence listing as outlined in Table 5, including eight native sequences and a consensus sequence identified by multiple sequence alignment of the eight native sequences (Sequence Alignment 4), as well as OSD1-1, OSD1-2, and OSD1-3 protein sequences from maize. OSD1 homologues present in the maize genome include, for example, OSD1-1 on chromosome 2 (SEQ ID NO: 4), OSD1-2 on chromosome 5 (SEQ ID NO: 6), and OSD1-3 on chromosome 5 (SEQ ID NO: 7). Table 4A shows a matrix of percent identities of the OSD1 protein sequences from monocotyledonous plants, and a phylogenetic tree showing the relationship between the sequences is shown in FIG. 4. The gene products of MiMe loci of the component of progression through the second division of meiosis include OSD1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the OSD1 proteins of SEQ ID NOs: 4 and 6-7. The gene products of MiMe loci of the component of progression through the second division of meiosis also include: a) OSD1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the OSD1-1 protein of SEQ ID NO: 4; b) OSD1-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the OSD1-2 protein of SEQ ID NO: 6; a) OSD1-3 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the OSD1-3 protein of SEQ ID NO: 7.

There is an abundance of known OSD1 gene products from maize including all of the following, which sequences are hereby incorporated by reference in their form as of the effective filing date. Exemplary OSD1 gene products from maize include those of, by way of example, Zea mays (A0A1D6HBV9, A0A1D6HBWO, B4FCP3, B4FG75, and B6T7U1).

By way of example only, a gene product of the component of progression through the second division of meiosis is also exemplified by a CYCA1 protein, also known as CYCLIN-A1 or TARDY ASYNCHRONOUS MEIOSIS (TAM), and specifically by the CYCA1 protein sequences, sequence alignments, and percent identities described in “MiMe Gene Product Sequences” below. A representative CYCA1 protein sequence from maize (SEQ ID NOs: 10 and 13) are provided in the sequence listing as outlined in Table 5, including native sequences and a consensus sequence identified by multiple sequence alignment of the native sequences (Sequence Alignment 5). Table 4B shows a matrix of percent identities of the OSD1 protein sequences from monocotyledonous plants. The gene products of MiMe loci of the component of progression through the second division of meiosis include CYCA1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the CYCA1 protein of SEQ ID NOs: 10 and 13.

There is an abundance of known CYCA1 gene products from maize including all of the following, which sequences are hereby incorporated by reference in their form as of the effective filing date. Exemplary CYCA1 gene products from maize include those of, by way of example, Zea mays (NP_001105387.2, NP_001288521.1 and XP 008656316.2).

By way of example only, a gene product of the component of progression through the second division of meiosis is also exemplified by a TDM1 protein, and specifically by the TDM1 protein sequences and percent identities described herein. A representative TDM1 protein sequence from maize (SEQ ID NO: 11) is provided in Table 5. The gene products of MiMe loci of the component of progression through the second division of meiosis include TDM1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the TDM1 protein of SEQ ID NO: 11. The gene products of MiMe loci of the component of progression through the second division of meiosis also include TDM1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the protein of SEQ ID NO: 11.

There is an abundance of known TDM1 gene products from maize including all of the following, which sequences are hereby incorporated by reference in their form as of the effective filing date. Exemplary TDM1 gene products from maize include those of, by way of example, Zea mays (NP_001170740.1, NP_001141607.1 and XP 008651103.1).

Components of Progression Through the First Division of Meiosis

In some embodiments, the complete, partial, or partially-complemented MiMe genotype comprises one or more MiMe alleles conferring decreased expression of one or more MiMe loci of a component of progression through the first division of meiosis. Components of progression through the first division of meiosis include, for example, both (a) MiMe loci that encode gene products that are required for progression through the first division of meiosis and (b) MiMe loci that that are associated with first division restitution mechanisms, since the effective end result of both is as if the first division of meiosis did not occur, with the resulting gametes of each containing non-sister chromatids. First division restitution (also known as nuclear restitution) mechanisms are known in the art and described in Peloquin et al. (1999. Meiotic mutants in potato: valuable variants. Genetics 153: 1493-1499) and Brownfield and Kohler (2010. Unreduced gamete formation in plants: mechanisms and prospects. J Exp Bot 62:5, 1659-1668). In certain embodiments, the one or more MiMe loci of the component of progression through the first division of meiosis comprise PS1, JASON, or a combination thereof. One of skill in the art will understand that MiMe loci of the component of progression through the first division of meiosis are not limited to PS1 and JASON, and may include any loci encoding gene products required for progression through the first division of meiosis or associated with first division restitution mechanisms. By way of example only, a gene product of the component of progression through the first division of meiosis is exemplified by PS1 protein or a PS1-like protein, and specifically by the PS1 and PS1-like protein sequences and percent identities described herein. A representative PS1 protein sequence (SEQ ID NO: 12) is provided in the sequence listing as outlined in Table 5. The gene products of MiMe loci of the component of progression through the first division of meiosis include: a) PS1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PS1 protein of SEQ ID NO:12

There is an abundance of known PS1 gene products from maize including all of the following, which sequences are hereby incorporated by reference in their form as of the effective filing date. Exemplary PS1 gene products from maize include those of, by way of example, Zea mays (NP_001348386.1, XP 035820777.1 and XP 008668271.1).

By way of example only, a gene product of the component of progression through the first division of meiosis is also exemplified by a JASON protein (e.g., a JASON-1 or a JASON-2 protein), and specifically by the JASON protein sequences and percent identities described herein. Representative JASON protein sequences (SEQ ID. 8 and 9), including a representative JASON-1 protein sequence (SEQ ID NO: 8) and a representative JASON-2 protein sequence (SEQ ID NO: 9), are provided in the sequence listing as outlined in Table 5. The gene products of MiMe loci of the component of progression through the first division of meiosis include: a) JASON-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the JASON-1 protein of SEQ ID NO: 8; b) JASON-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the JASON-2 protein of SEQ ID NO: 9.

There is an abundance of known JASON gene products from maize including all of the following, which sequences are hereby incorporated by reference in their form as of the effective filing date. Exemplary JASON gene products from maize include those of, by way of example, Zea mays (NP_001132267.1, XP 008647301.1 and NP_001130670.1).

The sequences of the gene products listed above may be accessed using the given accession numbers in the RefGen, UniProt, and RefSeq databases.

Exemplary Populations of Polyploid Seed

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize. In certain embodiments, the population was obtained from a single maize plant or a set of maize plants (e.g., a set of genetically uniform maize plants, e.g., a set of genetically uniform F1 hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, or at least about 95%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. The subpopulation of genetically uniform polyploid maize seed may have any complete MiMe genotype, partial MiMe genotype, or partially-complemented MiMe genotype described herein. In certain embodiments, the subpopulation of genetically uniform polyploid maize seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8, SWITCH1/DYAD, or a combination thereof; the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof; the one or more MiMe loci of the component of progression through the second division of meiosis comprise OSD1, CYCA1, TDM1, PC1, PC2, FC, or any combination thereof; and/or the one or more MiMe loci of the component of progression through the first division of meiosis comprise PS1, JASON, or a combination thereof. In other variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, TDM1, or any combination thereof; and/or the one or more MiMe loci of the component of progression through the first division of meiosis comprise PS1. In some embodiments, the subpopulation of genetically uniform polyploid maize seed comprises one or more polynucleotide sequences selected from the group consisting of SEQ ID NOs: 108-111. In certain embodiments, the subpopulation of genetically uniform polyploid maize seed comprises a) a MiMe allele at one or more OSD1-2 loci, each independently comprising a polynucleotide sequence selected from the group consisting of SEQ ID NOs: 108 and 109; b) a MiMe allele at a REC8 locus comprising the polynucleotide sequence of SEQ 11) NO: 110: and/or c) a MiMe allele at a SPO11-1 locus comprising the polynucleotide sequence of SEQ ID NO: 111. In some variations. each the one or more OSD1-2 loci, each of the one or more REC8 loci. and/or each of the one or more SPO11-1 loci are present on a different homologous chromosome.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize. In certain embodiments, the population of maize seed was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, or at least about 95%) as measured by the Jaccard similarity coefficient. In one embodiment, the population of polyploid maize seed has an average pairwise genetic uniformity of at least 99% or about 100% as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In one embodiment, the population of polyploid maize seed comprises the subpopulation of genetically uniform polyploid maize seed in an amount of at least about 98% of the total number of seeds, wherein each pair of seeds in the subpopulation of genetically uniform polyploid maize seed has a pairwise identity of 100%. The subpopulation of genetically uniform polyploid maize seed may have any complete MiMe genotype, partial MiMe genotype, or partially-complemented MiMe genotype described herein. In certain embodiments, the subpopulation of genetically uniform polyploid maize seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, or a combination thereof; and/or the one or more MiMe loci of the component of progression through the first division of meiosis comprise PS1. In some embodiments, the subpopulation of genetically uniform polyploid maize seed comprises one or more polynucleotide sequences selected from the group consisting of SEQ ID NOs: 108-111. In certain embodiments, the subpopulation of genetically uniform polyploid maize seed comprises a) a MiMe allele at one or more OSD1-2 loci, each independently comprising a polynucleotide sequence selected from the group consisting of SEQ ID NOs: 108 and 109; b) a MiMe allele at a REC8 locus comprising the polynucleotide sequence of SEQ ID NO: 110; and/or c) a MiMe allele at a SPO11-1 locus comprising the polynucleotide sequence of SEQ ID NO: 111. In some variations, each the one or more OSD1-2 loci, each of the one or more REC8 loci. and/or each of the one or more SPO11-1 loci are present on a different homologous chromosome.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the subpopulation of genetically uniform polyploid maize seed comprises a) a rec8 allele comprising the polynucleotide sequence of SEQ ID NO: 110; and/or b) one or more osd1 alleles, each comprising a polynucleotide sequence selected from the group consisting of SEQ ID NOS: 108 and 109.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the subpopulation of genetically uniform polyploid maize seed comprises a) a rec8 allele comprising the polynucleotide sequence of SEQ ID NO: 110; and/or b) one or more osd1 alleles, each comprising a polynucleotide sequence selected from the group consisting of SEQ ID NOS: 108 and 109.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficientln some variations, the one or more OSD1 loci comprise OSD1-1, OSD1-2, and/or OSD1-3. In certain variations, the one or more OSD1 loci comprise OSD1-1 and OSD1-2. In some variations, the subpopulation of genetically uniform polyploid maize seed comprises a) a rec8 allele comprising the polynucleotide sequence of SEQ ID NO: 110; b) one or more osd1 alleles, each comprising a polynucleotide sequence selected from the group consisting of SEQ ID NOS: 108 and 109; and/or c) a spo1 1-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient In some variations, the one or more OSD1 loci comprise OSD1-1, OSD1-2, and/or OSD1-3. In certain variations, the one or more OSD1 loci comprise OSD1-1 and OSD1-2. In some variations, the subpopulation of genetically uniform polyploid maize seed comprises a) a rec8 allele comprising the polynucleotide sequence of SEQ ID NO: 110; b) one or more osd1 alleles, each comprising a polynucleotide sequence selected from the group consisting of SEQ ID NOS: 108 and 109; and/or c) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the subpopulation of genetically uniform polyploid maize seed comprises a) a rec8 allele comprising the polynucleotide sequence of SEQ ID NO: 110; and/or b) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the subpopulation of genetically uniform polyploid maize seed comprises a) a rec8 allele comprising the polynucleotide sequence of SEQ ID NO: 110; and/or b) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, the subpopulation of genetically uniform polyploid seed comprises a rec8 allele comprising the polynucleotide sequence of SEQ ID NO: 110.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, the subpopulation of genetically uniform polyploid maize seed comprises a rec8 allele comprising the polynucleotide sequence of SEQ ID NO: 110.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the subpopulation of genetically uniform polyploid maize seed comprises a) a rec8 allele comprising the polynucleotide sequence of SEQ ID NO: 110; and/or b) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the subpopulation of genetically uniform polyploid maize seed comprises a) a rec8 allele comprising the polynucleotide sequence of SEQ ID NO: 110; and/or b) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, subpopulation of genetically uniform polyploid maize seed comprises a rec8 allele comprising the polynucleotide sequence of SEQ ID NO: 110.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, the subpopulation of genetically uniform polyploid maize seed comprises a rec8 allele comprising the polynucleotide sequence of SEQ ID NO: 110.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the subpopulation of genetically uniform polyploid maize seed comprises a) a rec8 allele comprising the polynucleotide sequence of SEQ ID NO: 110; b) one or more osd1 alleles, each comprising a polynucleotide sequence selected from the group consisting of SEQ ID NOs: 108 and 109; and/or c) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, the subpopulation of genetically uniform polyploid maize seed comprises a) a rec8 allele comprising the polynucleotide sequence of SEQ ID NO: 110; and/or b) a spol 1-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In some variations, the one or more PAIR1 loci comprise PAIR1-1 and/or PAIR1-2. In some variations, the subpopulation of genetically uniform polyploid maize seed comprises a) a rec8 allele comprising the polynucleotide sequence of SEQ ID NO: 110; and/or b) a spo11-1 allele comprising the polynucleotide sequence of SEQ ID NO: 111.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of maize and one or more polynucleotide sequences selected from the group consisting of SEQ ID NOs: 108-111. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is triploid, tetraploid, or pentaploid. In one embodiment, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In certain embodiments, the subpopulation of genetically uniform polyploid maize seed comprises a) a MiMe allele at each of one or more OSD1-2 loci, each independently comprising a polynucleotide sequence selected from the group consisting of SEQ ID NOs: 108 and 109; b) a MiMe allele at a REC8 locus comprising the polynucleotide sequence of SEQ ID NO: 110; and/or c) a MiMe allele at SPO11-1 locus Comprising the polynucleotide sequence of SEQ ID NO:111. In some variations, each the one or more OSD1-2 loci, each of the one or more REC8 loci, and/or each of the one or more SPO11-1 loci are present on a different homologous chromosome.

Methods of Producing Populations of Polyploid Maize Seed

In another aspect, provided herein are methods of producing a population of polyploid maize seed comprising three or more haplotypes wherein at least 50% of the population of polyploid maize seed are genetically uniform. In some embodiments, the population of polyploid maize seed comprises a subpopulation of genetically uniform polyploid maize seed in an amount of at least 50%, the genetically uniform polyploid maize seed comprising the three or more haplotypes. In some embodiments, the method comprises (a) providing clonal gametes from a pair of parent MiMe maize plants that together comprise three or more haplotypes; and (b) crossing the clonal gametes to produce the population of polyploid maize seed. In other embodiments, the method comprises (a) providing clonal gametes from a parent MiMe maize plant; (b) providing haploid gametes from a homozygous parent non-MiMe maize plant; and (c) crossing the clonal gametes with the haploid gametes to produce the population of polyploid maize seed. In other embodiments, the method comprises (a) providing clonal gametes from a parent MiMe maize plant; (b) providing haploid gametes from a homozygous parent non-MiMe maize plant; and (c) crossing the clonal gametes with the haploid gametes to produce the population of polyploid maize seed; wherein the clonal gametes and the haploid gametes together comprise three or more haplotypes. In still other embodiments, the method comprises (a) providing clonal gametes from a parent MiMe maize plant; (b) providing unreduced, non-clonal gametes from a homozygous parent maize plant; and (c) crossing the clonal gametes with the unreduced, non-clonal gametes to produce the population of polyploid maize seed; wherein the clonal gametes and the unreduced, non-clonal gametes together comprise three or more haplotypes.

In another aspect, provided herein are methods of producing a population of polyploid maize seed comprising a partially-complemented MiMe genotype wherein at least 50% of the population of polyploid maize seed are genetically uniform. In some embodiments, provided herein are methods of producing a population of polyploid maize seed comprising a subpopulation of genetically uniform polyploid maize seed in an amount of at least 50% of the total number of seeds, the subpopulation of genetically uniform polyploid maize seed comprising a partially-complemented MiMe genotype wherein at least 50% of the population of polyploid maize seed are genetically uniform. In some embodiments, the method comprises (a) providing clonal gametes from a first parent MiMe maize plant; (b) providing clonal gametes from a second parent MiMe maize plant; and (c) crossing the clonal gametes to produce the population of polyploid maize seed comprising a partially-complemented MiMe genotype. In certain embodiments, the polyploid maize seed (e.g., the subpopulation of genetically uniform polyploid maize seed) comprises one, two, three, or more haplotypes.

In some embodiments, at least 50% of the population of polyploid maize seed produced are genetically uniform. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid maize seed produced are genetically uniform. In some embodiments, at least 50% of the population of polyploid maize seed produced are genetically uniform, wherein the polyploid maize seed comprises two, three, or more haplotypes. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid maize seed produced are genetically uniform, wherein the polyploid maize seed comprises two, three, or more haplotypes.

In some embodiments, the population of polyploid maize seed comprises a subpopulation of genetically uniform polyploid maize seed in an amount of at least 50% of the total number of seeds. In some variations, the population of polyploid maize seed comprises a subpopulation of genetically uniform polyploid maize seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of seeds. In some embodiments, the subpopulation of genetically uniform polyploid maize seed comprises two, three, or more haplotypes. In some variations, the population of polyploid maize seed comprises a subpopulation of genetically uniform polyploid maize seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of seeds, wherein the genetically uniform polyploid maize seed comprises two, three, or more haplotypes. In certain variations, the subpopulation of genetically uniform polyploid maize seed has a complete MiMe genotype, a partial MiMe genotype, or a partially-complemented MiMe genotype.

Providing Clonal Gametes

In some embodiments, the method of producing a population of polyploid maize seed comprises providing clonal gametes. In some embodiments, providing clonal gametes comprises growing a maize plant with a complete MiMe genotype that exhibits a MiMe phenotype and allowing the maize plant to grow to a reproductive stage until clonal gametes form. In other embodiments, providing clonal gametes comprises generating a maize plant with a complete MiMe genotype that exhibits a MiMe phenotype and growing said maize plant to a reproductive stage until clonal gametes form. In some variations, providing clonal gametes further comprises collecting pollen comprising clonal gametes from a maize plant with a complete MiMe genotype that exhibits a MiMe phenotype.

In some embodiments, the method of producing a population of polyploid maize seed comprises providing clonal gametes from one or more parent MiMe maize plants. In some variations, the method of producing a population of polyploid maize seed comprises providing clonal gametes from a pair of parent MiMe maize plants. In some embodiments, providing clonal gametes comprises growing one or more parent MiMe maize plants and allowing them to grow to a reproductive stage until clonal gametes form. In other embodiments, providing clonal gametes comprises generating one or more parent MiMe maize plants and growing them to a reproductive stage until clonal gametes form. In some variations, providing clonal gametes further comprises collecting pollen comprising clonal gametes from one or more parent MiMe maize plants. In some additional variations, the pair of parent MiMe maize plants are diploid, triploid, tetraploid, pentaploid, hexaploid, heptaploid, octaploid, or any combination thereof.

In some embodiments, the method of producing a population of polyploid maize seed comprises providing clonal gametes from a pair of parent MiMe maize plants that together comprise three or more haplotypes. In some variations, the pair of parent MiMe maize plants together comprise three or more haplotypes, four or more haplotypes, five or more haplotypes, six or more haplotypes, seven or more haplotypes, or eight or more haplotypes. In additional variations, the pair of parent MiMe maize plants together comprise three, four, five, six, seven, or eight haplotypes. In some embodiments, the first parent MiMe maize plant comprises one or more haplotypes and the second parent MiMe maize plant comprises one or more haplotypes, wherein the parent MiMe maize plants together comprise two or more haplotypes. In some embodiments, the first parent MiMe maize plant comprises one haplotype and the second parent MiMe maize plant comprises two or more haplotypes, wherein the parent MiMe maize plants together comprise three or more haplotypes. In other embodiments, each of the parent MiMe maize plants comprises two or more haplotypes, wherein the parent MiMe maize plants together comprise four or more haplotypes.

Parent MiMe Maize Plant Genotypes

In some embodiments, each of the parent MiMe maize plants has a complete MiMe genotype. In certain embodiments, the complete MiMe genotype comprises one or more genetic modifications resulting in decreased expression of one or more MiMe loci. In other embodiments, the complete MiMe genotype comprises one or more genetic modifications resulting in decreased expression of two or more MiMe loci. In yet another embodiment, the complete MiMe genotype comprises one or more genetic modifications resulting in decreased expression of three or more MiMe loci. In some variations, the MiMe loci may include, but are not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one variation, the complete MiMe genotype comprises one or more genetic modifications resulting in decreased expression of REC8. In a second variation, the complete MiMe genotype comprises one or more genetic modifications resulting in decreased expression of OSD1. In a third variation, the complete MiMe genotype comprises one or more genetic modifications resulting in decreased expression of PAIR1. In a fourth variation, the complete MiMe genotype comprises one or more genetic modifications resulting in decreased expression of SPO11-1, SPO11-2, or a combination thereof. In a fifth variation, the complete MiMe genotype comprises one or more genetic modifications resulting in decreased expression of REC8 and SPO11-1. In a sixth variation, the complete MiMe genotype comprises one or more genetic modifications resulting in decreased expression of REC8 and OSD1. In a seventh variation, the complete MiMe genotype comprises one or more genetic modifications resulting in decreased expression of REC8 and PAIR1. In an eighth variation, the complete MiMe genotype comprises one or more genetic modifications resulting in decreased expression of OSD1 and SPO11-1. In a ninth variation, the complete MiMe genotype comprises one or more genetic modifications resulting in decreased expression of OSD1 and PAIR1. In a tenth variation, the complete MiMe genotype comprises one or more genetic modifications resulting in decreased expression of REC8, OSD1, and PAIR1. In an eleventh variation, the complete MiMe genotype comprises one or more genetic modifications resulting in decreased expression of REC8, OSD1, and SPO11-1. In a twelfth variation, the complete MiMe genotype comprises one or more genetic modifications resulting in decreased expression of PS1 and SPO11-1. In a thirteenth variation, the complete MiMe genotype comprises one or more genetic modifications resulting in decreased expression of PS1 and SY3. The complete MiMe genotype may comprise one or more genetic modifications resulting in decreased expression of any combination of MiMe loci described herein or known in the art, wherein a maize plant that has the complete MiMe genotype exhibits a MiMe phenotype. In some embodiments, the complete MiMe genotype may comprise one or more genetic modifications resulting in non-expression of any combination of MiMe loci described here or known in the art wherein a maize plant that has the complete MiMe genotype exhibits a MiMe phenotype. In further embodiments, the complete MiMe genotype may comprise one or more genetic modifications resulting in decreased expression, non-expression, or a combination thereof of any combination of MiMe loci described here or known in the art, wherein a maize plant that has the complete MiMe genotype exhibits a MiMe phenotype. The parent MiMe maize plant may have any complete MiMe genotype known in the art or described herein, including, but not limited to, complete MiMe genotypes comprising MiMe alleles resulting in decreased expression of any of the MiMe loci described herein. Specific examples of complete MiMe genotypes are shown in Table 6.

In some embodiments, the complete MiMe genotype comprises one or more genetic modifications resulting in decreased expression of REC8, SWITCH1/DYAD, or a combination thereof. In some variations, the complete MiMe genotype comprises one or more genetic modifications resulting in decreased expression of REC8, SWITCH1/DYAD, or a combination thereof and one or more genetic modifications resulting in decreased expression of one or more additional MiMe loci. In additional variations, the complete MiMe genotype comprises one or more genetic modifications resulting in decreased expression of REC8, SWITCH1/DYAD, or a combination thereof and one or more genetic modifications resulting in decreased expression of one or more additional MiMe loci, including, but not limited to, OSD1, CYCA1, TDM1, PC1, PC2, and FC. In yet additional variations, the complete MiMe genotype comprises one or more genetic modifications resulting in decreased expression of REC8, SWITCH1/DYAD, or a combination thereof; one or more genetic modifications resulting in decreased expression of OSD1, CYCA1, TDM1, PC1, PC2, FC, or any combination thereof; and one or more genetic modifications resulting in decreased expression of one or more additional MiMe loci, including, but not limited to, PAIR1, SPO11-1, SPO11-2, PRD1, PRD2, DFO, MTOPVIB, DSY1, SY1, SY2, SY3, and SY4.

In some embodiments, the complete MiMe genotype comprises one or more genetic modifications resulting in decreased expression of one or more MiMe loci including, but not limited to, OSD1, CYCA1, TDM1, PC1, PC2, and FC. In some variations, the complete MiMe genotype comprises one or more genetic modifications resulting in decreased expression of OSD1, CYCA1, TDM1, PC1, PC2, FC, or any combination thereof and further comprises one or more genetic modifications resulting in decreased expression of one or more additional MiMe loci, including, but not limited to, PAIR1, SPO11-1, SPO11-2, PRD1, PRD2, DFO, MTOPVIB, DSY1, SY1, SY2, SY3, and SY4.

In some embodiments, the complete MiMe genotype comprises one or more genetic modifications resulting in decreased expression of one or more MiMe loci including, but not limited to, PAIR1, SPO11-1, SPO11-2, PRD1, PRD2, DFO, MTOPVIB, DSY1, SY1, SY2, SY3, and SY4. In some variations, the complete MiMe genotype comprises one or more genetic modifications resulting in decreased expression of PAIR1, SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1 and JASON.

In some embodiments, the parent MiMe maize plant has a complete MiMe genotype comprising MiMe alleles conferring decreased expression of MiMe loci of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the parent MiMe maize plant has a complete MiMe genotype comprising MiMe alleles conferring decreased expression of MiMe loci of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the parent MiMe maize plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. The parent MiMe maize plant may have any complete MiMe genotype known in the art or described herein, including, but not limited to, complete MiMe genotypes comprising MiMe alleles resulting in decreased expression of any of the MiMe loci described herein.

Parent MiMe Maize Plant Complementary Genotypes

In some embodiments, each of the parent MiMe maize plants has a complete MiMe genotype comprising MiMe alleles conferring decreased expression of MiMe loci of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, (a) the first parent MiMe maize plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) the second parent MiMe maize plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (c) at least one of the MiMe loci having only MiMe alleles of the first MiMe component of the first parent MiMe maize plant is the same as at least one of the MiMe loci having only MiMe alleles of the first MiMe component of the second parent MiMe maize plant. In some variations, at least one of the MiMe loci having only MiMe alleles of the third MiMe component of the first parent MiMe maize plant is the same as at least one of the MiMe loci having only MiMe alleles of the third MiMe component of the second parent MiMe maize plant. In other variations, the one or more MiMe loci having only MiMe alleles of the third MiMe component of the first parent MiMe maize plant are distinct from the one or more MiMe loci having only MiMe alleles of the third MiMe component of the second parent MiMe maize plant. In some embodiments, (a) the first parent MiMe maize plant has only MiMe alleles at one or more MiMe loci of a component of sister chromatid cohesion during the first division of meiosis, only MiMe alleles at a first MiMe locus of a component of DNA double strand breakage during meiotic recombination, only non-MiMe alleles at a second MiMe locus of the component of DNA double strand breakage during meiotic recombination, and only MiMe alleles at one or more MiMe loci of a component of progression through the second division of meiosis; (b) the second parent MiMe maize plant has only MiMe alleles at one or more MiMe loci of the component of sister chromatid cohesion during the first division of meiosis, only non-MiMe alleles at the first MiMe locus of the component of DNA double strand breakage during meiotic recombination, only MiMe alleles at the second MiMe locus of component of DNA double strand breakage during meiotic recombination, and only MiMe alleles at one or more MiMe loci of the of a component of progression through the second division of meiosis; and (c) at least one of the MiMe loci having only MiMe alleles of the component of sister chromatid cohesion during the first division of meiosis of the first parent MiMe maize plant is the same as at least one of the MiMe loci having only MiMe alleles of the component of sister chromatid cohesion during the first division of meiosis of the second parent MiMe maize plant. In some variations, at least one of the MiMe loci having only MiMe alleles of the component of progression through the second division of meiosis of the first parent MiMe maize plant is the same as at least one of the MiMe loci having only MiMe alleles of the component of progression through the second division of meiosis of the second parent MiMe maize plant. In other variations, the one or more MiMe loci having only MiMe alleles of the component of progression through the second division of meiosis of the first parent MiMe maize plant are distinct from the one or more MiMe loci having only MiMe alleles of the component of progression through the second division of meiosis of the second parent MiMe maize plant. Exemplary MiMe loci of each of said MiMe components are extensively described below. In certain embodiments, (a) the first parent MiMe maize plant has only MiMe alleles at REC8, only MiMe alleles at SPO11-1, only non-MiMe alleles at PAIR1, and only MiMe alleles at OSD1; and (b) the second parent MiMe maize plant has only MiMe alleles at REC8, only non-MiMe alleles at SPO11-1, only MiMe alleles at PAIR1, and only MiMe alleles at OSD1.

In other embodiments, each of the parent MiMe maize plants has a complete MiMe genotype comprising MiMe alleles conferring decreased expression of MiMe loci of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the parent MiMe maize plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. In some embodiments, (a) the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (b) the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component; and (c) at least one of the MiMe loci having only MiMe alleles of the first MiMe component of the first parent MiMe maize plant is the same as at least one of the MiMe loci having only MiMe alleles of the first MiMe component of the second parent MiMe maize plant. In certain embodiments, (a) the first parent MiMe maize plant has only MiMe alleles at one or more MiMe loci of a component of DNA double strand breakage during meiotic recombination, only MiMe alleles at a first MiMe locus of a component of progression through the first division of meiosis, and only non-MiMe alleles at a second MiMe locus of the component of progression through the first division of meiosis; (b) the second parent MiMe maize plant has only MiMe alleles at one or more MiMe loci of the component of DNA double strand breakage during meiotic recombination, only non-MiMe alleles at the first MiMe locus of the component of progression through the first division of meiosis, and only MiMe alleles at the second MiMe locus of the component of progression through the first division of meiosis; and (c) at least one of the MiMe loci having only MiMe alleles of the component of DNA double strand breakage during meiotic recombination of the first parent MiMe maize plant is the same as at least one of the MiMe loci having only MiMe alleles of the component of DNA double strand breakage during meiotic recombination of the second parent MiMe maize plant. In certain embodiments, (a) the first parent MiMe maize plant has only MiMe alleles at one or more MiMe loci of a component of progression through the first division of meiosis, only MiMe alleles at a first MiMe locus of a the component of DNA double strand breakage during meiotic recombination, and only non-MiMe alleles at a second MiMe locus of the component of DNA double strand breakage during meiotic recombination; (b) the second parent MiMe maize plant has only MiMe alleles at one or more MiMe loci of the component of progression through the first division of meiosis, only non-MiMe alleles at the first MiMe locus of the component of DNA double strand breakage during meiotic recombination, and only MiMe alleles at the second MiMe locus of the component of DNA double strand breakage during meiotic recombination; and (c) at least one of the MiMe loci having only MiMe alleles of the component of progression through the first division of meiosis of the first parent MiMe maize plant is the same as at least one of the MiMe loci having only MiMe alleles of the component of progression through the first division of meiosis of the second parent MiMe maize plant. Exemplary MiMe loci of each of said MiMe components are extensively described below. The parent MiMe maize plant may have any complete MiMe genotype known in the art or described herein, including, but not limited to, complete MiMe genotypes comprising MiMe alleles resulting in decreased expression of any of the MiMe loci described herein.

In some embodiments, the first parent MiMe maize plant has a complete MiMe genotype comprising MiMe alleles conferring decreased expression of one or more MiMe loci of a first, second, and third MiMe component, wherein the first MiMe component is (2) a component of DNA double strand breakage during meiotic recombination, the second MiMe component is (1) a component of sister chromatid cohesion during the first division of meiosis and the third MiMe component is (3) a component of progression through the second division of meiosis, and the second parent MiMe maize plant has a complete MiMe genotype comprising MiMe alleles conferring decreased expression of MiMe loci of the first MiMe component and a fourth MiMe component, wherein the fourth MiMe component is (4) a component of progression through the first division of meiosis. In some variations, the second parent MiMe maize plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. In certain embodiments, (a) the first parent MiMe maize plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination; (b) the second parent MiMe maize plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of a fourth MiMe component; and (c) at least one of the MiMe loci having only MiMe alleles of the first MiMe component of the first parent MiMe maize plant is the same as at least one of the MiMe loci having only MiMe alleles of the first MiMe component of the second parent MiMe maize plant. In some variations, (a) the first parent MiMe maize plant has only MiMe alleles at one or more MiMe loci of a component is a component of DNA double strand breakage during meiotic recombination, only MiMe alleles one or more MiMe loci of a component of sister chromatid cohesion during the first division of meiosis, only MiMe alleles at one or more MiMe loci of a component of progression through the second division of meiosis, and only non-MiMe alleles at one or more MiMe loci of a component of progression through the first division of meiosis; and (b) the second parent MiMe maize plant has only MiMe alleles at the one or more MiMe loci of the component of DNA double strand breakage during meiotic recombination, only non-MiMe alleles at the one or more MiMe loci of the component of sister chromatid cohesion during the first division of meiosis, only non-MiMe alleles at the one or more MiMe loci of the component of progression through the second division of meiosis, and only MiMe alleles at the one or more MiMe loci of the component of progression through the first division of meiosis. Exemplary MiMe loci of each of said MiMe components are extensively described below. In one variation, (a) the first parent MiMe maize plant has only MiMe alleles at SPO11-1, only MiMe alleles at REC8, only MiMe alleles at OSD1, and only non-MiMe alleles at PS1 and JASON; (b) the second parent MiMe maize plant has only MiMe alleles at SPO11-1, only non-MiMe alleles at REC8, only non-MiMe alleles at OSD1, and only MiMe alleles at PS1 or JASON.

In some embodiments, the first parent MiMe maize plant, the second parent MiMe maize plant, or both has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some variations, the one or more OSD1 loci comprise OSD1-1, OSD1-2, and/or OSD1-3, and/or the one or more PAIR1 loci comprise PAIR1-1 and/or PAIR1-2.

In some embodiments, the first parent MiMe maize plant, the second parent MiMe maize plant, or both has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some variations, the one or more OSD1 loci comprise OSD1-1, OSD1-2, and/or OSD1-3.

In some embodiments, the first parent MiMe maize plant, the second parent MiMe maize plant, or both has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1.

In some embodiments, the first parent MiMe maize plant, the second parent MiMe maize plant, or both has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some variations, the one or more PAIR1 loci comprise PAIR1-1 and/or PAIR1-2.

In some embodiments, the first parent MiMe maize plant, the second parent MiMe maize plant, or both has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1.

In some embodiments, the first parent MiMe maize plant, the second parent MiMe maize plant, or both has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIRLIn some variations, the one or more PAIR1 loci comprise PAIR1-1 and/or PAIR1-2.

In some embodiments, the first parent MiMe maize plant, the second parent MiMe maize plant, or both comprise one or more polynucleotide sequences selected from the group consisting of SEQ ID NOs: 108-111. In certain embodiments the first parent MiMe maize plant, the second parent MiMe maize plant, or both comprises a) a MiMe allele at one or more OSD1-2 loci, each independently comprising a polynucleotide sequence selected from the group consisting of SEQ ID NOs: 108 and 1109 a MiMe allele at a REC8 locus comprising the polynucleotide sequence of SEQ ID NO: 110: and/or c) a MiMe allele at a SPO11-1 locus comprising the polynucleotide sequence of SEQ ID NO: 111. In some Variations, each the one or more OSD1-2 foci, each of the one or more REC8 loci. and/or each of the one or more SPO11-1 loci are present on a different homologous chromosome.

Providing Haploid Gametes

In some embodiments, the method of producing a population of polyploid maize seed comprises providing haploid gametes from a homozygous parent non-MiMe maize plant. In some embodiments, providing haploid gametes comprises growing one or more homozygous parent non-MiMe maize plants and allowing them to grow to a reproductive stage until haploid gametes form. In some variations, providing haploid gametes further comprises collecting haploid pollen from one or more homozygous parent non-MiMe maize plants. In some additional variations, the one or more homozygous parent non-MiMe maize plants may be haploid, monoploid, diploid, triploid, tetraploid, pentaploid, hexaploid, heptaploid, octaploid, or any combination thereof. In some embodiments, the homozygous parent non-MiMe maize plant is diploid and the haploid gametes are monoploid gametes.

In some embodiments, the method of producing a population of polyploid maize seed comprises (a) providing clonal gametes from a parent MiMe maize plant; and (b) providing haploid (e.g., monoploid) gametes from a homozygous parent non-MiMe maize plant, wherein the clonal gametes and the haploid (e.g., monoploid) gametes together comprise three or more haplotypes. In some variations, the clonal gametes and the haploid gametes together comprise three or more haplotypes, four or more haplotypes, five or more haplotypes, six or more haplotypes, seven or more haplotypes, or eight or more haplotypes. In additional variations the clonal gametes and the haploid gametes together comprise three, four, five, six, seven, or eight haplotypes. In some embodiments, the haploid gametes comprise one haplotype and the clonal gametes comprise two or more haplotypes, wherein the clonal gametes and the haploid gametes together comprise three or more haplotypes.

Providing Unreduced, Non-Clonal Gametes

In some embodiments, the method of producing a population of polyploid maize seed comprises providing unreduced, non-clonal gametes. In some embodiments, the method of producing a population of polyploid maize seed comprises providing unreduced, non-clonal gametes from one or more parent maize plants. In certain embodiments, providing unreduced, non-clonal gametes comprises: growing a parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis; and allowing the parent maize plant to grow to a reproductive stage until unreduced, non-clonal gametes form. In other embodiments, providing unreduced, non-clonal gametes comprises: generating a parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis; and growing said parent maize plant to a reproductive stage until unreduced, non-clonal gametes form. In some variations, providing unreduced, non-clonal gametes further comprises collecting pollen comprising unreduced, non-clonal gametes from a parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. The MiMe loci of a of a component of progression through the first division of meiosis may be any loci of a component of progression through the first division of meiosis known in the art or described herein, including, but not limited to, PS1 and JASON. The MiMe loci of a component of a component of progression through the second division of meiosis may be any loci of a component of a component of progression through the second division of meiosis known in the art or described herein, including, but not limited to, OSD1, CYCA1, TDM1, PC1, PC2, and FC. In certain embodiments, the parent maize plant is a plant of an inbred maize line. In some variations, the parent maize plant is diploid, triploid, tetraploid, pentaploid, hexaploid, heptaploid, octaploid, or any combination thereof.

Crossing Clonal Gametes

In some embodiments, the method of producing a population of polyploid maize seed comprises crossing the clonal gametes to produce the population of polyploid maize seed. In some variations, crossing the clonal gametes to produce the population of polyploid maize seed comprises contacting pollen comprising clonal gametes with the stigma of a pistil comprising clonal gametes. In some variations, crossing the clonal gametes to produce the population of polyploid maize seed comprises contacting pollen comprising clonal gametes of a first maize plant with the stigma of a pistil comprising clonal gametes of a second maize plant. In some embodiments, crossing the clonal gametes to produce the population of polyploid maize seed comprises contacting pollen comprising clonal gametes from a first parent MiMe maize plant with the stigma of a pistil comprising clonal gametes of a second parent MiMe maize plant. In some embodiments, the population of polyploid maize seed is produced by crossing the clonal gametes and allowing seeds to form.

In other embodiments, the method of producing a population of polyploid maize seed comprises crossing clonal gametes with haploid gametes to produce the population of polyploid maize seed. In one variation, crossing the clonal gametes with the haploid gametes to produce the population of polyploid maize seed comprises contacting pollen comprising clonal gametes with the stigma of a pistil comprising haploid gametes. In another variation, crossing the clonal gametes with the haploid gametes to produce the population of polyploid maize seed comprises contacting pollen comprising haploid gametes with the stigma of a pistil comprising clonal gametes. In some embodiments, crossing the clonal gametes with the haploid gametes to produce the population of polyploid maize seed comprises contacting pollen comprising clonal gametes from a parent MiMe maize plant with the stigma of a pistil comprising haploid gametes of a homozygous parent non-MiMe maize plant. In other embodiments, crossing the clonal gametes with the haploid gametes to produce the population of polyploid maize seed comprises contacting pollen comprising haploid gametes from a homozygous parent non-MiMe maize plant with the stigma of a pistil comprising clonal gametes of a parent MiMe maize plant. In some variations, the homozygous parent non-MiMe maize plant and the haploid gametes are monoploid gametes. In some embodiments, the population of polyploid maize seed is produced by crossing the clonal gametes with the haploid gametes and allowing seeds to form.

In yet other embodiments, the method of producing a population of polyploid maize seed comprises crossing clonal gametes with unreduced, non-clonal gametes to produce the population of polyploid maize seed. In one variation, crossing the clonal gametes with the unreduced, non-clonal gametes to produce the population of polyploid maize seed comprises contacting pollen comprising clonal gametes with the stigma of a pistil comprising unreduced, non-clonal gametes. In another variation, crossing the clonal gametes with the unreduced, non-clonal gametes to produce the population of polyploid maize seed comprises contacting pollen comprising unreduced, non-clonal gametes with the stigma of a pistil comprising clonal gametes. In some embodiments, crossing the clonal gametes with the unreduced, non-clonal gametes to produce the population of polyploid maize seed comprises contacting pollen comprising clonal gametes from a parent MiMe maize plant with the stigma of a pistil comprising unreduced, non-clonal gametes of a homozygous parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, crossing the clonal gametes with the unreduced, non-clonal gametes to produce the population of polyploid maize seed comprises contacting the stigma of a pistil comprising clonal gametes from a parent MiMe maize plant with pollen comprising unreduced, non-clonal gametes of a homozygous parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, the population of polyploid maize seed is produced by crossing the clonal gametes with the unreduced, non-clonal gametes and allowing seeds to form.

In some embodiments, crossing clonal gametes to produce the population of polyploid seed comprises (a) collecting pollen from a first parent MiMe maize plant having only MiMe alleles at the one or more MiMe loci of the component of DNA double strand breakage during meiotic recombination, only non-MiMe alleles at the one or more MiMe loci of the component of sister chromatid cohesion during the first division of meiosis, only non-MiMe alleles at the one or more MiMe loci of the component of progression through the second division of meiosis, and only MiMe alleles at the one or more MiMe loci of the component of progression through the first division of meiosis; and (b) contacting the pollen from the first parent MiMe maize plant with a stigma of a second parent MiMe maize plant having only MiMe alleles at one or more MiMe loci of a component is a component of DNA double strand breakage during meiotic recombination, only MiMe alleles one or more MiMe loci of a component of sister chromatid cohesion during the first division of meiosis, only MiMe alleles at one or more MiMe loci of a component of progression through the second division of meiosis, and only non-MiMe alleles at one or more MiMe loci of a component of progression through the first division of meiosis.

Methods of Breeding Polyploid Hybrid Maize Lines Comprising Three or More Haplotypes

In yet another aspect, provided herein are methods of breeding a polyploid hybrid maize line comprising three or more haplotypes, comprising obtaining a set of maize lines; breeding the lines using traditional maize breeding methods to produce a set of candidate maize lines; and selecting two or more candidate maize lines together comprising three or more haplotypes for crossing. In some embodiments, after the selection of candidate maize lines, the methods further comprise generating two parent MiMe maize plants from the two or more candidate maize lines; providing clonal gametes from each of the parent MiMe maize plants; and crossing the clonal gametes to produce a hybrid polyploid seed comprising the three or more haplotypes. In alternative embodiments, after the selection of candidate maize lines, the methods further comprise generating a single parent MiMe maize plant from one of the two or more candidate maize lines; providing clonal gametes from the parent MiMe maize plant; providing haploid (e.g., monoploid) gametes from a homozygous parent non-MiMe maize plant of one of the two or more candidate maize lines; and crossing the clonal gametes with the haploid (e.g., monoploid) gametes to produce a hybrid polyploid seed. In yet additional embodiments, after the selection of candidate maize lines, the methods further comprise generating a single parent MiMe maize plant from one of the two or more candidate maize lines; providing clonal gametes from the parent MiMe maize plant; providing unreduced, non-clonal gametes from homozygous parent maize plant of one of the two or more candidate maize lines; and crossing the clonal gametes with the unreduced, non-clonal gametes to produce a hybrid polyploid seed. In some embodiments, after the crossing of the clonal gametes or the crossing of the clonal gametes with the haploid (e.g., monoploid) gametes, or crossing of the clonal gametes with unreduced, non-clonal gametes, the methods further comprise growing the hybrid polyploid seed to produce a hybrid polyploid maize plant and evaluating one or more characteristics of the hybrid polyploid maize plant.

In still another aspect, provided herein are methods of breeding hybrid polyploid maize plants, comprising obtaining a set of maize lines; breeding the lines using traditional maize breeding methods to produce a set of candidate maize lines; selecting two or more candidate maize lines for crossing; and generating a first parent MiMe maize plant and a second parent MiMe maize plants from the two or more candidate maize lines. In some embodiments, the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; and the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe maize plant is the same as at least one of the MiMe loci having only MiMe alleles of the first MiMe component of the second parent MiMe maize plant. In some embodiments, the first and second parent MiMe maize plants further have only MiMe alleles at one or more MiMe loci of a third MiMe component, wherein the one or more MiMe loci having only MiMe alleles of the third MiMe component of the first and second parent MiMe maize plants are the same or different. In certain embodiments, the parent MiMe maize plants together comprise two, three, or more haplotypes. In some embodiments, the methods further comprise providing clonal gametes from each of the parent MiMe maize plants; crossing the clonal gametes to produce a polyploid seed; growing the polyploid seed to produce a hybrid polyploid maize plant; and evaluating one or more characteristics of the hybrid polyploid maize plant.

Obtaining Maize Lines

In some embodiments, the method of breeding a polyploid hybrid maize line comprising two, three, or more haplotypes comprises obtaining a set of maize lines. In preferred embodiments, the set of maize lines is genetically diverse and comprises a large and diverse pool of haplotypes. In some embodiments, the method of breeding a polyploid hybrid maize line comprising two, three, or more haplotypes comprises obtaining a set of maize lines of the same or related species of maize plant. The set of maize lines may be obtained from any source and by any methods known in the art. In some embodiments, the set of maize lines is obtained from sources including, but not limited to, natural diversity, existing breeding programs, or any combination thereof.

In some embodiments, the set of maize plant lines is a genetically diverse founder population of maize plants. These maize plants may be collected from existent diploid germplasm sources such as wild species, progenitor species and landraces or from recent diploid inbred line-based breeding efforts (Jansky et al. 2016. Reinventing potato as a diploid inbred line-based crop. Crop Science 56, no. 4: 1412-1422.). In some instances, these maize plants may have high genetic load and may not have undergone the narrowing of genetic variability attributable to the elite selection practices imposed on modern cultivated materials. As such, many possess traits promoting their fitness within non-agrarian environments in the case of wild progenitors, or have been adapted to vastly differing cultivation practices and agricultural environments in the case of landraces. In either instance, these founding maize plant materials may contain suites of both desirable and undesirable agronomic characteristics that may be recombined, selected, and complemented to develop a commercially viable product.

In some embodiments, the method of breeding a polyploid hybrid maize line comprising two, three, or more haplotypes comprises obtaining a set of diploid maize lines. The diploid maize lines may be obtained by any methods known in the art or described herein.

In certain embodiments, the obtaining a set of maize lines comprises creating reduced-ploidy maize lines by haploid induction of polyploid maize lines. Haploid induction may be achieved maize by, for example, interspecific hybridization with a haploid inducer, intraspecific hybridization with a haploid inducer, or modification of CENH3 activity or expression (e.g. by RNA interference and/or expression of a modified CENH3 protein). Methods of haploid induction are well known in the literature, and examples are described Wan, et al. (1989. Efficient production of doubled haploid plants through colchicine treatment of anther-derived maize callus. Theor. Appl. Genet., 77:889-892.) and Ren et al. (2017. Novel technologies in doubled haploid line development. Plant Biotechnol J 15, 1361-1370.) and references cited therein. In some variations, the obtaining a set of maize lines comprises haploid induction of one or more tetraploid lines to create one or more diploid lines. In other variations, the obtaining a set of maize lines comprises haploid induction of hexaploid, octaploid, or higher even-ploidy lines to create one or more lines of half the original ploidy.

Heterotic Groups

In some embodiments, the method of breeding a polyploid hybrid maize line comprises organizing the set of maize lines into two, three, or more heterotic groups, wherein each heterotic group comprises a haplotype, and wherein the haplotypes are grouped based on observed or predicted heterotic performance when combined in the hybrid polyploid maize plant. In some embodiments, the method of breeding a polyploid hybrid maize line comprises organizing the set of maize lines into four or more heterotic groups. In some variations, the method of breeding a polyploid hybrid maize line comprises organizing the set of maize lines into five or more heterotic groups, six or more heterotic groups, seven or more heterotic groups, or eight or more heterotic groups.

The obtained set of maize lines may be organized by assigning their membership into complementary heterotic groups based upon both their empirically-defined Endosperm Balance Number for the genus in question (Ortiz and Ehlenfeldt. 1992. The importance of Endosperm Balance Number in potato breeding and the evolution of tuber-bearing Solanum species. Euphytica 60, 105-113; Arisumi. 1982. Endosperm balance numbers among New Guinea-Indonesian Impatiens species, Journal of Heredity, 73:3, 240-242; Birchler. 1993. Dosage analysis of maize endosperm development. Annu Rev Genet. 27:181-204; Nishiyama et al. 2007. Embryological studies on cross-incompatibility between 2× and 4× in Brassica, Japanese Journal of Genetics, 41:1, 27-42) and heterotic patterns observed from preliminary estimation or prediction of their combining ability for traits and environments of interest at the diploid and polyploid levels. In this context, the combining ability is an estimation of the value of a plant as a parent as inferred by progeny testing in an established factorial or hierarchical mating design. Combining ability at the polyploid hybrid level is of primary importance; however, sufficient diploid hybrid performance is necessary to permit the diploid hybrid to serve as a parent for seed production. Furthermore, assessment of combining abilities at the simplified diploid level will better inform predictive models of genetic architecture.

In defining heterotic groups, the main goal is identifying subpopulations of the set of maize lines based on employment of a clustering procedure that maximizes some measure of interpopulation combining ability. Exhaustive evaluation of all possible parental combinations for the final polyploid hybrid, as in the case of a factorial mating scheme, is infeasible for all but a trivial number of potential parents (i.e. even a partial diallel ignoring reciprocal crosses scales at (n+3)!/(4!(n−1)!) crosses per n parents). Yet, hierarchical mating schemes necessitate an understanding and judgment of which set of “testers” or analogous constructs should serve as a relevant and efficient basis or frame of reference for inferring combining ability. The sample of testers selected invariably biases perceptions of existing heterotic patterns. Furthermore, the relative importances of traits and environments of interest used to infer these combining abilities are dynamic and depend upon market trends. As such, predictive modeling is essential and the process of assigning and refining heterotic groups and testers to represent them is one of iterative improvement and refinement throughout repeated cycles of the breeding process. Nonetheless, once preliminary heterotic group membership is assigned, interpopulation improvement of the diploid germplasm and development of polyploid hybrid maize plants may proceed.

Breeding Maize Lines

In some embodiments, the method of breeding a polyploid hybrid maize line comprising three or more haplotypes comprises breeding the lines using traditional plant breeding methods to produce a set of candidate lines of maize. The method may comprise breeding the lines using any traditional plant breeding method known in the art or described herein. In some embodiments, the breeding of the lines comprises reciprocal recurrent selection. In additional embodiments, the breeding of the lines comprises inbreeding one or more of the lines to homozygosity. In some variations, the breeding of the lines comprises crossing, selfing (self-pollinating), and backcrossing the lines to produce candidate lines. In additional variations, the breeding of the lines comprises crossing pairs of the lines to generate an F1 (first filial) generation, followed by several generations of selfing (generating F2, F3, etc.). In yet additional variations, the breeding of the lines comprises backcrossing (BC) steps, whereby the offspring is backcrossed to one of the parental lines, termed the recurrent parent.

There are numerous steps that may be taken in breeding the lines of maize using traditional plant breeding methods to produce a set of candidate lines of maize. The choice of breeding method depends on the mode of plant reproduction and the heritability of the trait(s) being improved. Backcross breeding may be used to transfer one or a few favorable genes for a highly heritable trait into a desirable line. This approach has been used extensively for breeding disease-resistant lines. Various recurrent selection techniques may be used to improve quantitatively inherited traits controlled by numerous genes. The use of recurrent selection in self-pollinating crops depends on the ease of pollination, the frequency of successful hybrids from each pollination, and the number of hybrid offspring from each successful cross. A breeder can initially select and cross two or more parental lines, followed by repeated selfing and selection, producing many new genetic combinations. Moreover, a breeder can generate multiple different genetic combinations by crossing, selfing, generating mutations, or any combination thereof. A plant breeder can then select which lines to select as candidate lines. Recurrent selection techniques are reviewed in Vasal et al. (2004. Population Improvement Strategies for Crop Improvement. In: Plant Breeding. Springer, p 391-406).

The development of candidate lines for the methods described herein may include obtaining parental lines, crossing of these lines, and evaluating the crosses. Pedigree breeding and recurrent selection breeding methods may be used to develop candidate lines from breeding populations. Breeding programs may combine desirable traits from two or more varieties or various broad-based sources into breeding pools from which lines are developed by selfing and selection of desired phenotypes. The new lines may be further crossed with other lines and the hybrids from these crosses may be evaluated for potential selection as candidate lines.

Choice of breeding or selection methods depends on the mode of plant reproduction and the heritability of the trait(s) being improved. For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

In some embodiments, the breeding of the maize lines comprises inbreeding one or more of the lines to homozygosity. In some variations, inbreeding a maize line to homozygosity may comprise selfing plants of the line for two or more generations, such as for five to seven generations, to produce an inbred or homozygous maize line. Homozygous maize lines may also be developed by the production of double haploids. Double haploids are produced by generating a haploid plant from a heterozygous plant and the doubling of the genome of the haploid plant to produce a completely homozygous individual. The process of generating a haploid plant is also known as haploid induction. Haploid induction can be achieved in a variety of plants using methods well-known in the art and described herein. After a haploid plant is generated, genome doubling may occur spontaneously or may be achieved artificially using, for example, colchicine, amiprophos-mehtyl (APM), oryzalin, pronamide, trifluralin, or nitrous oxide. Methods of producing double haploids are well known in the literature, and examples are described Wan, et al. (1989. Efficient production of doubled haploid plants through colchicine treatment of anther-derived maize callus. Theor. Appl. Genet., 77:889-892.) and Ren et al. (2017. Novel technologies in doubled haploid line development. Plant Biotechnol J 15, 1361-1370.) and references cited therein.

Pedigree breeding is used commonly for the improvement of self-pollinating crops or inbred lines of cross-pollinating crops. Two parents which possess favorable, complementary traits are crossed to produce an F1 population. An F2 population is produced by selfing one or several F1 s or by intercrossing two F1 s (sib mating). Selection of the best individuals may begin in the F2 population; then, beginning in the F3, the best individuals in the best families may be selected. Replicated testing of families, or hybrid combinations involving individuals of these families, may follow in the F4 generation to improve the effectiveness of selection for traits with low heritability. At an advanced stage of inbreeding (i.e., F6 and F7), the best lines or mixtures of phenotypically similar lines may be tested for potential selection as candidate lines.

Mass and recurrent selections can be used to improve lines of either self- or cross-pollinating crops. A genetically variable population of heterozygous individuals may be either identified or created by intercrossing several different parents. The best plants are selected based on individual superiority, outstanding progeny, or heterotic performance. The selected plants are intercrossed to produce a new population in which further cycles of selection are continued.

Backcross breeding may be used to transfer genes for a simply inherited, highly heritable trait into a desirable homozygous line that is the recurrent parent. The source of the trait to be transferred is called the donor parent. The resulting plant is expected to have the attributes of the recurrent parent and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generational advancement is completed.

In addition to phenotypic observations, the genotype of a plant can also be examined during breeding to produce candidate lines. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of lines as parental varieties or ancestors of a plant by providing a means of tracking genetic profiles through crosses.

Mutation breeding may also be used in the breeding of maize lines to produce candidate lines. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into an existing line by traditional breeding techniques. Details of mutation breeding can be found in Principles of Cultivar Development by Fehr, Macmillan Publishing Company, 1993.

Additional non-limiting examples of breeding methods that may be used include, without limitation, those found in Allard (1960. Principles of Plant Breeding, John Wiley and Son, pp. 115-161). Simmonds (1979. Principles of Crop Improvement, Longman Group Limited), Sneep (1979. Plant Breeding Perspectives, Unipub), and Fehr and Walt (1987. Principles of Cultivar Development, pp. 261-286).

In certain embodiments, breeding the lines of maize comprises generating and maintaining one or more maize lines having complete or partial MiMe genotypes. The one or more maize lines having complete or partial MiMe genotypes may be maintained via selfing, apomixis, cell culture, or any combination thereof. The MiMe alleles of the one or more maize lines having partial MiMe genotypes may be propagated across breeding cycles to reduce the number of required editing or transgenesis events to introduce partial or complete MiMe genotypes into candidate lines of maize. In some embodiments, each of the heterotic groups may be further divided into two subgroups, resulting in each heterotic group containing a small subgroup of maize lines having a partial MiMe genotype, maintained through outbreeding and stabilizing selection for the partial MiMe genotype and a wild-type meiosis phenotype, and another larger subgroup of maize lines that do not comprise MiMe alleles. The subgroup containing the maize lines having a partial MiMe genotype may be slower-cycling during breeding, as genotyping and stabilizing selection for the partial MiMe genotype and wild-type meiosis phenotype may be undertaken in some or all progeny. Within this subgroup, a subset of all possible intragroup crosses, which are expected to bear some offspring possessing the partial MiMe genotype and a wild-type meiosis phenotype, is selected and realized by considering the following criteria: 1) per se performance of the maize line, 2) genotype or pedigree-based estimation or prediction of breeding value and combining ability with regard to complementing the alternate subgroup within the heterotic group as well as the other heterotic groups at the polyploid hybrid level, and 3) management of inbreeding with respect to both subgroups of the heterotic group. After genotyping and selecting the subset of realized recombinant diploid progeny having a partial MiMe genotype and a wild-type meiosis phenotype, these maize plants may serve in the following breeding roles: 1) field evaluations to improve the estimation or prediction of breeding values and combining abilities, 2) testers to aid heterotic group (re)assignment, 3) progenitors in recurrent cycles of diploid intergroup improvement to increase desirable allele frequencies and linkage disequilibria, and/or 4) great-grandparents in tetraploid hybrid maize development.

In some embodiments, breeding the lines of maize comprises (i) introducing a partial MiMe genotype into one or more lines of maize within a heterotic group to produce a maize line having a partial MiMe genotype, wherein the maize line having a partial MiMe genotype comprises one or more haplotypes, (ii) crossing the maize line having the partial MiMe genotype with itself or another maize line, and (iii) propagating the maize line having the partial MiMe genotype by selecting for the partial MiMe genotype in the offspring of the cross of step (ii). In some variations, step (ii) comprises crossing the maize line having the partial MiMe genotype with itself. In other variations, step (ii) comprises crossing the maize line having the partial MiMe genotype with another plant within the same heterotic group or a different heterotic group.

In some embodiments, haploid induction may be used to maintain haploid, diploid, or polyploid maize lines having a complete MiMe genotype. In certain embodiments, maintaining a polyploid maize line having a complete MiMe genotype comprises crossing a polyploid maize having a complete MiMe genotype with a haploid inducer to produce progeny of the same ploidy as the plant having the complete MiMe genotype. In one variation, maintaining a polyploid maize line having a complete MiMe genotype comprises crossing a tetraploid maize plant having a complete MiMe genotype with a haploid inducer to produce tetraploid progeny.

Selecting Candidate Maize Lines

In some embodiments, the method of breeding a polyploid hybrid maize line comprising two, three, or more haplotypes comprises selecting two or more candidate maize lines together comprising two, three, or more haplotypes. In some variations, the method of breeding a hybrid polyploid plant comprises selecting two or more candidate maize lines together comprising three or more haplotypes or four or more haplotypes. In some embodiments, one or more of the candidate maize lines are inbred maize lines. In additional embodiments, one or more of the candidate maize lines are hybrid maize lines. In certain embodiments, the selecting of candidate maize lines is guided by the one or more characteristics of a hybrid polyploid plant comprising the two, three, or more haplotypes comprised by the two or more candidate maize lines. In certain embodiments, the selecting of candidate maize lines is guided by the one or more characteristics of a hybrid polyploid plant comprising the two or more haplotypes, three or more haplotypes, or four or more haplotypes comprised by the two or more candidate maize lines. In some variations, the selecting of candidate maize lines is guided by the observed or predicted heterotic performance of the two or more haplotypes, three or more haplotypes, or four or more haplotypes comprised by the two or more candidate maize lines.

Generating Parent MiMe Plants

In some embodiments, the method of breeding a polyploid hybrid maize line comprising two, three, or more haplotypes comprises generating one or more parent MiMe maize plants from one or more of the two or more candidate maize lines, wherein the parent MiMe maize plant has a complete MiMe genotype. In some variations, the method of breeding a polyploid hybrid maize line comprising two, three, or more haplotypes comprises generating two parent MiMe maize plants from two of the two or more candidate maize lines, wherein each of the parent MiMe maize plants has a complete MiMe genotype. In some embodiments, the method of breeding a hybrid polyploid maize plant comprises generating a first parent MiMe maize plant from one of the candidate maize lines and generating a second parent MiMe maize plant from one of the candidate maize lines, wherein the parent MiMe maize plant has a complete MiMe genotype. In some variations, the method of breeding a hybrid polyploid maize plant comprises generating two parent MiMe maize plants from two of the two or more candidate maize lines, wherein the parent MiMe maize plant has a complete MiMe genotype. In some embodiments, the complete MiMe genotypes of the one or more parent MiMe maize plants comprise alleles that are naturally-occurring, introduced via genetic modification, or a combination thereof. The one or more parent MiMe maize plants may have any complete MiMe genotype known in the art or described herein. The generating of the parent MiMe maize plants may comprise introducing any of the parent MiMe maize plant genotypes described herein (see “Parent MiMe Maize Plant Genotypes” and “Parent MiMe Maize Plant Complementary Genotypes”).

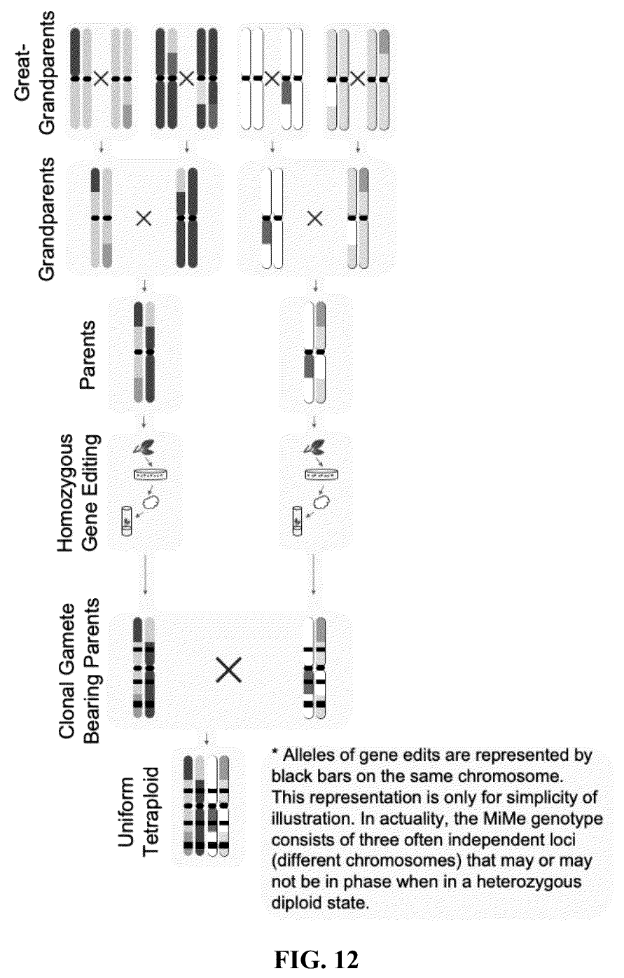

In certain embodiments, the generating of the one or more parent MiMe maize plants comprises introducing a complete MiMe genotype directly into one or more candidate maize lines to produce the one or more parent MiMe maize plants. In some variations, the generating of the two parent MiMe maize plants comprises introducing a complete MiMe genotype directly into two or more candidate maize lines to produce the two parent MiMe maize plants, for example, as shown in FIG. 12, 13, or 19.

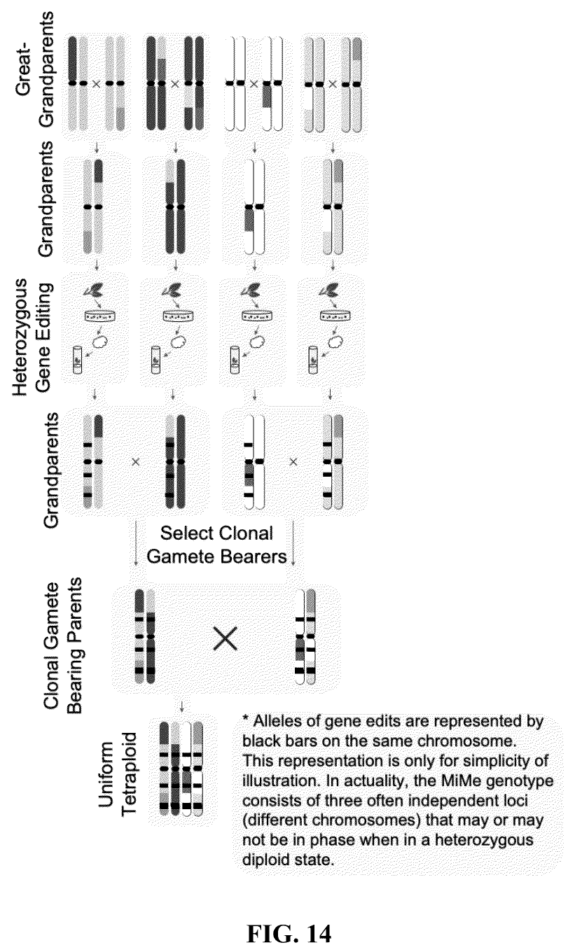

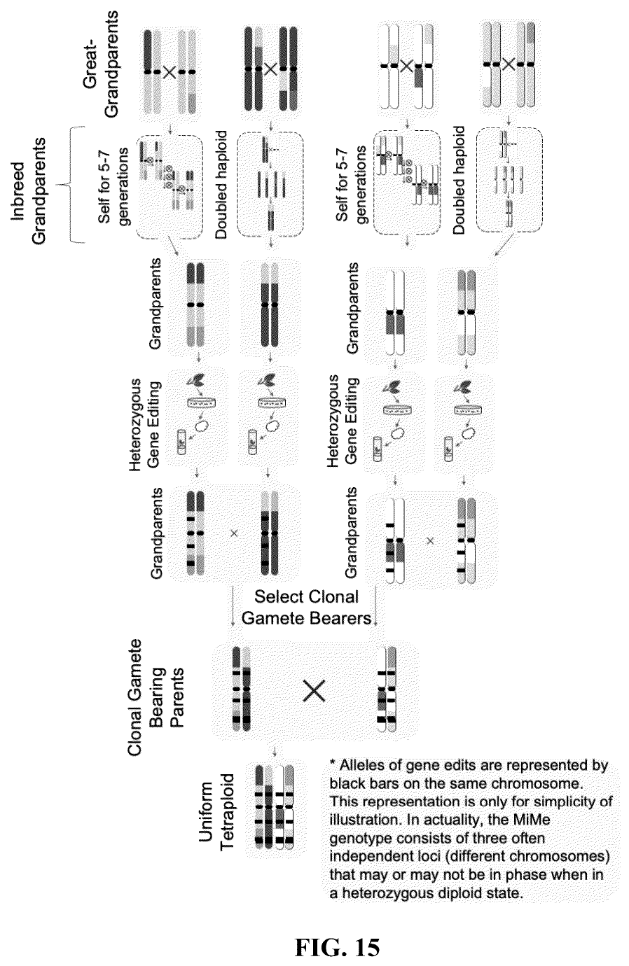

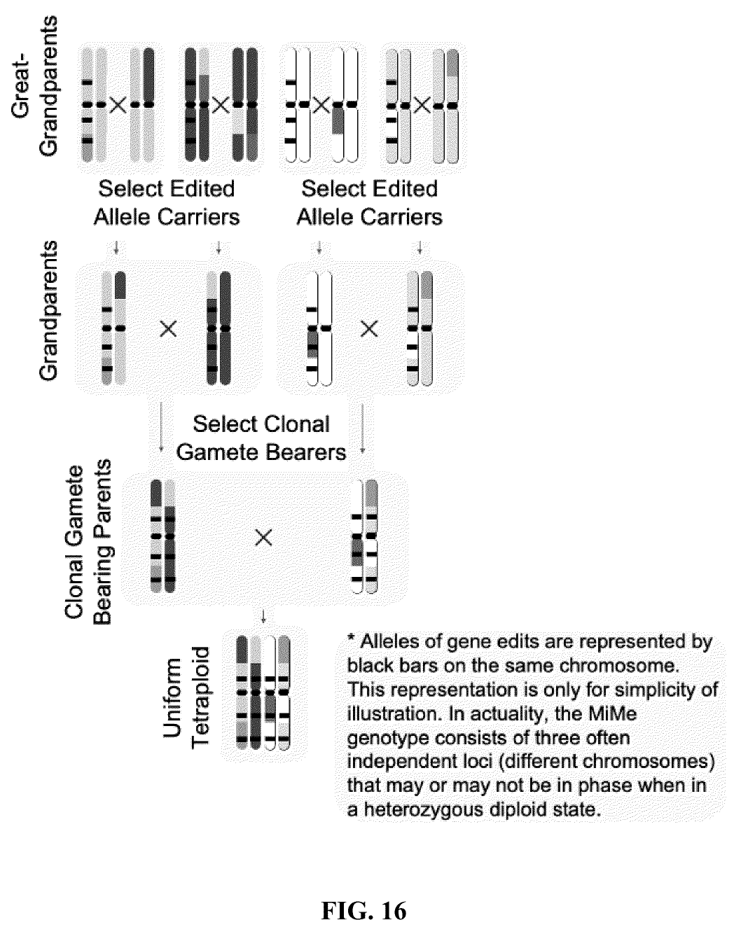

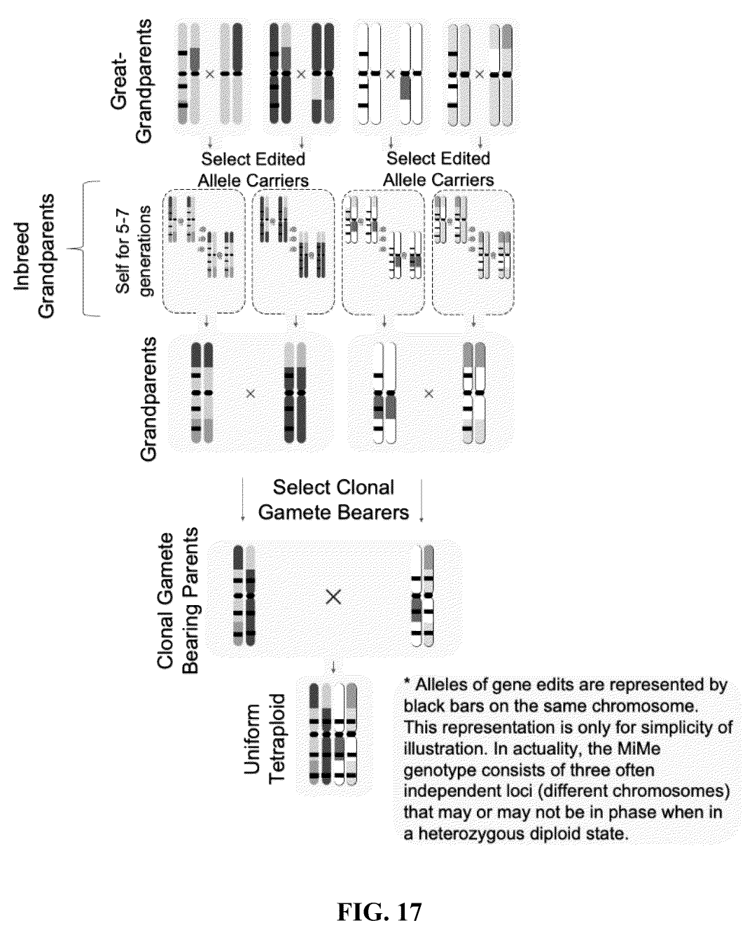

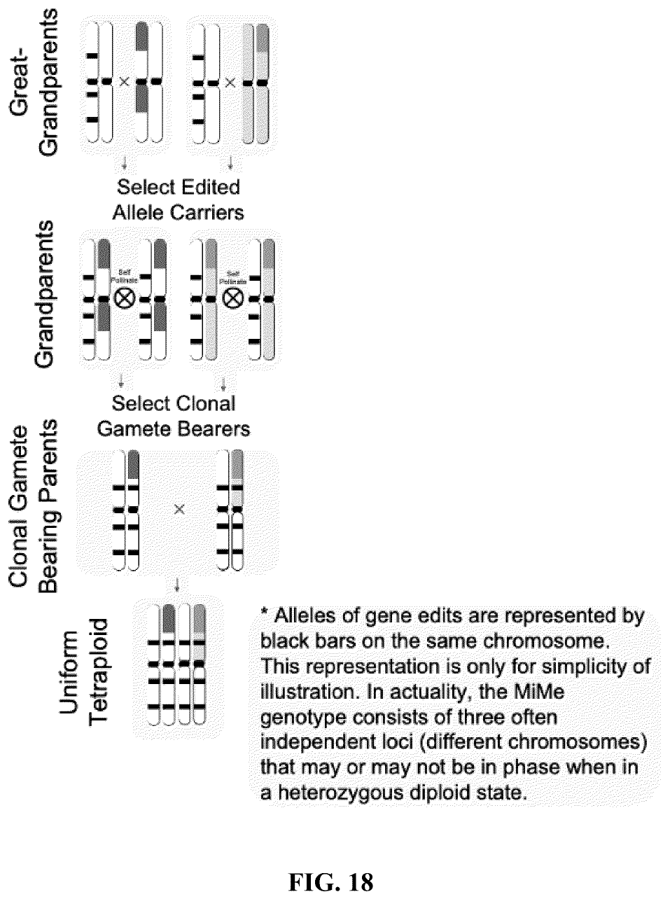

In certain embodiments, the generating of the one or more parent MiMe maize plants comprises introducing a partial MiMe genotype into two or more candidate maize lines to produce two or more grandparent non-MiMe maize plants each having a partial MiMe genotype and crossing said grandparent non-MiMe maize plants each having a partial MiMe genotype to produce the one or more parent MiMe maize plants, for example, as shown in FIG. 14 or 15. In some embodiments, the generating of the two parent MiMe maize plants comprises introducing a partial MiMe genotype into four candidate maize lines to produce four grandparent non-MiMe maize plants each having a partial MiMe genotype, and crossing pairs of said grandparent non-MiMe maize plants each having a partial MiMe genotype to produce the two parent MiMe maize plants, for example, as shown in FIG. 14 or 15. In other embodiments, the generating of the two parent MiMe maize plants comprises introducing a partial MiMe genotype into two candidate maize lines to produce two grandparent non-MiMe maize plants each having a partial MiMe genotype, crossing said grandparent non-MiMe maize plants each having a partial MiMe genotype to produce the first parent MiMe maize plant, and introducing a complete MiMe genotype directly into a third candidate maize line to produce the second parent MiMe maize plant. In additional embodiments, the generating of the one or more parent MiMe maize plants comprises introducing a partial MiMe genotype into a candidate line to produce a great-grandparent non-MiMe maize plant having a partial MiMe genotype, crossing the great-grandparent non-MiMe maize plant having a partial MiMe genotype with one or more candidate maize lines together comprising one or more haplotypes to produce a grandparent non-MiMe maize plant having a partial MiMe genotype and comprising two or more haplotypes, and crossing or selfing the grandparent non-MiMe maize plant having the partial MiMe genotype and comprising two or more haplotypes to produce a parent MiMe maize plant comprising two or more haplotypes, for example, as shown in any one of FIGS. 16-18. In one variation, the generating of the two parent MiMe maize plants comprises introducing a partial MiMe genotype into a candidate maize line to produce a great-grandparent non-MiMe maize plant having a partial MiMe genotype, crossing the great-grandparent non-MiMe maize plant having a partial MiMe genotype with two candidate maize lines together comprising two or more haplotypes to produce two grandparent non-MiMe maize plants together comprising three or more haplotypes, and selfing each of the grandparent non-MiMe maize plants together comprising three or more haplotypes to produce two parent MiMe maize plants together comprising three or more haplotypes, for example, as shown in FIG. 17 or 18. In one variation, the generating of the two parent MiMe maize plants comprises introducing a partial MiMe genotype into a candidate maize line to produce a great-grandparent non-MiMe maize plant having a partial MiMe genotype, crossing the great-grandparent non-MiMe maize plant having a partial MiMe genotype with two candidate maize lines together comprising two or more haplotypes to produce two grandparent non-MiMe maize plants together comprising three or more haplotypes, and crossing pairs of the grandparent non-MiMe maize plants together comprising three or more haplotypes to produce two parent MiMe maize plants together comprising three or more haplotypes, for example, as shown in FIG. 16. Any combination of methods for generating one or more parent MiMe maize plants described herein can be used in any combination to generate parent MiMe maize plants for use in the methods described herein. The grandparent non-MiMe maize plant or great-grandparent non-MiMe maize plant may have any partial MiMe genotype known in the art or described herein, including, but not limited to, partial MiMe genotypes comprising MiMe alleles resulting in decreased expression of any of the MiMe loci described herein.

In some embodiments, the grandparent non-MiMe maize plant has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the grandparent non-MiMe maize plant has a partial MiMe genotype comprising (a) one or more MiMe alleles conferring decreased expression of one or more MiMe loci of each of a first and second MiMe component; and (a) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. The grandparent non-MiMe maize plant or great-grandparent non-MiMe maize plant may have any partial MiMe genotype known in the art or described herein, including, but not limited to, partial MiMe genotypes comprising MiMe alleles resulting in decreased expression of any of the MiMe loci described herein.

In some embodiments, the generating of the parent MiMe maize plants comprises introducing a complete MiMe genotype into two candidate maize lines wherein (a) the first parent MiMe maize plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) generating a second parent MiMe maize plant from one of the candidate maize lines, wherein the second parent MiMe maize plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; (c) at least one of the MiMe loci having only MiMe alleles of the first MiMe component of the first parent MiMe maize plant is the same as at least one of the MiMe loci having only MiMe alleles of the first MiMe component of the second parent MiMe maize plant; and (d) either (1) at least one of the MiMe loci having only MiMe alleles of the third MiMe component of the first parent MiMe maize plant is the same as at least one of the MiMe loci having only MiMe alleles of the third MiMe component of the second parent MiMe maize plant, or (2) the one or more MiMe loci having only MiMe alleles of the third MiMe component of the first parent MiMe maize plant are distinct from the one or more MiMe loci having only MiMe alleles of the third MiMe component of the second parent MiMe maize plant. In some embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. Exemplary MiMe loci of each of said MiMe components are extensively described herein.

In other embodiments, the generating of the parent MiMe maize plants comprises introducing a complete MiMe genotype into two candidate maize lines wherein (a) the first parent MiMe maize plant has only MiMe alleles at one or more MiMe loci of a first MiMe component, only MiMe alleles at a first MiMe locus of a second MiMe component, and only non-MiMe alleles at a second MiMe locus of the second MiMe component; (b) the second parent MiMe maize plant has only MiMe alleles at one or more MiMe loci of the first MiMe component, only non-MiMe alleles at the first MiMe locus of the second MiMe component, and only MiMe alleles at the second MiMe locus of the second MiMe component; and (c) at least one of the MiMe loci having only MiMe alleles of the first MiMe component of the first parent MiMe maize plant is the same as at least one of the MiMe loci having only MiMe alleles of the first MiMe component of the second parent MiMe maize plant. In some embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. Exemplary MiMe loci of each of said MiMe components are extensively described herein.

In some embodiments, the grandparent non-MiMe maize plant or the great-grandparent non-MiMe maize plant has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more OSD1 loci comprise OSD1-1, OSD1-2, and/or OSD1-3, and/or the one or more PAIR1 loci comprise PAIR1-1 and/or PAIR1-2.

In some embodiments, the grandparent non-MiMe maize plant or the great-grandparent non-MiMe maize plant has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the one or more OSD1 loci comprise OSD1-1, OSD1-2, and/or OSD1-3.

In some embodiments, the grandparent non-MiMe maize plant or the great-grandparent non-MiMe maize plant has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the grandparent non-MiMe maize plant or the great-grandparent non-MiMe maize plant has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more PAIR1 loci comprise PAIR1-1 and/or PAIR1-2.

In some embodiments, the grandparent non-MiMe maize plant or the great-grandparent non-MiMe maize plant has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the grandparent non-MiMe maize plant or the great-grandparent non-MiMe maize plant has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the one or more PAIR1 loci comprise PAIR1-1 and/or PAIR1-2.

In some embodiments, the grandparent non-MiMe maize plant or the great-grandparent non-MiMe maize plant comprises one or more polynucleotide sequences selected from the group consisting of SEQ ID NOs: 108-111. In certain embodiments the grandparent non-MiMe maize plant or the great-grandparent non-MiMe maize plant comprises a) a MiMe allele t one or more OSD1-2 loci, each independently comprising a polynucleotide sequence selected from the group consisting of SEQ ID NOs: 108 and 109; b) a allele at a REC8 locus comprising the polynucleotide sequence of SEQ ID NO: 110; and/or c) a MiMe allele at a SPO11-1 locus comprising the polynucleotide sequence of SEQ ID NO: 111. In some variations, each the one or more OSD1-2 loci, each of the one or more REC8 loci, and/or each of the one or more SPO11-1 loci are present on a different homologous chromosome.

Methods of Introducing Genetic Modifications

In some embodiments, the method of breeding a polyploid hybrid maize line comprising two, three, or more haplotypes comprises generating one or more parent MiMe maize plants from one or more of the two or more candidate maize lines, wherein the parent MiMe maize plant has a complete MiMe genotype, and wherein the complete MiMe genotype comprises alleles that are introduced via genetic modification. Generating a parent MiMe maize plant having a complete MiMe genotype may comprise introducing a complete MiMe genotype via genetic modification directly into a candidate maize line to produce the parent MiMe maize plant; introducing a partial MiMe genotype via genetic modification into two candidate maize lines to produce two grandparent non-MiMe maize plants each having a partial MiMe genotype, and crossing the grandparent non-MiMe maize plants each having a partial MiMe genotype to produce the parent MiMe maize plant; or a combination thereof. The MiMe alleles of a complete or partial MiMe genotype may be introduced via genetic modification by any means known in the art. In some embodiments, the genetic modifications are introduced by gene editing, transgenesis, or a combination thereof.

In some embodiments, the methods described herein comprise introducing one or more genetic modifications into one or more MiMe loci that result in the decreased expression of the one or more MiMe loci. In some embodiments, decreased expression a MiMe locus may be achieved by modifying or replacing nucleotide sequences of interest (such as a regulatory elements), gene disruption, gene knockout, gene knockdown, gene knock-in, gene silencing (including, e.g., by inserting and/or expressing an inverted repeat into a gene of interest), RNA interference (including, e.g., by insertion and/or expression of an RNA interference construct), expression of a repressor protein (e.g. dCas9), modification of methylation status of gene loci, modification of splicing sites, introducing alternate splicing sites, or any combination thereof. In some variations, the genetic modification is introduced into the first 70%, the first 60%, the first 50%, the first 40%, the first 30%, the first 20%, or the first 10% of the nucleotides of the coding sequence of the MiMe locus following the start codon in the 3′ direction. In certain variations, the genetic modification is introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In certain variations, the genetic modification is introduced into a non-coding element of the MiMe locus (e.g., a promoter, an enhancer, a terminator, an intron, or the like).

In some embodiments, the decreased expression of each of the one or more of the MiMe loci is independently achieved by introducing an insertion, a deletion, one or more nucleotide changes, or an inversion into the MiMe locus that that results in decreased expression of the MiMe locus. In some variations, the insertion, the deletion, the one or more nucleotide changes, or the inversion is introduced into the first 70%, the first 60%, the first 50%, the first 40%, the first 30%, the first 20%, or the first 10% of the nucleotides of the coding sequence of the MiMe locus following the start codon in the 3′ direction. In certain variations, the insertion, the deletion, the one or more nucleotide changes, or the inversion is introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some variations, the insertion, the deletion, the one or more nucleotide changes, or the inversion eliminates expression (e.g., eliminates activity) of the MiMe locus. In some variations, the activity of the MiMe locus is eliminated by a premature stop codon introduced into the first 70%, the first 60%, the first 50%, the first 40%, the first 30%, the first 20%, or the first 10% of the nucleotides of the coding sequence of the MiMe locus following the start codon in the 3′ direction. In certain variations, the activity of the MiMe locus is eliminated by a premature stop codon introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction.

In certain embodiments, the genetic modifications are introduced by gene editing. Any of several gene editing methods known in the art may be used to introduce the genetic modifications of the complete or partial MiMe genotype. In some variations, gene editing is performed with one or more natural or engineered nucleases including, but not limited to, RNA-guided nucleases, meganucleases, zinc finger nucleases (ZFNs), and transcription activator-like effector-based nucleases (TALENs). In further variations, gene editing is performed with RNA-guided nucleases including, but not limited to, Clustered Regularly Interspaced Short Palindromic Repeats (CRISPR) associated nucleases. Methods of gene editing are numerous, well-known and routine in the art, and are described in U.S. Ser. No. 17/045,747, U.S. Ser. No. 16/977,020, and U.S. Ser. No. 16/961,396, which are herein incorporated in their entirety.

An engineered nuclease may be a guided nuclease, which may function as a ribonucleoprotein (RNP) complex with a guide RNA. According to some embodiments, a guided nuclease may be selected from the group consisting of Casl, Cas1B, Cast, Cas3, Cas4, Cas5, Cash, Cas7, Cas8, Cas9 (also known as Csn1 and Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, Cpfl, CasX, CasY, CasZ, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions thereof). According to some embodiments, a guided nuclease is a Cas9 or Cpfl enzyme. The DNA construct or molecule encoding a guided nuclease, or the guided nuclease itself, may be delivered with or without a guide nucleic acid.

For guided nucleases, a guide nucleic acid molecule may be further provided to direct the guided nuclease to a target site in the genome of the plant via base-pairing or hybridization to cause a DSB or nick at or near the target site. The guide nucleic acid may be transformed or introduced into a plant cell or tissue as a guide nucleic acid molecule, or as a recombinant DNA molecule, construct or vector comprising a transcribable DNA sequence encoding the guide nucleic acid operably linked to a promoter or plant-expressible promoter (e.g., a maize-expressible promoter). The promoter may be a constitutive promoter, a tissue-specific or tissue-preferred promoter, a developmental stage promoter, or an inducible promoter.

In some embodiments, the guide nucleic acid comprises a first segment comprising a nucleotide sequence that is complementary to a sequence in a target nucleic acid and a second segment that interacts with a guided nuclease protein. In some embodiments, the first segment of a guide comprising a nucleotide sequence that is complementary to a sequence in a target nucleic acid corresponds to a CRISPR RNA (crRNA or crRNA repeat). In some embodiments, the second segment of a guide comprising a nucleic acid sequence that interacts with a guided nuclease protein corresponds to a trans-acting CRISPR RNA (tracrRNA). In some embodiments, the guide nucleic acid comprises two separate nucleic acid molecules (a polynucleotide that is complementary to a sequence in a target nucleic acid and a polynucleotide that interacts with a guided nuclease protein) that hybridize with one another. In other embodiments, the guide nucleic acid is a single polynucleotide. In some embodiments, the guide nucleic acid may comprise DNA, RNA or a combination of DNA and RNA.

A protospacer-adjacent motif (PAM) may be present in the genome immediately adjacent and upstream to the 5′ end of the genomic target site sequence complementary to the targeting sequence of the guide RNA, immediately downstream (3′) to the sense (+) strand of the genomic target site (relative to the targeting sequence of the guide RNA) as known in the art. See, e.g., Wu, X. et al. 2014. Target specificity of the CRISPR-Cas9 system, Quant Biol. 2(2): 59-70. The genomic PAM sequence on the sense (+) strand adjacent to the target site (relative to the targeting sequence of the guide RNA) may comprise any known PAM sequence, including, for example, 5′-NGG-3′. However, the corresponding sequence of the guide nucleic acid (immediately downstream (3′) to the targeting sequence of the guide RNA) may generally not be complementary to the genomic PAM sequence.

The guide nucleic acid may typically be a non-coding RNA molecule that does not encode a protein. The targeting sequence of the guide nucleic acid may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The targeting sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the targeting sequence, a guide nucleic acid may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide nucleic acids for genome editing and site-directed integration at a target site within the genome of a maize plant using a guided nuclease are known in the art.

An engineered nuclease may be a site-specific nuclease. Several site-specific nucleases, such as recombinases, zinc finger nucleases (ZFNs), meganucleases, and TALENs, are not nucleic acid-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-nucleic acid-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such genomic locus to knockout or knockdown expression of the gene via repair of the DSB or nick, which may lead to the creation of a mutation or insertion of a sequence at the site of the DSB or nick, through cellular repair mechanisms, which may be guided by a donor template molecule.

In some embodiments, a site-specific nuclease is a recombinase. A recombinase may be a serine recombinase attached to a DNA recognition motif, a tyrosine recombinase attached to a DNA recognition motif, or other recombinase enzyme known in the art. A recombinase or transposase may be a DNA transposase or recombinase attached or fused to a DNA binding domain. Non-limiting examples of recombinases include a tyrosine recombinase attached, etc., to a DNA recognition motif provided herein is selected from the group consisting of a Cre recombinase, a gin recombinase, a Flp recombinase, and a Tnpl recombinase. In an aspect, a Cre recombinase or a Gin recombinase provided herein is tethered to a zinc-finger DNA-binding domain, or a transcription activator-like effector (TALE) DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

A site-specific nuclease may be a zinc finger nuclease (ZFN). ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger alpha-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., US Patent App. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Without being limited by any theory, because the DNA-binding specificities of zinc finger domains can be re-engineered using one of various methods, customized ZFNs can theoretically be constructed to target nearly any target sequence (e.g., at or near a gene in a maize plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick.

A site-specific nuclease may be a TALEN enzyme. TALENs are artificial restriction enzymes generated by fusing the TALE DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Transcription activator-like effectors (TALEs) can be engineered to bind practically any DNA sequence, such as at or near the genomic locus of a gene in a maize plant. TALEs have a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and Fold variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al., Nucleic Acids Research (2012) 40: W117-122.; Cermak et al., Nucleic Acids Research (2011) 39:e82; and tale-nt.cac.cornelledu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-Msol, I-SceI, I-AniI, and I-Dmol. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a maize plant, such as at or near the genomic locus of a gene. In another aspect, a meganuclease provided herein is capable of generating a targeted DSB.

In some embodiments, gene editing comprises (a) inducing a DSB in the genome of a cell at a cleavage site at or near a recognition site for a natural or engineered nuclease by expressing in the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site and/or a downstream flanking DNA region having homology to the DNA region downstream of the preselected site for allowing homologous recombination between said flanking region or regions and said DNA region or regions flanking said preselected site; and (c) selecting a cell wherein said repair nucleic acid molecule has been used as a template for making a modification of said genome at said preselected site. In other embodiments, gene editing comprises (a) inducing a DSB in the genome of a cell at a cleavage site at or near a recognition site for a natural or engineered nuclease by introducing into the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site and/or a downstream flanking DNA region having homology to the DNA region downstream of the preselected site for allowing homologous recombination between said flanking region or regions and said DNA region or regions flanking said preselected site; and (c) selecting a cell wherein said repair nucleic acid molecule has been used as a template for making a modification of said genome at said preselected site.

As used herein, a repair nucleic acid molecule is a single-stranded or double-stranded DNA molecule or RNA molecule that is used as a template for modification of the genomic DNA at the preselected site in the vicinity of or at the cleavage site. As used herein, use as a template for modification of the genomic DNA, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two ends of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

In some embodiments, gene editing comprises base editing. As used herein, base editing refers to the chemical modification of at least one nucleotide in a DNA or RNA sequence, resulting in an altered DNA sequence. Base editing may be achieved using a fusion protein, referred to as a base editor, comprising a site-specific nuclease and a base-editing enzyme. The base editor may comprise any suitable site-specific nuclease, such as the site-specific nucleases described herein, which has been modified to reduce or abolish nuclease activity of the site-specific nuclease. The base-editing enzyme may be any enzyme capable of chemically modifying a nucleotide, including, for example, a deaminase such as a cytidine deaminase, a cytosine deaminase, an adenosine deaminase, an adenine deaminase, or variants thereof. The base editor may also comprise one or more additional protein domains and/or one or more linker sequences linking the site-directed nuclease, the base-editing enzyme, and any other additional protein domains. Base editing is reviewed, for example, in Porto et al. 2020 (“Base editing: advances and therapeutic opportunities.” Nature Reviews Drug Discovery 19.12: 839-859).

In some embodiments, the genetic modifications introduced by gene editing result in the decreased expression (including non-expression or altered activity) of one or more MiMe loci. In gene editing, the introduction of a DSB or nick may be used to introduce targeted genetic modifications in the genome of a maize plant. According to this approach, genetic modifications, such as deletions, insertions, inversions and/or substitutions may be introduced at a target site via imperfect repair of the DSB or nick to produce a knock-out or knock-down of a gene, or to produce a MiMe component with altered activity. Such genetic modifications may be generated by imperfect repair of the targeted locus even without the use of a donor template molecule, and can result in decreased expression (including non-expression or altered activity) of an endogenous gene product. For example, genetic modifications may be produced by an indel (insertion or deletion of nucleotide bases in a target DNA sequence through NHEJ), or by specific removal of sequence that reduces or completely destroys the function of a sequence or motif at or near the targeting site, or which results in an altered activity of a MiMe component such as the production of a dominant-negative MiMe component, a constitutively active MiMe component, a null mutant, or the like. Such embodiments may comprise a deletion or insertion which alters one or more post-translational modifications on the one or more MiMe components. The post-translational modifications can include phosphorylation, glycosylation, ubiquitination, nitrosylation, methylation, acetylation, lipidation and the like. Altered activity in a MiMe component can be achieved, for example, by deleting or otherwise disrupting one or more phosphorylation sites (e.g., Tyrosine phosphorylation site or Serine/Threonine phosphorylation site). In further embodiments, the motif which is disrupted is a proteolytic cleavage site. A knockout of a gene may be achieved by inducing a DSB or nick at or near the endogenous locus of the gene that results in non-expression of the gene product, whereas a knockdown of a gene may be achieved in a similar manner by inducing a DSB or nick at or near the endogenous locus of the gene that is repaired imperfectly at a site that does not affect the coding sequence of the gene in a manner that would eliminate the function of the gene product. For example, the site of the DSB or nick within the endogenous locus may be in the upstream or 5′ region of the gene (e.g., a promoter and/or enhancer sequence) to affect or reduce its level of expression. Similarly, such targeted knockout or knockdown mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knockout mutations of a gene may be achieved by substituting, inserting, deleting or inverting at least a portion of the gene, including, but not limited to, by introducing a frame shift or premature stop codon into a protein coding sequence of the gene. A deletion of a portion of a gene may also be introduced by generating DSBs or nicks at two target sites and causing a deletion of the intervening target region flanked by the target sites.

In some embodiments, the genetic modifications are introduced by transgenesis. Transgenes may include, but are not limited to, one or more protein-coding sequences operably linked to a plant-expressible promoter (e.g., a maize expressible promoter), one or more transcribable DNA sequences encoding an RNA molecule operably linked to a plant-expressible promoter (e.g., a maize expressible promoter), a gene of interest, a marker gene, or any combination thereof. Methods for the introduction of transgenes in maize plants are well-known and routine in the art. In some embodiments, transgenesis comprises (a) inducing a DSB in the genome of a cell at a cleavage site at or near a recognition site for a natural or engineered nuclease by expressing in the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site, a downstream flanking DNA region having homology to the DNA region downstream of the preselected site, and a transgene region flanked by the upstream and downstream flanking DNA regions and comprising the transgene to be inserted at the preselected site; and (c) selecting a cell wherein said repair nucleic acid molecule has been used as a template for making a modification of said genome at said preselected site. In other embodiments, transgenesis comprises (a) inducing a DSB in the genome of a cell at a cleavage site at or near a recognition site for a natural or engineered nuclease by introducing into the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site, a downstream flanking DNA region having homology to the DNA region downstream of the preselected site, and a transgene region flanked by the upstream and downstream flanking DNA regions and comprising the transgene to be inserted at the preselected site; and (c) selecting a cell wherein said repair nucleic acid molecule has been used as a template for making a modification of said genome at said preselected site.

In some embodiments, the genetic modification comprises introducing proteins, nucleic acids, or a combination thereof into a maize cell. The introduction of the proteins, nucleic acids, or combination thereof into the maize cell may be achieved by any of several means known and routinely-used in the art. In some embodiments, the introduction of the proteins, nucleic acids, or combination thereof into the maize cell comprises isolating protoplasts, transfecting the protoplasts, encapsulating the protoplasts, and regenerating maize plants from the protoplasts. In other embodiments, the introduction of the proteins, nucleic acids, or combination thereof into the maize cell comprises biolistic transformation. In certain embodiments, the introduction of the proteins, nucleic acids, or combination thereof into the maize cell comprises isolating immature maize embryos, bombarding the embryos with particles comprising nucleic acids, and regenerating maize plants from the immature embryos. Numerous additional transformation methods may be used to introduce the proteins, nucleic acids, or combination thereof into a suitable maize plant or maize cell. Transformation methods include the use of liposomes, electroporation, chemicals that increase free DNA uptake, injection of the DNA directly into the maize plant (cell) such as microinjection, particle gun bombardment, transformation using viruses or pollen and microprojection. Methods may be selected from the calcium/polyethylene glycol method for protoplasts (Krens et al. (1982) Nature 296: 72-74; Negrutiu et al. (1987) Plant. Mol. Biol. 8: 363-373); electroporation of protoplasts (Shillito et al. (1985) Bio/Technol. 3: 1099-1102); microinjection into plant material (Crossway et al. (1986) Mol. Gen. Genet. 202: 179-185); DNA or RNA-coated particle bombardment (Klein et al. (1987) Nature 327: 70) infection with (non-integrative) viruses and the like.

In some embodiments, generating the parent MiMe maize plant, the grandparent non-MiMe maize plant, the great-grandparent non-MiMe maize plant, or any combination thereof comprises introducing an RNA-guided nuclease system (e.g., a CRISPR system) into a cell of a candidate maize line, wherein the RNA-guided nuclease system (e.g., a CRISPR system) is configured to bind to one or more target sequences of each of one or more MiMe loci. Exemplary target sequences for specific MiMe loci are provided in the sequence listing as outlined in Table 5, and also in Examples 2 and 3. In certain embodiments, the one or more target sequences comprise a polynucleotide sequence selected from the group consisting of SEQ ID NOs: 14-60, or a variant thereof comprising 1, 2, 3, 4, or 5 nucleotide substitutions.

In certain embodiments, the CRISPR system comprises one or more nucleic acid molecules (e.g., one or more plasmids) encoding an expression cassette for expressing a Cas enzyme, and expression cassette for expressing a targeting RNA (e.g., a crRNA or gRNA molecule), or a combination thereof. In some variations, the targeting RNA and the Cas enzyme are encoded on separate nucleic acid molecules (e.g., separate plasmids). In other variations, the targeting RNA and the Cas enzyme are encoded on the same nucleic acid molecule (e.g., the same plasmid). In certain embodiments, the CRISPR system comprises ribonucleoproteins (RNPs) comprising a Cas enzyme complexed with a targeting RNA (e.g., a crRNA or gRNA molecule). Exemplary gRNA protospacer sequences for specific MiMe loci are provided in the sequence listing as outlined in Table 5, and also in Example 2. In certain embodiments, the targeting RNA (e.g., crRNA or gRNA) comprises a polynucleotide sequence selected from the group consisting of SEQ ID NOs: 61-107, or a variant thereof comprising 1, 2, 3, 4, or 5 nucleotide substitutions.

In some embodiments, introducing the CRISPR system into the cell of the candidate maize line results in an insertion, a deletion, one or more nucleotide changes, or an inversion that results in decreased expression of the targeted MiMe locus. The one or more MiMe loci may comprise any MiMe loci known in the art or described herein. In some variations, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the genomic locus following the start codon in the 3′ direction. In certain variations, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some variations, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the genomic locus. In some variations, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction, thereby eliminating expression (e.g., activity) of the genomic locus.

Crossing Clonal Gametes to Produce Hybrid Polyploid Seed

In some embodiments, the method of breeding a polyploid hybrid maize line comprises providing clonal gametes from two parent MiMe maize plants and crossing the clonal gametes to produce a hybrid polyploid seed comprising two, three, or more haplotypes. In other embodiments, the method of breeding a polyploid hybrid maize line comprises providing clonal gametes from a parent MiMe maize plant, providing haploid gametes from a homozygous parent non-MiMe maize plant, and crossing the clonal gametes with the haploid gametes to produce a hybrid polyploid seed. In some variations, the homozygous parent non-MiMe maize plant is diploid and the haploid gametes are monoploid gametes. In yet other embodiments, the method of breeding a polyploid hybrid maize line comprises providing clonal gametes from a parent MiMe maize plant, providing unreduced, non-clonal gametes from a homozygous parent maize plant, and crossing the clonal gametes with the unreduced, non-clonal gametes to produce a hybrid polyploid seed. Methods for providing clonal gametes, providing haploid gametes, providing unreduced, non-clonal gametes, crossing clonal gametes, crossing clonal gametes with haploid gametes, and crossing clonal gametes with unreduced, non-clonal gametes are described herein. In some embodiments, the method of breeding a polyploid hybrid maize line comprises growing the hybrid polyploid seed to produce a hybrid polyploid maize plant.

In some embodiments, the method of breeding a polyploid hybrid maize line comprises crossing the clonal gametes to produce the hybrid polyploid seed. In some variations, crossing the clonal gametes to produce the hybrid polyploid seed comprises contacting pollen comprising clonal gametes with the stigma of a pistil comprising clonal gametes. In some variations, crossing the clonal gametes to produce the hybrid polyploid seed comprises contacting pollen comprising clonal gametes of a first maize plant with the stigma of a pistil comprising clonal gametes of a second maize plant. In some embodiments, crossing the clonal gametes to produce the hybrid polyploid seed comprises contacting pollen comprising clonal gametes from a first parent MiMe maize plant with the stigma of a pistil comprising clonal gametes of a second parent MiMe maize plant. In some variations, the homozygous parent non-MiMe maize plant is diploid and the haploid gametes are monoploid gametes. In some embodiments, the hybrid polyploid seed is produced by crossing the clonal gametes and allowing seeds to form.

In other embodiments, the method of breeding a polyploid hybrid maize line comprises crossing the clonal gametes with the haploid gametes to produce the hybrid polyploid seed. In one variation, crossing the clonal gametes with the haploid gametes to produce the hybrid polyploid seed comprises contacting pollen comprising clonal gametes with the stigma of a pistil comprising haploid gametes. In another variation, crossing the clonal gametes with the haploid gametes to produce the hybrid polyploid seed comprises contacting pollen comprising haploid gametes with the stigma of a pistil comprising clonal gametes. In some embodiments, crossing the clonal gametes with the haploid gametes to produce the hybrid polyploid seed comprises contacting pollen comprising clonal gametes from a parent MiMe maize plant with the stigma of a pistil comprising haploid gametes of a homozygous parent non-MiMe maize plant. In other embodiments, crossing the clonal gametes with the haploid gametes to produce the hybrid polyploid seed comprises contacting pollen comprising haploid gametes from a homozygous parent non-MiMe maize plant with the stigma of a pistil comprising clonal gametes of a parent MiMe maize plant. In some embodiments, the population of polyploid seed is produced by crossing the clonal gametes with the haploid gametes and allowing seeds to form.

In yet other embodiments, the method of breeding a polyploid hybrid maize line comprises crossing clonal gametes with unreduced, non-clonal gametes to produce the hybrid polyploid seed. In one variation, crossing the clonal gametes with the unreduced, non-clonal gametes to produce the hybrid polyploid seed comprises contacting pollen comprising clonal gametes with the stigma of a pistil comprising unreduced, non-clonal gametes. In another variation, crossing the clonal gametes with the unreduced, non-clonal gametes to produce the hybrid polyploid seed comprises contacting pollen comprising unreduced, non-clonal gametes with the stigma of a pistil comprising clonal gametes. In some embodiments, crossing the clonal gametes with the unreduced, non-clonal gametes to produce the hybrid polyploid seed comprises contacting pollen comprising clonal gametes from a parent MiMe maize plant with the stigma of a pistil comprising unreduced, non-clonal gametes of a homozygous parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, crossing the clonal gametes with the unreduced, non-clonal gametes to produce the hybrid polyploid seed comprises contacting the stigma of a pistil comprising clonal gametes from a parent MiMe maize plant with pollen comprising unreduced, non-clonal gametes of a homozygous parent maize plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, the hybrid polyploid seed is produced by crossing the clonal gametes with the unreduced, non-clonal gametes and allowing seeds to form.

Evaluating Characteristics and Heterotic Performance

In some embodiments, the method of breeding a polyploid hybrid maize line comprises evaluating one or more characteristics of the hybrid polyploid maize plant. Methods for evaluating maize plant characteristics are numerous and well-known in the art. The one or more characteristics evaluated may include, but are not limited to, plant height, plant size, plant vigor, fruit yield, crop yield, disease resistance, pest resistance, and the like. The maize plants to be evaluated may then be grown under different geographical, climatic, and soil conditions, and further selections can be made during, and at the end of, the growing season. Promising advanced breeding lines are thoroughly tested and compared to appropriate standards in environments representative of the commercial target area(s) for three years at least. The best lines are candidates for new commercial cultivars. These processes, which lead to the final step of marketing and distribution, usually take from five to ten years from the time the first cross or selection is made.

In some embodiments, the one or more characteristics includes the heterotic performance of the two, three, or more haplotypes of the polyploid hybrid maize plant. In some embodiments, the heterotic performance of the two, three, or more haplotypes of the polyploid hybrid maize plant is used to guide the breeding of maize lines, the selecting of candidate maize lines, or both. In some embodiments, the heterotic performance is predicted via genome prediction modeling.

In some embodiments, the method of breeding a polyploid hybrid maize line comprises repeating the steps of the method using the one or more characteristics of the hybrid polyploid maize plant evaluated to guide the breeding of, the selecting of candidate maize lines, or both. In some variations, the method comprises repeating the steps of the method two, three, four, five, six, seven, eight, nine, or ten times or more. In additional variations, the repeating of the steps of the method iteratively informs a genome prediction model for improved prediction of heterotic performance. In certain variations, the improved prediction of heterotic performance allows for rapid combination of haplotypes with strong heterotic performance and acceleration of breeding programs.

Inventory and Maintenance of Maize Lines

In some embodiments, the method of breeding a polyploid maize line comprises maintaining the lines of maize. In some variations, the lines of maize are maintained via selfing, apomixis, cell culture, or any combination thereof. Additional methods of maintaining lines of maize are well-known in the art. In some embodiments, the method of breeding a polyploid maize line comprises maintaining an inventory of lines of maize from which haplotypes may be selected for rapid deterministic stacking of the haplotypes. In some variations, the inventory of lines comprises one or more maize lines having complete or partial MiMe genotypes that are maintained through hybridization with a haploid inducer, or a combination thereof. In additional variations, the inventory of lines may include, but is not limited to, the set of lines, candidate lines, lines comprising one or more MiMe alleles, lines having a partial MiMe genotype, lines having a complete MiMe genotype, grandparent non-MiMe maize plants having a partial MiMe genotype, parent MiMe maize plants, homozygous parent non-MiMe maize plants, or any combination thereof maintained by seed propagation, tissue culture, hybridization with a haploid inducer or any combination thereof.

In some embodiments, haploid induction may be used to maintain polyploid maize lines having a complete MiMe genotype. In certain embodiments, maintaining the inventory of lines of maize comprises crossing a polyploid maize plant having a complete MiMe genotype with a haploid inducer to produce progeny of the same ploidy as the maize plant having the complete MiMe genotype. In one variation, maintaining the inventory of lines of maize comprises crossing a tetraploid maize plant having a complete MiMe genotype with a haploid inducer to produce tetraploid progeny.

Genetically Modified Plants, Plant Parts, Plant Cells, and Processed Plant Products

In some aspects, provided herein are genetically modified maize plants, plant parts, and plant cells grown from a population of polyploid maize seed or a subpopulation of genetically uniform polyploid maize seed described herein. Also provided herein are processed maize products derived from the genetically modified maize plants, plant parts, or plant cells provided herein. In some embodiments, the genetically modified maize plant parts, genetically modified plant cells, and processed maize products provided herein are non-regenerable.

In some embodiments, genetically modified maize plants and genetically modified plant parts are provided herein. The genetically modified maize plants and plant parts may be grown from a population of polyploid maize seed or a subpopulation of genetically uniform polyploid maize seed comprising three or more haplotypes and one or more genetic modifications resulting in decreased expression of one or more MiMe loci described herein. Alternatively, the genetically modified maize plants and plant parts may be regenerated from a genetically modified maize plant cell wherein the genetically modified maize plant cell comprises three or more haplotypes and one or more genetic modifications resulting in decreased expression of one or more MiMe loci. Genetically modified maize plants can be obtained from a genetically modified maize seed. Genetically modified maize plant parts can be obtained by cutting, snapping, grinding or otherwise disassociating the part from the maize plant. The maize plant part may be any plant part known in the art, including, but not limited to, a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a seed, a cob, a glume, a husk, a leaf sheath, a ligule, a trichome, or a portion thereof. In certain embodiments, a genetically modified maize plant part provided herein is a non-regenerable portion of a genetically modified maize plant part. As used in this context, a “non-regenerable” portion of a genetically modified maize plant part refers to a portion that cannot be induced to form a whole maize plant (e.g., through in vitro culture) or that cannot be induced to form a whole maize plant that is capable of sexual and/or asexual reproduction. A non-regenerable portion of a genetically modified maize plant part may be a portion of a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a cob, a glume, a husk, a leaf sheath, a ligule, a trichome, or a portion thereof.

In some embodiments, a non-regenerable or non-propagating maize plant cell is provided herein. As used in this context, a “non-regenerable maize plant cell” is a cell which cannot be regenerated into a whole maize plant that is capable of sexual and/or asexual reproduction through in vitro culture. The non-regenerable maize cell may be in a maize plant or plant part described herein. The non-regenerable maize cell may be a cell in a seed, or in the seedcoat of said seed. Mature maize plant organs, including a mature leaf, a mature stem or a mature root, contain at least one non-regenerable cell. In certain embodiments, the non-regenerable maize plant cell is a somatic cell.

Also provided herein is a maize cell culture or tissue culture of non-regenerable or regenerable maize cells or tissue of a genetically modified maize plant or genetically modified plant part described herein, wherein the non-regenerable or regenerable maize cells comprise three or more haplotypes and one or more genetic modifications resulting in decreased expression of one or more MiMe loci described herein. Preferably, the regenerable maize cells are derived from embryos, protoplasts, meristematic cells, callus, pollen, leaves, anthers, stems, pericarps, roots, root tips, seeds, flowers, cotyledons, and/or hypocotyls of a genetically modified maize plant or a genetically modified plant part grown from a population of polyploid maize seed or a subpopulation of genetically uniform polyploid maize seed described herein.

In some embodiments, provided herein is a processed maize product derived from a genetically modified maize plant, plant part, or plant cell described herein comprising three or more haplotypes and one or more genetic modifications resulting in decreased expression of one or more MiMe loci. In certain embodiments, the processed maize product contains sufficient nucleic acid (e.g., DNA or RNA) and/or protein material from the genetically modified maize plant, plant part, or plant cell to detect nucleic acid and/or protein sequences corresponding to the three haplotypes, the one or more genetic modifications resulting in decreased expression of one or more MiMe loci, or both. In some embodiments, the processed maize product is non-regenerable, i.e., cannot be induced to form a whole maize plant or that cannot be induced to form a whole maize plant that is capable of sexual and/or asexual reproduction.

A processed maize product may be a seed, a grain, a root, a vegetable, or any maize plant part described herein, and may be blended as a commodity or other product which moves through commerce and is derived from a genetically modified maize plant or a genetically modified plant part. In some embodiments, the commodity or other product can be tracked through commerce by detecting nucleic acid and/or protein sequences of the genetically modified maize plant or plant part from which they were obtained. In certain embodiments, the processed maize product comprises a detectable amount of nucleotide and/or protein sequences corresponding to the three or more haplotypes and/or to the one or more genetic modifications resulting in decreased expression of one or more MiMe loci. In certain embodiments, the commodity or other maize product is produced in or maintained in the genetically modified maize plant or plant part from which the commodity or other product has been obtained. Such commodities or other products of commerce include, but are not limited to, maize plant parts, fresh corn, canned corn, dehydrated corn, starch, hulls, hominy, popcorn, cereal, grain, margarine, fermented alcoholic beverage, biofuel, gluten, corn syrup, table syrup, candy, confections, soft drinks, ice cream, shoe polish, corn sugar, infant formulas, dietetic foods, caramel coloring, vinegar, lactic acid, tanning mixtures, brewing additive, artificial silk, edible starch, dextrin, mucilage, glue, textile sizing, food sauces, fireworks, industrial starch, laundry starch, filler in paper, cosmetics, explosives, germ, oil cake or meal, cattle feed, plastic resin, rubber substitutes, erasers, elastic heels, soap, glycerin, soluble corn oil, cloth coloring, salad oils, cooking oils, medicinal oils, animal feed, paper, wallboard, filling material, fuel, charcoal, industrial solvent, biomass, oil, meal, food starch, syrup, sugar, animal feed, flour, flakes, bran, processed seed, and seed. The processed maize product may be a food product that is processed by any means known in the art, e.g., canned, steamed, boiled, fried, blanched and/or frozen etc. The maize product may be produced for any purpose(s) or industry, including but not limited to human consumption, agriculture, animal consumption, dietary supplement, food product ingredient, pharmaceutical, textile, wood, paper, adhesive, binder, texture agent, filler, biofuel production.

In some embodiments, provided herein is a genetically modified maize plant, plant part, or plant cell comprising: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, provided herein is a genetically modified maize plant, plant part, or plant cell comprising: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof. In certain embodiments, the genetically modified maize plant part is a non-regenerable plant part. In certain embodiments, the genetically modified maize plant cell is a non-regenerable plant cell. Also provided herein is germplasm of the genetically modified maize plant, the germplasm comprising the complete or partial MiMe genotype. Further provided herein is a genetically altered plant genome derived from the genetically modified maize plant, the genetically altered plant genome comprising the complete or partial MiMe genotype.

In some embodiments, provided herein is a genetically modified maize plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, provided herein is a genetically modified maize plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In other variations, the one or more MiMe loci of the component of progression through the first division of meiosis comprise PS1, JASON, or a combination thereof. In certain embodiments, the genetically modified maize plant part is a non-regenerable plant part. In certain embodiments, the genetically modified maize plant cell is a non-regenerable plant cell. Also provided herein is germplasm of the genetically modified maize plant, the germplasm comprising the complete or partial MiMe genotype. Further provided herein is a genetically altered plant genome derived from the genetically modified maize plant, the genetically altered plant genome comprising the complete or partial MiMe genotype.

In some embodiments, provided herein is a genetically modified maize plant, plant part, or plant cell comprising: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC. In certain embodiments, the genetically modified maize plant part is a non-regenerable plant part. In certain embodiments, the genetically modified maize plant cell is a non-regenerable plant cell. Also provided herein is germplasm of the genetically modified maize plant, the germplasm comprising the MiMe alleles of the genetically modified maize plant. Further provided herein is a genetically altered plant genome derived from the genetically modified maize plant, the genetically altered plant genome comprising the germplasm comprising the MiMe alleles of the genetically modified maize plant.

In some embodiments, provided herein is a genetically modified maize plant, plant part, or plant cell comprising: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In yet additional variations, the MiMe locus of the component of progression through the first division of meiosis of the third haplotype is PS1 or JASON. In still additional variations, the MiMe locus of the component of progression through the second division of meiosis of the third haplotype is OSD1, CYCA1, TDM1, PC1, PC2 or FC. In certain embodiments, the genetically modified maize plant part is a non-regenerable plant part. In certain embodiments, the genetically modified plant cell is a non-regenerable plant cell. Also provided herein is germplasm of the genetically modified maize plant, the germplasm comprising the MiMe alleles of the genetically modified maize plant. Further provided herein is a genetically altered plant genome derived from the genetically modified maize plant, the genetically altered plant genome comprising the germplasm comprising the MiMe alleles of the genetically modified maize plant.

In some embodiments, provided herein is a genetically modified maize plant, plant part, or plant cell having a partially-complemented MiMe genotype. In some embodiments, the genetically modified maize plant, plant part, or plant cell has a partially-complemented MiMe genotype comprising: (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. Also provided herein is germplasm of the genetically modified maize plant, the germplasm comprising the partially-complemented MiMe genotype. Further provided herein is a genetically altered plant genome derived from the genetically modified maize plant, the genetically altered plant genome comprising the germplasm comprising the partially-complemented MiMe genotype.

In some embodiments, provided herein is a genetically modified maize plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof. Also provided herein is germplasm of the genetically modified maize plant, the germplasm comprising the partially-complemented MiMe genotype. Further provided herein is a genetically altered plant genome derived from the genetically modified maize plant, the genetically altered plant genome comprising the germplasm comprising the partially-complemented MiMe genotype.

In some embodiments, provided herein is a genetically modified maize plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof. Also provided herein is germplasm of the genetically modified maize plant, the germplasm comprising the partially-complemented MiMe genotype. Further provided herein is a genetically altered plant genome derived from the genetically modified maize plant, the genetically altered plant genome comprising the germplasm comprising the partially-complemented MiMe genotype.

In some embodiments which may be combined with any of the preceding embodiments, the genetically modified maize plant, plant part, or plant cell is diploid, triploid, tetraploid, pentaploid, hexaploid, heptaploid, or octaploid. In additional embodiments which may be combined with any of the preceding embodiments, the genetically modified maize plant, plant part, or plant cell comprises two, three, four, or more haplotypes of the same or related species of maize.

In certain embodiments, which may be combined with any of the previous embodiments, the maize plant part is a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a seed, a cob, a glume, a husk, a leaf sheath, a ligule, a trichome, or a portion thereof.

In some embodiments, provided herein is a processed maize product derived from the genetically modified maize plant, plant part, or plant cell of any one of the preceding embodiments. In certain embodiments, the processed maize product comprises a detectable amount of the one or more MiMe alleles of the genetically modified maize plant, plant part, or plant cell. In certain embodiments, the maize product is selected from the group consisting of plant biomass, oil, meal, food starch, syrup, animal feed, flour, flakes, bran, lint, hulls, processed seed, puree, juice, juice concentrate, pulp, pomace, preserve, or sauce. In some embodiments, the processed maize product is non-regenerable. In certain embodiments, the processed maize product contains sufficient nucleic acid (e.g., DNA or RNA) and/or protein material from the genetically modified maize plant, plant part, or plant cell to detect nucleic acid and/or protein sequences corresponding to the three haplotypes, the one or more genetic modifications resulting in decreased expression of one or more MiMe loci, or both.

In some embodiments, provided herein is a processed maize product derived from a genetically modified maize plant, plant part, or plant cell comprising: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, provided herein is a processed maize product derived from a genetically modified maize plant, plant part, or plant cell comprising: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof. In certain embodiments, the processed maize product comprises sufficient nucleic acid (e.g., DNA or RNA) and/or protein material from the genetically modified maize plant, plant part, or plant cell to detect nucleic acid and/or protein sequences corresponding to the three haplotypes, the MiMe alleles, or both in the processed maize product. In certain embodiments, the processed maize product is non-regenerable.

In some embodiments, provided herein is a processed maize product derived from a genetically modified maize plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, provided herein is a processed maize product derived from a genetically modified maize plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In other variations, the one or more MiMe loci of the component of progression through the first division of meiosis comprise PS1, JASON, or a combination thereof. In certain embodiments, the processed maize product comprises sufficient nucleic acid (e.g., DNA or RNA) and/or protein material from the genetically modified maize plant, plant part, or plant cell to detect nucleic acid and/or protein sequences corresponding to the three haplotypes, the MiMe alleles, or both in the processed maize product. In certain embodiments, the processed maize product is non-regenerable.

In some embodiments, provided herein is a processed maize product derived from a genetically modified maize plant, plant part, or plant cell comprising: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC. In certain embodiments, the processed maize product comprises sufficient nucleic acid (e.g., DNA or RNA) and/or protein material from the genetically modified maize plant, plant part, or plant cell to detect nucleic acid and/or protein sequences corresponding to the three haplotypes, the MiMe alleles, or both in the processed maize product. In certain embodiments, the processed maize product is non-regenerable.

In some embodiments, provided herein is a processed maize product derived from a genetically modified maize plant, plant part, or plant cell comprising: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In yet additional variations, the MiMe locus of the component of progression through the first division of meiosis of the third haplotype is PS1 or JASON. In still additional variations, the MiMe locus of the component of progression through the second division of meiosis of the third haplotype is OSD1, CYCA1, TDM1, PC1, PC2 or FC. In certain embodiments, the processed maize product comprises sufficient nucleic acid (e.g., DNA or RNA) and/or protein material from the genetically modified maize plant, plant part, or plant cell to detect nucleic acid and/or protein sequences corresponding to the three haplotypes, the MiMe alleles, or both in the processed maize product. In certain embodiments, the processed plant product is non-regenerable.

In some embodiments, provided herein is a processed maize product derived from a genetically modified maize plant, plant part, or plant cell having a partially-complemented MiMe genotype. In some embodiments, a processed maize product derived from a genetically modified maize plant, plant part, or plant cell having a partially-complemented MiMe genotype comprising: (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. In some variations of the foregoing embodiments, the processed maize product is non-regenerable.

In some embodiments, provided herein is a processed maize product derived from a genetically modified maize plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof. In some variations of the foregoing embodiments, the processed maize product is non-regenerable.

In some embodiments, provided herein is a processed maize product derived from a genetically modified maize plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof. In some variations of the foregoing embodiments, the processed maize product is non-regenerable.

In some embodiments, provided herein is a genetically modified maize plant, plant part, or plant cell, or a processed maize product derived therefrom, having a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the genetically modified maize plant, plant part, or plant cell comprises a) a rec8 allele comprising the polynucleotide sequence of SEQ ID NO: 110; and/or b) one or more osd1 alleles, each comprising a polynucleotide sequence selected from the group consisting of SEQ ID NOS: 108 and 109. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein is a genetically modified maize plant, plant part, or plant cell, or a processed maize product derived therefrom, having a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the genetically modified maize plant, plant part, or plant cell comprises a) a rec8 allele comprising the polynucleotide sequence of SEQ ID NO: 110; and/or b) one or more osd1 alleles, each comprising a polynucleotide sequence selected from the group consisting of SEQ ID NOS: 108 and 109. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein is a genetically modified maize plant, plant part, or plant cell, or a processed maize product derived therefrom, having a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the one or more OSD1 loci comprise OSD1-1, OSD1-2, and/or OSD1-3. In certain variations, the one or more OSD1 loci comprise OSD1-1 and OSD1-2. In some variations, the genetically modified maize plant, plant part, or plant cell, optionally comprising a) a rec8 allele comprising the polynucleotide sequence of SEQ ID NO: 110; b) one or more osd1 alleles, each comprising a polynucleotide sequence selected from the group consisting of SEQ ID NOS: 108 and 109; and/or c) a spo11-1 allele comprising the polynucleotide sequence of SEQ ID NO: 111. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein a genetically modified maize plant, plant part, or plant cell, or a processed maize product derived therefrom, having a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the one or more OSD1 loci comprise OSD1-1, OSD1-2, and/or OSD1-3. In certain variations, the one or more OSD1 loci comprise OSD1-1 and OSD1-2. In some variations, the genetically modified maize plant, plant part, or plant cell, optionally comprises a) a rec8 allele comprising the polynucleotide sequence of SEQ ID NO: 110; b) one or more osd1 alleles, each comprising a polynucleotide sequence selected from the group consisting of SEQ ID NOS: 108 and 109; and/or c) a spo11-1 allele comprising the polynucleotide sequence of SEQ ID NO: 111. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein a genetically modified maize plant, plant part, or plant cell, or a processed maize product derived therefrom, having a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the genetically modified maize plant, plant part, or plant cell, optionally comprises a) a rec8 allele comprising the polynucleotide sequence of SEQ ID NO: 110; and/or b) a spo11-1 allele comprising the polynucleotide sequence of SEQ ID NO: 111. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein is a genetically modified maize plant, plant part, or plant cell, or a processed maize product derived therefrom, having a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the genetically modified maize plant, plant part, or plant cell, optionally comprises a) a rec8 allele comprising the polynucleotide sequence of SEQ ID NO: 110; and/or b) a spo11-1 allele comprising the polynucleotide sequence of SEQ ID NO: 111. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein a genetically modified maize plant, plant part, or plant cell, or a processed maize product derived therefrom, having a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell comprises a rec8 allele comprising the polynucleotide sequence of SEQ ID NO: 110. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein a genetically modified maize plant, plant part, or plant cell, or a processed maize product derived therefrom, having a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell comprises a rec8 allele comprising the polynucleotide sequence of SEQ ID NO: 110. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein is a genetically modified maize plant, plant part, or plant cell, or a processed maize product derived therefrom, having a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the genetically modified maize plant, plant part, or plant cell comprises a) a rec8 allele comprising the polynucleotide sequence of SEQ ID NO: 110; and/or b) a spo11-1 allele comprising the polynucleotide sequence of SEQ ID NO: 111. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein is a genetically modified maize plant, plant part, or plant cell, or a processed maize product derived therefrom, having a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the genetically modified maize plant, plant part, or plant cell comprises a) a rec8 allele comprising the polynucleotide sequence of SEQ ID NO: 110; and/or b) a spo11-1 allele comprising the polynucleotide sequence of SEQ ID NO: 111. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein is a genetically modified maize plant, plant part, or plant cell, or a processed maize product derived therefrom, having a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell, optionally comprising a rec8 allele comprises the polynucleotide sequence of SEQ ID NO: 110. Also provided herein is germplasm of the genetically modified maize plant, the germplasm comprising the complete MiMe genotype. Further provided herein is a genetically altered plant genome derived from the genetically modified maize plant, the genetically altered plant genome comprising the complete MiMe genotype.

In some embodiments, provided herein is a genetically modified maize plant, plant part, or plant cell, or a processed maize product derived therefrom, having a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell comprises a rec8 allele comprising the polynucleotide sequence of SEQ ID NO: 110. Also provided herein is germplasm of the genetically modified maize plant, the germplasm comprising the partial MiMe genotype. Further provided herein is a genetically altered plant genome derived from the genetically modified maize plant, the genetically altered plant genome comprising the partial MiMe genotype.

In some embodiments, provided herein is a genetically modified maize plant, plant part, or plant cell, or a processed maize product derived therefrom, having a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the one or more OSD1 loci comprise OSD1-1, OSD1-2, and/or OSD1-3, and/or the one or more PAIR1 loci comprise PAIR1-1 and/or PAIR1-2. In certain variations, the one or more OSD1 loci comprise OSD1-1 and OSD1-2. In some variations, the genetically modified maize plant, plant part, or plant cell comprises a) a rec8 allele comprising the polynucleotide sequence of SEQ ID NO: 110; b) one or more osd1 alleles, each comprising a polynucleotide sequence selected from the group consisting of SEQ ID NOs: 108 and 109; and/or c) a spo11-1 allele comprising the polynucleotide sequence of SEQ ID NO: 111. Also provided herein is germplasm of the genetically modified maize plant, the germplasm comprising the partially complemented MiMe genotype. Further provided herein is a genetically altered plant genome derived from the genetically modified maize plant, the genetically altered plant genome comprising the partially complemented MiMe genotype.

In some embodiments, provided herein is a genetically modified maize plant, plant part, or plant cell, or a processed maize product derived therefrom, having a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell comprises a) a rec8 allele comprising the polynucleotide sequence of SEQ ID NO: 110; and/or b) a spol 1-1 allele comprising the polynucleotide sequence of SEQ ID NO: 111. Also provided herein is germplasm of the genetically modified maize plant, the germplasm comprising the partially complemented MiMe genotype. Further provided herein is a genetically altered plant genome derived from the genetically modified maize plant, the genetically altered plant genome comprising the partially complemented MiMe genotype.

In some embodiments, provided herein is a genetically modified maize plant, plant part, or plant cell, or a processed maize product derived therefrom, having a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the genetically modified maize plant, plant part, or plant cell, or processed maize product derived therefrom, is triploid, tetraploid, pentaploid, hexaploid, heptaploid, or octoploid. In some variations, the one or more PAIR1 loci comprise PAIR1-1 and/or PAIR1-2. In some variations, the genetically modified maize plant, plant part, or plant cell comprises a) a rec8 allele comprising the polynucleotide sequence of SEQ ID NO: 110; and/or b) a spo11-1 allele comprising the polynucleotide sequence of SEQ ID NO: 111. Also provided herein is germplasm of the genetically modified maize plant, the germplasm comprising the partially complemented MiMe genotype. Further provided herein is a genetically altered plant genome derived from the genetically modified maize plant, the genetically altered plant genome comprising the partially complemented MiMe genotype.

Sequences of MiMe Loci, Gene Products, and Alleles

+Sequence Alignment 1: REC8 protein sequences from monocotyledonous plants.
REC8 Monocots
Homologs identified by BLAST of Oryza sativa Os85g50420.1 from MSU -
Ose1 Release 7 map to monocot NOSI RefSeg, GenBank end UniProtK8 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MFYSIQLLARKAPLGQIWNAATLIIXKINRKRLDKIDEIKICECILNASVPMALRLSGILGGVVIVYERKVXLLYDDVSRLLICENZANXIXPVVDHTVL |
| Aegilops_tauschii_subsp._tauschii:(RefSeq: | .........................A...............................................................K.R.A...... |
| XP_020178692.1) | |
| Brachypodium_distachyon:(RefSeq:XP_003567819.1) | .........................A........................................................VD.....R.R........ |
| Hordeum volgare subsp.vulgare:(XP 044984305.1) | ..................I....FKS....R....................................KX...............L....K.R......L. |
| Nuse_acuminata_subsp._malaccensis:(RefScg: | .......................MRA.M..RK..............................................T.F.V...A..KVKT.S.P... |
| XP_018681669.1) | |
| Oryza sativa_Japonica_Group:(RefSeq: | .........................S.....................................A.......A........F........RVK..A.P... |
| XP_015638292.1) | |
| Sorghum_bicolor:(RefSeq:XP_021302730.1) | ...........Q.............S................................................S.......T......R.K.AT.T... |
| Triticum_aestivum:(UniProt:A8A3557687) | .........................A.......V.......................................................K.R.A...... |
| Zea_mays (RefSeq:XP_008648328.1) | .........................S................................................T.......T......R.K..T.P... |
| | 110 120 130 140 150 160 170 180 190 200 |
| | ----:----\|----:----\|----:----\|----:----\|----:----\|----:----\|----:----\|----:----\|----:----\|----:----\| |
| Consensus | PRGKAQAKYEAVTYPENKMDMEVEQPMLFSDTDGATARFRGMRLEDLDEQYVNVHILDDDFSRADXHHQAEAVNIT_VDHFESGLAETDXFNRFERFDIA |
| Aegilops_tauschii_subsp._touschii:(RefSeq: | .................A........V..T..............................I....R.......................T.......... |
| XP_G20178692.1) | |
| Brachypodium_distachyon:(RetSes:XP_08356/819.1) | .........K.......M..............N--.......S.D..G................EH.......................V.......... |
| Hordeum_vulgare_subsp._volgare:(XP_011934305.1) | .................M....L.R.V..TN..--.........................I....R..................F....I.T........ |
| Muss_acuminata_sabsp._malaccensis:(RefSeq: | .RA.....F.......-YV...P....M.P.ASI...A.QR...D..E.H.I.ID.R...L--.GND...PE.....FET.G..V....LY.H.....VG |
| XP_018681669.1) | |
| Oryza_sative_Japonica_Group:(RefSeq: | ....T............I...D........EA.--.T...........D..I............EN...D.E.....A...G...G...V.........T |
| XP_015638092.1) | |
| Sorghum_bicolor;(RefSeq;XP_021382730.1) | ................KSIN.V.....F..E...--...............F....G.....H..HR.................F....V.......... |
| Triticum_aestivum:(UniProt:40A3652687) | .................AV.......VF.T..Y--.T.......................I....R.......................I.......... |
| Zee_mays:(RefSeq:XP_008648320.1) | ....T.............IN.V.....F..EP..--.K..R.G.........Q.........H..DR...K..................L..H....... |
| | 210 220 230 240 250 260 270 280 290 300 |
| | ----:----\|----:----\|----:----\|----:----\|----:----\|----:----\|----:----\|----:----\|----:----\|----:----\| |
| Consensus | DDDTTVNITPDXHPQAPSTLVPSPPRXEDPPQQQEQXYAAPSPXXEEPQQGDXLYKKEQEEQKMKEQQPXKXSKRKARGKGPQVIMD [symbol] QIMIPGNIYQT |
| Aegilops_tauschii_subsp._tauschii:(RefSeg: | ......H..L.G..E...........P.........C-.....IH.......S.--.......TT....T.RA...............-.......V..S |
| XP_020178692.1) | |
| Brachypodium_distachyon:(RefSeq:XP_003567819.1) | ...........E........A....K-.........H......HQ.......PS--.D........R..NRP............T...-.T......... |
| Horseum_vulgare_subsp._vulgare:(XP_044984305.1) | ....IF.....G..E...........P...F...Q.R-.....IR.......S.--.......T..K..T.RA...............-..........S |
| Misa_aruminata_suisp._malarcensis:(RefSeq: | ..F.HT.F..QFF..FFA..T..I.HFDFTRTETHIHS.VFNHQT..KFFQRA---NDFDV.R---...V---....HR.PSHR...DR.:........\| |
| XP_018681669.1) | |
| Oryza_sativa_Japonica_Group:(RefSeq: | ...A.F.V...G...V..N.......Q..S.....NHH..S..IH..A...GAS..N...Q....G...A.S....K.R.DDE.M...D.......Y... |
| XP_015633692.1) | |
| Sorghum_bicolor:(RefSeq:XP_021302730.1) | ..E.....S..E..KV..........Q......E..Y-...FLVR......GP------.....--K.TP.A......REL.........M......... |
| Triticum_aestivum:(UniProt:A0A3852667) | ......H..L.G..E...........P.........C......IR.......S.--.......TT....T.PA............I..-.......V..S |
| Zea_mays:(RefSeq:XP 008548328.1) | ..E........EY..V....I.....Q..I...E.PY......VHG.....GP---ED......--K..P.A......WEV.R.......M......... |
| | 310 320 330 340 350 360 370 380 390 400 |
| | ----:----\|----:----\|----:----\|----:----\|----:----\|----:----\|----:----\|----:----\|----:----\|----:----\| |
| Consensus | WLKDPSSLXSKRRXVXSLRKINPIRSIKIGDLMDLPPVALISXSXKSX_EJYYPKQUKQLWKECTEVKSPKXSSSXGDKSSSSSQEXQFRNSPPXXFPPQ |
| Argilops_tauschii_subsp._tadschii:(RefSeq: | ........I....Q.P.--.....KA....F......S..M.Q.DD.Q-.T...Q..........K..P..P...S..........K.....-----... |
| NP_Q20178692.1) | |
| Brachypodium_distruhyon:(RefSeq:XP_083567819.1) | .....L..I....R.S.--.....QT.......E........Y.EK.P..L....................S..PG.--K.P....Q.S...-----... |
| Hordeum_wolgere_subsp._vulgare:(XP_044984305.1) | ........T....Q.R.--.....QA..M.E......ST.MCC.DD.Q-.I...Q..R.......K.NP..P...S.V........T.....-----S.. |
| Nusa_acuminata_subsp._malaccensis:(RefSeg: | ..Q.T.DIV...GRTQC..SV.....T..SN...........GLCMFPAKVH..SP..E..RK....N----I.PS....PPAQ.REVTETL-------- |
| XP_010601069.1) | |
| Oryza_sativa_Japonice_Group:(RefSeq: | ........IT..HRIN.--.V.L......R.....L..S...SLEK.P..F....E...............AP..G.--QQ...P.Q.Q..L..QA..T. |
| XP_015638292.1) | |
| Sorghum_bicolor:(RefSeq:XP_021302730.1) | ....A...V....K.N.--NF.F...T..S...I....S...HDNS.S-.L....P......D......T.V..CG---QR.....Q.......HE.... |
| Triticum_aestivum:(UniProt:A0A385/68?) | ........I....Q.K.--.....KA....E......S..M.H.ND.Q-.I...Q..........K..I..H...S..........K....P-----S.. |
| Zea_mays:(RefSeq:XP_098648328.1) | ....A...V....KLN.--NF.F...T..S...HI.......HDN_FS-.LC...P......D......T.A..GG---QR.....P..K..-----... |
| | 410 420 430 440 450 460 470 480 490 500 |
| | ----:----\|----:----\|----:----\|----:----\|----:----\|----:----\|----:----\|----:----\|----:----\|----:----\| |
| Consensus | PQGXYQXEMGAQPMDFTPMDFIDGIEKLRANKSGLEXEXVXDXXHGDHSVTPGSPAGLSRRSASSSGGSGRGAFJPLDPEIQLPSGSGRSKAXQHSSGRS |
| Aegilops_tauschii_subsp._tauschii: | ...D-.N....................M......-.F.G.F.GP...P.......-................G.L......L.Q.....A..R.L..... |
| (RefSeq:XP_020178692.1) | |
| Brachypodium_distruhyon:(RefSeq:XP_083567819.1) | ...E..G.....-----..........I.G....-.Y.R.D.AL...........-..................V.......FY..G.....R....... |
| Hordeum_volgare_subsp._vulgare:(XP_044984305.1) | ...D .N..... ......L..EM.......GF.G.F.GPL.A........ ................G.L..G...P.QF.D..A..K.L..... |
| Musa_acuminata_subsp._malaccensis:(RefSeq: | -LEEV.G.I.SN-----SL.VS--.......LEN.DFQGFD.AFSM..F.....S..Q.SK.MP..-...H-..M..E.......--V....K....SK. |
| XP_018681559.1) | |
| Oryza_sativa_Japonica_Group:(RefSeq: | ..VDNDR...FH-----.V..A.D.....G.T..-.YGRDY.AF.S.........-...............-G.TQ....V....--.....-.....K. |
| XP_015638092.1) | |
| Sorghum_bicolor:(RefSeq:XP_021302730.1) | AG.E.EM.T.GL-----..............M.A-.YDRAY.TL.S.........-..............Q-..I...A.V...........GR...A.. |
| Triticum_aestivum;(UniProt:A8A365Z567) | ...D-.N....................M......-.F.G.F.GP...P.......-..................L........Q.....A..R.L..... |
| Zea_mays:(RefSeq:XP_088648328.1) | AG.E.EM.T.GL-----...L..........M.A-KYDRAYNIL.S........................S-..IQ....V...........G....A.. |
| | 510 520 530 540 550 560 570 580 590 600 |
| | ----:----\|----:----\|----:----\|----:----\|----:----\|----:----\|----:----\|----:----\|----:----\|----:----\| |
| Consensus | LGNLDPVEEEFPLEQEVRDFKLRRLSDXGPIPDLELEETEPTQTPYXKQSXPAXDXITESIHSYLKLHFDSPXAPQSESLSQLXHGMTTAXAARLFYQTC |
| Aegilops_tauschii_subsp._tauschii:(RefSeq: | ............M...G.E........IE.....-NV........FT...S.-P.H.............E..D..P.......TY..N..Q......... |
| XP_020178692.1) | |
| Brachypodium_distachyon:(RefSeq:XP_003567819.1) | ..........S......KG........I....E.-.....L.....H..PS.-T.QV.............A.D..L.......TY.....R......... |
| Hordeum_valgare_subsp._valgere:(XP_044984305.1) | ......I.....M...G.......V.EF......-M.........FS...S.-P.H.............E.AD..L.....H.T...N..Q......... |
| Masa_acuminsta_subsp.malaccensis:(RefSeq: | FR........L..Q.D..GS.I....ET.....F.....G.....VTPP.N..V.NT.LL.RTH......T.G.......N..AF..YKRK..Q...... |
| XP_018681669.1) | |
| Oryza_sativa_Japonica_Group:(RefSeq: | F...........F...L....M.....V......-...I.......E.K.N.-I.QV.Q...........T.G.S........A......K.......A. |
| XP_815638092.1) | |
| Sorghum_bicolor:(RefSeq:XP_021302730.1) | .....T...D....L..N.V.M.....YA.....-...........ERR.N.-M.KV..A.M.H........G........H.A....K.R........T |
| Triticum_aestivum:(UniProt:ABA3B5Z687) | ............M...E.E........ME.....-MV........FM...S.-P.H.............E..D..P.......TY..N..Q......... |
| Zes_mays:(RefSeq:XP_008648328.1} | .....T...D...........M.....YV.................ERR.N.-M.K...T.Q.H......T.GV.......H.A....K.R.......IA |
| | 610 620 630 |
| | ----:----\|----:----\|----:----\|-- |
| Consensus | VLATXDXIKVTQXEPXGXELYGDILISRGXXM |
| Aegilops_tauschii_subsp._tauschii:(RefSeq: | ....L.R.....V..-----..P......AN. |
| XP_020178692.1) | |
| Brachypodium_distachyon:(RefSeq:XP_003567819.1) | ....L.R.....V..-----..A......LN. |
| Hordeum_volgare_subsp._vulgare:(XP_044984305.1) | ....R.H.....D.A-----..P.H..K.AN. |
| Moss_acuminata_subsp.malaccensis;(RefSeq: | ..V.C.F...Q.H.A-----....S....PK. |
| XP_018681559.1) | |
| Oryza_sativa_Japonica_Group:(RefSeq: | ....H.F...N.L..-----.........PK. |
| XP_015638092.1) | |
| Sorghum_bicolor:(RefSeq:XP_021302730.1) | ....F.Y.N...LK.H.E...........SKI |
| Triticum_aestivum;(UniProt:A0A3857587) | ....L.R.....V..-----..P......AN. |
| Zee_ways:(RefSeq:XP_088548328.1} | ....C.Y.....L.RK.D...........LK. |

[symbol]

indicates data missing or illegible when filed

TABLE 1
Percent identities of REC8 protein sequences from monocotyledonous plants.

| | Musa | | | Oryza | | | |
|---|---|---|---|---|---|---|---|
| | acuminata | | | sativa | | Hordeum | |
| | subsp. | Zea | Sorghum | Japonica | Brachypodium | vulgare | |
| | malaccensis: | mays: | bicolor: | Group: | distachyon: | subsp. | Triticum |
| REC8 | (RefSeq: | (RefSeq: | (RefSeq: | (RefSeq: | (RefSeq: | vulgare: | aestivum: |
| Monocot | XP_{—} | XP_{—} | XP_{—} | XP_{—} | XP_{—} | (XP_{—} | (UniProt: |
| % ID | 018681669.1) | 008648328.1) | 021302730.1) | 015638093.1) | 003567819.1) | 044984305.1) | A0A3B5Z6B7) |
| Zea mays: | 55.863 | | | | | | |
| (RefSeq: | | | | | | | |
| XP_008648328.1) | | | | | | | |
| Sorghum | 53.71 | 86.721 | | | | | |
| bicolor: | | | | | | | |
| (RefSeq: | | | | | | | |
| XP_021302730.1) | | | | | | | |
| Oryza | 52.674 | 67.585 | 68.182 | | | | |
| sativa | | | | | | | |
| Japonica | | | | | | | |
| Group: | | | | | | | |
| (RefSeq: | | | | | | | |
| XP_015638093.1) | | | | | | | |
| Brachypodium | 55.628 | 72.386 | 72.683 | 70.425 | | | |
| distachyon: | | | | | | | |
| (RefSeq: | | | | | | | |
| XP_003567819.1) | | | | | | | |
| Hordeum | 51.545 | 64.542 | 65.579 | 63.725 | 75.413 | | |
| vulgare | | | | | | | |
| subsp. | | | | | | | |
| vulgare: | | | | | | | |
| (XP_044984305.1) | | | | | | | |
| Triticum_aestivum: | 52.903 | 67.585 | 68.821 | 65.964 | 79.378 | 86.7 | |
| (UniProt: | | | | | | | |
| A0A3B5Z6B7) | | | | | | | |
| Aegilops | 53.065 | 68.071 | 68.821 | 67.099 | 80.524 | 88.177 | 97.039 |
| tauschii | | | | | | | |
| subsp. | | | | | | | |
| tauschii: | | | | | | | |
| (RefSeq: | | | | | | | |
| XP_020178692.1) | | | | | | | |

Sequnce Alignment 2: SPO11-1 sequences from monocotyledonous plants.
SP011-1 Monocots
Homologs identified by BLAST of Oryza sativa 0s03g54091.1 from MSU -
Osel Release 7 map to menecet NCBI RefSeg, GenBenk end UniProtKB protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MNAGRWIDIXKRRRATVLLAAFVPSFXDDDERRGRRRLEEAALSDLLHXIKGLVXWWVAEVSAGRSPSXALHRYQNYCAXADSAAAAASPSTCACSYDAP |
| Aegilops_tauschii:(RefSeq:XP_020158624.1) | .-..S----G........---------................--...K.....G.....I.......L..........S..A................. |
| Brachyoudium_distachyon:(RefSeq:XP_003559449.1) | .-E..----E........--------D....L.....EQ....--...K.....G......G......V..M......-----TS.....P......... |
| Hordeum_vulgare_subsp._volgare: | .-..-----G........---------.............S..--...K.....G.....L.......L..........---.................. |
| (UniProt:A0A287SD60) | |
| Musa_acuminata_subsp.malaccensis: | ..V......DAQA.PIF.........AQATT.MAGG.VSSSSP....QR...F.RSL.EDL.ND.P..V..D..R...--------HDPSGN.T.GDNL. |
| (XP_009080133.2) | |
| Oryzs_sstiva_Japonica_Group: | .-...----E....VAA.---------.GE..--...Q....T--...R.R...R......A.....TV.........----.S.S..AA.P......V. |
| (RefSeq:NP_001391702.1) | |
| Sorghum_bicolor:(RefSeq:XP_002466440.1) | .-...----D.....AP.---------EG..QQL...Q.....--..RR.....R...E..A......IV....R...----...DS....P......V. |
| Triticum_aestivum:(UniProt.A0A3BGLTD1) | .-..S----G........---------................--...K.....G.....I.......L..........C.-.................. |
| Zea_mays:(RefSeq:NP_001347894.1) | .-...----D.....AP.---------EG..QQL.........--..RR.....R.I.E..A......IV....R...----.S.DS....P......I. |
| | 110 120 130 140 150 160 170 180 190 200 |
| | ----:----\|----:----\|----:----\|----:----\|----:----\|----:----\|----:----\|----:----\|----:----\|----:----\| |
| Consensus | VGTDVLSLERKEFHASRLNVLLRVLLVVQQLLQZNKHCSKADIYYMYPSIFVEQAVVDRAINDICILFKCSRHNLNVVPVAKGLVNGWIRFVEGEKKVYC |
| Aegilops_tauschii:(RefSeq:XP_020158624.1) | .................................E...............M.....I............................................ |
| Brachypodium_dictarbyon:(RefSeq:XP_003559999.1) | ....T.C...........S......F.......Q.................T................................................ |
| Hordeum_volgare_subsp._vaigere: | .................................E...............M.....I............................................ |
| (UniProt:A0A287SD60) | |
| Musa_acuminata_subsp._malaccensis: | N.KEII.-VER.S..Y..L..F....II.....E...G........H.AL.L..........N.........QH......GR......L..L.AGR.I.. |
| (XP_009300133.2) | |
| Oryza_sativa_Japonica_Group: | .........HRGS....................Q.................Q.............V.........................L....E... |
| (RefSeq:NP_001091702.1) | |
| Sorghum_bicolor:(RefSeq.XP_002466440.1) | .........HNDY.T..............K...Q...................V.............L............V................... |
| Triticum_aestivum:(UniProt:A0A3BGLTD1) | .................................E...............M.....I............................................ |
| Zea_mays:(RefSeq:NP_001347894.1) | .........H.DY.I..........H.......Q...................V..........................V..........M........ |
| | 210 220 230 240 250 260 270 280 290 300 |
| | ----:----\|----:----\|----:----\|----:----\|----:----\|----:----\|----:----\|----:----\|----:----\|----:----\| |
| Consensus | ITNVNAAFXIPVSIEAIKDVVSVAHYILVVEKETVFQRLANDKFCERNRCIVITGRGYPDIPTRRFLRYLVEQLHLPAYCLVDSDPYGFDILATYKFGSM |
| Aegilops_tauschii:(RefSeq:XP_070158678.1) | ........P........................A............K..................................................... |
| Brachypodium_distachyon:(RefSeq:XP_003559449.1) | ........S......T...I........................................V.......H............................... |
| Hordeum_vulgare_subsp._vulgare: | .....T..P........................A...........KK...........................R......................... |
| (UniProt:A0A287SD60) | |
| Musa_acuminata_subsp.malaccensis: | .NSPSTVYP...CL. [symbol] VV.I....RF.I.....S........R.....H...........V......CL..QH....Y.....C........MV.R.... |
| (XP_009080133.2) | |
| Oryza_sativa_Japonica_Group: | V.......S...............D...I.................................................V.....A..............L |
| (RefSeq:NP_001391702.1) | |
| Sorghum_bicolor:(RefSeq:XP_002466440.1) | ........S...D......................................................................................L |
| Triticum_aestivum:(UniProt:A0A3BGLTD1) | ..... [symbol] ..P........................A............K..................................................... |
| Zea_mays:(RefSeq:NP_001347894.1) | ..S.....S...D...........................................................L..........................L |
| | 310 320 330 340 350 360 370 380 390 400 |
| | ----:----\|----:----\|----:----\|----:----\|----:----\|----:----\|----:----\|----:----\|----:----\|----:----\| |
| Consensus | QLAYDANLLRVPEIRWLGVFTSDFEEYCLPDCCLLXLSSEDARKXEGILTRCYLHREAPEWRLELEAMLZKGVEFEIEALSASSISFLSQEYIPQKIKLG |
| Aegilops_tauschii:(RefSeq:XP_070158678.1) | .......V.........................Q.Q........L..........K.......K.....EM........................Q.R.. |
| Brachypodium_distachyon:(RefSeq:XP_003559449.1) | ............G..................Y...D......K.L.................A......E.............................. |
| Hordeum_vulgare_subsp._vulgare: | .M.....V.........................Q.P.....T..L..................K..E..E...........S.................. |
| (UniProt:A0A287SD60) | |
| Musa_acuminata_subsp.malaccensis: | .M............S.....H..LQK.H...R...E.T.D.KK.A.AM.L....QK...K..M...V..QT.......S..FN.L....-....T..HD. |
| (XP_009080133.2) | |
| Oryza_sativa_Japonica_Group: | .......F....D............D.R.......H........A....S.........Q.........Q............C......E....K...Q. |
| (RefSeq:NP_001391702.1) | |
| Sorghum_bicolor:(RefSeq:XP_002466440.1) | ...H........D......................H..P.....A.R..A.......V....S......Q............N......D........Q. |
| Triticum_aestivum:(UniProt:A0A3BGLTD1) | .......I.........................Q.Q........L..........K.......K.....EM........................Q.R.. |
| Zea_mays:(RefSeq:NP_001347894.1) | ...H........D......................H..P.....A....A............S......Q............N......H.......... |
| | --- |
| Consensus | RXI |
| Aegilops_tauschii:(RefSeq:XP_070158678.1) | .Y. |
| Brachypodium_distachyon:(RefSeq:XP_003559449.1) | .H. |
| Hordeum_vulgare_subsp._vulgare: | .Y. |
| (UniProt:A0A287SD60) | |
| Musa_acuminata_subsp.malaccensis: | SYL |
| (XP_009080133.2) | |
| Oryza_sativa_Japonica_Group: | .H. |
| (RefSeq:NP_001391702.1) | |
| Sorghum_bicolor:(RefSeq:XP_002466440.1) | MHL |
| Triticum_aestivum:(UniProt:A0A3BGLTD1) | .Y. |
| Zea_mays:(RefSeq:NP_001347894.1) | MHL |

[symbol]

indicates data missing or illegible when filed

TABLE 2
Percent identities of SPO11-1 protein sequences from monocotyledonous plants.

| | | | | Oryza | | | |
|---|---|---|---|---|---|---|---|
| | Musa | | | sativa | | | |
| | acuminata | Zea | Sorghum | Japonic | | Brachypodium | |
| | subsp. | mays: | bicolor: | Group: | Aegilops | distachyon: | Triticum |
| SPO11-1 | malaccensis: | (RefSeq: | (RefSeq: | (RefSeq: | tauschii: | (RefSeq: | aestivum: |
| Monocot | (XP_{—} | XP_{—} | XP_{—} | XP_{—} | (UniProt: | XP_{—} | (UniProt: |
| % ID | 009380133.2) | 008643458.1) | 021304292.1) | 015630525.1) | M8BE82) | 003559449.1) | A0A3B6LTD1) |
| Zea mays: | 42.384 | | | | | | |
| (RefSeq: | | | | | | | |
| XP_008643458.1) | | | | | | | |
| Sorghum | 48.25 | 80.867 | | | | | |
| bicolor: | | | | | | | |
| (RefSeq: | | | | | | | |
| XP_021304292.1) | | | | | | | |
| Oryza sativa | 50.877 | 56.522 | 65.274 | | | | |
| Japonic Group: | | | | | | | |
| (RefSeq: | | | | | | | |
| XP_015630525.1) | | | | | | | |
| Aegilops | 44.717 | 49.533 | 54.948 | 53.671 | | | |
| tauschii: | | | | | | | |
| (UniProt: | | | | | | | |
| M8BE82) | | | | | | | |
| Brachypodium | 57.895 | 62.557 | 71.354 | 73.958 | 66.076 | | |
| distachyon: | | | | | | | |
| (RefSeq: | | | | | | | |
| XP_003559449.1) | | | | | | | |
| Triticum | 57.463 | 61.327 | 69.43 | 74.352 | 72.362 | 86.563 | |
| aestivum: | | | | | | | |
| (UniProt: | | | | | | | |
| A0A3B6LTD1) | | | | | | | |
| Hordeum | 58 | 61.327 | 69.531 | 73.698 | 69.949 | 87.013 | 94.301 |
| vulgare | | | | | | | |
| subsp. | | | | | | | |
| vulgare: | | | | | | | |
| (UniProt: | | | | | | | |
| A0A287SD60) | | | | | | | |

Sequence Alignment 3: PAIR1 protein sequences from monocotyledonous plants.
PAIR1 Monocots
Homologs identified by BLAST of Oryza sativa 0s03g01590,1 from MSU -
Osal Release 7 map to monocot NCBI RefSeq, GenBank and UniProtKa protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MXGRYGEYNCPRQQRIQNRSASHSITPLLLGCSSGIGDHHCRRRLLAPARSREXXMKLKINKACDLXSISVLPPRRTGGSSGAGGGXXXSXAAAVAAGSQ |
| Musa_acuminata_subsp._malaccensis:(RefSeq: | .-------------------------------------------------------..R.......G.........S..M.SG.D.LGR.Q.SQIRSQ.. |
| XP_018685297.1) | |
| Triticumaestivum:(UniProt:A0A386C7X9) | .-------------------------------------------------------..........A..........R....G...GGT.AP..AS.AQ. |
| Hordeum_vulgare_subsp._vulgare:(RefSeq: | .-------------------------------------------------------..T......IA...............G.---GRMS...AQQQQ. |
| XP_044983266.1) | |
| Aegilops_tauschii:(Uniprot:N1R219) | .------------------------------------------------------AMSG..SSIPQTDRL.------------------DWLS.WKL.II |
| Brachypodium_distachyon:(RefSeq:XP_014751501.1) | .--------------------------------------------------------------------------------------------------- |
| Sorghum_bicolor:(UniProt:A0A1B6QR58) | .A...................................................QA...........G.............G...E--AG.S......... |
| Zeamays:(RefSeq:XP_008660580.1) | .-------------------------------------------------------..........G.............G....--AG.S..P...... |
| Zeamays:(GenBank:ONL92656.1) | .T.--------------------------------------------------AG...........S.............G...---SSAA......... |
| Oryza_sativa_Japonica_Group:(RefSeq: | .-------------------------------------------------------...M.....IA................----SA.GSV...VA.. |
| XP_015632198.1) | |
| | 110 120 130 140 150 160 170 180 190 200 |
| | ----:----\|----:----\|----:----\|----:----\|----:----\|----:----\|----:----\|----:----\|----:----\|----:----\| |
| Consensus | QRSQPMSQXSXSQGXGXXXSXXXXXGASFSQGMGSGGASFSQGRGASFSQSXSTGGAAAAFSQGGGGGGGGASFSQGRGASFSQGSGXGGAXXXXSXGGG |
| Musa_acuminata_subsp._malaccensis:(RefSeq: | .S-------------------------------------------------------------------------------------------------- |
| XP_018685297.1) | |
| Triticum_aestivum:(Uniprot:A0A386C7X9) | ....S...K.F...I.ASF.QGIGS.----------------------------------------------------.........G...AFSQ-G... |
| Hordeum_vulgare_subsp._vulgare:(RefSeq: | ....S...K.F...G.GVG.FSQSV..........................S...................................T...VAAF.Q... |
| XP_644983266.1) | |
| Aegilops_tauschii:(UniProt:N1R219) | V.ATYL---------------------------------------------------------------------------------------------- |
| Brachypodium_distachyon:(RefSeq:XP_014751501.1) | ---------------------------------------------------------------------------------------------------- |
| Sorghum_bicolor:(UniProt:A0A1B6QR58) | .......HSQQ.---------------------------------------F------------------------------------------------ |
| Zeamays:(RefSeq:XP_008660580.1) | ........H.------------------------------------------------------------------------------------------ |
| Zeamays:(GenBank:ONL92656.1) | .....L..Q.------------------------------------------------------------------------------------------ |
| Oryza_sativa_Japonica_Group:(RefSeq: | P....L..SQQ.---------------------------------------------------------------------------------------- |
| XP_015632198.1) | |
| | 210 220 230 240 250 260 270 280 290 300 |
| | ----:----\|----:----\|----:----\|----:----\|----:----\|----:----\|----:----\|----:----\|----:----\|----:----\| |
| Consensus | XGSXGAXFSQGXGGGGAAFSQGXXGGGXSFSQGGGXSSLLHSQSQLSQASLDENELSCHLASPARDORFGLHDKDSSKRMXSXPXSSASSCVREESQLQL |
| Musa_acuminata_subsp._malaccensis:(RefSeq: | ------------------....I------------------.L.....S.FE.P.V--------N.....SQE..N.L.KI.SLAPI.--LT........ |
| XP_018685297.1) | |
| Triticum_aestivum:(Uniprot:A0A386C7X9) | G.AA---....G..........GG...A.......SA....A...........S....LSQA.......AMQE-.P....S.YLA......M.D...... |
| Hordeum_vulgare_subsp._vulgare:(RefSeq: | A..G..D....R..........GG...A.......SG....A.....E...N.....Q---A.......S.QE-.....TP.Y.A...-.....D..... |
| XP_644983266.1) | |
| Aegilops_tauschii:(UniProt:N1R219) | -------------------.------------------------T.VPSPIVTSICH.-----VG..G.....-......PPF.P....RAHE.PQ.Q.P |
| Brachypodium_distachyon:(RefSeq:XP_014751501.1) | ------P---------------------------------------LE...GQT...QQ.PA..H..I....--MV.SQIP.F.PN..-...Q....Q.. |
| Sorghum_bicolor:(UniProt:A0A1B6QR58) | --.-----------------..VM.--S-----..A...........................T..............K.P.L.V...-........... |
| Zeamays:(RefSeq:XP_008660580.1) | ------------------....VV.-------S..A....................T.............F..-.L..K.T.L.V...-........... |
| Zeamays:(GenBank:ONL92656.1) | ------------------....AV.--S-----..A....................T..........G.R...-....K.T.L.VT..-........... |
| Oryza_sativa_Japonica_Group:(RefSeq: | ------------------....A------------SA........F..V...D...T.-.P..T.........-......S.L.A...-..A........ |
| XP_015632198.1) | |
| | 310 320 330 340 350 360 370 380 390 400 |
| | ----:----\|----:----\|----:----\|----:----\|----:----\|----:----\|----:----\|----:----\|----:----\|----:----\| |
| Consensus | AKXPSNPXHRWNXPSLXDXRSFXELHSXLFECLTGSGLCGVXXNEDVERKFQHLASSVHKVGMVLDSVQNDVMQLNRAMKEASLDSGSIQQKXVVLXLSL |
| Musa_acuminata_subsp._malaccensis:(RefSeq: | TRAS..VM....AA.MS.N.-.-------------------QVS.EL.H.LR.IE..INR...I.....S....V...A..L..EVEG.R..VS---.-- |
| XP_018685297.1) | |
| Triticum_aestivum:(Uniprot:A0A386C7X9) | ..I.T..I...S-..FL.S.C--------------------QV........L......Q.M..I.............G............K.F.--L--- |
| Hordeum_vulgare_subsp._vulgare:(RefSeq: | TTI.T..I...S-...P.S.------------------.--QI..E..............L..I.............S............K.F.------ |
| XP_644983266.1) | |
| Aegilops_tauschii:(UniProt:N1R219) | VIV..STTL...-..PT.T.-C-------------------RV...A..R....SG.............T..I....T..D...K..VV...FD---.-- |
| Brachypodium_distachyon:(RefSeq:XP_014751501.1) | ..ISM.TI-P.RN.C.AES.------------------.-QVA...A.HR..Q..N..CRIE..........I....S........D...R.V.L.DN.. |
| Sorghum_bicolor:(UniProt:A0A1B6QR58) | ..T....V....-...P.G.--C-----------------.VPT.........M............................A.....M...I..----- |
| Zeamays:(RefSeq:XP_008660580.1) | ..TS...V....-...P.G..-------------------.GPT.........M...............T............T.........I.L----- |
| Zeamays:(GenBank:ONL92656.1) | ..TS...V....-...P.S.Y.I....F..........T..VPA.........M.................T..........A.........I..----- |
| Oryza_sativa_Japonica_Group:(RefSeq: | ..L....V....-..IA.T..------G-------------QVT................M...V....S...................R..IA.----- |
| XP_015632198.1) | |
| | 410 420 430 440 450 460 470 480 490 500 |
| | ----:----\|----:----\|----:----\|----:----\|----:----\|----:----\|----:----\|----:----\|----:----\|----:----\| |
| Consensus | QKILMVXDLEAGSIQQNDXLLEXSLQQILKGQDDLKALXEXSTKSWXDQLXVLNSHXXKLBEISSXLSXLPKQXZXDLXQLXGDIFRIFTKXMEXIVRAI |
| Musa_acuminata_subsp._malaccensis:(RefSeq: | --------------------..N.M..MV.WE..I..FIGG.LT.IS...IKIS.-SG.VN..A.AVAT.QE.MFSR.AR.ECEVC.F.-SEK.VRESG. |
| XP_018685297.1) | |
| Triticum_aestivum:(Uniprot:A0A386C7X9) | --------------------..D..K............LKG.....P...GI.---TR..D....I..V....VQTEFE.FK.....F...E..GV...V |
| Hordeum_vulgare_subsp._vulgare:(RefSeq: | ------------------L-..D..K........I...F.G..E..P...S.....TR..D.MT.I..A..NHLQAEFG..K..TY.....E..G.A.G. |
| XP_644983266.1) | |
| Aegilops_tauschii:(UniProt:N1R219) | --------------------..DT..K.I...N...V.A.N.....L...N.I..RTS..N.M.LVV.VC...VQA..RE.H.....VL..D..GV..D. |
| Brachypodium_distachyon:(RefSeq:XP_014751501.1) | ......Q.G.........V..DN..........G..E.L.GG....P...S.....TST.S....I..VWQEEIRA..R..H.........E.KGV.... |
| Sorghum_bicolor:(UniProt:A0A1B6QR58) | ------L--------------DT...KM..........V.SN...IS...T.....SN..D....T..I....IET..K.QHS......R.D..E....V |
| Zeamays:(RefSeq:XP_008660580.1) | --------.------------DAL..KN..........V.SN...IS...T....YSN..D....T..I....IETN.K.QQS.T....R.D..E....V |
| Zeamays:(GenBank:ONL92656.1) | --------------------.DT.M.KN....G...V.V.SW..NIA...A.....SN..V....A..V....IER..K.QQS......RWW..E.A..V |
| Oryza_sativa_Japonica_Group:(RefSeq: | --------.------------.S...............FGS...H.P..TS....LGS..N....T.AT.QT.MQA--R..Q..QTTVLNSNASK-SNE. |
| XP_015632198.1) | |
| | 510 520 530 540 550 560 570 580 590 600 |
| | ----:----\|----:----\|----:----\|----:----\|----:----\|----:----\|----:----\|----:----\|----:----\|----:----\| |
| Consensus | RSLNXXXXXXXQMXXDOSCTTNGRPLMNQLPVDRNERPQVNQTPEVTRMSXTPVATMVXQTXVASLVNQTPVARGSPLMNQXPXANXXXXMNQTXVANGR |
| Musa_acuminata_subsp._malaccensis:(RefSeq: | K.S.MQHSVKF.SP----------------------------M--------------------------------------------------------- |
| XP_018685297.1) | |
| Triticum_aestivum:(Uniprot:A0A386C7X9) | ...DSRLPGVM..LA.......ÅK....HTAMENE----S------------------------P.M...Q..E.......G.V.DRRPQKKP.A..... |
| Hordeum_vulgare_subsp._vulgare:(RefSeq: | ..I.TRED.-M..LAE......AKT...---------------------------------Q-------.A..H.......T.V.YGSTL...GA..D.S |
| XP_644983266.1) | |
| Aegilops_tauschii:(UniProt:N1R219) | ...STK-P.VMP,LP.H--------------------------V---------------------------..K......KMSV..ERPT..-------- |
| Brachypodium_distachyon:(RefSeq:XP_014751501.1) | ....SR-P.AL..LE.R..YP.E.TWSS-----Q--------------------------V---------Q..D.......I.L..ERLK..-------- |
| Sorghum_bicolor:(UniProt:A0A1B6QR58) | ....SKID.-I..PT..R..I.............................Q.......N..S.........A.N.RH.VS.I.A..GKTLVS..S..... |
| Zeamays:(RefSeq:XP_008660580.1) | ....NKID.-I..PT...................----------N-----------------------ER.QVNQT.EVTRVSQTPVATMA..RP..K.. |
| Zeamays:(GenBank:ONL92656.1) | K...SKID.-I..PTE.R..............................--V-------S..T.......A.A.N.RH..SKT.A..GKSV.R..PAE... |
| Oryza_sativa_Japonica_Group:(RefSeq: | S.TLATLQTQH.---------------------------------A---------------------------DIRQ.RCDVFRVFTKEM---------- |
| XP_015632198.1) | |
| | 610 620 630 640 650 660 670 680 690 700 |
| | ----:----\|----:----\|----:----\|----:----\|----:----\|----:----\|----:----\|----:----\|----:----\|----:----\| |
| Consensus | XLXSXQTPVAXGXPLXXNQXXXANGXXXXXQVPXXAANGXPXXNQTPVAXGRPXMXXQXXXXXGRSQMNQIPVAXGHPHTXXXXPAXXVXPAPLYXPXXX |
| Musa_acuminata_subsp._malaccensis:(RefSeq: | ----------S.NYMDIKESLEG.IEEKK-------VSAA.LMSN---KQRS.PFKKEGKLESFK.KLTE------------------------------ |
| XP_018685297.1) | |
| Triticum_aestivum:(Uniprot:A0A386C7X9) | PKRKA..A..N.R.QRK..AAG..WRPHRKKPTEI...SR.QRHL.R..N...QV-K.TEAAD....T..K.E.N.----KALI.VTKPI.V.V.YHR.- |
| Hordeum_vulgare_subsp._vulgare:(RefSeq: | P.MNH.G... [symbol] .R.Q-KR.RPA...RSQRKKQTPV.SGRAQRKR.R...N...QV-K.AQTAD.......K.E.N.EALMKQVL..TQ.A.....VQR.- |
| XP_644983266.1) | |
| Aegilops_tauschii:(UniProt:N1R219) | -----.....K.S..-M.KMSV..ERPTMN.T.--V...R.LM......N.S.MIIN.ARVGN....LK.T..-------T------------------- |
| Brachypodium_distachyon:(RefSeq:XP_014751501.1) | -----....SI.E..-M.LAVV...SPLIN.S.--V...RSQM......N...Q.-DRTPVAN..PH.E..--------.TNLF..RSSCSTK------- |
| Sorghum_bicolor:(UniProt:A0A1B6QR58) | S.M.-.V.T.N.K..-T..ASL...GSLMS...--....K.LT..I.APK...M.-R.KTGER..P........S..T..N.I-..PE.H.....F.ASA |
| Zeamays:(RefSeq:XP_008660580.1) | H.V.-...A.N.K..-T..TSV...RSLMS...--....K.LT..IA.PK...M.-R.KTAEG..P........D.....N.IHAPEVRH.V...C.--- |
| Zeamays:(GenBank:ONL92656.1) | H.V.-...A.N.KT.-KR.TSVT..ISLMS...--....K.VVI.I.APK...M.-R.KTAES..TE..E....S.R...N.I-.IAE.H.....C.A.Q |
| Oryza_sativa_Japonica_Group:(RefSeq: | -----EGV.RAIRSVNSRPAAMQMMADQSY...--VS..WTQI......A..SP.-NRAPVAA...R...L.E-------T.VLS.HL.Y..KVT----- |
| XP_015632198.1) | |
| | 710 720 730 740 750 760 770 780 790 800 |
| | ----:----\|----:----\|----:----\|----:----\|----:----\|----:----\|----:----\|----:----\|----:----\|----:----\| |
| Consensus | KXAADPKPKVEQGKXKAXPGAQKLXGSXXXRPVXPKQEEXAXXXVWXZXPAXKXEKVVXIIIDSDDDSDXEGGGSEXXASCVILXXXXXGXGXEXGXXEX |
| Musa_acuminata_subsp._malaccensis:(RefSeq: | -----..HT.KHKHII--.--H------------T....F-----------------.RV.V.L.EE..---..----I..L.VNKG--------TGKKS |
| XP_018685297.1) | |
| Triticum_aestivum:(Uniprot:A0A386C7X9) | --.V.L..R...WMVP------..A.PSYS..AP..EQ.L.IQ...TEA.IN.MQATLG.L......G.Y..AD.DWS......KLEAGCP.D.VVAE.A |
| Hordeum_vulgare_subsp._vulgare:(RefSeq: | --KEGVQ..A...MS..GT.....A..GDS..AS..EQ.L.IQ.D.AEA.IN.A--MLA.L....EEEEE-.....GN....L.KSTEA.-----.GVAM |
| XP_644983266.1) | |
| Aegilops_tauschii:(UniProt:N1R219) | ----.AM.QRD---------------------LT.E-------------------------.NGRPQK.------------------------------- |
| Brachypodium_distachyon:(RefSeq:XP_014751501.1) | --......NI...DV..TT--P..F..SYM--LA...G.VLNR...QQE.TK.AP--.T.M.......E---.H----..W...NTEPADEMK.ASRE.G |
| Sorghum_bicolor:(UniProt:A0A1B6QR58) | .A.....A..DD..L.VL.--...T..RSA--.T.....A.ST.F---SR.AAT....IF.E.......---VRA---S.....RSSGS.A.AGA.ER.C |
| Zeamays:(RefSeq:XP_008660580.1) | --..G.R....E..LR.V.--...T.CRSR--.T.....A.TNTKVVVGR.AAT....IF.D.D.S..----....AL......RPAPL.----S.AG.C |
| Zeamays:(GenBank:ONL92656.1) | -V.T......DEE.L..L.--...TS.RSR--.A.....A.NT..I--SR.AAT....IF.D.....N.-------VR......RPSGS.---SL.ER.C |
| Oryza_sativa_Japonica_Group:(RefSeq: | ----.L........V..A.--..PFA.SYY.-.A.....V.IR...IQV..K.AP--.S...E.....E-------GR......KTE-------T.SK.W |
| XP_015632198.1) | |
| | 810 820 830 840 850 860 870 |
| | ----:----\|----:----\|----:----\|----:----\|----:----\|----:----\|----:----\|-- |
| Consensus | XXXXXGAEEXLEILRRARXIDXTALPDVMXXKRRMGKAAPERREMQAXVXXXXPXKRTXXPNPKYDAZXXXX |
| Musa_acuminata_subsp._malaccensis:(RefSeq: | SFMEEVQ.DT.R...K..-----------M.K.---------.Q.NSITVV--------------------- |
| XP_018685297.1) | |
| Triticum_aestivum:(Uniprot:A0A386C7X9) | --TSE.EG.A........------------...--------...AN.NANATVLV------------Q-DQR |
| Hordeum_vulgare_subsp._vulgare:(RefSeq: | AEEAM.EG.A........----------K-R..--------.E.AN.V.LD-----------------H--- |
| XP_644983266.1) | |
| Aegilops_tauschii:(UniProt:N1R219) | ------------------------------------------------------------------------ |
| Brachypodium_distachyon:(RefSeq:XP_014751501.1) | MQLLWS.RKRSRRK.ET.C..A......RG.K.........AQ.EE.VHVEAI.A...CR.......EQWTA |
| Sorghum_bicolor:(UniProt:A0A1B6QR58) | DLM.V....SQ..M....---K---------..--------......T.ASMP.-----E---------VGH |
| Zeamays:(RefSeq:XP_008660580.1) | DLV.V....SQ.......------------...--------......I.ACMP.----E----------LGY |
| Zeamays:(GenBank:ONL92656.1) | DLM.V....SQ..P....K------------..T-------......T.GVACM----P----------LPA |
| Oryza_sativa_Japonica_Group:(RefSeq: | KVT.Q.T..G........------------...--------.....SI.LAS-------------------- |
| XP_015632198.1) | |

[symbol]

indicates data missing or illegible when filed

TABLE 3
Percent identities of PAIR1 protein sequences from monocotyledonous plants.

| | Musa | | Hordeum | | | | | |
|---|---|---|---|---|---|---|---|---|
| | acuminata | | vulgare | | | | | |
| | subsp. | | subsp. | | Brachypodium | | Zea | |
| | malaccensis: | Triticum | vulgare: | Aegilops | distachyon: | Sorghum | mays: | Zea |
| PAIR1 | (RefSeq: | aestivum: | (RefSeq: | tauschii: | (RefSeq: | bicolor: | (RefSeq: | mays: |
| Monocot | XP_{—} | (UniProt: | XP_{—} | (UniProt: | XP_{—} | (UniProt: | XP_{—} | (GenBank: |
| % ID | 018685297.1) | A0A3B6C7X9) | 044983266.1) | N1R2L9) | 014751501.1) | A0A1B6QR58) | 008660580.1) | ONL92656.1) |
| Triticum | 23.184 | | | | | | | |
| aestivum: | | | | | | | | |
| (UniProt: | | | | | | | | |
| A0A3B6C7X9) | | | | | | | | |
| Hordeum | 22.141 | 57.324 | | | | | | |
| vulgare subsp. | | | | | | | | |
| vulgare: | | | | | | | | |
| (RefSeq: | | | | | | | | |
| XP_044983266.1) | | | | | | | | |
| Aegilops | 18.502 | 25.701 | 22.895 | | | | | |
| tauschii: | | | | | | | | |
| (UniProt: | | | | | | | | |
| N1R2L9) | | | | | | | | |
| Brachypodium | 17.04 | 30.976 | 28.61 | 29.61 | | | | |
| distachyon: | | | | | | | | |
| (RefSeq: | | | | | | | | |
| XP_014751501.1) | | | | | | | | |
| Sorghum | 22.687 | 38.108 | 35.525 | 24.434 | 29.184 | | | |
| bicolor: | | | | | | | | |
| (UniProt: | | | | | | | | |
| A0A1B6QR58) | | | | | | | | |
| Zea mays: | 27.083 | 42.178 | 39.08 | 27.098 | 31.013 | 66.269 | | |
| (RefSeq: | | | | | | | | |
| XP_008660580.1) | | | | | | | | |
| Zea mays: | 24.598 | 38.937 | 36.156 | 24.959 | 29.602 | 69.4 | 67.903 | |
| (GenBank: | | | | | | | | |
| ONL92656.1) | | | | | | | | |
| Oryza sativa | 29.354 | 38.735 | 36.75 | 27.006 | 32.721 | 39.493 | 44.658 | 42.165 |
| Japonica | | | | | | | | |
| Group: | | | | | | | | |
| (RefSeq: | | | | | | | | |
| XP_015632198.1) | | | | | | | | |

Sequence Alignment 4: OSD1 protein sequences from monocotyledonous plants.
OSD1 Monocots
Homologs identified by BLAST of Oryza sativa Os02g37350.1 from MSU -
Osa1 Release 7 map to monocot NCBI RefSeq, and UniProtK8 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MSYTNFSXBKXMXEVRXASAXALADXSGGXXGGGFFIRARVASPGAVXXXGAVKPLARAVXSPSSNKENVPPXWAARAXPXKRRSPLPEWYPRTPLRDIT |
| Musa_acuminata_subsp._malaccensis:(RefSeq: | .-----------A.T.QGVNRG-RETT.SATNL..W...---AAYLASRT.SRSTRVQS.VFATDD....T.SR...RRGRT.K................ |
| XP_009415567.1) | |
| Brachypodium distachyon:(UniProt:I1I835) | .-----------V...TV..T.V.ER.----........-.E.....VE...A.R....PLT..........A..VATTQ-....T.......S...... |
| Hordeum vulgare subsp. vulgare:(UniProt:F2EGV1) | .-----------A...I...T.A..R...--.....V..-.V.....VD.........L.P...........A...WTA.-....F.............. |
| Triticum aestivum:(UniProt:A0A3B6PLJ3) | .-----------A...I...T.V..R.----.....V..-.......VD...........P...........A...WTA.-....S.............. |
| Aegilops tauschii:(UniProt:M80G24) | .-----------A...M...A.V..R.----.....V..-.......VD...........P...........A...W.A.-....S.............. |
| Sorghum bicolor:(UniProt:C5XWE8) | .-----------PQL.T...PV..RN.----T..I..........-----..........RTHF........VG....K.-.......D....S...... |
| Oryza sativa:(RefSeq:XP_015635088) | .-----------P.M.DSK.T..GEL...---.......-......LAAR.PG......FIR..N.......V..VK.TAT.......D........... |
| Zea mays:(UniProt:84F675) | .-----------P.S.DGRSED...L...VG........-......LAVR.VR......YI...R....LL.V..L.VT.-TK.....G........... |
| Zea mays:(GenBank:AQK72186.1) | .------RNF..L...T.R.P....I.----.....M.T-.E.....LVM....RP..QFL...........VG.F..T.-...T...D........... |
| Zea mays:(GenBank:AQK72188.1) | .......QDY..PQL.T...P...SN.----A.............TSQA...A.......RT.-----------....K.-.......D....V...... |
| | 110 120 130 140 150 160 170 180 190 200 |
| | ----:----\|----:----\|----:----\|----:----\|----:----\|----:----\|----:----\|----:----\|----:----\|----:----\| |
| Consensus | SIXKAXERRRSRLRXAAARQQJQWXEXSXSSEPXDXEYSSEPENPAQTDZXXHSXPDXSXXXTXETLGXXVXXXXXXXXVAKSATCLAEGXXXXXSXASD |
| Musa_acuminata_subsp._malaccensis:(RefSeq: | V.VN.L....M.V.A..TAR.RTSDPE-----.-----------------AA----------VEKG.---LDSSSIPAAAGS.SVSAT.QPPQIC.SSNA |
| XP_009415567.1) | |
| Brachypodium distachyon:(UniProt:I1I835) | ..I..V..-KNL..D......L..T.D.--...EN---------...A.QDV.RRTPPTNGTLAAAAVASDPAGSAQAVAST....V...TLKA---.TG |
| Hordeum vulgare subsp. vulgare:(UniProt:F2EGV1) | ..I..V..-K...QD....H.L..DDEED....D.E.............EGI.----R.TAP. [symbol] ...CV--GSAQVVETT.A.....G..KCATVVK... |
| Triticum aestivum:(UniProt:A0A3B6PLJ3) | .VI..V..-K....D.V....L..D.ED-....E.---------.....EGI.----G.TAP. [symbol] ....--.GSAQVVETT.A.....G..ESTTVVK... |
| Aegilops tauschii:(UniProt:M80G24) | .VI..V..-K....D......L..D.ED-....E.---------.....EGI.----G.TAP. [symbol] ....--.GWAKVVETT.A.....G..ESTTVVK... |
| Sorghum bicolor:(UniProt:C5XWE8) | ..V..L.K-KN..EED....HI..N.D.--PQ.V.---------.TT.VHAE..D..SQSTQ.Q....V-.ASPGSTSA..WNV.SV..DKQEAS.SP.- |
| Oryza sativa:(RefSeq:XP_015635088) | A.A..IQ.--....I...Q.RS.TP.Q-----NT.----------------P.------CTEVRDS.DV-EPGINSTQI..TP.SS..KDSLKIF.SP.- |
| Zea mays:(UniProt:84F675) | A.A..IQ.S...I--...Q.RS.RI.Q.SQ.VNVTT--------...AEQDA.------IAEASHAVASG-SGSTEREA.. [symbol] P..V..DD [symbol] LNVS.L.A- |
| Zea mays:(GenBank:AQK72186.1) | ..V..I.......QN...Q..I..T.DP--.RSV.---------------PI--------TPVQAEQ.G-.PTTVDGQG.GSP....ED.KLKTS.YP.S |
| Zea mays:(GenBank:AQK72188.1) | ..V..L.K-.N..EED....HI.SN.D.--.Q.V.P------------TTAE.. [symbol] ..SQSTQ.Q..P.AVASGPSSTSA.. [symbol] RV.SV...KQEAT----- |
| | 210 220 230 240 250 260 |
| | ----:----\|----:----\|----:----\|----:----\|----:----\|----:----\|----: |
| Consensus | DCSLQXPSXKPNDFSXXDKVEKKLSSSIEQEEKMVRRNLKXTPDPKAAQPSKXPAVQRRTLMSMR |
| Musa_acuminata_subsp._malaccensis:(RefSeq: | SSPRED.------.DQPTEY..N.EIY.GEM.RL.TE....S.M.P.KRA-------K...I... |
| XP_009415567.1) | |
| Brachypodium distachyon:(UniProt:I1I835) | .....T..RQG.H.ALS.LL..L.A..........CQ..TLG-.........TQ........... |
| Hordeum vulgare subsp. vulgare:(UniProt:F2EGV1) | .....S..R----..GE.D...Q.AN..HE.....S......-....TRT..T............ |
| Triticum aestivum:(UniProt:A0A3B6PLJ3) | .....S..R----..GE.D...Q.AN..QE..N..S......-....TR...T............ |
| Aegilops tauschii:(UniProt:M80G24) | .....ST.R----..GEVG...Q.A...DA.....S......-..E.TR...T............ |
| Sorghum bicolor:(UniProt:C5XWE8) | ..-..MAPS......PA.-L...M.............HM.E.--........L-V....I..... |
| Oryza sativa:(RefSeq:XP_015635088) | ET..VT..K-.M..VLL.DM.......................KAA......R-.I......... |
| Zea mays:(UniProt:84F675) | EG.. [symbol] T.PK----.MDPA-AD....G...KV..L..K.....--S [symbol] ...A.RR-.T...N..... |
| Zea mays:(GenBank:AQK72186.1) | .....ATPS.....ALA.-.......................--S.......R-TI...V..... |
| Zea mays:(GenBank:AQK72188.1) | .....VAPS......PA.-.-....G...........HM.E.-H........V-V....I..... |

[symbol]

indicates data missing or illegible when filed

TABLE 4A
Percent identities of OSD1 sequences from monocotyledonous plants.

| | Musa | | | | | | | | |
|---|---|---|---|---|---|---|---|---|---|
| | acuminata | | Hordeum | | | | | | |
| | subsp. | | vulgare | Triticum | | | Oryza | | |
| | malaccensis: | Brachypodium | subsp. | aestivum: | Aegilops | Sorghum | sativa: | Zea | Zea |
| OSD1 | (RefSeq: | distachyon: | vulgare: | (UniProt: | tauschii: | bicolor: | (RefSeq: | mays: | mays: |
| Monocot | XP_{—} | (UniProt: | (UniProt: | A0A3B6 | (UniProt: | (UniProt: | XP_{—} | (UniProt: | (GenBank: |
| % ID | 009415567.1) | I1IB35) | F2EGV1) | PLJ3) | M8CG24) | C5XWE8) | 015635088) | B4FG75) | AQK72186.1) |
| Brachypodium | 26.033 | | | | | | | | |
| distachyon: | | | | | | | | | |
| (UniProt: | | | | | | | | | |
| I1IB35) | | | | | | | | | |
| Hordeum | 29.098 | 55.645 | | | | | | | |
| vulgare | | | | | | | | | |
| subsp. | | | | | | | | | |
| vulgare: | | | | | | | | | |
| (UniProt: | | | | | | | | | |
| F2EGV1) | | | | | | | | | |
| Triticum | 29.614 | 60.169 | 86.192 | | | | | | |
| aestivum: | | | | | | | | | |
| (UniProt: | | | | | | | | | |
| A0A3B6PLJ3) | | | | | | | | | |
| Aegilops | 28.755 | 59.322 | 82.427 | 93.805 | | | | | |
| tauschii: | | | | | | | | | |
| (UniProt: | | | | | | | | | |
| M8CG24) | | | | | | | | | |
| Sorghum | 25.726 | 47.458 | 45.565 | 48.729 | 49.153 | | | | |
| bicolor: | | | | | | | | | |
| (UniProt: | | | | | | | | | |
| C5XWE8) | | | | | | | | | |
| Oryza | 29.596 | 47.28 | 44.262 | 46.154 | 46.154 | 48.101 | | | |
| sativa: | | | | | | | | | |
| (RefSeq: | | | | | | | | | |
| XP_015635088) | | | | | | | | | |
| Zea | 27.778 | 41.975 | 41.736 | 42.553 | 42.979 | 40.664 | 55.508 | | |
| mays: | | | | | | | | | |
| (UniProt: | | | | | | | | | |
| B4FG75) | | | | | | | | | |
| Zea | 26.407 | 51.037 | 48.594 | 51.055 | 52.321 | 54.393 | 54.545 | 47.083 | |
| mays: | | | | | | | | | |
| (GenBank: | | | | | | | | | |
| AQK72186.1) | | | | | | | | | |
| Zea | 22.4 | 44.715 | 40.385 | 42.972 | 43.373 | 72.764 | 42.276 | 38.34 | 50 |
| mays: | | | | | | | | | |
| (GenBank: | | | | | | | | | |
| AQK72188.1) | | | | | | | | | |

Sequence Alignment 5: CYCA1 protein sequences from monocotyledonous plants.
CYCA1 Monocots
Homologs identified by BLAST of Arabidopsis thallana AT1677398 from AGI -
TAIRIE map to monocot NCBI RefSeq, GenBank and UniProtKB protein databases.

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MSSXAAXXRSSSSSXSTAASAXAKRPAXXEGAGGAXXXMAXXAAAXQQQAKKRVALGMITTNVAAAAXGRAAVGGGCEKVAPXTTGNAKLMEATSAAPVK |
| Zea_mays:(RefSeq:MP_001288521.1) | ..TI..SR......-.---..T.....IAD.P..PK-AT.A-------............-....-RG...S...SL.N.MAP.SS-....PT---V.L. |
| Zea_mays:(RefSeq:WP_001105387.2) | ..TH..SR......------.V.....IA...TK.A-GPGPT..----.....T..V...-....PGARV.----AV.....PV..-....P...G..L. |
| Oryza_sativa_Japonica_Group: | ...NL.AS.R....S.V..A.A.....VG..G..GG-GK.AAG..A---.......S..S-....GGG--.----------.GKA......L.A...... |
| (RefSeq:XP_015638726.1) | |
| Sorghum_bicolor:(RefSeq:XP_002455447.1) | ..TI..SR......-----..A.....IS.....PK-V..A-Q------...........-....-RG...V....L.NA..P..S-....P.---V.L. |
| Brachypodium_distachyon:(RefSeq:XP_083566077,1) | ...T..AS.RL...AA.---.T.....MA.N....K-P.GRM..---..P........L.-....G.V..T----......VV.A...R.H....TV... |
| Brachypodium_distachyon:(RefSeq:XP_083566221.1) | ...N..AS.RL..AT----T.T.....MA.S....M-LVDPV.V---.............-.I..G.M..T----......VA.P...R.H.T....... |
| Aegilops_tauschii_subsp._strangulata:(RefSeq: | ...NS.AP.RF..AM..---ST.....VP...RA.A-GP.AAQQ----..........L........A...----....I.VV.....R........... |
| XP_020187359.1) | |
| Oryza_sativa_Japonica_Group:) | .--------------------A.....AG.--..GK-A..GA..-----.......V...-......------------------N...F....W..... |
| (RefSeq:XP_025279970.1 | |
| Hordeum_vulgara_subsp._vulgare:(XP_044975533,1) | ...NP.AP.RF..AM..---.T.....VP...A..RPA.GPA...P............L.-......G...----....I.VV.A...R.S...A..... |
| Triticum_aestivum:(RefSeq:(XP_044356183.1) | ...NS.AP.RF..AM..---ST.....VP...RA.AGP..AQQ-----..........L........A...----....I.VV.....R........... |
| Oryza_sativa_Japonica_Group: | ...SL.SR......-------A.....AG..G.K.AAG..A-------............-......------------------N...F....W..... |
| (RefSeq:XP_025878069.1) | |
| Sorghum_bicolor:(RefSeq:XP_002454820,3) | ..TR..SR......------.A.....MA...KA.GTA..--Q-----....A.......-..V..PA....---AL.....PV..-.........V.L. |
| Sorghum_bicolor:(RefSeq:XP_821311446.1) | ..TF..SR.....-------.AS....MD.N.A.GPRWTRPQ------.T..........-....P.P..R.---AV.....PA.TR....P......L. |
| Musa_acuminata_subsp._malaccensis:(RefSeq: | .NRRPSISS.A.AAS-----.S.....AT.S.SKMPAAL.GQ------.......A...S-.QSKV.R [symbol] P.---------R.LGAKG.NI.-T..S.ST. |
| XP_018672008.1) | |
| | 110 120 130 140 150 160 170 180 190 200 |
| | ----:----\|----:----\|----:----\|----:----\|----:----\|----:----\|----:----\|----:----\|----:----\|----:----\| |
| Consensus | KGSLATSARMVXXMIRESAVKSASXKPAPXXERHXSSXXCKEXVXPPPKYPTXVPXAXXAPAXVPCSSFVSPEHSEDSVSXDETMSTCDSMKSPDFEYID |
| Zea_mays:(RefSeq:MP_001288521.1) | .P.......S.SSS.....D.P..I....PVA..G.AT-.RHMNV.......IADVPSR...L.S.TGL....R......S.................V. |
| Zea_mays:(RefSeq:WP_001105387.2) | .P...-N..TI----....A....I....PV...D..SE..HN.L----...T.HVPSR...L..Y.......R.R....T................... |
| Oryza_sativa_Japonica_Group: | .....-.G...GT.-.A.......A....AI...E.AT-...S.L..-...SI..T.AL..VT.........M.......V.............E..... |
| (RefSeq:XP_015638726.1) | |
| Sorghum_bicolor:(RefSeq:XP_002455447.1) | .P...-...S.SSI-..Q......I....PV...G.AI-..HN.P..-....IADVPSH...L...TGL....R...F..I.D................. |
| Brachypodium_distachyon:(RefSeq:XP_083566077,1) | .....-.....IT.-.......V.TR.T.VT.CRG.TT--QKEIV....L.AAM.IV--..PI.........R..A..M.T................... |
| Brachypodium_distachyon:(RefSeq:XP_083566221.1) | .....-.VQ.AIA.-.SL......TR.T.----------KA.DIV.......VM.I.AS.--I...C..A..Q..E..I.T......S............ |
| Aegilops_tauschii_subsp._strangulata:(RefSeq: | ..A.P-...HASA.-........FT....VT...E..V-...S.-..R....V..I.VP.--VT.F............I.T.....S............. |
| XP_020187359.1) | |
| Oryza_sativa_Japonica_Group: | .....-.G...CT.-.V.......A....AI...E.AP-...S.I..-..LSI..T.AP..VT.........M.......V.....M............. |
| (RefSeq:XP_025279970.1) | |
| Hordeum_vulgara_subsp._vulgare:(XP_044975533,1) | ....P-G...ANA.-.........T....VT...EG.V-...SAP..R....V..I.VP.--VI.F............I.T.....S............. |
| Triticum_aestivum:(RefSeq:(XP_044356183.1) | ..A.P-....ASA.-........FT....VT...E..V-...S.-..R....V..I.VP.--VI.F............I.T.....S............. |
| Oryza_sativa_Japonica_Group: | .....-.G...GT.-.V.......T...SAI...E.AP-...S.L..-..LRI..T.AP..VT.........M.......V................... |
| (RefSeq:XP_025878069.1) | |
| Sorghum_bicolor:(RefSeq:XP_002454820,3) | .P...-N...ASSE-.........I....PVP.LD..TRP.HN.----P..AT.MVPSR...L.........AR......I................... |
| Sorghum_bicolor:(RefSeq:XP_821311446.1) | .PYP.-N....SSE-.........I..G.PV.CLC..TM.QKHNV.......T.MVPSR...L......M...R......M.....I..........F.. |
| Musa_acuminata_subsp._malaccensis:(RefSeq: | ....S-.Y.DTRSG-GS...T..AI..DTNK.SNK..L--SINNG.KIT..AC---------L....TLK.....R....L..........R........ |
| XP_018672008.1) | |
| | 210 220 230 240 250 260 270 280 290 300 |
| | ----:----\|----:----\|----:----\|----:----\|----:----\|----:----\|----:----\|----:----\|----:----\|----:----\| |
| Consensus | NGDSSSVLXSLQRRANENLRISEDRDVEEXKWKKDAPAPMEIDXICDWDWNYEDPQLCATLASDIYWHLREAETRKSKAPSTDFMETIQKDVNPSKRAIL |
| Zea_mays:(RefSeq:MP_001288521.1) | .Q.T.-M.A.....TS.H...........M.R..N.V......R.....SE.......................K.--...................... |
| Zea_mays:(RefSeq:WP_001105387.2) | ..GC.-M.A......D.H..T........M....NG.......S..E..S.L.......A.............MK.--.......K.............. |
| Oryza_sativa_Japonica_Group: | ........G....................T.......S.....Q................................--...................... |
| (RefSeq:XP_015638726.1) | |
| Sorghum_bicolor:(RefSeq:XP_002455447.1) | .Q...-M.A.....T..H...T.......M....N.I......R......E.......................K.--...................... |
| Brachypodium_distachyon:(RefSeq:XP_083566077,1) | .....-..D.......A.......S...GA......T......T..........T.....................--..A...L.KM............ |
| Brachypodium_distachyon:(RefSeq:XP_083566221.1) | ....L-..D...Q...A......ES...GT......TT.....N.S...D..K........P........DT....--..AS..L..M...I........ |
| Aegilops_tauschii_subsp._strangulata:(RefSeq: | .....-L.D.............D..T..GA......A......NV....D..........................--......L............... |
| XP_020187359.1) | |
| Oryza_sativa_Japonica_Group: | ........G....................T..N....S.....Q................................--...................... |
| (RefSeq:XP_025279970.1) | |
| Hordeum_vulgara_subsp._vulgare:(XP_044975533,1) | .....-L.D.............D..T..GT......T......NV....D..........................--......L............... |
| Triticum_aestivum:(RefSeq:(XP_044356183.1) | .....-L.D.............D..T..GA......A......NV....D..........................--......L............... |
| Oryza_sativa_Japonica_Group: | ........G....................T.......S.....Q................................--H........L............ |
| (RefSeq:XP_025878069.1) | |
| Sorghum_bicolor:(RefSeq:XP_002454820,3) | .....-M.A......D.H......T....N....N........RV.....DL......................K.--...................... |
| Sorghum_bicolor:(RefSeq:XP_821311446.1) | .....-..A....Q...H.....E-----N....N....F...H......D......................MK.............H.S......... |
| Musa_acuminata_subsp._malaccensis:(RefSeq: | K..C.-.IT..E.Q..K..H...HAAEAGS.LSI.V.MM..V.D.I...T.HD...F.T...G...R...I...K.--........K....I.A...S.. |
| XP_018672008.1) | |
| | 310 320 330 340 350 360 370 380 390 400 |
| | ----:----\|----:----\|----:----\|----:----\|----:----\|----:----\|----:----\|----:----\|----:----\|----:----\| |
| Consensus | IDWLVEVAEEYRLVPDTLYLTVNYIDRYLEGNEINRQRLQLLEVACHLIAAKVEEICAPQVEEFCYITDNTYERDEVLENEASVLNYLKFEXTARTAKCE |
| Zea_mays:(RefSeq:MP_001288521.1) | ..................................S...........................................D..................... |
| Zea_mays:(RefSeq:WP_001105387.2) | .............A...................................................................................... |
| Oryza_sativa_Japonica_Group: | ...........................................................................................Y........ |
| (RefSeq:XP_015638726.1) | |
| Sorghum_bicolor:(RefSeq:XP_002455447.1) | .......S......................................................................D..T...K.............. |
| Brachypodium_distachyon:(RefSeq:XP_083566077,1) | .........................................................................K....D.................P... |
| Brachypodium_distachyon:(RefSeq:XP_083566221.1) | .......S............................................ [symbol] ....................K..........I............... |
| Aegilops_tauschii_subsp._strangulata:(RefSeq: | .........................................................................K....D..................... |
| XP_020187359.1) | |
| Oryza_sativa_Japonica_Group: | ...........................................................................................Y........ |
| (RefSeq:XP_025279970.1) | |
| Hordeum_vulgara_subsp._vulgare:(XP_044975533,1) | .........................................................................K....D..................... |
| Triticum_aestivum:(RefSeq:(XP_044356183.1) | .........................................................................K....D..................... |
| Oryza_sativa_Japonica_Group: | .....................................................K.............................................. |
| (RefSeq:XP_025878069.1) | |
| Sorghum_bicolor:(RefSeq:XP_002454820,3) | .................................................................................................... |
| Sorghum_bicolor:(RefSeq:XP_821311446.1) | ..................................D........T........................S......D................A...P... |
| Musa_acuminata_subsp._malaccensis:(RefSeq: | ...........................................S.............................K...FQ...D..K...Y......V... |
| XP_018672008.1) | |
| | 410 420 430 440 450 460 470 480 490 500 |
| | ----:----\|----:----\|----:----\|----:----\|----:----\|----:----\|----:----\|----:----\|----:----\|----:----\| |
| Consensus | LRREVRAADVIDEDPALHLEFLANYYAELSLLEYSLESYPPSLVAASAIFLAKFILQPTKKPWNSTLATYTQYKPSELSDCVKALHELFSVGPGSYLPAI |
| Zea_mays:(RefSeq:MP_001288521.1) | ....A....A...............I.................I........R.......Y...............K..E........C...S....... |
| Zea_mays:(RefSeq:WP_001105387.2) | ....A.S..A...............I.................I........R.V.....Y..................E...T....S........... |
| Oryza_sativa_Japonica_Group: | ......V...S.......................N.........................H.............S......................... |
| (RefSeq:XP_015638726.1) | |
| Sorghum_bicolor:(RefSeq:XP_002455447.1) | ....A....A...............I........N........I........R.......Y...........................C...S.T..... |
| Brachypodium_distachyon:(RefSeq:XP_083566077,1) | ......V..............................A..........V..S........C.............A...C..................... |
| Brachypodium_distachyon:(RefSeq:XP_083566221.1) | ....................S..L..I..........V..........L..S........S.............A...L....E.Q...L.A...K.... |
| Aegilops_tauschii_subsp._strangulata:(RefSeq: | ...............H.....................A.............S......A.H.................L..................... |
| XP_020187359.1) | |
| Oryza_sativa_Japonica_Group: | ......V...S.......................N.........................H.............S......................... |
| (RefSeq:XP_025279970.1) | |
| Hordeum_vulgara_subsp._vulgare:(XP_044975533,1) | ...............M.....................A.............S......A.H.................L..................... |
| Triticum_aestivum:(RefSeq:(XP_044356183.1) | ...............H.....................A.............S......A.H.................L..................... |
| Oryza_sativa_Japonica_Group: | ......V...S.......................N.......................A.H.............S..............L.......... |
| (RefSeq:XP_025878069.1) | |
| Sorghum_bicolor:(RefSeq:XP_002454820,3) | ....A.S..A.............S..........N........I........R.......Y......S................................ |
| Sorghum_bicolor:(RefSeq:XP_821311446.1) | ....A....A........................N........I....V...KYV.....Y....................................... |
| Musa_acuminata_subsp._malaccensis:(RefSeq: | ....I....GS..V...Q.....S....I........C.A...I........R.....A.R...A..D...L....D............CTSS.N..... |
| XP_018672008.1) | |
| | 510 520 530 |
| | ----:----\|----:----\|----:----\|- |
| Consensus | REKYSQHKYKFVAKKQCPPSIPAEFFRDATC |
| Zea_mays:(RefSeq:MP_001288521.1) | ...................Q..T.....T.. |
| Zea_mays:(RefSeq:WP_001105387.2) | ...................Q.........A. |
| Oryza_sativa_Japonica_Group: | ....T..........P......T........ |
| (RefSeq:XP_015638726.1) | |
| Sorghum_bicolor:(RefSeq:XP_002455447.1) | ...................Q..T........ |
| Brachypodium_distachyon:(RefSeq:XP_083566077,1) | ...................LV..D....... |
| Brachypodium_distachyon:(RefSeq:XP_083566221.1) | ...........A.......MV..DY.C..A. |
| Aegilops_tauschii_subsp._strangulata:(RefSeq: | ............G.....T.V........A. |
| XP_020187359.1) | |
| Oryza_sativa_Japonica_Group: | ....T..........H....V.S........ |
| (RefSeq:XP_025279970.1) | |
| Hordeum_vulgara_subsp._vulgare:(XP_044975533,1) | ............G.....T.V........A. |
| Triticum_aestivum:(RefSeq:(XP_044356183.1) | ............G.....T.V........A. |
| Oryza_sativa_Japonica_Group: | ....T..........P......T.....S.. |
| (RefSeq:XP_025878069.1) | |
| Sorghum_bicolor:(RefSeq:XP_002454820,3) | ...................Q..T.....T.- |
| Sorghum_bicolor:(RefSeq:XP_821311446.1) | .............R........T.....V.W |
| Musa_acuminata_subsp._malaccensis:(RefSeq: | ...............Y..A.....Y.Q..RN |
| XP_018672008.1) | |

[symbol]

indicates data missing or illegible when filed

TABLE 4B
Percent identities of CYCA1 sequences from monocotyledonous plants.

| | | | Oryza_{—} | | | | Aegilops_{—} |
|---|---|---|---|---|---|---|---|
| | | | sativa_{—} | | | | tauschii_{—} |
| | Zea_{—} | Zea_{—} | Japonica_{—} | Sorghum_{—} | Brachypodium_{—} | Brachypodium_{—} | subsp._{—} |
| | mays: | mays: | Group: | bicolor: | distachyon: | distachyon: | strangulata: |
| TAM | (RefSeq: | (RefSeq: | (RefSeq: | (RefSeq: | (RefSeq: | (RefSeq: | (RefSeq: |
| monocot | NP_{—} | NP_{—} | XP_{—} | XP_{—} | XP_{—} | XP_{—} | XP_{—} |
| % ID | 001288521.1) | 001105387.2) | 015630726.1) | 002455447.1) | 003566077.1) | 003566081.1) | 020187359.1) |
| Zea_{—} | 80.271 | | | | | | |
| mays: | | | | | | | |
| (RefSeq: | | | | | | | |
| NP_001105387.2) | | | | | | | |
| Oryza_{—} | 74.618 | 75.483 | | | | | |
| sativa_{—} | | | | | | | |
| Japonica_{—} | | | | | | | |
| Group: | | | | | | | |
| (RefSeq: | | | | | | | |
| XP_015630726.1) | | | | | | | |
| Sorghum_{—} | 90.196 | 80.897 | 76.923 | | | | |
| bicolor: | | | | | | | |
| (RefSeq: | | | | | | | |
| XP_002455447.1) | | | | | | | |
| Brachypodium_{—} | 72.692 | 72.763 | 77.457 | 73.359 | | | |
| distachyon: | | | | | | | |
| (RefSeq: | | | | | | | |
| XP_003566077.1) | | | | | | | |
| Brachypodium_{—} | 66.667 | 67.446 | 71.869 | 67.636 | 81.518 | | |
| distachyon: | | | | | | | |
| (RefSeq: | | | | | | | |
| XP_003566081.1) | | | | | | | |
| Aegilops_{—} | 72.692 | 75.292 | 79.191 | 74.517 | 81.518 | 76.998 | |
| tauschii_{—} | | | | | | | |
| subsp._{—} | | | | | | | |
| strangulata: | | | | | | | |
| (RefSeq: | | | | | | | |
| XP_020187359.1) | | | | | | | |
| Oryza_{—} | 72.394 | 73.529 | 88.78 | 75.049 | 76.07 | 70.76 | 77.626 |
| sativa_{—} | | | | | | | |
| Japonica_{—} | | | | | | | |
| Group: | | | | | | | |
| (RefSeq: | | | | | | | |
| XP_025879970.1) | | | | | | | |
| Hordeum_{—} | 72.137 | 74.324 | 79.271 | 73.563 | 82.592 | 78.101 | 93.992 |
| vulgare_{—} | | | | | | | |
| subsp._{—} | | | | | | | |
| vulgare: | | | | | | | |
| (XP_044975533.1) | | | | | | | |
| Triticum_{—} | 72.692 | 74.757 | 78.846 | 74.71 | 81.942 | 77.043 | 98.826 |
| aestivum: | | | | | | | |
| (RefSeq: | | | | | | | |
| XP_044356181.1) | | | | | | | |
| Oryza_{—} | 74.419 | 75.342 | 89.784 | 76.953 | 76.311 | 71.401 | 79.029 |
| sativa_{—} | | | | | | | |
| Japonica_{—} | | | | | | | |
| Group: | | | | | | | |
| (RefSeq: | | | | | | | |
| XP_025878069.1) | | | | | | | |
| Sorghum_{—} | 82.364 | 86.22 | 77.264 | 84.932 | 74.806 | 68.544 | 76.311 |
| bicolor: | | | | | | | |
| (RefSeq: | | | | | | | |
| XP_002454880.1) | | | | | | | |
| Sorghum_{—} | 77.649 | 79.183 | 73.18 | 78.876 | 72.78 | 66.538 | 71.622 |
| bicolor: | | | | | | | |
| (RefSeq: | | | | | | | |
| XP_021311446.1) | | | | | | | |
| Musa_{—} | 61.315 | 62.109 | 63.938 | 62.451 | 61.644 | 59.687 | 62.695 |
| acuminata_{—} | | | | | | | |
| subsp._{—} | | | | | | | |
| malaccensis: | | | | | | | |
| (RefSeq: | | | | | | | |
| XP_018678008.1) | | | | | | | |

| | Oryza_{—} | | | Oryza_{—} | | |
|---|---|---|---|---|---|---|
| | sativa_{—} | Hordeum_{—} | | sativa_{—} | | |
| | Japonica_{—} | vulgare_{—} | Triticum_{—} | Japonica_{—} | Sorghum_{—} | Sorghum_{—} |
| | Group: | subsp._{—} | aestivum: | Group: | bicolor: | bicolor: |
| TAM | (RefSeq: | vulgare: | (RefSeq: | (RefSeq: | (RefSeq: | (RefSeq: |
| monocot | XP_{—} | (XP_{—} | XP_{—} | XP_{—} | XP_{—} | XP_{—} |
| % ID | 025879970.1) | 044975533.1) | 044356181.1) | 025878069.1) | 002454880.1) | 021311446.1) |
| Zea_{—} | | | | | | |
| mays: | | | | | | |
| (RefSeq: | | | | | | |
| NP_001105387.2) | | | | | | |
| Oryza_{—} | | | | | | |
| sativa_{—} | | | | | | |
| Japonica_{—} | | | | | | |
| Group: | | | | | | |
| (RefSeq: | | | | | | |
| XP_015630726.1) | | | | | | |
| Sorghum_{—} | | | | | | |
| bicolor: | | | | | | |
| (RefSeq: | | | | | | |
| XP_002455447.1) | | | | | | |
| Brachypodium_{—} | | | | | | |
| distachyon: | | | | | | |
| (RefSeq: | | | | | | |
| XP_003566077.1) | | | | | | |
| Brachypodium_{—} | | | | | | |
| distachyon: | | | | | | |
| (RefSeq: | | | | | | |
| XP_003566081.1) | | | | | | |
| Aegilops_{—} | | | | | | |
| tauschii_{—} | | | | | | |
| subsp._{—} | | | | | | |
| strangulata: | | | | | | |
| (RefSeq: | | | | | | |
| XP_020187359.1) | | | | | | |
| Oryza_{—} | | | | | | |
| sativa_{—} | | | | | | |
| Japonica_{—} | | | | | | |
| Group: | | | | | | |
| (RefSeq: | | | | | | |
| XP_025879970.1) | | | | | | |
| Hordeum_{—} | 77.799 | | | | | |
| vulgare_{—} | | | | | | |
| subsp._{—} | | | | | | |
| vulgare: | | | | | | |
| (XP_044975533.1) | | | | | | |
| Triticum_{—} | 77.67 | 94.186 | | | | |
| aestivum: | | | | | | |
| (RefSeq: | | | | | | |
| XP_044356181.1) | | | | | | |
| Oryza_{—} | 91.498 | 77.992 | 79.377 | | | |
| sativa_{—} | | | | | | |
| Japonica_{—} | | | | | | |
| Group: | | | | | | |
| (RefSeq: | | | | | | |
| XP_025878069.1) | | | | | | |
| Sorghum_{—} | 75.734 | 74.952 | 76.848 | 78.193 | | |
| bicolor: | | | | | | |
| (RefSeq: | | | | | | |
| XP_002454880.1) | | | | | | |
| Sorghum_{—} | 72.32 | 71.401 | 72.147 | 73.633 | 82.813 | |
| bicolor: | | | | | | |
| (RefSeq: | | | | | | |
| XP_021311446.1) | | | | | | |
| Musa_{—} | 63.636 | 62.33 | 63.014 | 64.484 | 63.796 | 61.719 |
| acuminata_{—} | | | | | | |
| subsp._{—} | | | | | | |
| malaccensis: | | | | | | |
| (RefSeq: | | | | | | |
| XP_018678008.1) | | | | | | |

TABLE 5
Table of SEQ ID NOs.

| SEQ | | | Sequence |
|---|---|---|---|
| ID | | Plant | Source |
| NO. | Sequence | Type | (Accession) |
| 1 | REC8 protein | monocot | Zea mays |
| | | | (RefSeq: XP_008648328.1) |
| 2 | SPO11-1 protein | monocot | Zea mays |
| | | | (RefSeq: NP_001347894.11) |
| 3 | PAIR1-1 protein | monocot | Zea mays |
| | | | (RefSeq: XP_008660580.1) |
| 4 | OSD1-1 protein | monocot | Zea mays |
| | | | (UniProt: B4FG75) |
| 5 | PAIR1-2 protein | monocot | Zea mays |
| | | | (GenBank: ONL92656.1) |
| 6 | OSD1-2 protein | monocot | Zea mays |
| | | | (GenBank: AQK72186.1) |
| 7 | OSD1-3 protein | monocot | Zea mays |
| | | | (GenBank: AQK72188.1) |
| 8 | JASON-1 protein | monocot | Zea mays |
| | | | (RefSeq: NP_001130670.1) |
| 9 | JASON-2 protein | monocot | Zea mays |
| | | | (RefSeq: NP_001130438.1) |
| 10 | CYCLIN-A1 protein | monocot | Zea mays |
| | | | (GenBank: NP_001288521.1) |
| 11 | TDM1 protein | monocot | Zea mays |
| | | | (RefSeq: NP_001141923.1) |
| 12 | PS1 protein | monocot | Zea mays |
| | | | (RefSeq: NP_001348386.1) |
| 13 | CYCLIN-A1 | monocot | Zea mays |
| | (CYCA1) protein | | (RefSeq: NP_001105387.2) |
| 14 | OSD1-1 target | monocot | Zea mays |
| | sequence | | |
| 15 | OSD1-1 target | monocot | Zea mays |
| | sequence | | |
| 16 | OSD1-1 target | monocot | Zea mays |
| | sequence | | |
| 17 | OSD1-1 target | monocot | Zea mays |
| | sequence | | |
| 18 | OSD1-1 target | monocot | Zea mays |
| | sequence | | |
| 19 | OSD1-1 target | monocot | Zea mays |
| | sequence | | |
| 20 | OSD1-1 target | monocot | Zea mays |
| | sequence | | |
| 21 | OSD1-1 target | monocot | Zea mays |
| | sequence | | |
| 22 | OSD1-1 target | monocot | Zea mays |
| | sequence | | |
| 23 | OSD1-1 target | monocot | Zea mays |
| | sequence | | |
| 24 | OSD1-2 target | monocot | Zea mays |
| | sequence | | |
| 25 | OSD1-2 target | monocot | Zea mays |
| | sequence | | |
| 26 | OSD1-2 target | monocot | Zea mays |
| | sequence | | |
| 27 | OSD1-2 target | monocot | Zea mays |
| | sequence | | |
| 28 | OSD1-2 target | monocot | Zea mays |
| | sequence | | |
| 29 | OSD1-2 target | monocot | Zea mays |
| | sequence | | |
| 30 | OSD1-2 target | monocot | Zea mays |
| | sequence | | |
| 31 | OSD1-2 target | monocot | Zea mays |
| | sequence | | |
| 32 | OSD1-2 target | monocot | Zea mays |
| | sequence | | |
| 33 | REC8 target sequence | monocot | Zea mays |
| 34 | REC8 target sequence | monocot | Zea mays |
| 35 | REC8 target sequence | monocot | Zea mays |
| 36 | REC8 target sequence | monocot | Zea mays |
| 37 | REC8 target sequence | monocot | Zea mays |
| 38 | REC8 target sequence | monocot | Zea mays |
| 39 | REC8 target sequence | monocot | Zea mays |
| 40 | REC8 target sequence | monocot | Zea mays |
| 41 | REC8 target sequence | monocot | Zea mays |
| 42 | SPO11-1 target | monocot | Zea mays |
| | sequence | | |
| 43 | SPO11-1 target | monocot | Zea mays |
| | sequence | | |
| 44 | SPO11-1 target | monocot | Zea mays |
| | sequence | | |
| 45 | SPO11-1 target | monocot | Zea mays |
| | sequence | | |
| 46 | SPO11-1 target | monocot | Zea mays |
| | sequence | | |
| 47 | SPO11-1 target | monocot | Zea mays |
| | sequence | | |
| 48 | SPO11-1 target | monocot | Zea mays |
| | sequence | | |
| 49 | SPO11-1 target | monocot | Zea mays |
| | sequence | | |
| 50 | SPO11-1 target | monocot | Zea mays |
| | sequence | | |
| 51 | SPO11-1 target | monocot | Zea mays |
| | sequence | | |
| 52 | SPO11-1 target | monocot | Zea mays |
| | sequence | | |
| 53 | SPO11-1 target | monocot | Zea mays |
| | sequence | | |
| 54 | SPO11-1 target | monocot | Zea mays |
| | sequence | | |
| 55 | SPO11-1 target | monocot | Zea mays |
| | sequence | | |
| 56 | SPO11-1 target | monocot | Zea mays |
| | sequence | | |
| 57 | SPO11-1 target | monocot | Zea mays |
| | sequence | | |
| 58 | SPO11-1 target | monocot | Zea mays |
| | sequence | | |
| 59 | SPO11-1 target | monocot | Zea mays |
| | sequence | | |
| 60 | SPO11-1 target | monocot | Zea mays |
| | sequence | | |
| 61 | OSD1-1-targeting | monocot | Zea mays |
| | region of gRNA | | |
| 62 | OSD1-1-targeting | monocot | Zea mays |
| | region of gRNA | | |
| 63 | OSD1-1-targeting | monocot | Zea mays |
| | region of gRNA | | |
| 64 | OSD1-1-targeting | monocot | Zea mays |
| | region of gRNA | | |
| 65 | OSD1-1-targeting | monocot | Zea mays |
| | region of gRNA | | |
| 66 | OSD1-1-targeting | monocot | Zea mays |
| | region of gRNA | | |
| 67 | OSD1-1-targeting | monocot | Zea mays |
| | region of gRNA | | |
| 68 | OSD1-1-targeting | monocot | Zea mays |
| | region of gRNA | | |
| 69 | OSD1-1-targeting | monocot | Zea mays |
| | region of gRNA | | |
| 70 | OSD1-1-targeting | monocot | Zea mays |
| | region of gRNA | | |
| 71 | OSD1-2-targeting | monocot | Zea mays |
| | region of gRNA | | |
| 72 | OSD1-2-targeting | monocot | Zea mays |
| | region of gRNA | | |
| 73 | OSD1-2-targeting | monocot | Zea mays |
| | region of gRNA | | |
| 74 | OSD1-2-targeting | monocot | Zea mays |
| | region of gRNA | | |
| 75 | OSD1-2-targeting | monocot | Zea mays |
| | region of gRNA | | |
| 76 | OSD1-2-targeting | monocot | Zea mays |
| | region of gRNA | | |
| 77 | OSD1-2-targeting | monocot | Zea mays |
| | region of gRNA | | |
| 78 | OSD1-2-targeting | monocot | Zea mays |
| | region of gRNA | | |
| 79 | OSD1-2-targeting | monocot | Zea mays |
| | region of gRNA | | |
| 80 | REC8-targeting | monocot | Zea mays |
| | region of gRNA | | |
| 81 | REC8-targeting | monocot | Zea mays |
| | region of gRNA | | |
| 82 | REC8-targeting | monocot | Zea mays |
| | region of gRNA | | |
| 83 | REC8-targeting | monocot | Zea mays |
| | region of gRNA | | |
| 84 | REC8-targeting | monocot | Zea mays |
| | region of gRNA | | |
| 85 | REC8-targeting | monocot | Zea mays |
| | region of gRNA | | |
| 86 | REC8-targeting | monocot | Zea mays |
| | region of gRNA | | |
| 87 | REC8-targeting | monocot | Zea mays |
| | region of gRNA | | |
| 88 | REC8-targeting | monocot | Zea mays |
| | region of gRNA | | |
| 89 | SPO11-1-targeting | monocot | Zea mays |
| | region of gRNA | | |
| 90 | SPO11-1-targeting | monocot | Zea mays |
| | region of gRNA | | |
| 91 | SPO11-1-targeting | monocot | Zea mays |
| | region of gRNA | | |
| 92 | SPO11-1-targeting | monocot | Zea mays |
| | region of gRNA | | |
| 93 | SPO11-1-targeting | monocot | Zea mays |
| | region of gRNA | | |
| 94 | SPO11-1-targeting | monocot | Zea mays |
| | region of gRNA | | |
| 95 | SPO11-1-targeting | monocot | Zea mays |
| | region of gRNA | | |
| 96 | SPO11-1-targeting | monocot | Zea mays |
| | region of gRNA | | |
| 97 | SPO11-1-targeting | monocot | Zea mays |
| | region of gRNA | | |
| 98 | SPO11-1-targeting | monocot | Zea mays |
| | region of gRNA | | |
| 99 | SPO11-1-targeting | monocot | Zea mays |
| | region of gRNA | | |
| 100 | SPO11-1-targeting | monocot | Zea mays |
| | region of gRNA | | |
| 101 | SPO11-1-targeting | monocot | Zea mays |
| | region of gRNA | | |
| 102 | SPO11-1-targeting | monocot | Zea mays |
| | region of gRNA | | |
| 103 | SPO11-1-targeting | monocot | Zea mays |
| | region of gRNA | | |
| 104 | SPO11-1-targeting | monocot | Zea mays |
| | region of gRNA | | |
| 105 | SPO11-1-targeting | monocot | Zea mays |
| | region of gRNA | | |
| 106 | SPO11-1-targeting | monocot | Zea mays |
| | region of gRNA | | |
| 107 | SPO11-1-targeting | monocot | Zea mays |
| | region of gRNA | | |
| 108 | osd1-2 allele | monocot | Zea mays, PED-MN-MiMe |
| 109 | osd1-2 allele | monocot | Zea mays, PED-MN-MiMe |
| 110 | rec8 allele | monocot | Zea mays, PED-MN-MiMe |
| 111 | spo11 allele | monocot | Zea mays, PED-MN-MiMe |

TABLE 6
Exemplary combinations of in MiMe alleles of parent
MiMe plants having a complete MiMe genotype.

| Genotype | Complete MiMe Genotype |
|---|---|
| 1 | rec8, spo11-1, cyca1 |
| 2 | rec8, pair1, cyca1 |
| 3 | rec8, pair1, spo11-1, cyca1 |
| 4 | rec8, spo11-1, TDM1* |
| 5 | rec8, pair1, TDM1* |
| 6 | rec8, pair1, spo11-1, TDM1* |
| 7 | rec8, spo11-1, TDM1*, cyca1 |
| 8 | rec8, pair1, TDM1*, cyca1 |
| 9 | rec8, pair1, spo11-1, TDM1*, cyca1 |
| 10 | spo11-1, ps1 |
| 10 | spo11-1, ps1, ps1-like |
| 11 | spo11-1, ps1-like |
| 12 | spo11-1, ps1-like, jason |
| 13 | spo11-1, jason |
| 14 | spo11-1, ps1, ps1-like, jason |
| 15 | pair1, ps1 |
| 16 | pair1, spo11-1, ps1 |
| 17 | spo11-1, ps1, ps1-like |
| 18 | pair1, spo11-1, ps1, ps1-like |
| 19 | pair1, ps1-like |
| 20 | pair1, spo11-1, ps1-like |
| 21 | pair1, ps1-like, jason |
| 22 | pair1, spo11-1, ps1-like, jason |
| 23 | pair1, jason |
| 24 | pair1, spo11-1, jason |
| 25 | pair1, ps1, ps1-like, jason |
| 26 | pair1, spo11-1, ps1, ps1-like, jason |
| 27 | rec8, spo11-1, osd1 |
| 28 | rec8, pair1, osd1 |
| 29 | rec8, pair1, spo11-1, osd1 |

*Dominant mutation

### ENUMERATED EMBODIMENTS

The following enumerated embodiments are representative of some aspects of the invention.

1. A population of polyploid maize seed comprising a subpopulation of genetically uniform polyploid maize seed in an amount of at least 50% of the total number of seeds, the genetically uniform polyploid maize seed comprising three or more haplotypes of the same or related species of plant, wherein the population was obtained from a single plant or a set of F1 hybrids.

2. The population of embodiment 1, wherein the subpopulation of genetically uniform polyploid maize seed is triploid, tetraploid, pentaploid, hexaploid, heptaploid, or octaploid.

3. The population of embodiment 1 or 2, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

4. The population of any one of embodiments 1-3, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

5. The population of embodiment 1-4, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

6. The population of any one of embodiments 1-5, wherein the subpopulation of genetically uniform polyploid maize seed has a complete MiMe genotype comprising:
(A)
MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components.

7. The population of any one of embodiments 1-5, wherein the subpopulation of genetically uniform polyploid maize seed has a partial MiMe genotype comprising:
(A)
(a) one or more MiMe alleles conferring decreased expression of one or more MiMe loci of each of a first, second, and third MiMe component; and
(b) one or more non-MiMe alleles at the one or more MiMe loci of each of the first, second, and third MiMe component,
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
   (a) one or more MiMe alleles conferring decreased expression of one or more MiMe loci of each of the first and second MiMe component; and
   (b) one or more non-MiMe alleles at the one or more MiMe loci of each of the first and second MiMe component,
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

8. The population of any one of embodiments 1-5, wherein the subpopulation of genetically uniform polyploid maize seed has a partially-complemented MiMe genotype comprising:
(A)
   (a) only MiMe alleles at one or more MiMe loci of a first MiMe component;
   (b) one or more MiMe alleles and one or more non-MiMe alleles at a first MiMe locus of a second MiMe component, and one or more MiMe alleles and one or more non-MiMe alleles at a second MiMe locus of the second MiMe component; and
   (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
(B)
   (a) only MiMe alleles at one or more MiMe loci of a first MiMe component; and
   (b) one or more MiMe alleles and one or more non-MiMe alleles at a first MiMe locus of a second MiMe component, and one or more MiMe alleles and one or more non-MiMe alleles at a second MiMe locus of the second MiMe component,
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
(C)
   (a) only MiMe alleles at one or more MiMe loci of a first MiMe component, wherein the first MiMe component is a component of DNA double strand breakage during meiotic recombination;
   (b) one or more MiMe alleles and one or more non-MiMe alleles at one or more MiMe loci of a second MiMe component;
   (c) one or more MiMe alleles and one or more non-MiMe alleles at one or more MiMe loci of a third MiMe component; and
   (d) one or more MiMe alleles and one or more non-MiMe alleles at one or more MiMe loci of a fourth MiMe component,
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

9. The population of any one of embodiments 6-8f, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

10. The population of any one of embodiments 1-9, wherein the subpopulation of genetically uniform polyploid maize seed comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

11. The population of any one of embodiments 1-9, wherein the subpopulation of genetically uniform polyploid maize seed comprises one or more polynucleotide sequences selected from the group consisting of SEQ ID NOs: 108-111.

12. The population of any one of embodiments 1-11, wherein germination of a seed of the subpopulation of genetically uniform polyploid maize seed results in a sterile plant that produces inviable gametes, seedless fruit, or a combination thereof.

13. The population of any one of embodiments 1-12, wherein the population of polyploid maize seed comprises three or more haplotypes from the same species of maize.

14. A method of producing the population of polyploid maize seed of any one of embodiments 1-13, the method comprising:
(a) generating a first parent MiMe plant and a second parent MiMe plant by introducing genetic modifications into one or more candidate lines to produce MiMe alleles in germplasm of a first parent MiMe plant, a second parent MiMe plant, and/or progenitors thereof, wherein the first parent MiMe plant and the second parent MiMe plant each comprise a complete MiMe genotype, and each of the progenitors comprises a partial MiMe genotype, wherein if progenitors are generated, the progenitors are further crossed to generate the first parent MiMe plant, the second parent MiMe plant, or both;
(b) providing clonal gametes from the first parent MiMe plant and the second parent MiMe plant that together comprise the three or more haplotypes; and
(c) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

15. The method of embodiment 14, wherein the subpopulation of genetically uniform polyploid maize seed has a partially-complemented MiMe genotype, wherein:
(A)
(a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
(b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
(a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
(b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
(a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
(b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

16. A method of producing the population of polyploid maize seed of any one of embodiments 1-13, the method comprising:
(a) generating a first parent MiMe plant by introducing genetic modifications into one or more candidate lines to produce MiMe alleles in germplasm of the first parent MiMe plant or progenitors thereof, wherein the first parent MiMe plant comprises a complete MiMe genotype, and each of the progenitors comprises a partial MiMe genotype, wherein if progenitors are generated, the progenitors are further crossed to generate the first parent MiMe plant;
(b) providing clonal gametes from the first parent MiMe plant;
(c) providing haploid gametes from a homozygous parent non-MiMe plant; and
(d) crossing the clonal gametes with the haploid gametes to produce the population of polyploid maize seed, wherein the clonal gametes and the haploid gametes together comprise three or more haplotypes, optionally wherein step (a) comprises generating the first parent MiMe plant, the second parent MiMe plant, or both.

17. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant and a second parent MiMe plant from the two or more candidate lines that together comprise the three or more haplotypes;
(e) providing clonal gametes from each of the first and second parent MiMe plants;
(f) crossing the clonal gametes to produce a hybrid polyploid maize seed comprising the three or more haplotypes;
(g) growing the hybrid polyploid maize seed to produce a hybrid polyploid plant comprising three or more haplotypes; and
(h) evaluating one or more characteristics of the hybrid polyploid plant;
the method optionally further comprising
(i) repeating steps (b)-(h) or steps (c)-(h) using the one or more characteristics of the hybrid polyploid plant evaluated in step (h) to guide the breeding of lines of step (b), the selecting of candidate lines of step (c), or both.

18. The method of embodiment 17, wherein:
(A)
   (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
   (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
   (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
   (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
   (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
   (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

19. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant from one of the two or more candidate lines;
(e) providing clonal gametes from the first parent MiMe plant;
(f) providing haploid gametes from a homozygous parent non-MiMe plant of one of the two or more candidate lines;
(g) crossing the clonal gametes with the haploid gametes to produce a hybrid polyploid maize seed;
(h) growing the hybrid polyploid maize seed to produce a hybrid polyploid plant; and
(i) evaluating one or more characteristics of the hybrid polyploid plant,
wherein the first parent MiMe plant and the homozygous parent non-MiMe plant together comprise three or more haplotypes, wherein the crossing of step (g) results in the hybrid polyploid maize seed comprising three or more haplotypes, and wherein the growing of step (h) results in the hybrid polyploid plant comprising three or more haplotypes;
the method optionally further comprising
(j) repeating steps (b)-(i) or steps (c)-(i) using the one or more characteristics of the hybrid polyploid plant evaluated in step (i) to guide the breeding of lines of step (b), the selecting of candidate lines of step (c), or both.

20. The method of any one of embodiments 14-19, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octaploid.

21. The method of any one of embodiments 14-20, wherein generating the first parent MiMe plant, the second parent MiMe plant, or both comprises:
(1) introducing a complete MiMe genotype directly into two candidate lines to produce the first parent MiMe plant, the second parent MiMe plant, or both;
(2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant, optionally wherein generating the first parent MiMe plant, the second parent MiMe plant, or both further comprises introducing a complete MiMe genotype directly into a third candidate line to produce the second parent MiMe plant; or
(3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants,
optionally wherein generating the first parent MiMe plant, the second parent MiMe plant, or both further comprises propagating the first parent MiMe plant, the second parent MiMe plant, the grandparent non-MiMe plants, or any combination thereof to scale production of homogenous seed.

22. The method of any one of embodiments 14-21, wherein the first parent MiMe plant, the second parent MiMe plant, the hybrid polyploid plant, or any combination thereof has a complete MiMe genotype comprising:
(A)
MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components.

23. The method of any one of embodiments 17-22, wherein each grandparent non-MiMe plant, the hybrid polyploid plant, or a combination thereof has a partial MiMe genotype comprising:
(A)
   (a) one or more MiMe alleles conferring decreased expression of one or more MiMe loci of each of a first, second, and third MiMe component; and
   (b) one or more non-MiMe alleles at the one or more MiMe loci of each of the first, second, and third MiMe component,
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
   (a) one or more MiMe alleles conferring decreased expression of one or more MiMe loci of each of the first and second MiMe component; and
   (b) one or more non-MiMe alleles at the one or more MiMe loci of each of the first and second MiMe component,
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

24. The method of embodiment 18, 22, or 23, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

25. The method of any one of embodiments 17-24, wherein the hybrid polyploid plant comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1; (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

26. The method of any one of embodiments 17-25, wherein one or more of the candidate lines of step (c) are inbred lines, hybrid lines, or a combination thereof.

27. The method of any one of embodiments 21-26, wherein the complete MiMe genotype, the partial MiMe genotype, or both are introduced by gene editing, transgenesis, or a combination thereof.

28. The method of embodiment 22-27, wherein the decreased expression of one or more of the MiMe loci is each independently achieved by gene disruption, gene knockout, gene knockdown, gene silencing, RNA interference, or induction of methylation.

29. The method of embodiment 22-28, wherein the decreased expression of one or more of the MiMe loci is each independently achieved by introducing into each candidate line or a progenitor thereof an insertion, a deletion, one or more nucleotide changes, or an inversion that results in decreased expression of the MiMe locus, optionally including a step of selection for decreased expression of the MiMe locus.

30. The method of embodiment 29, wherein the insertion, the deletion, the one or more nucleotide changes, or the inversion eliminates expression of the MiMe locus,
optionally wherein the expression of the MiMe locus is eliminated by a premature stop codon present in the 70%, the first 60%, the first 50%, the first 40%, the first 30%, the first 20%, or the first 10% of the nucleotides of the coding sequence of the MiMe locus following the start codon in the 3′ direction.

31. A method of producing a population of polyploid maize seed comprising:
(a) providing clonal gametes from a pair of parent MiMe plants together comprising three or more haplotypes that were selected using the method of breeding of any one of embodiments 17, 18, and 20-30 based upon the polyploid plant comprising the three or more haplotypes having one or more desired characteristics; and
(b) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

32. A method of producing a population of polyploid maize seed comprising:
(a) selecting three or more haplotypes using the method of breeding of any one of embodiments 19-30 based upon the polyploid plant comprising said three or more haplotypes having one or more desired characteristics;
(b) providing clonal gametes from a parent MiMe plant;
(c) providing haploid gametes from a homozygous parent non-MiMe plant;
(d) crossing the clonal gametes with the haploid gametes to produce the population of polyploid maize seed;
wherein the parent MiMe plant and the homozygous parent non-MiMe plant together comprise the three or more haplotypes selected in step (a), wherein the crossing of step (d) results in a population of polyploid maize seed comprising a subpopulation of genetically uniform polyploid maize seed in an amount of at least 50% of the total number of seeds, the subpopulation of genetically uniform polyploid maize seed comprising the three or more haplotypes.

33. The method of embodiment 31 or 32, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

34. The method of any one of embodiments 31-33, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

35. The method of any one of embodiments 31-34, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

36. A genetically modified plant, plant part, or plant cell comprising:
(A) three or more haplotypes; and
(B)
   (i) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   ii) a partial MiMe genotype comprising:
      (a) one or more MiMe alleles conferring decreased expression of one or more MiMe loci of each of a first, second, and third MiMe component; and
      (b) one or more non-MiMe alleles at the one or more MiMe loci of each of the first, second, and third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   iii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components;
   iv) a partial MiMe genotype comprising:
      (a) one or more MiMe alleles conferring decreased expression of one or more MiMe loci of each of the first and second MiMe component; and
      (b) one or more non-MiMe alleles at the one or more MiMe loci of each of the first and second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components;
   v) a partially complemented MiMe genotype comprising:
      (a) only MiMe alleles at one or more MiMe loci of a first MiMe component;
      (b) one or more MiMe alleles and one or more non-MiMe alleles at a first MiMe locus of a second MiMe component, and one or more MiMe alleles and one or more non-MiMe alleles at a second MiMe locus of the second MiMe component; and
      (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   vi) a partially complemented MiMe genotype comprising:
      (a) only MiMe alleles at one or more MiMe loci of a first MiMe component; and
      (b) one or more MiMe alleles and one or more non-MiMe alleles at a first MiMe locus of a second MiMe component, and one or more MiMe alleles and one or more non-MiMe alleles at a second MiMe locus of the second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
   vii) a partially complemented MiMe genotype comprising:
      (a) only MiMe alleles at one or more MiMe loci of a first MiMe component, wherein the first MiMe component is a component of DNA double strand breakage during meiotic recombination;
      (b) one or more MiMe alleles and one or more non-MiMe alleles at one or more MiMe loci of a second MiMe component;
      (c) one or more MiMe alleles and one or more non-MiMe alleles at one or more MiMe loci of a third MiMe component; and
      (d) one or more MiMe alleles and one or more non-MiMe alleles at one or more MiMe loci of a fourth MiMe component,
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

37. The genetically modified plant, plant part, or plant cell of embodiment 36, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

38. The genetically modified plant, plant part, or plant cell of embodiment 36 or 37, comprising:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

39. The genetically modified plant, plant part, or plant cell of any one of embodiments 36-38, comprising one or more polynucleotide sequences selected from the group consisting of SEQ ID NOs: 108-111.

40. The genetically modified plant, plant part, or plant cell of any one of embodiments 36-39, wherein:
(i) the genetically modified plant part is a non-regenerable plant part; or
(ii) the genetically modified plant cell is a non-regenerable plant cell.

41. The genetically modified plant, plant part, or plant cell of any one of embodiments 36-40, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.

42. A processed plant product derived from the genetically modified plant, plant part, or plant cell of any one of embodiments 36-41 wherein the processed plant product comprises a detectable amount of the one or more MiMe alleles of the genetically modified plant, plant part, or plant cell.

43. The processed plant product of embodiment 42, wherein the processed plant product:
(i) is selected from the group consisting of plant biomass, oil, meal, food starch, syrup, animal feed, flour, flakes, bran, lint, hulls, and processed seed; and/or
(ii) is non-regenerable.

44. Germplasm of the population of polyploid maize seeds of any one of embodiments 1-13.

45. A population of polyploid maize seed comprising a subpopulation of genetically uniform polyploid maize seed in an amount of at least 50% of the total number of seeds, the genetically uniform polyploid maize seed comprising three or more haplotypes of the same or related species of plant, wherein the population was obtained from a single plant or a set of F1 hybrids, wherein the subpopulation of genetically uniform polyploid maize seed comprise (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or (B) (2) a germplasm genetic modification means for preventing DNA double strand breakage during meiotic recombination, and (4) a germplasm genetic modification means for preventing progression through the first division of meiosis;
wherein each of the germplasm genetic modifications are (i) a complete set that achieves the inhibition, (ii) a partial set that does not achieve inhibition, or (iii) partially complemented at one or more of the genetic modification means and achieve sterility.

46. The population of embodiment 45, wherein the subpopulation of genetically uniform polyploid maize seed is triploid, tetraploid, pentaploid, hexaploid, heptaploid, or octaploid.

47. The population of embodiment 45 or 46, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

48. The population of any one of embodiments 45-47, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

49. The population of embodiment 45-48, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

50. The population of any one of embodiments 45-49, wherein the subpopulation of genetically uniform polyploid maize seed has a complete MiMe genotype comprising the complete set of germplasm genetic modification means as follows:
(A)
(1) the germplasm genetic modification means for inhibiting sister chromatid cohesion during the first division of meiosis comprise only MiMe alleles at one or more MiMe loci of a component of sister chromatid cohesion during the first division of meiosis, (2) the germplasm genetic modification means for inhibiting DNA double strand breakage during meiotic recombination comprise only MiMe alleles at one or more MiMe loci of a component of DNA double strand breakage during meiotic recombination, and (3) the germplasm genetic modification means for inhibiting progression through the second division of meiosis comprise only MiMe alleles at one or more Mime loci of progression through the second division of meiosis; or
(B)
(2) the germplasm genetic modification means for inhibiting DNA double strand breakage during meiotic recombination comprise only MiMe alleles at one or more MiMe loci of a component of DNA double strand breakage during meiotic recombination, and (4) the germplasm genetic modification means for inhibiting progression through the first division of meiosis comprise only MiMe alleles at one or more MiMe loci of a component of progression through the first division of meiosis.

51. The population of any one of embodiments 45-49, wherein the subpopulation of genetically uniform polyploid maize seed has a partial MiMe genotype comprising the partial set of germplasm genetic modification means as follows:
(A)
(1) the germplasm genetic modification means for inhibiting sister chromatid cohesion during the first division of meiosis comprise at least one MiMe allele and at least one non-MiMe allele at one or more MiMe loci for a component of sister chromatid cohesion during the first division of meiosis comprise, (2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (3) the germplasm genetic modification means for inhibiting progression through the second division of meiosis comprise at least one MiMe allele and at least one non-MiMe allele at one or more MiMe loci for a component of progression through the second division of meiosis; or
(B)
(2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (4) the germplasm genetic modification means for inhibiting progression through the first division of meiosis comprise at least one MiMe allele and at least one non-MiMe allele at one or more MiMe loci for a component of progression through the first division of meiosis.

52. The population of any one of embodiments 45-49, wherein the subpopulation of genetically uniform polyploid maize seed has a partially-complemented MiMe genotype comprising the partially complemented set of germplasm genetic modification means as follows:
(A)
   (1) the germplasm genetic modification means for inhibiting sister chromatid cohesion during the first division of meiosis comprises at least one MiMe allele at at least a first MiMe locus of a component of sister chromatid cohesion during the first division of meiosis, (2) the germplasm genetic modification means for inhibiting DNA double strand breakage during meiotic recombination comprises at least one MiMe allele at at least a first MiMe locus of a component of DNA double strand breakage during meiotic recombination, and (3) the germplasm genetic modification means for inhibiting progression through the second division of meiosis comprises at least one MiMe allele at at least a first MiMe locus of a component of progression through the second division of meiosis,
   wherein at least one of (1), (2), and (3) comprise one or more MiMe alleles and one or more non-MiMe alleles at the first MiMe locus of the component, and one or more MiMe alleles and one or more non-MiMe alleles at a second MiMe locus of the component, and wherein one or both of the others of (1), (2), and (3) comprise only MiMe alleles at at least the first MiMe locus of the component; or
(B)
   (2) the germplasm genetic modification means for inhibiting DNA double strand breakage during meiotic recombination comprises at least one MiMe allele at at least a first MiMe locus of a component of DNA double strand breakage during meiotic recombination, and (4) the germplasm genetic modification means for preventing progression through the first division of meiosis comprises at least one MiMe allele at at least a first MiMe locus of a component of progression through the first division of meiosis, wherein one of (2) and (4) comprises one or more MiMe alleles and one or more non-MiMe alleles at the first MiMe locus of the component, and one or more MiMe alleles and one or more non-MiMe alleles at a second MiMe locus of the component, and the other of (2) and (4) comprises only MiMe alleles at at least the first MiMe locus of the component; or
(C)
   (2) the germplasm genetic modification means for inhibiting DNA double strand breakage during meiotic recombination comprise at least one MiMe allele at at least a first MiMe locus of a component of DNA double strand breakage during meiotic recombination;
   (1) the germplasm genetic modification means for inhibiting sister chromatid cohesion during the first division of meiosis comprises at least one MiMe allele and at least one non-MiMe allele at one or more MiMe loci for a component of sister chromatid cohesion during the first division of meiosis;
   (3) the germplasm genetic modification means for inhibiting progression through the second division of meiosis comprises at least one MiMe allele and at least one non-MiMe allele at one or more MiMe loci for a component of progression through the second division of meiosis; and
   (4) the germplasm genetic modification means for inhibiting progression through the first division of meiosis comprise at least one MiMe allele and at least one non-MiMe allele at one or more MiMe loci for a component of progression through the first division of meiosis.

53. The population of any one of embodiments 50-52, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

54. The population of any one of embodiments 45-53, wherein the subpopulation of genetically uniform polyploid maize seed comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1; (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

55. The population of any one of embodiments 45-54, wherein the subpopulation of genetically uniform polyploid maize seed comprises one or more polynucleotide sequences selected from the group consisting of SEQ ID NOs: 108-111.

56. The population of any one of embodiments 45-55, wherein germination of a seed of the subpopulation of genetically uniform polyploid maize seed results in a sterile plant that produces inviable gametes, seedless fruit, or a combination thereof.

57. The population of any one of embodiments 45-56, wherein the population of polyploid maize seed comprises three or more haplotypes from the same species of maize.

58. A method of producing the population of polyploid maize seed of any one of embodiments 45-57, the method comprising:
(0) generating a first parent MiMe plant and a second parent MiMe plant by introducing genetic modifications into germplasms of one or more candidate lines of a plant to generate the first parent MiMe plant, the second parent MiMe plant, and/or progenitors thereof, wherein the first parent MiMe plant and the second parent MiMe plant together comprise three or more haplotypes, and wherein each of the first parent MiMe plant, the second parent MiMe plant, and/or the progenitors thereof comprises:
   (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
   (B) (2) a germplasm genetic modification means for preventing DNA double strand breakage during meiotic recombination, and (4) a germplasm genetic modification means for preventing progression through the first division of meiosis;
   wherein each of the germplasm genetic modifications are (i) a complete set that achieves the inhibition, or (ii) a partial set that does not achieve inhibition;
wherein if progenitors are generated, the progenitors are further crossed to generate the first parent MiMe plant, the second parent MiMe plant, or both, and wherein the generating the first parent MiMe plant, the second parent MiMe plant, and/or the progenitors thereof comprises selecting for plants comprising the germplasm genetic modification means after introducing the germplasm genetic modification means;
(a) providing clonal gametes from the first parent MiMe plant and the second parent MiMe plant that together comprise the three or more haplotypes; and
(b) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

59. The method of embodiment 58, wherein the subpopulation of genetically uniform polyploid maize seed has a partially-complemented MiMe genotype, wherein:
(A)
(a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
(b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant; wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
(a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
(b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
(a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
(b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
wherein at least one of the MiMe loci having only MiMe alleles of the first MiMe component of the first parent MiMe plant is the same as at least one of the MiMe loci having only MiMe alleles of the first MiMe component of the second parent MiMe plant;
wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

60. A method of producing the population of polyploid maize seed of any one of embodiments 45-57, the method comprising:
(0) generating a first parent MiMe plant by introducing genetic modifications into germplasms of one or more candidate lines of a plant to generate the first parent MiMe plant or progenitors thereof, and wherein each of the first parent MiMe plant or the progenitors thereof comprises:
   (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
   (B) (2) a germplasm genetic modification means for preventing DNA double strand breakage during meiotic recombination, and (4) a germplasm genetic modification means for preventing progression through the first division of meiosis;
   wherein each of the germplasm genetic modifications are (i) a complete set that achieves the inhibition, (ii) a partial set that does not achieve inhibition, or (iii) partially complemented at one or more of the genetic modification means and achieve sterility;
wherein if progenitors are generated, the progenitors are further crossed to generate the first parent MiMe plant, and wherein the generating the first parent MiMe plant and/or the progenitors thereof comprises selecting for plants comprising the germplasm genetic modification means after introducing the germplasm genetic modification means;
(a) providing clonal gametes from the first parent MiMe plant;
(b) providing haploid gametes from a homozygous parent non-MiMe plant; and
(c) crossing the clonal gametes with the haploid gametes to produce the population of polyploid maize seed,
wherein the clonal gametes and the haploid gametes together comprise three or more haplotypes, optionally wherein step (a) comprises generating the first parent MiMe plant, the second parent MiMe plant, or both.

61. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant and a second parent MiMe plant from the two or more candidate lines that together comprise the three or more haplotypes;
(e) providing clonal gametes from each of the first and second parent MiMe plants;
(f) crossing the clonal gametes to produce a hybrid polyploid maize seed comprising the three or more haplotypes;
(g) growing the hybrid polyploid maize seed to produce a hybrid polyploid plant comprising three or more haplotypes; and
(h) evaluating one or more characteristics of the hybrid polyploid plant;
the method optionally further comprising
(i) repeating steps (b)-(h) or steps (c)-(h) using the one or more characteristics of the hybrid polyploid plant evaluated in step (h) to guide the breeding of lines of step (b), the selecting of candidate lines of step (c), or both.

62. The method of embodiment 61, wherein:
(A)
   (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
   (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
   (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
   (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
   (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
   (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

63. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant from one of the two or more candidate lines;
(e) providing clonal gametes from the first parent MiMe plant;
(f) providing haploid gametes from a homozygous parent non-MiMe plant of one of the two or more candidate lines;
(g) crossing the clonal gametes with the haploid gametes to produce a hybrid polyploid maize seed;
(h) growing the hybrid polyploid maize seed to produce a hybrid polyploid plant; and
(i) evaluating one or more characteristics of the hybrid polyploid plant,
wherein the first parent MiMe plant and the homozygous parent non-MiMe plant together comprise three or more haplotypes, wherein the crossing of step (g) results in the hybrid polyploid maize seed comprising three or more haplotypes, and wherein the growing of step (h) results in the hybrid polyploid plant comprising three or more haplotypes;
the method optionally further comprising
(j) repeating steps (b)-(i) or steps (c)-(i) using the one or more characteristics of the hybrid polyploid plant evaluated in step (i) to guide the breeding of lines of step (b), the selecting of candidate lines of step (c), or both.

64. The method of any one of embodiments 58-64, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octaploid.

65. The method of any one of embodiments 58-64, wherein generating the first parent MiMe plant, the second parent MiMe plant, or both comprises:
(1) introducing a complete MiMe genotype directly into two candidate lines to produce the first parent MiMe plant, the second parent MiMe plant, or both;
(2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant, optionally wherein generating the first parent MiMe plant, the second parent MiMe plant, or both further comprises introducing a complete MiMe genotype directly into a third candidate line to produce the second parent MiMe plant; or
(3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants,
optionally wherein generating the first parent MiMe plant, the second parent MiMe plant, or both further comprises propagating the first parent MiMe plant, the second parent MiMe plant, the grandparent non-MiMe plants, or any combination thereof to scale production of homogenous seed.

66. The method of any one of embodiments 58-65, wherein the first parent MiMe plant, the second parent MiMe plant, the hybrid polyploid plant, or any combination thereof has a complete MiMe genotype comprising:
(A)
MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
   MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components.

67. The method of any one of embodiments 61-66, wherein each grandparent non-MiMe plant, the hybrid polyploid plant, or a combination thereof has a partial MiMe genotype comprising:
(A)
   (a) one or more MiMe alleles conferring decreased expression of one or more MiMe loci of each of a first, second, and third MiMe component; and
   (b) one or more non-MiMe alleles at the one or more MiMe loci of each of the first, second, and third MiMe component,
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
   (a) one or more MiMe alleles conferring decreased expression of one or more MiMe loci of each of the first and second MiMe component; and
   (b) one or more non-MiMe alleles at the one or more MiMe loci of each of the first and second MiMe component,
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

68. The method of embodiment 18, 66, or 67, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

69. The method of any one of embodiments 61-68, wherein the hybrid polyploid plant comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

70. The method of any one of embodiments 61-69, wherein one or more of the candidate lines of step (c) are inbred lines, hybrid lines, or a combination thereof.

71. The method of any one of embodiments 65-70, wherein the complete MiMe genotype, the partial MiMe genotype, or both are introduced by gene editing, transgenesis, or a combination thereof.

72. The method of embodiment 66-71, wherein the decreased expression of one or more of the MiMe loci is each independently achieved by gene disruption, gene knockout, gene knockdown, gene silencing, RNA interference, or induction of methylation.

73. The method of embodiment 66-71, wherein the decreased expression of one or more of the MiMe loci is each independently achieved by introducing into each candidate line an insertion, a deletion, one or more nucleotide changes, or an inversion that results in decreased expression of the MiMe locus, optionally including a step of selection for decreased expression of the MiMe locus.

74. The method of embodiment 73, wherein the insertion, the deletion, the one or more nucleotide changes, or the inversion eliminates expression of the MiMe locus, optionally wherein the expression of the MiMe locus is eliminated by a premature stop codon present in the 70%, the first 60%, the first 50%, the first 40%, the first 30%, the first 20%, or the first 10% of the nucleotides of the coding sequence of the MiMe locus following the start codon in the 3′ direction.

75. A method of producing a population of polyploid maize seed comprising:
(a) providing clonal gametes from a pair of parent MiMe plants together comprising three or more haplotypes that were selected using the method of breeding of any one of embodiments 61, 62, and 64-74 based upon the polyploid plant comprising the three or more haplotypes having one or more desired characteristics; and
(b) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

76. A method of producing a population of polyploid maize seed comprising:
(a) selecting three or more haplotypes using the method of breeding of any one of embodiments 63-74 based upon the polyploid plant comprising said three or more haplotypes having one or more desired characteristics;
(b) providing clonal gametes from a parent MiMe plant;
(c) providing haploid gametes from a homozygous parent non-MiMe plant;
(d) crossing the clonal gametes with the haploid gametes to produce the population of polyploid maize seed;
wherein the parent MiMe plant and the homozygous parent non-MiMe plant together comprise the three or more haplotypes selected in step (a), wherein the crossing of step (d) results in a population of polyploid maize seed comprising a subpopulation of genetically uniform polyploid maize seed in an amount of at least 50% of the total number of seeds, the subpopulation of genetically uniform polyploid maize seed comprising the three or more haplotypes.

77. The method of embodiment 75 or 76, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

78. The method of any one of embodiments 75-77, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

79. The method of any one of embodiments 75-78, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

80. A genetically modified plant, plant part, or plant cell comprising:
(A) three or more haplotypes; and
(B)
   (i) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   ii) a partial MiMe genotype comprising:
      (a) one or more MiMe alleles conferring decreased expression of one or more MiMe loci of each of a first, second, and third MiMe component; and
      (b) one or more non-MiMe alleles at the one or more MiMe loci of each of the first, second, and third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   iii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components;
   iv) a partial MiMe genotype comprising:
      (a) one or more MiMe alleles conferring decreased expression of one or more MiMe loci of each of the first and second MiMe component; and
      (b) one or more non-MiMe alleles at the one or more MiMe loci of each of the first and second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components;
   v) a partially complemented MiMe genotype comprising:
      (a) only MiMe alleles at one or more MiMe loci of a first MiMe component;
      (b) one or more MiMe alleles and one or more non-MiMe alleles at a first MiMe locus of a second MiMe component, and one or more MiMe alleles and one or more non-MiMe alleles at a second MiMe locus of the second MiMe component; and
      (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   vi) a partially complemented MiMe genotype comprising:
      (a) only MiMe alleles at one or more MiMe loci of a first MiMe component; and
      (b) one or more MiMe alleles and one or more non-MiMe alleles at a first MiMe locus of a second MiMe component, and one or more MiMe alleles and one or more non-MiMe alleles at a second MiMe locus of the second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
   vii) a partially complemented MiMe genotype comprising:
      (a) only MiMe alleles at one or more MiMe loci of a first MiMe component, wherein the first MiMe component is a component of DNA double strand breakage during meiotic recombination;
      (b) one or more MiMe alleles and one or more non-MiMe alleles at one or more MiMe loci of a second MiMe component;
      (c) one or more MiMe alleles and one or more non-MiMe alleles at one or more MiMe loci of a third MiMe component; and
      (d) one or more MiMe alleles and one or more non-MiMe alleles at one or more MiMe loci of a fourth MiMe component,
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

81. The genetically modified plant, plant part, or plant cell of embodiment 80, wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

82. The genetically modified plant, plant part, or plant cell of embodiment 80 or 81, comprising:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

83. The genetically modified plant, plant part, or plant cell of any one of embodiments 80-82, comprising one or more polynucleotide sequences selected from the group consisting of SEQ ID NOs: 108-111.

84. The genetically modified plant, plant part, or plant cell of any one of embodiments 80-83, wherein:
(i) the genetically modified plant part is a non-regenerable plant part; or
(ii) the genetically modified plant cell is a non-regenerable plant cell.

85. The genetically modified plant, plant part, or plant cell of any one of embodiments 80-84, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.

86. A processed plant product derived from the genetically modified plant, plant part, or plant cell of any one of embodiments 80-85 wherein the processed plant product comprises a detectable amount of the one or more MiMe alleles of the genetically modified plant, plant part, or plant cell.

87. The processed plant product of embodiment 86, wherein the processed plant product:
(i) is selected from the group consisting of plant biomass, oil, meal, food starch, syrup, animal feed, flour, flakes, bran, lint, hulls, and processed seed; and/or
(ii) is non-regenerable.

88. Germplasm of the population of polyploid maize seeds of any one of embodiments 45-59.

89. A population of polyploid maize seed comprising a subpopulation of genetically uniform polyploid maize seed in an amount of at least 50% of the total number of seeds, the genetically uniform polyploid maize seed comprising three or more haplotypes of the same or related species of plant, wherein the population was obtained from a single plant or a set of F1 hybrids, wherein the subpopulation of genetically uniform polyploid maize seed comprise
(A) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the MiMe alleles comprises a germplasm genetic modification,
wherein at least one seed, or a plant grown therefrom, of the population of polyploid maize seed has been selected for lack of expression of each of the one or more MiMe loci, and
wherein the subpopulation of genetically uniform polyploid maize seeds comprises (i) a complete MiMe genotype comprising only MiMe alleles at each of the one or more MiMe loci, (ii) a partial MiMe genotype comprising at least one MiMe allele and at least one non-MiMe allele at each of the one or more MiMe loci, or (iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

90. The population of embodiment 89, wherein the subpopulation of genetically uniform polyploid maize seed is triploid, tetraploid, pentaploid, hexaploid, heptaploid, or octaploid.

91. The population of embodiment 89 or 90, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

92. The population of any one of embodiments 89-91, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

93. The population of embodiment 89-92, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

94. The population of any one of embodiments 89-93, wherein the subpopulation of genetically uniform polyploid maize seed has a partially-complemented MiMe genotype comprising:
(A)
   (a) only MiMe alleles at one or more MiMe loci of a first MiMe component;
   (b) one or more MiMe alleles and one or more non-MiMe alleles at a first MiMe locus of a second MiMe component, and one or more MiMe alleles and one or more non-MiMe alleles at a second MiMe locus of the second MiMe component; and
   (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
   wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
(B)
   (a) only MiMe alleles at one or more MiMe loci of a first MiMe component; and
   (b) one or more MiMe alleles and one or more non-MiMe alleles at a first MiMe locus of a second MiMe component, and one or more MiMe alleles and one or more non-MiMe alleles at a second MiMe locus of the second MiMe component,
   wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
(C)
   (a) only MiMe alleles at one or more MiMe loci of a first MiMe component, wherein the first MiMe component is the component of DNA double strand breakage during meiotic recombination;
   (b) one or more MiMe alleles and one or more non-MiMe alleles at one or more MiMe loci of a second MiMe component;
   (c) one or more MiMe alleles and one or more non-MiMe alleles at one or more MiMe loci of a third MiMe component; and
   (d) one or more MiMe alleles and one or more non-MiMe alleles at one or more MiMe loci of a fourth MiMe component,
   wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.

95. The population of any one of embodiments 89-94, wherein the subpopulation of genetically uniform polyploid maize seed comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

96. The population of any one of embodiments 89-95, wherein the subpopulation of genetically uniform polyploid maize seed comprises one or more polynucleotide sequences selected from the group consisting of SEQ ID NOs: 108-111.

97. The population of any one of embodiments 89-96, wherein germination of a seed of the subpopulation of genetically uniform polyploid maize seed results in a sterile plant that produces inviable gametes, seedless fruit, or a combination thereof.

98. The population of any one of embodiments 89-97, wherein the population of polyploid maize seed comprises three or more haplotypes from the same species of maize.

99. A method of producing the population of polyploid maize seed of any one of embodiments 89-98, the method comprising:
(a) generating a first parent MiMe plant and a second parent MiMe plant by introducing genetic modifications into one or more candidate lines to produce MiMe alleles in germplasm of a first parent MiMe plant, a second parent MiMe plant, and/or progenitors thereof, wherein the first parent MiMe plant and the second parent MiMe plant each comprise a complete MiMe genotype, and each of the progenitors comprises a partial MiMe genotype, wherein if progenitors are generated, the progenitors are further crossed to generate the first parent MiMe plant, the second parent MiMe plant, or both;
(b) providing clonal gametes from the first parent MiMe plant and the second parent MiMe plant that together comprise the three or more haplotypes; and
(c) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

100. The method of embodiment 99, wherein the subpopulation of genetically uniform polyploid maize seed has a partially-complemented MiMe genotype, wherein:
(A)
   (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
   (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
   (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
   (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
   (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
   (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

101. A method of producing the population of polyploid maize seed of any one of embodiments 1-98, the method comprising:
(a) generating a first parent MiMe plant by introducing genetic modifications into one or more candidate lines to produce MiMe alleles in germplasm of the first parent MiMe plant or progenitors thereof, wherein the first parent MiMe plant comprises a complete MiMe genotype, and each of the progenitors comprises a partial MiMe genotype, wherein if progenitors are generated, the progenitors are further crossed to generate the first parent MiMe plant;
(b) providing clonal gametes from the parent MiMe plant;
(c) providing haploid gametes from a homozygous parent non-MiMe plant; and
(d) crossing the clonal gametes with the haploid gametes to produce the population of polyploid maize seed,
wherein the clonal gametes and the haploid gametes together comprise three or more haplotypes, optionally wherein step (a) comprises generating the first parent MiMe plant, the second parent MiMe plant, or both.

102. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant and a second parent MiMe plant from the two or more candidate lines that together comprise the three or more haplotypes;
(e) providing clonal gametes from each of the first and second parent MiMe plants;
(f) crossing the clonal gametes to produce a hybrid polyploid maize seed comprising the three or more haplotypes;
(g) growing the hybrid polyploid maize seed to produce a hybrid polyploid plant comprising three or more haplotypes; and
(h) evaluating one or more characteristics of the hybrid polyploid plant,
wherein the first parent MiMe plant, the second parent MiMe plant, and the hybrid polyploid plant each independently comprise:
(A) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1,SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the MiMe alleles comprises a germplasm genetic modification,
wherein the method comprises selecting the first parent MiMe plant, the second parent MiMe plant, the hybrid polyploid plant, or any combination thereof for lack of expression of the one or more MiMe loci, and wherein the first parent MiMe plant and the second parent MiMe plant each independently comprise a complete MiMe genotype comprising only MiMe alleles at each of the one or more MiMe loci, and
wherein the hybrid polyploid plant comprises (i) a complete MiMe genotype comprising only MiMe alleles at each of the one or more MiMe loci, or (ii) a partially complemented MiMe genotype comprising only MiMe alleles at a first MiMe locus of the one or more MiMe loci and at least one MiMe allele and at least one non-MiMe allele at a second MiMe locus of the one or more MiMe loci;
the method optionally further comprising
(i) repeating steps (b)-(h) or steps (c)-(h) using the one or more characteristics of the hybrid polyploid plant evaluated in step (h) to guide the breeding of lines of step (b), the selecting of candidate lines of step (c), or both.

103. The method of embodiment 102, wherein:
(A)
   (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
   (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
(B)
   (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
   (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
(C)
   (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
   (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

104. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant from one of the two or more candidate lines;
(e) providing clonal gametes from the first parent MiMe plant;
(f) providing haploid gametes from a homozygous parent non-MiMe plant of one of the two or more candidate lines;
(g) crossing the clonal gametes with the haploid gametes to produce a hybrid polyploid maize seed;
(h) growing the hybrid polyploid maize seed to produce a hybrid polyploid plant; and
(i) evaluating one or more characteristics of the hybrid polyploid plant,
wherein the first parent MiMe plant and the homozygous parent non-MiMe plant together comprise three or more haplotypes, wherein the crossing of step (g) results in the hybrid polyploid maize seed comprising three or more haplotypes, and wherein the growing of step (h) results in the hybrid polyploid plant comprising three or more haplotypes;
wherein the first parent MiMe plant and the hybrid polyploid plant each independently comprise:
(A) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the MiMe alleles comprises a germplasm genetic modification,
wherein the method comprises screening the first parent MiMe plant, the hybrid polyploid plant, or any combination thereof for lack of expression of the one or more MiMe loci, and
wherein the first parent MiMe comprises a complete MiMe genotype comprising only MiMe alleles at each of the one or more MiMe loci, the parent non-MiMe plant comprises only non-MiMe alleles at each of the one or more MiMe loci, and the hybrid polyploid plant comprises a partial MiMe genotype comprising at least one MiMe allele and at least one non-MiMe allele at each of the one or more MiMe loci, the method optionally further comprising
(j) repeating steps (b)-(i) or steps (c)-(i) using the one or more characteristics of the hybrid polyploid plant evaluated in step (i) to guide the breeding of lines of step (b), the selecting of candidate lines of step (c), or both.

105. The method of any one of embodiments 101-104, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octaploid.

106. The method of any one of embodiments 101-105, wherein generating the first parent MiMe plant, the second parent MiMe plant, or both comprises:
(1) introducing a complete MiMe genotype directly into two candidate lines to produce the first parent MiMe plant, the second parent MiMe plant, or both;
(2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant, optionally wherein generating the first parent MiMe plant, the second parent MiMe plant, or both further comprises introducing a complete MiMe genotype directly into a third candidate line to produce the second parent MiMe plant; or
(3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants, optionally wherein generating the first parent MiMe plant, the second parent MiMe plant, or both further comprises propagating the first parent MiMe plant, the second parent MiMe plant, the grandparent non-MiMe plants, or any combination thereof to scale production of homogenous seed.

107. The method of any one of embodiments 102-106, wherein the hybrid polyploid plant comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1; (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

108. The method of any one of embodiments 102-107, wherein one or more of the candidate lines of step (c) are inbred lines, hybrid lines, or a combination thereof.

109. The method of any one of embodiments 102-108, wherein the complete MiMe genotype, the partial MiMe genotype, or both are introduced by gene editing, transgenesis, or a combination thereof.

110. The method of embodiment 102-109, wherein the decreased expression of one or more of the MiMe loci is each independently achieved by gene disruption, gene knockout, gene knockdown, gene silencing, RNA interference, or induction of methylation.

111. The method of embodiment 102-110, wherein the decreased expression of one or more of the MiMe loci is each independently achieved by introducing into each candidate line or a progenitor thereof an insertion, a deletion, one or more nucleotide changes, or an inversion that results in decreased expression of the MiMe locus, optionally including a step of selection for decreased expression of the MiMe locus.

112. The method of embodiment 111, wherein the insertion, the deletion, the one or more nucleotide changes, or the inversion eliminates expression of the MiMe locus, optionally wherein the expression of the MiMe locus is eliminated by a premature stop codon present in the 70%, the first 60%, the first 50%, the first 40%, the first 30%, the first 20%, or the first 10% of the nucleotides of the coding sequence of the MiMe locus following the start codon in the 3′ direction.

113. A method of producing a population of polyploid maize seed comprising:
(a) providing clonal gametes from a pair of parent MiMe plants together comprising three or more haplotypes that were selected using the method of breeding of any one of embodiments 102, 103, and 105-112 based upon the polyploid plant comprising the three or more haplotypes having one or more desired characteristics; and
(b) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

114. A method of producing a population of polyploid maize seed comprising:
(a) selecting three or more haplotypes using the method of breeding of any one of embodiments 104-112 based upon the polyploid plant comprising said three or more haplotypes having one or more desired characteristics;
(b) providing clonal gametes from a parent MiMe plant;
(c) providing haploid gametes from a homozygous parent non-MiMe plant;
(d) crossing the clonal gametes with the haploid gametes to produce the population of polyploid maize seed;
wherein the parent MiMe plant and the homozygous parent non-MiMe plant together comprise the three or more haplotypes selected in step (a), wherein the crossing of step (d) results in a population of polyploid maize seed comprising a subpopulation of genetically uniform polyploid maize seed in an amount of at least 50% of the total number of seeds, the subpopulation of genetically uniform polyploid maize seed comprising the three or more haplotypes.

115. The method of embodiment 113 or 114, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

116. The method of any one of embodiments 113-115, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

117. The method of any one of embodiments 113-116, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

118. A genetically modified plant, plant part, or plant cell comprising:
(A) three or more haplotypes; and
(B)
   (a) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSYJ, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
   (b) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
   wherein each of the of the MiMe alleles comprises a germplasm genetic modification comprising introduction of a premature in-frame stop codon present in the first 30% of the nucleotides of the coding sequence of the MiMe locus following the start codon in the 3′ direction, thereby generating a MiMe allele, and
   wherein the subpopulation of genetically uniform polyploid maize seeds comprises (i) a complete MiMe genotype comprising only MiMe alleles at each of the one or more MiMe loci, (ii) a partial MiMe genotype comprising at least one MiMe allele and at least one non-MiMe allele at each of the one or more MiMe loci, or (iii) a partially complemented MiMe genotype comprising only MiMe alleles at a first MiMe locus of the one or more MiMe loci and at least one MiMe allele and at least one non-MiMe allele at a second MiMe locus of the one or more MiMe loci
   wherein the partially-complemented MiMe genotype comprises:
   (i)
      (a) only MiMe alleles at one or more MiMe loci of a first MiMe component;
      (b) one or more MiMe alleles and one or more non-MiMe alleles at a first MiMe locus of a second MiMe component, and one or more MiMe alleles and one or more non-MiMe alleles at a second MiMe locus of the second MiMe component; and
      (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
      wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
   (ii)
      (a) only MiMe alleles at one or more MiMe loci of a first MiMe component; and
      (b) one or more MiMe alleles and one or more non-MiMe alleles at a first MiMe locus of a second MiMe component, and one or more MiMe alleles and one or more non-MiMe alleles at a second MiMe locus of the second MiMe component,
      wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
   (iii)
      (a) only MiMe alleles at one or more MiMe loci of a first MiMe component, wherein the first MiMe component is the component of DNA double strand breakage during meiotic recombination;
      (b) one or more MiMe alleles and one or more non-MiMe alleles at one or more MiMe loci of a second MiMe component;
      (c) one or more MiMe alleles and one or more non-MiMe alleles at one or more MiMe loci of a third MiMe component; and
      (d) one or more MiMe alleles and one or more non-MiMe alleles at one or more MiMe loci of a fourth MiMe component,
      wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.

119. The genetically modified plant, plant part, or plant cell of embodiment 118, comprising:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid maize seed is from a maize plant and the one or more OSD1 loci comprise OSD1-1 and OSD1-2;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

120. The genetically modified plant, plant part, or plant cell of embodiment 118 or 119, comprising one or more polynucleotide sequences selected from the group consisting of SEQ ID NOs: 108-111.

121. The genetically modified plant, plant part, or plant cell of any one of embodiments 118-120, wherein:
(i) the genetically modified plant part is a non-regenerable plant part; or
(ii) the genetically modified plant cell is a non-regenerable plant cell.

122. The genetically modified plant, plant part, or plant cell of any one of embodiments 118-121, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.

123. A processed plant product derived from the genetically modified plant, plant part, or plant cell of any one of embodiments 118-122 wherein the processed plant product comprises a detectable amount of the one or more MiMe alleles of the genetically modified plant, plant part, or plant cell.

124. The processed plant product of embodiment 123, wherein the processed plant product:
(i) is selected from the group consisting of plant biomass, oil, meal, food starch, syrup, animal feed, flour, flakes, bran, lint, hulls, and processed seed; and/or
(ii) is non-regenerable.

125. Germplasm of the population of polyploid maize seeds of any one of embodiments 89-98.

126. A method of producing comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of F1 hybrids, the method comprising:
(1)
   (a) generating a first parent MiMe plant and a second parent MiMe plant;
   (b) providing clonal gametes from the first parent MiMe plant and the second parent MiMe plant that together comprise the three or more haplotypes; and
   (c) crossing the clonal gametes to produce the population of polyploid seed,
or
(2)
   (a) generating a first parent MiMe plant;
   (b) providing clonal gametes from the parent MiMe plant;
   (c) providing haploid gametes from a homozygous parent non-MiMe plant; and
   (d) crossing the clonal gametes with the haploid gametes to produce the population of polyploid seed,
wherein the generating in step (a) comprises introducing the following MiMe alleles (A) or (B) at the following MiMe loci by (i) introducing genetic modifications in the MiMe loci and selecting for lack of expression at the MiMe loci, (ii) introducing an expression construct for a silencing RNA targeting the MiMe loci, or (iii) introducing a natural MiMe allele or selecting a first parent MiMe plant, a second parent MiMe plant or a progenitor with the natural allele for use in combination with (i) and/or (ii) for other MiMe alleles; wherein genetic modifications and/or expression constructs are introduced into one or more candidate lines to produce MiMe alleles in germplasm of a first parent MiMe plant, a second parent MiMe plant, and/or progenitors thereof, wherein the first parent MiMe plant and the second parent MiMe plant each comprise a complete MiMe genotype, and each of the progenitors comprises a partial MiMe genotype, wherein if progenitors are generated, the progenitors are further crossed to generate the first parent MiMe plant, the second parent MiMe plant, or both:
   (A) (1) one or more MiMe alleles at one or more MiMe loci of a component of sister chromatid cohesion during the first division of meiosis comprising REC8 (i.e., a gene encoding a protein having at least 60% sequence identity to one of NP 001105829.1, XP 008648327.1, XP 008648329.1, and XP 008648328.1 or to XP 006347252.1), SWITCH1/DYAD (i.e., a gene encoding a protein having at least 60% sequence identity to one of NP_001139538.1, XP 008662288.1 and C0RWW9 or to M1BMI9), or a combination thereof, (2) one or more MiMe alleles at one or more MiMe loci of a component of DNA double strand breakage during meiotic recombination comprising PAIR1 (i.e., a gene encoding a protein having at least 60% sequence identity to one of XP 008660580.1, A0A1D6JK92, A0A1D6JK93, A0A1D6JK94, and A0A1D6PM18 or to one of XP 006339791.2 and MOZGU5), SPO11-1 (i.e., a gene encoding a protein having at least 60% sequence identity to one of NP 001347894.1, XP 008643457.1, XP 008643458.1, XP 008643459.1, XP 020408860.1, and XP 008643458.1 or to one of XP 006346146.1, M1C0B8, and M1CP72), SPO11-2 (i.e., a gene encoding a protein having at least 60% sequence identity to one of XP 020406911.1, NP_001298099.1 and NP_001141583.1 or to one of XP 006344018.1, XP 006367265.1 and M1CP72), PRD1 (i.e., a gene encoding a protein having at least 60% sequence identity to one of XP 020399554.1, XP 020399553.1 and A0A1D6P5Q4 or to one of XP 015163123.1 and M1CA99), PRD2 (i.e., a gene encoding a protein having at least 60% sequence identity to one of NP_001130070.1, XP 035822500.1 and A0A3L6F902 or to one of XP 015162530.1 and M1AS84), DFO (i.e., a gene encoding a protein having at least 60% sequence identity to one of XP 020397706.1, A0A1D6QG87 and A0A3L6EXP8 or to one of XP 006350870.1 and M1CZR2), MTOPVIB (i.e., a gene encoding a protein having at least 60% sequence identity to one of XP 008645058.1, A0A3L6EMU7 and A0A1D6GVU9 or to one of XP 015166906.1, XP 015166907.1 and M1CGP5), DSY1, SY1, SY2, SY3, SY4, or any combination thereof, and (3) one or more MiMe alleles at one or more MiMe loci of the component of progression through the second division of meiosis comprising OSD1 (i.e., a gene encoding a protein having at least 60% sequence identity to one of A0A1D6HBV9, A0A1D6HBWO, B4FCP3, B4FG75, and B6T7U1 or to one of XP 006351336.1), CYCA1 (i.e., a gene encoding a protein having at least 60% sequence identity to one of NP_001105387.2, NP_001288521.1 and XP 008656316.2 or to one of XP 006351137.1, XP 006351136.1 and XP 006351138.1), TDM1 (i.e., a gene encoding a protein having at least 60% sequence identity to one of NP_001170740.1, NP_001141607.1 and XP 008651103.1 or to one of XP 006350746.1, XP 006340757.1 and XP 006360343.1), PC1, PC2, FC, or any combination thereof; or
   (B) (2) one or more MiMe alleles at one or more MiMe loci of the component of DNA double strand breakage during meiotic recombination comprising PAIR1 (i.e., a gene encoding a protein having at least 60% sequence identity to one of), SPO11-1 (i.e., a gene encoding a protein having at least 60% sequence identity to one of), SPO11-2, PRD1, PRD2, DFO, MTOPVIB, DSY1, SY1, SY2, SY3, SY4, or any combination thereof, and (4) one or more MiMe alleles at one or more MiMe loci of the component of progression through the first division of meiosis comprise PS1 (i.e., a gene encoding a protein having at least 60% sequence identity to one of NP_001348386.1, XP 035820777.1 and XP 008668271.1 or to one of XP 006351164.1, XP 006353632.1 and M1AJ10), JASON (i.e., a gene encoding a protein having at least 60% sequence identity to one of NP_001132267.1, XP 008647301.1 and NP_001130670.1 or to one of XP 006352001.1, XP 015160469.1 and M1CT79), or a combination thereof;
wherein the subpopulation of genetically uniform polyploid seeds comprises (i) a complete MiMe genotype comprising only MiMe alleles at each of the one or more MiMe loci, (ii) a partial MiMe genotype comprising at least one MiMe allele and at least one non-MiMe allele at each of the one or more MiMe loci, or (iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

127. The method of embodiment 126, wherein the subpopulation of genetically uniform polyploid seed is triploid, tetraploid, pentaploid, hexaploid, heptaploid, or octaploid.

128. The method of embodiment 126 or 127, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.

129. The population of any one of embodiments 126-128, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.

130. The population of embodiment 126-129, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.

131. The population of any one of embodiments 126-130, wherein the subpopulation of genetically uniform polyploid seed has a partially-complemented MiMe genotype comprising:
(A)
   (a) only MiMe alleles at one or more MiMe loci of a first MiMe component;
   (b) one or more MiMe alleles and one or more non-MiMe alleles at a first MiMe locus of a second MiMe component, and one or more MiMe alleles and one or more non-MiMe alleles at a second MiMe locus of the second MiMe component; and
   (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
   wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
(B)
   (a) only MiMe alleles at one or more MiMe loci of a first MiMe component; and
   (b) one or more MiMe alleles and one or more non-MiMe alleles at a first MiMe locus of a second MiMe component, and one or more MiMe alleles and one or more non-MiMe alleles at a second MiMe locus of the second MiMe component,
   wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
(C)
   (a) only MiMe alleles at one or more MiMe loci of a first MiMe component, wherein the first MiMe component is the component of DNA double strand breakage during meiotic recombination;
   (b) one or more MiMe alleles and one or more non-MiMe alleles at one or more MiMe loci of a second MiMe component;
   (c) one or more MiMe alleles and one or more non-MiMe alleles at one or more MiMe loci of a third MiMe component; and
   (d) one or more MiMe alleles and one or more non-MiMe alleles at one or more MiMe loci of a fourth MiMe component,
   wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.

132. The method of any one of embodiments 126-131, wherein the subpopulation of genetically uniform polyploid seed comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPOT 1-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

133. The population of any one of embodiments 126-132, wherein the subpopulation of genetically uniform polyploid seed comprises one or more polynucleotide sequences selected from the group consisting of SEQ ID NOs: 108-111.

### EXAMPLES

The presently disclosed subject matter will be better understood by reference to the following Examples, which are provided as exemplary of the inventions, and not by way of limitation.

### Example 1: General Methods

GM: Methods

GM1: Identifying Homologs of Genes Underlying MiMe-Like Phenotype Across Plant Species

Core genes involved in the conversion of meiosis to mitosis (mitosis instead of meiosis or MiMe) were identified through alignment with any of the canonical reference sequences for REC8, OSD1 (e.g., OSD1-2, and/or OSD1-3 from maize), PAIR1 PAIR1-1 and/or PAIR1-2 from maize), SPO11-1, JASON (e.g., JASON 1 and JASON-2 from maize), CYCA (also known as CYCLIN-A1 or as TARDY ASYNCHRONOUS MEIOSES (TAM)), TDM1, and/or PS1 as shown in Table 5 (SEQ IDs 1-12). Amino acid sequences of the protein isoforms predicted from open reading frames of these genes were aligned to the NCBI RefSeq (O'Leary et al. (2016) Reference sequence (RefSeq) database at NCBI, current status, taxonomic expansion, and functional annotation. Nucleic Acids Res. 44(D1): D733-D745), UniProtKB Swiss-Prot and TrEMBL (The UniProt Consortium (2019) UniProt: a worldwide hub of protein knowledge, Nucleic Acids Res. 47(D1): D506—D515, https://doi.org/10.109,3/nar/gky1049) protein databases. These alignments were performed using default parameters (gap opening penalty=11, gap extension penalty=1, E-value=10, word size=3, max score=25, query filter=SEG, substitution matrix=BLOSUM 62) in Protein-Protein BLAST 2.11.0+(O'Leary et al. (2016)). For example, core MiMe genes in a plant's genome may be identified through alignment with the REC8, OSD1, PAIR1, and SPO11-1 sequences from Oryza sativa.

Candidate orthologs were further validated through alignments between the identified orthologs and canonical protein sequences in Table 5 using Clustal Omega—1.2.4 with default parameters (substitution matrix=GONNET) for protein-protein alignments. The canonical protein sequences used were those from Arabidopsis thaliana for dicots, and those from Oryza sativa for monocots. In some cases, the canonical protein sequences available were from Arabidopsis thaliana. Results were filtered for relevant species and were further manually curated to remove spurious alignments. Orthologs with an amino acid consensus greater than 50% when aligning maize queries against potential maize sequences were considered as candidate orthologs with a preference for the highest conservation of amino acid sequence and exon structure relative to the canonical set (SEQ ID NOs: 1-12, Table 5).

GM1.1: Identification of Target DNA Sequences for crRNA Design

The protein Blast results were aligned to a genomic database using BLAST's “tblastn” search, specifically the Protein Query-Translated Subject BLAST 2.11.0+ with default parameters (gap opening penalty=11, gap extension penalty=1, E-value=10, word size=3, max score=25, query filter=SEG, query genetic code=universal, substitution matrix=BLOSUM 62). The corresponding nucleotide sequences of identified orthologs and any putative paralogs were extracted from this search, including 5 kb upstream of each gene, using custom shell scripts. These sequences were then aligned to each other using Clustal Omega—1.2.4 with default parameters (substitution matrix=GONNET). Most likely candidate sequences, with the highest identity to the canonical sequences and with the most conserved exon structure were used to design CRISPR RNA (crRNA) for an appropriate CRISPR-associated (Cas) nuclease.

GM1.2: Design of crRNA for DNA Editing with a Cas Nuclease

For each species of interest, the most probable candidate sequences identified from the protein BLAST, tblastn, and Clustal Omega workflows were targeted for crRNA design in Geneious Prime 2020.0.3, with protospacer adjacent motif (PAM) sites near each candidate identified for an appropriate Cas nuclease. crRNA with high specificity targeting the first or second exons or promoter sequences were generally preferred, but high-scoring crRNA targeting later exons were also selected. Resulting sequences were exported and scaffolds for an appropriate Cas nuclease were added. Functional crRNAs were synthesized by IDT (Integrated DNA Technologies, Newark, NJ, USA) using standard RNA synthesis. The process was repeated for each target gene, including REC8, OSD1 (e.g., OSD1-1, OSD1-2, and/or OSD1-3 from maize), PAIR1 (e.g., PAIR1-1 and/or PAIR1-2 from maize), SPO11-1, JASON (e.g., JASON-1 and JASON-2 from maize), CYCA1 (e.g., CYCA1-1 and CYCA1-2 from maize), TDM1, and/or PS1 crRNA were screened for editing efficiency in protoplasts.

In monocots, editing was achieved by stable integration of two distinct DNA cassettes, one of which contained an appropriate codon-optimized Cas nuclease (i.e., the nuclease construct) and the other of which contained an array of crRNAs targeting MiMe alleles (i.e., the guide RNA construct). The DNA sequences corresponding to the crRNAs were inserted into a standard crRNA guide array between repeats, which were recognized and cleaved by either an appropriate ribonuclease or a ribozyme. The cassettes were each driven by their own plant ubiquitin promoter; however, plant U6 promoters or other suitable promoters with sufficient expression to achieve editing in the target plant were also tested and achieved similar results.

GM2: Ribonucleoprotein Preparation

To prepare ribonucleoproteins (RNPs), 2 μL of New England Biolabs buffer (NEBuffer™) 2.1 (10× stock) was placed into a 1.5 mL microcentrifuge tube with 10-600 pmol of crRNA and with an equal amount of the selected Cas nuclease protein. The final volume was adjusted to 20 μL using nuclease-free water. The solution was prepared fresh and used after a 15-minute incubation at room temperature.

GM3: Sequence-Based Edit Confirmation

Primers were designed to amplify each gene, and protoplasts were generated. Further details on plant selection, plant growth, protoplast generation, and protoplast transfection, are provided in the species-specific protocols in the Examples below.

Some transfected protoplasts were incubated at room temperature for 24 to 48 hours, then lysed and one or more long-range direct polymerase chain reactions (PCRs) were performed on the crude lysates. Other transfected protoplasts were regenerated, and DNA was then extracted from these protoplasts' regenerated callus, leaf, or other plant tissue. PCR products were pooled by transfection sample and a segWell™ (Beverly, MA, USA) library preparation was performed to generate an Illumina (Illumina, San Diego, CA, USA) library. Samples were loaded onto an Illumina iSeq (Illumina, San Diego, CA, USA) and sequenced with a paired-end 150 nt sequencing kit. Sequences were analyzed by aligning FASTQ files to reference sequences and mutations adjacent to target sites for each gene were tabulated relative to a control. Editing efficiency was calculated based on the frequency of observed mutations in the reads obtained for a given sample, and this information was used to calculate how many plants should be screened to identify the multi-gene knockouts required to induce the clonal gamete production.

PCR amplicons were used to prepare Illumina sequencing libraries using plexWell 96 kits (segWell™ Beverly, MA, USA) and libraries were sequenced on an Illumina iSeq (Illumina, San Diego, CA, USA). FASTQ data sets were aligned to the corresponding reference genomes using the BWA-MEM algorithm (Li, H. (2013) Aligning sequence reads, clone sequences and assembly contigs with BWA-MEM. arXiv preprint arXiv:1303.3997) and variants were visualized and quantified using custom scripts. Editing efficiency was calculated as the fraction of reads in an edited sample with on-target mutations relative to an unedited control. Editing efficiency was determined for each guide and used to estimate the minimum number of plants needed to recover the required multi-gene knockout.

GM4: Flow Cytometry

Flow cytometry was performed on parent plants and on the progeny using the methods of Galbraith et al. 1983 (Rapid flow cytometric analysis of the cell cycle in intact plant tissues. Science. 220(4601): 1049-1051). Further details on parent plant selection and methods of crossing are provided in the species-specific protocols in the Examples below. Briefly, intact nuclei were extracted, filtered, and stained with propidium iodide as per instructions of the CyStain® PI Absolute P kit (Sysmex America, Lincolnshire, IL, USA). DNA content of nuclei was determined by applying the samples to a BD Accuri C6 Flow Cytometer. Gating was performed and genomic DNA content was calculated by comparing the peak area for the sample to the known position of a control with known ploidy. The ploidy of the unknown samples was determined based on relative comparison to each control.

GM5: Analysis of Genetic Uniformity Using the Jaccard Similarity Coefficient

The Jaccard similarity coefficient was used to measure genetic uniformity of populations of seeds. The Jaccard index, Jaccard similarity index, or Jaccard similarity coefficient (Jaccard, P. (1908) Nouvelles Recherches sur la Distribution Florale. Bulletin de la Societe Vaudoise des Sciences Naturelles. Vol. 44) is a metric used to compare the similarity of two sets. In the context of molecular plant genetics, the Jaccard index, Jaccard similarity index, or Jaccard similarity coefficient, is commonly applied to quantify the pairwise genetic similarity or uniformity of plants based on the presence or absence of shared alleles at loci spread throughout the genome (Paz and Veilleux (1997). Genetic diversity based on randomly amplified polymorphic DNA (RAPD) and its relationship with the performance of diploid potato hybrids. Journal of the American Society for Horticultural Sci. 122(6): 740-747; Vosman et al. (2004). The establishment of ‘essential derivation’ among rose varieties, using AFLP. Theoretical and Applied Genetics. 109: 1718-1725; Noli et al. (2013). Criteria for the definition of similarity thresholds for identifying essentially derived varieties. Plant Breeding. 132(6): 525-531; Vijayakumar et al. (2021) High temperature induced changes in quality and yield parameters of tomato (Solanum lycopersicum L.) and similarity coefficients among genotypes using SSR markers. Heliyon. 7(2); Dalamu et al. (2023). Genetic Diversity and Population Structure Analyses Using Simple Sequence Repeat Markers and Phenotypic Traits in Native Potato Collection in India. Potato Research: 1-25).

The Jaccard similarity coefficient is defined as the ratio of the number of shared items to the total number of distinct items in the two sets. In the context of molecular plant genetics, it can be used to quantify the proportion of shared alleles between two plants.

The formula for calculating the Jaccard similarity coefficient is:

J(A,B)=|A∩B|/|A∪B|

Where A represents the set of unique alleles without duplicates in one plant, B the set of unique alleles without duplicates in the other plant, |A∩B| represents the number of shared alleles (the cardinality of the intersection) between the plants, and |A∪B| represents the number of distinct alleles (the cardinality of the union) between the plants.

This formula computes the cardinality of the intersection (common elements) of two sets (the shared alleles) divided by the cardinality of the union (all alleles) of the two sets (all distinct alleles present). The resulting value of the Jaccard similarity coefficient ranges from 0 to 1, where 0 indicates no shared alleles, and 1 indicates complete uniformity.

The average pairwise genetic uniformity of the populations of seed was calculated as the average Jaccard similarity coefficient of all possible pairs of plants within the population. We note that in the context of genetic pairwise similarity estimations, the size of A should be the same as, or very close to, the size of B to avoid misinterpretation.

Genotyping results found in the Examples below (such as in Example 2's section ZR4) were encoded so that each allele at each locus represents a unique member of the set of total alleles. The number of shared alleles between each pair of progeny were divided by the total number of distinct alleles present in the two plants, and the results are the Jaccard index, Jaccard similarity index, or Jaccard similarity coefficient.

GM6: Population Genotyping

All relevant parent and progeny plants were sequenced via whole genome shotgun (WGS) sequencing. DNA samples were sequenced by Novogene. Libraries were prepared using the NEBNext® Ultra™ II for DNA Library Prep kit, and paired-end 2×150 reads were sequenced on an Illumina NovaSeq 6000 or NovaSeq× plus. Germline SNPs and small indels were called in all relevant parents using a standard whole genome variant calling pipeline. WGS data was aligned to a reference genome with BWA-MEM (Li, H. (2013) Aligning sequence reads, clone sequences and assembly contigs with BWA-MEM. arXiv preprint arXiv:1303.3997). PCR duplicates were removed and SNPs and small indels were called and jointly genotyped with GATK (McKenna, A et al. (2010) The Genome Analysis Toolkit: a MapReduce framework for analyzing next-generation DNA sequencing data. Genome research, 20(9), pp. 1297-1303). Tri-allelic and tetra-allelic markers were established to genotype populations.

Tri-allelic (containing three alleles) markers were used when one parent was expected to contribute one distinct haplotype while the other parent was expected to contribute two distinct haplotypes. Pairs of SNPs that were close enough to be directly phased by 150 bp Illumina reads were used. Each SNP was unique/exclusive to one of the parents. One SNP/parent was homozygous while the other was heterozygous (REF/ALT or ALT1/ALT2). To genotype progeny, we looked for the presence of both ALT alleles (one from each parent) as well as the presence of the reference allele or second alternate allele of the heterozygous SNPs. If all alleles were observed and phasing was consistent with parental haplotypes, then the locus was genotyped as tri-allelic.

Tetra-allelic (containing four alleles) markers were used when both parents were expected to contribute two distinct haplotypes. Trios of SNPs that were close enough to be directly phased by 150 bp Illumina reads were used. One SNP was unique/exclusive to one parent and the two other SNPs were unique/exclusive to the other parent. All SNPs were heterozygous. The ALT alleles of the two SNPs exclusive to the same parent were on opposing haplotypes. To genotype progeny, we looked for the presence of all three ALT alleles (one from one parent and two from the other) as well as the presence of the reference allele or second alternate allele of the solo heterozygous SNPs. If all alleles were observed and phasing was consistent with parental haplotypes, then the locus was genotyped as tetra-allelic.

After establishing markers, markers were filtered, genotyped, and checked for quality across all progenies (via WGS data) using a combination of manual and custom automated techniques.

### Example 2: Generating Genetically Uniform Tetraploid Hybrid Maize

ZM: Methods

ZM1: Plant Materials for crRNA Guide Screening

For crRNA guide screening, seeds of Zea mays B73, Hi-II, or Fast-Flowering Mini-Maize (“FFMM-AT6”) (McCaw et al. (2020) Development of a Transformable Fast-Flowering Mini-Maize as a Tool for Maize Gene Editing. Frontiers in Genome Editing. doi: 10.3389/fgeed.2020.622227) were planted in a plug tray filled with PRO-MIX HP, covered with a humidome, and underlain with a seedling heating mat that maintained a soil temperature of about 30° C. After 4 days, when seedlings germinated enough to reach light, they were covered by a box to block light. When the second leaf was about 10 cm longer than the first leaf, it was removed and used for protoplast isolation.

ZM2: Protoplast Isolation

Approximately thirty minutes before digestion of the cell wall, 1-2 g of leaves were sliced into thin sections of about 0.5 mm in width and placed in a petri dish with 20 ml of digest solution (0.6M Mannitol, 10 mM MES (pH5.7), 1 mM CaCl₂, 5 mM (3-mercaptoethanol, 0.1% Bovine Serum Albumin, 1.5% Cellulase R10 and 0.3% Macerozyme R10). Leaf sections were then vacuum infiltrated for 30 minutes at room temperature, then incubated in the dark while shaking at 40 RPM for two hours. After two hours protoplasts were liberated by shaking at 80 RPM for 5 minutes. Protoplasts were then filtered through a 75 μM mesh and transferred to two round bottom tubes. The protoplasts were pelleted at 100×g for 5 minutes, then were washed twiceby resuspending in a cool wash solution (0.6M mannitol, 20mMKCl, 4 mM MES (pH5.7)) and pelleting. Finally, protoplasts were resuspended in 10 ml of wash solution and incubated on ice for one hour. Cells were quantified using a Biirker hemocytometer and stored on ice in the dark until transfection. A sample was also reserved to test cell viability using FDA staining as described by Larkin (1976. Purification and viability determinations of plant protoplasts. Planta 128(3): 213-216).

ZM3: Transfection

Immediately before transfection, a fresh polyethylene glycol (PEG) solution was prepared according to Cao et al. (2014. PEG-mediated transient gene expression and silencing system in maize mesophyll protoplasts: a valuable tool for signal transduction study in maize. Acta Physiologiae Plantarum. 36(5): 1271-1281.). Protoplasts were centrifuged at 100×g for 5 minutes and resuspended in a volume of MMG transformation buffer (4 mM MES, pH 5.7, 0.4 M mannitol and 15 mM MgCl₂) as described by Cao et al. (2014) to achieve a cell density of 1-2×10 6 protoplasts/mL. 15 μL of RNP was freshly prepared as described in Example 1's section GM2 and added to wells of a 48-well plate. Alternatively, 20 jug of plasmid DNA total in 15 μL of sterile deionized water was added to a 48-well plate. Next, 200 μL of protoplast suspension was mixed with the RNP solution (or plasmid DNA) by pipetting with wide bore tips, after which the approximately 215 μL of PEG solution was added and gently mixed by pipetting up and down with a wide bore tip until Schlieren lines were no longer visible. After a 20-minute incubation at room temperature, 800 μl of incubation solution (0.6 M mannitol, 4 mM KCl, 4 mM MES) was added to the approximately 215 μL of PEG solution and mixed again by pipetting. The protoplasts were then pelleted from the resulting solution at 100×g for 2 minutes, after which the plates were rotated and centrifuged again at 100×g for 2 minutes. The supernatant was then removed, and 2 mL of incubation solution (0.6 M mannitol, 4 mM KCl, 4 mM MES) was added to the wells. Protoplasts were resuspended according to Cao et al. 2014 and then incubated in the dark for 2 days at 27° C. After this incubation, 30 ul of cells were collected from the bottom of the wells and pipetted into a PCR plate where they were lysed, and DNA was extracted using the Platinum Direct Polymerase kit (#A44647100 ThermoFisher Scientific). A sample from each well was also evaluated for cell viability using FDA staining as described by Larkin (1976. Purification and viability determinations of plant protoplasts. Planta 128(3): 213-216).

ZM4: Plasmids for Biolistic Transformation

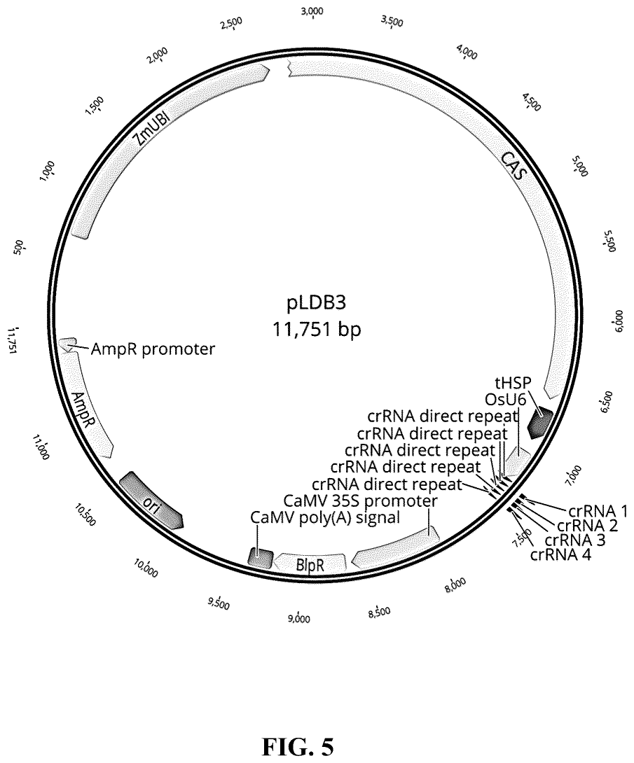

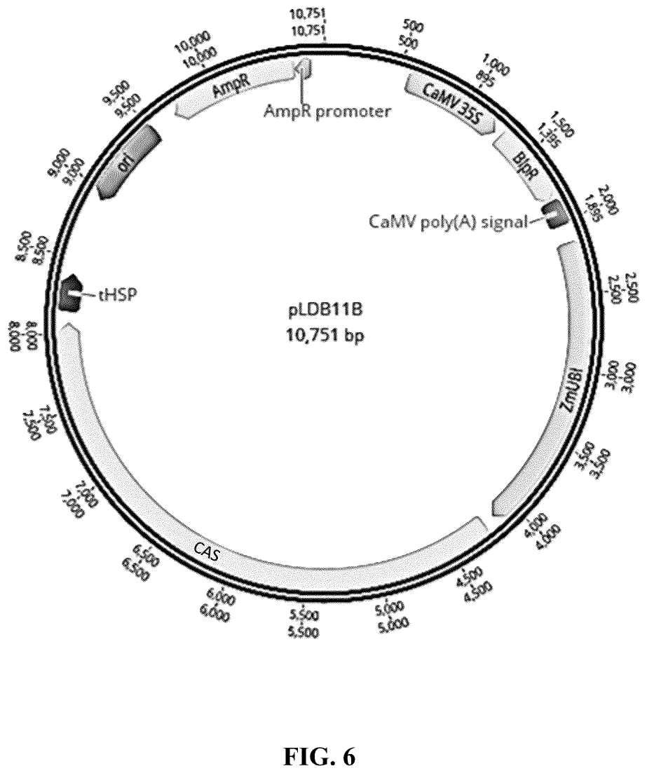

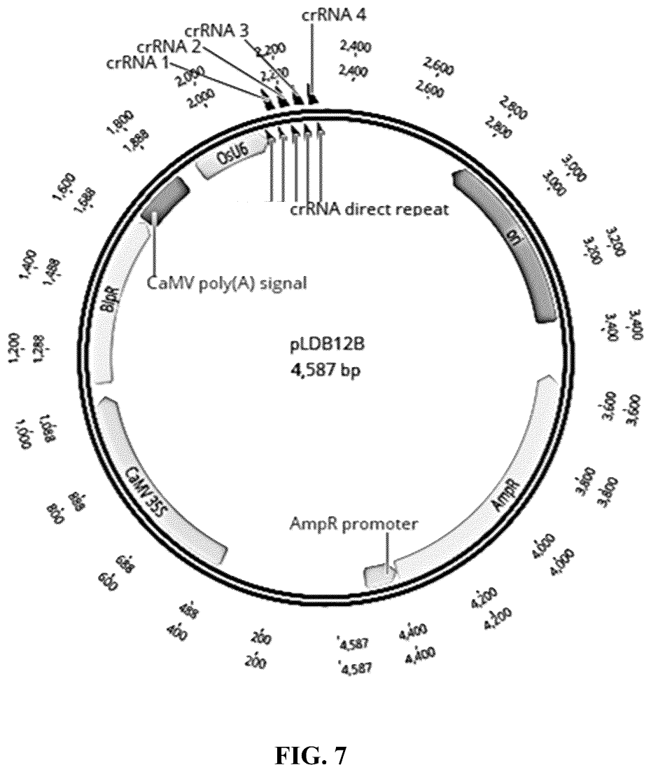

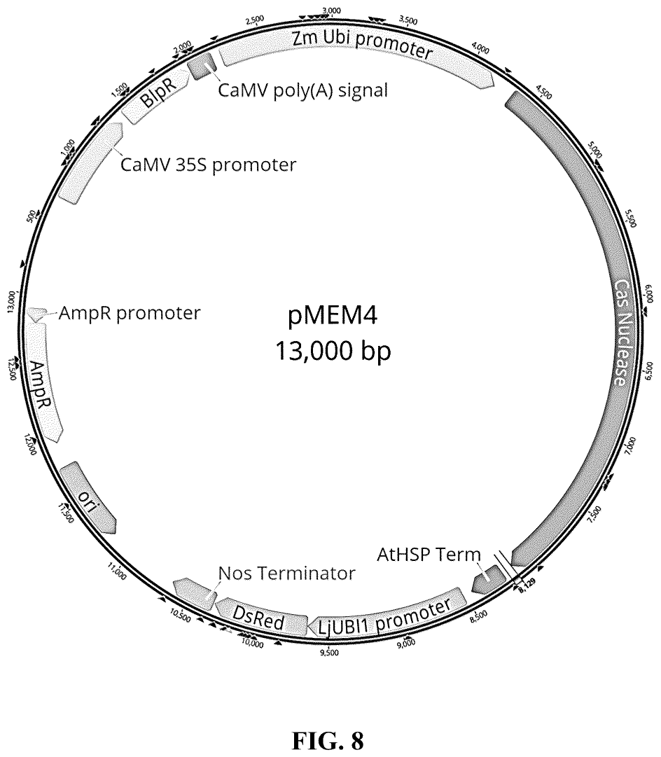

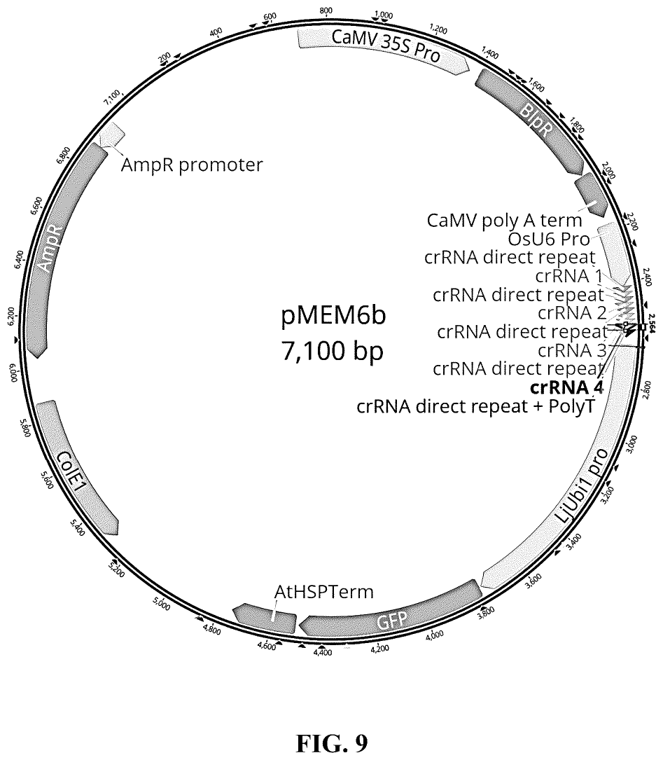

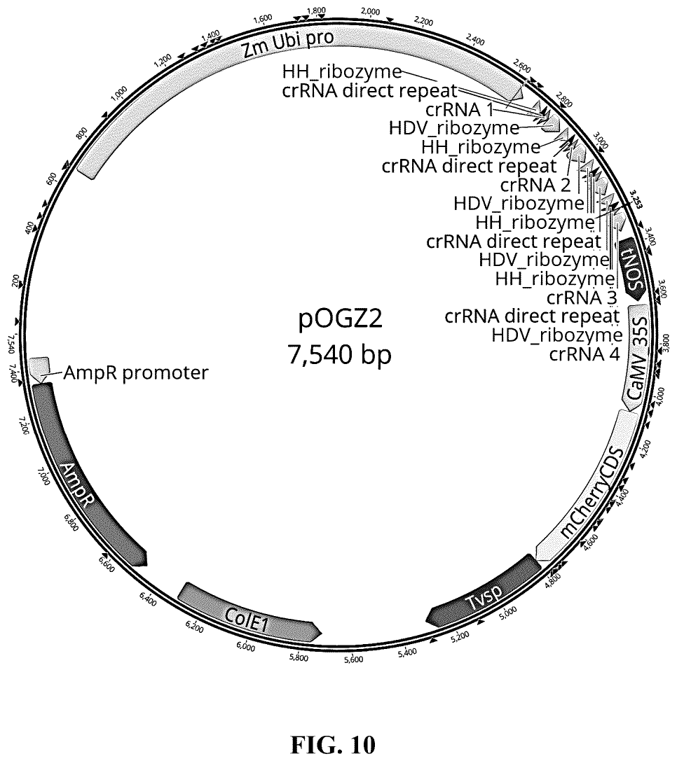

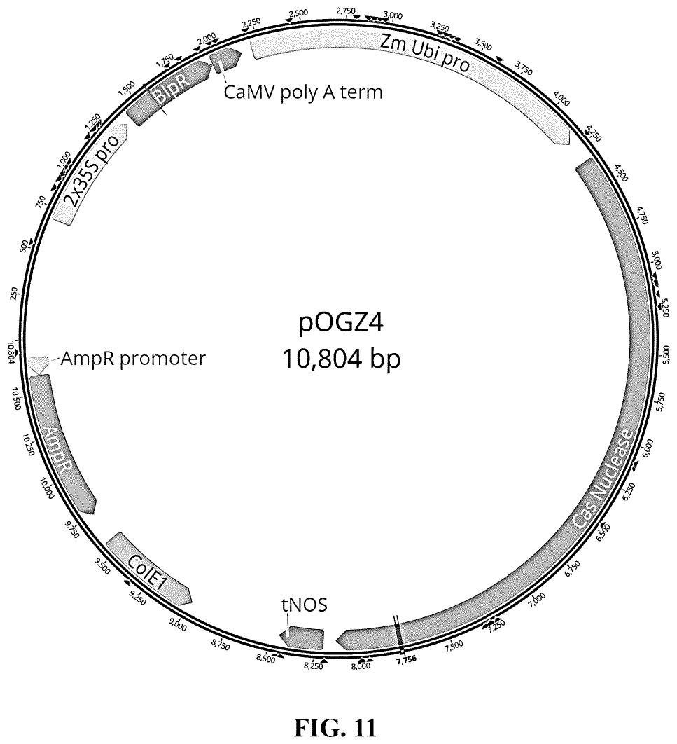

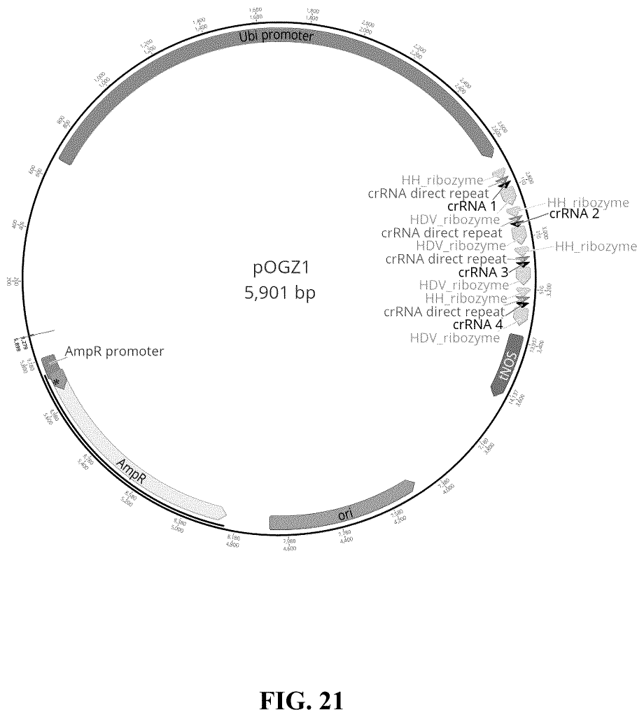

Plasmids were constructed for use in biolistic transformations of Zea mays. The most efficient crRNA guides were determined during guide screening in protoplasts, then guide constructs were designed placing those gRNAs between crRNA direct repeats or crRNA direct repeats and self-cleaving ribozyme sequences. These sequences were then synthesized and cloned into standard vectors. Plasmids encoding crRNA guides (“guide constructs”) included pOGZ2 (FIG. 10), pMEM6b (FIG. 9) and pOGZ1 (FIG. 21). Plasmids encoding a Cas nuclease (“nuclease construct”) were also synthesized including plasmids pLDB3 (FIG. 5), pLDB11B (FIG. 6), pLDB12B (FIG. 7), pMEM4 (FIG. 8) and pOGZ4 (FIG. 11). The components for each of these plasmids are listed in Table 7. For example, when transforming the combination of the guide construct pOGZ1 (FIG. 21) and nuclease encoding plasmid pMEM4 (FIG. 8), the MiMe loci which were targeted for editing were OSD1 (OSD1-1 and OSD1-2), SPOIL, and REC8. Additional plasmids were constructed to target OSD1-1, OSD1-2, OSD1-3, and combinations thereof.

Using this approach, the guide constructs shown in Table 7 can be readily modified to target any combination of MiMe loci that can result in a partial or complete MiMe genotype including, for example, REC8, OSD1-1, OSD1-2, OSD1-3, PAIR1-1, PAIR1-2, SPO11-1, JASON-1, JASON-2, CYCA1-1, CYCA1-2, TDM1, and/or PS1. Additionally, the separated guide construct and nuclease construct can be transformed into separate lines to achieve parent MiMe plants with various crossing schemes as described further below in section ZM9, or the guide construct and nuclease construct can be co-transformed into the same line to directly achieve a MiMe genotype (the “T0 approach”).

TABLE 7
Components of the plasmids used in biolistic experiments.

| | | Vector backbone components for |
|---|---|---|
| Plasmid | For plant expression | multiplication in E. coli |
| pLDB3 (FIG. 5) | ZmUBI: Maize Ubiquitin promoter | ori: origin of replication |
| | CAS: coding sequence of the selected | AmpR promoter: Ampicillin |
| | Cas nuclease | resistance promoter |
| | tHSP: terminator sequence from | AmpR: Ampicillin resistance |
| | Arabidopsis thaliana Heat shock | |
| | protein 18.2 | |
| | OsU6: RNA polymerase III promoter | |
| | from Oryza sativa | |
| | crRNA multiplex cassette containing | |
| | 4 crRNAs targeting REC8, OSD1-1, | |
| | OSD1-2, and SPO11 with poly-T | |
| | termination signal | |
| | CaMV 35S promoter: constitutive | |
| | promoter derived from the | |
| | Cauliflower mosaic virus | |
| | BlpR: Bialaphos resistance gene | |
| | CaMV poly(A) signal: terminator | |
| | from Cauliflower mosaic virus | |
| pLDB11B (FIG. 6) | ZmUBI: Maize Ubiquitin promoter | ori: origin of replication |
| | CAS: coding sequence of the selected | AmpR promoter: Ampicillin |
| | Cas nuclease | resistance promoter |
| | tHSP: terminator sequence from | AmpR: Ampicillin resistance |
| | Arabidopsis thaliana Heat shock | |
| | protein 18.2 | |
| | CaMV 35S: constitutive promoter | |
| | derived from the Cauliflower mosaic | |
| | virus | |
| | BlpR: Bialaphos resistance gene | |
| | CaMV poly(A) signal: terminator | |
| | from Cauliflower mosaic virus | |
| pLDB12B (FIG. 7) | OsU6: RNA polymerase III promoter | ori: origin of replication |
| | from Oryza sativa | |
| | crRNA multiplex cassette containing | AmpR promoter: Ampicillin |
| | 4 or more crRNAs targeting REC8, | resistance promoter |
| | OSD1-1, OSD1-2, and SPO11 with | |
| | poly-T termination signal | |
| | tHSP: terminator sequence from | AmpR: Ampicillin resistance |
| | Arabidopsis thaliana Heat shock | |
| | protein 18.2 | |
| | CaMV 35S: constitutive promoter | |
| | derived from the Cauliflower mosaic | |
| | virus | |
| | BlpR: Bialaphos resistance gene | |
| | CaMV poly(A) signal: terminator | |
| | from the Cauliflower mosaic virus | |
| pMEM4 (FIG. 8) | Zm Ubi promoter: Maize Ubiquitin | ori: origin of replication |
| | promoter | |
| | Cas Nuclease: coding sequence of the | AmpR promoter: Ampicillin |
| | selected Cas nuclease | resistance promoter |
| | AtHSP Term: terminator sequence | AmpR: Ampicillin resistance |
| | from Arabidopsis thaliana Heat | |
| | shock protein 18.2 | |
| | LjUBI1 promoter: Lotus japonicus | |
| | polyubiquitin promoter | |
| | DsRed: red fluorescent protein | |
| | reporter gene | |
| | Nos Terminator: Nopaline synthase | |
| | terminator from | |
| | Agrobacterium tumefaciens | |
| | CaMV 35S promoter: constitutive | |
| | promoter derived from the | |
| | Cauliflower mosaic virus | |
| | BlpR: Bialaphos resistance gene | |
| | CaMV poly(A) signal: terminator | |
| | from Cauliflower mosaic virus | |
| pMEM6b (FIG. 9) | OsU6 Pro: RNA polymerase III | ColE1: origin of replication |
| | promoter from Oryza sativa | |
| | crRNA multiplex cassette containing | AmpR promoter: Ampicillin |
| | 4 crRNAs targeting REC8, OSD1-1, | resistance promoter |
| | OSD1-2, and SPO11 with poly-T | |
| | termination signal | |
| | LjUBI1 pro: Lotus japonicus | AmpR: Ampicillin resistance |
| | polyubiquitin promoter | |
| | GFP: green fluorescent protein | |
| | reporter gene | |
| | CaMV 35S Pro: constitutive | |
| | promoter derived from the | |
| | Cauliflower mosaic virus | |
| | BlpR: Bialaphos resistance gene | |
| | CaMV poly A term: terminator from | |
| | Cauliflower mosaic virus | |
| pOGZ1 (FIG. 21) | Ubi promoter: Maize Ubiquitin | ori: origin of replication |
| | promoter | |
| | crRNA multiplex cassette containing | AmpR promoter: Ampicillin |
| | 4 or more crRNAs targeting REC8, | resistance promoter |
| | OSD1-1, OSD1-2, and SPO11 | |
| | HH_ribozyme and HDV_ribozyme: | AmpR: Ampicillin resistance |
| | non-coding RNA sequences that are | |
| | capable of self-cleavage | |
| | tNOS: Nopaline synthase terminator | |
| | from Agrobacterium tumefaciens | |
| pOGZ2 (FIG. 10) | Zm Ubi pro: Maize Ubiquitin | ColE1: origin of replication |
| | promoter | |
| | crRNA multiplex cassette containing | AmpR promoter: Ampicillin |
| | 4 or more crRNAs targeting REC8, | resistance promoter |
| | OSD1-1, OSD1-2, and SPO11 | |
| | HH_ribozyme and HDV_ribozyme: | AmpR: Ampicillin resistance |
| | non-coding RNA sequences that are | |
| | capable of self-cleavage | |
| | tNOS: Nopaline synthase terminator | |
| | from Agrobacterium tumefaciens | |
| | CaMV_35S: constitutive promoter | |
| | derived from the Cauliflower mosaic | |
| | virus | |
| | mCherryCDS: red fluorescent protein | |
| | reporter gene | |
| | Tvsp: Vegetative storage protein | |
| | terminator from Glycine max | |
| pOGZ4 (FIG. 11) | 2 × 35S pro: 35S promoter with | ColE1: origin of replication |
| | duplicated enhancer region from | |
| | Cauliflower Mosaic Virus | |
| | BlpR: Bialaphos resistance gene | AmpR promoter: Ampicillin |
| | | resistance promoter |
| | CaMV poly A term: terminator from | AmpR: Ampicillin resistance |
| | the Cauliflower mosaic virus | |
| | Zm Ubi pro: Maize Ubiquitin | |
| | promoter | |
| | Cas Nuclease: the chosen Cas | |
| | nuclease | |
| | tNOS: Nopaline synthase terminator | |
| | from Agrobacterium tumefaciens | |

ZM5: Plant Materials for Biolistic Transformation

Transformable diploid FFMM-AT6 and Hi-II were planted and grown with 16-hour/28° C. day and 8-hour/25° C. night cycles to produce ears containing immature embryos. Ears were harvested about 9 to 12 days after pollination and were surface sterilized using commercial bleach prior to performing embryo extraction as described by Wang et al. 2020 (Biolistic DNA delivery in maize immature embryos. Biolistic DNA Delivery in Plants: Methods and Protocols. pp. 177-195). Embryos were extracted and placed on either N6 Medium according to Wang and Frame 2009 (Wang, K. and Frame, B. (2009) Biolistic gun-mediated maize genetic transformation. Transgenic maize: methods and protocols. pp. 29-45) or 605T medium according to, for example, Jones et al. 2019 (Maize transformation using the morphogenic genes Baby Boom and Wuschel2. In Transgenic Plants. pp. 81-93. Humana Press, New York, NY) or, for example, Masters et al. 2020 (Agrobacterium Mediated Immature Embryo Transformation of Recalcitrant Maize Inbred Lines Using Morphogenic Genes. J. Vis. Exp. (156): e60782, doi:10.3791/60782). After that, embryos were incubated at 28° C. for 3 days. In some experiments, the incubation step described in the previous sentence was skipped and embryos were placed directly on N6 Osmotic Medium according to Wang et al. 2020.

ZM6: Biolistic Mediated Transformation

Biolistic mediated transformation was performed according to Wang et al. 2020 with the modifications noted below. Bombardment was performed on the day of embryo extraction. Embryos were extracted to N6 osmotic medium (Wang et al. 2020) and incubated at 27° C. for 4 hours prior to bombardment. For bombardment, 125 ng plasmid DNA per shot was loaded onto 0.1 mg gold particles using 0.1 μL of a transfection reagent, TransIT®-2020, scaled to the number of shots. Gold particles were first mixed by pipetting with plasmid DNA in 50 μl sterile deionized water, then TransIT®-2020 was added and mixed by pipetting again. This mixture was incubated on ice for 10 minutes with gentle vortexing every 1 minute. Gold particles were centrifuged for 1 minute at 100×g and the supernatant was removed. Gold particles were washed with 50 ul of 100% ethanol, then resuspended in 7 μl of 100% ethanol per shot and loaded onto macrocarriers. Embryos were bombarded at 450 psi using a Bio-Rad Biolistic® PDS-1000 biolistic device.

ZM7: Selection and Regeneration of Plants

For Hi-II callus, selection was performed according to Wang et al. 2020 with the modifications noted below. After bombardment, embryos were plated on N6 Selection I medium (1.5 mg/L bialaphos) and incubated at 27° C. in the dark for 2 weeks. After the 2-week incubation, callus was transferred to N6 Selection II (N6 SII) (3 mg/L bialaphos) and callus was further incubated for 2 weeks at 27° C. in the dark. Callus was transferred to fresh N6 SII medium every 2 weeks and checked for continual editing. Once sufficient editing was achieved, callus was moved directly to Regeneration II medium (Wang et al. 2020). Callus on Regeneration II medium was then incubated for 1 additional week at 27° C. in the dark, before being moved to a 16-hour day/8-hour night cycle in a lighted growth chamber with dim light and maintained at about 27° C. After 5 days of acclimation to the dim light, the light intensity was increased to about 100 ppfd light.

Once leafy shoots appeared, they were transferred to MS medium with 2 mg/L bialaphos as described in Wang et al. 2020. Once roots grew to a total length of >5 cm, the plantlets were transferred to soil. Leaf material was extracted and Illumina sequencing was performed to identify loss-of-function edits. Single plants were screened using the same technique as described in Example 1's section GM3 to identify individual events containing bi-allelic or mono-allelic conformations of each target gene including, for example, REC8, OSD1-1, OSD1-2, OSD1-3, PAIR1-1, PAIR1-2, SPO11-1, JASON-1, JASON-2, CYCA1, TDM1, PS1, or any combination thereof.

ZM8: Plant Growth and Crossing Conditions

Regenerated plants with confirmed edits in target MiMe alleles were transplanted from tissue culture into PRO-MIX HP substrate and acclimatized in a growth chamber with 16-hour, 25° C. day, and 8-hour, 20° C. night with 60% relative humidity and 16-hour day length. After two to three weeks, plants were transplanted to soil and grown under greenhouse conditions with a continued 16-hour daylength. Once plants produced flowers, they were outcrossed as male or female to desired lines to introgress the transgene or edited alleles.

ZM9: Crossing

Crossing Scheme I. In some experiments, the cross begins with a single rooted plant transformed with the nuclease construct (designated PED-MRN11) and a single rooted plant transformed with guide construct (designated PED-MRG12). PED-MRN11 can be backcrossed to inbred lines designated PED-MR-A and PED-MR-C, respectively. Hybrid plants containing the nuclease construct pLDB11 can be selected, further backcrossed, and then designated PED-A-MRN11 and PED-C-MRN11, respectively. Separately, PED-MRG11 can be backcrossed to inbred lines designated PED-MR-B and PED-MR-D, respectively. Hybrid plants containing the guide construct pLDB12 are selected, further backcrossed, and then designated PED-B-MRG12 and PED-D-MRG12, respectively. PED-A-MRN11 can then be crossed to PED-B-MRG12 and plants containing both the nuclease construct and the guide construct and a complete MiMe genotype comprising bi-allelic edits in the target MiMe loci selected and designated PED-AB-MiMe1. Similarly, PED-C-MRN11 can be crossed to PED-D-MRG12, and progeny plants containing both the nuclease construct and the guide construct and a complete MiMe genotype comprising homozygous or bi-allelic edits in the target MiMe loci selected and designated PED-CD-MiMe2. PED-AB-MiMe1 can then be crossed to PED-CD-MiMe2, resulting in a genetically uniform hybrid population of tetraploid maize with four haplotypes designated PED-ABCD-Tet1.

Crossing Scheme 2. In some experiments, the cross begins with two rooted plants, each bearing mono-allelic mutations in MiMe alleles and generated as discussed in Crossing Scheme 1, but by selecting for mono-allelic mutations in MiMe alleles (sometimes referred to here as a “HetMiMe” plant) instead of bi-allelic mutations. These plants are designated PED-MR2A or PED-MR2B respectively, grown to maturity, and then crossed to each of the inbred lines PED-MR-E, PED-MR-F, PED-MR-G, and PED-MR-H. Seeds from the resulting hybrids are screened for mono-allelic nonfunctional mutations (HetMiMe plants) as described in Example 1's section GM3, and plants are again grown to maturity. Plants with the mono-allelic edits can be identified and backcrossed to produce lines with the respective mono-allelic mutations in each respective inbred background designated as PED-E-HetMiMe, PED-F-HetMiMe, PED-G-HetMiMe, and PED-H-HetMiMe. From the plants in each of these HetMiMe populations, heterozygous knockout plants and lines are then screened and intercrossed to make PED-E-HetMiMe×PED-F-HetMiMe hybrids and PED-G-HetMiMe×PED-H-HetMiMe hybrids. These hybrids are then screened, and two individual plants bearing a complete MiMe genotype (comprising bi-allelic mutations in the MiMe alleles) are identified and designated PED-EF-MiMe1 and PED-GH-MiMe2. PED-EF-MiMe1 can then be crossed to PED-GH-MiMe2, resulting in a genetically uniform hybrid population of tetraploid maize with four haplotypes designated PED-EFGH-Tet1.

Crossing Scheme 3. In some experiments, the cross began with one rooted F1 hybrid designated PED-MR3A-JK, bearing mono-allelic mutations in some MiMe alleles and bi-allelic mutations in other MiMe alleles and generated as discussed in Crossing Scheme 1, but by selecting for this configuration of mutations in MiMe alleles (instead of selecting for bi-allelic mutations). PED-MR3A-JK was then grown to maturity, selfed, and the resulting progeny were screened for plants with full bi-allelic mutations in the MiMe alleles. Separately, an inbred diploid designated PED-MR3B-LL was doubled in ploidy by using colchicine in tissue culture. PED-MR3A-JK was then crossed to PED-MR3B-LL, resulting in a genetically uniform hybrid population of tetraploid maize with three haplotypes designated PED-JKLL-Tetl.

ZM10: Genotyping

High-quality genomic DNA was extracted from maize plants using Qiagen Dneasy Plant Mini kits (QIAGEN, Germantown, MD, USA). Purity was determined using a spectrophotometer, Nanodrop™ One-C (Thermo Fisher), and quantified with a fluorometer, Qubit™ Flex Fluorometer (Thermo Fisher). Samples were adjusted to a final concentration of 20 ng/μL. Markers were established and populations were genotyped as described in Example 1's section GM6. Alternatively, maize plants may be genotyped using targeted Genotyping By Sequencing (tGBS) (Ott et al. (2017) tGBS® genotyping-by-sequencing enables reliable genotyping of heterozygous loci. Nucleic acids research 45(21): e178-e178.).”

ZR: Results

ZR1: Guide Screening

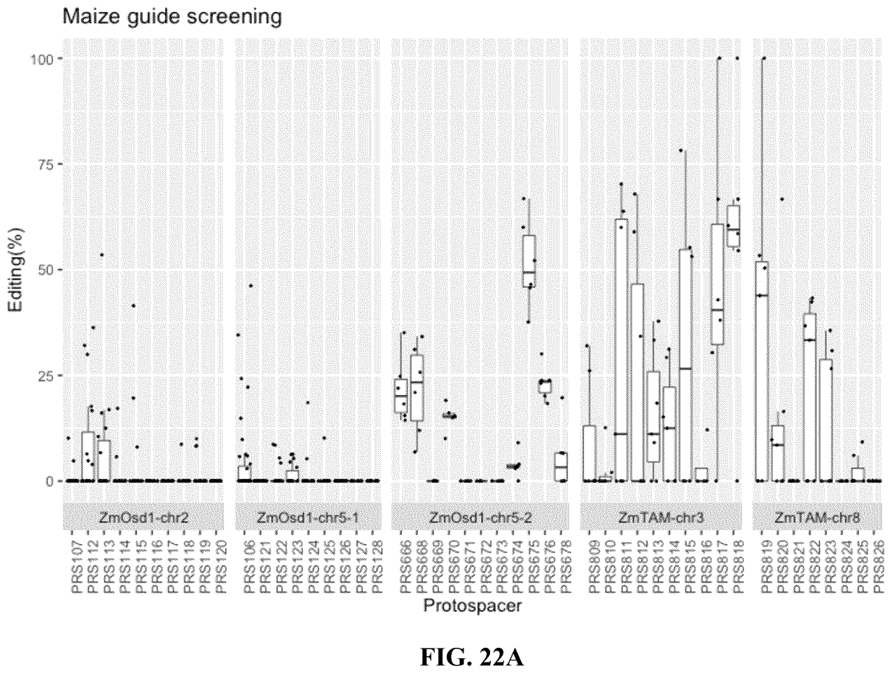

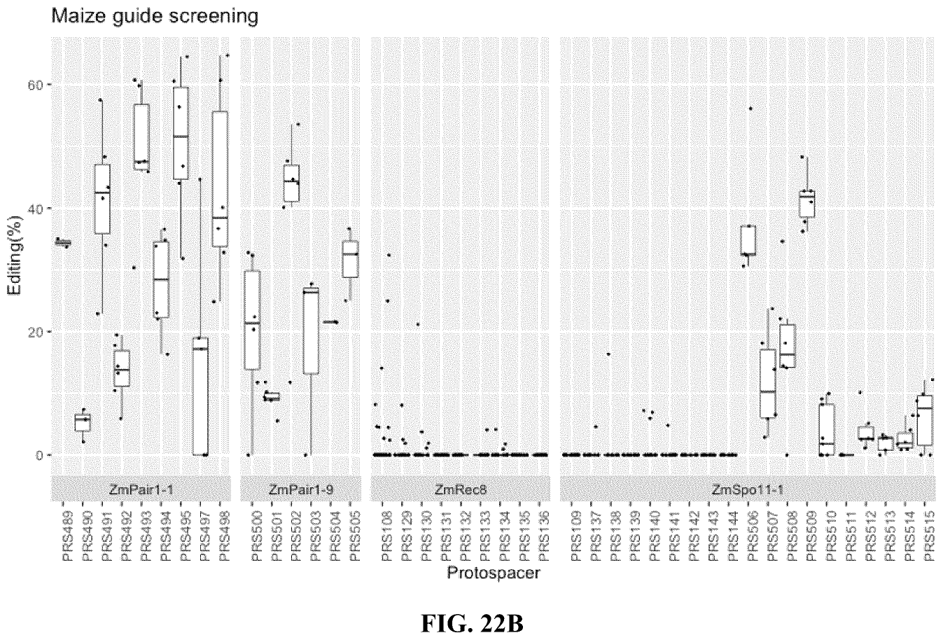

Guide RNAs were screened by RNP transfection or plasmid transfection as described in section ZM3. For maize protoplasts, a total of 10 guide RNAs were screened that targeted conserved regions of OSD1-1 (Table 8, SEQ ID NOs: 107-120). A total of 9 guide RNAs were screened that targeted conserved regions of OSD1-2 (Table 8, SEQ ID NOs: 24-32). A total of 9 guide RNAs were screened that targeted conserved regions of REC8 (Table 8, SEQ ID NOs: 33-41). A total of 19 guide RNAs were screened that targeted conserved regions of SPO11-1 (Table 8, SEQ ID NOs: 42-60). Editing efficiency was calculated by sequencing as described in Example 1's section GM3, and the resulting data presented in FIG. 22A and FIG. 22B. Based on this data, candidate guide RNAs were produced, as illustrated in Table 9. Guide RNA SEQ ID NO: 62 was chosen for editing OSD1-1; guide RNA SEQ ID NO: 71 was chosen for editing OSD1-2; guide RNA SEQ ID NO: 82 was chosen for editing REC8; guide RNA SEQ ID NO: 98 was chosen for editing SPO11-1.

TABLE 8
Sequences for conserved regions of
MiMe genes targeted in maize.

| SEQ ID | | |
|---|---|---|
| NO. | PRSG# | Target DNA sequence |
| 14 | PRS107 | TGGTGGTGTTGGTGGTGGAG |
| 15 | PRS112 | TCAGGAGGGTGGCCTCGCCG |
| 16 | PRS113 | GGCGTTGAGGGTCACACCGA |
| 17 | PRS114 | TCGGTGTGACCCTCAACGCC |
| 18 | PRS115 | CAATGGCCGTAATATCACGG |
| 19 | PRS116 | ATCAGGAGGGTGGCCTCGCC |
| 20 | PRS117 | GTCGGTGTGACCCTCAACGC |
| 21 | PRS118 | CTGCAGAGCCCCGAGATGCC |
| 22 | PRS119 | CTGGATGGTACCCCAGGACG |
| 23 | PRS120 | GCAATGGCCGTAATATCACG |
| 24 | PRS106 | GAGGATGGCAAGCTGAAGAC |
| 25 | PRS121 | ACGATTGATGTGATGTCACG |
| 26 | PRS122 | CCTGACTGGTACCCGAGGAC |
| 27 | PRS123 | AAGATGCTTGAAGTGAGGAC |
| 28 | PRS124 | ACAGCACCATTCACCAGCAC |
| 29 | PRS125 | CCATCCTCCAAACAGGTTGC |
| 30 | PRS126 | AGCTTGCCATCCTCCAAACA |
| 31 | PRS127 | CCGATCTGTGGATCCAATAA |
| 32 | PRS128 | TCCTCCTCTCAATTGCCTGC |
| 33 | PRS108 | CGGTTGATCTTCGAGTGGAG |
| 34 | PRS129 | ATGATGTCGAGCTTGTCGAG |
| 35 | PRS130 | GAACCCCTCGGTGCCCATGG |
| 36 | PRS131 | TCGAGCCGTTTGCGGTTGAT |
| 37 | PRS132 | CTCTCGTACACGATCACCAC |
| 38 | PRS133 | ACCTTCCTCTCGTACACGAT |
| 39 | PRS134 | CTGTAGAGAAGCTTCACCTT |
| 40 | PRS135 | GAGTGGAGCGTCGCCGCCAT |
| 41 | PRS136 | TCGCGCGGAAGGCGCCGCTC |
| 42 | PRS109 | ATCCTGCGGAGGAGGAGCGC |
| 43 | PRS137 | TTCTGCTGCAGGAGCTGCTG |
| 44 | PRS138 | CTCCAGCCTCCGCCGCAACT |
| 45 | PRS139 | GATCCTGCGGAGGAGGAGCG |
| 46 | PRS140 | GCTCCCTGCAGAAGTAGCAG |
| 47 | PRS141 | AAGTGCAGCCGGCACAATCT |
| 48 | PRS142 | GAGCGGCGCCGCTCGTCGCC |
| 49 | PRS143 | AAGAGTATGCAGATATCGTT |
| 50 | PRS144 | GAGCAGTGCTTGTTCTGCTG |
| 51 | PRS506 | GATGTATTCGTACTGAGGCG |
| 52 | PRS507 | ATTGCCTCAATGTCAACAGG |
| 53 | PRS508 | CACATATCTTGGCTCCCTGC |
| 54 | PRS509 | GTGATGGGCTGGATAAGATT |
| 55 | PRS510 | TGGAGGGCGAAAAGAAAGTG |
| 56 | PRS511 | CCTTCTGTATATTCAGGCTT |
| 57 | PRS512 | TCCATTCCTGTTGACATTGA |
| 58 | PRS513 | TTCTCTTGCAGATGTTGTTA |
| 59 | PRS514 | CAGTGTTCCAGCGTTTGGCC |
| 60 | PRS515 | ACAGAACTTGTCATTGGCCA |

TABLE 9
Sequences for guide RNAs targeting
MiMe genes in maize.

| SEQ ID | | |
|---|---|---|
| NO. | PRSG# | Guide RNA sequence |
| 61 | PRS107G | UGGUGGUGUUGGUGGUGGAG |
| 62 | PRS112G | UCAGGAGGGUGGCCUCGCCG |
| 63 | PRS113G | GGCGUUGAGGGUCACACCGA |
| 64 | PRS114G | UCGGUGUGACCCUCAACGCC |
| 65 | PRS115G | CAAUGGCCGUAAUAUCACGG |
| 66 | PRS116G | AUCAGGAGGGUGGCCUCGCC |
| 67 | PRS117G | GUCGGUGUGACCCUCAACGC |
| 68 | PRS118G | CUGCAGAGCCCCGAGAUGCC |
| 69 | PRS119G | CUGGAUGGUACCCCAGGACG |
| 70 | PRS120G | GCAAUGGCCGUAAUAUCACG |
| 71 | PRS106G | GAGGAUGGCAAGCUGAAGAC |
| 72 | PRS121G | ACGAUUGAUGUGAUGUCACG |
| 73 | PRS122G | CCUGACUGGUACCCGAGGAC |
| 74 | PRS123G | AAGAUGCUUGAAGUGAGGAC |
| 75 | PRS124G | ACAGCACCAUUCACCAGCAC |
| 76 | PRS125G | CCAUCCUCCAAACAGGUUGC |
| 77 | PRS126G | AGCUUGCCAUCCUCCAAACA |
| 78 | PRS127G | CCGAUCUGUGGAUCCAAUAA |
| 79 | PRS128G | UCCUCCUCUCAAUUGCCUGC |
| 80 | PRS108G | CGGUUGAUCUUCGAGUGGAG |
| 81 | PRS129G | AUGAUGUCGAGCUUGUCGAG |
| 82 | PRS130G | GAACCCCUCGGUGCCCAUGG |
| 83 | PRS131G | UCGAGCCGUUUGCGGUUGAU |
| 84 | PRS132G | CUCUCGUACACGAUCACCAC |
| 85 | PRS133G | ACCUUCCUCUCGUACACGAU |
| 86 | PRS134G | CUGUAGAGAAGCUUCACCUU |
| 87 | PRS135G | GAGUGGAGCGUCGCCGCCAU |
| 88 | PRS136G | UCGCGCGGAAGGCGCCGCUC |
| 89 | PRS109G | AUCCUGCGGAGGAGGAGCGC |
| 90 | PRS137G | UUCUGCUGCAGGAGCUGCUG |
| 91 | PRS138G | CUCCAGCCUCCGCCGCAACU |
| 92 | PRS139G | GAUCCUGCGGAGGAGGAGCG |
| 93 | PRS140G | GCUCCCUGCAGAAGUAGCAG |
| 94 | PRS141G | AAGUGCAGCCGGCACAAUCU |
| 95 | PRS142G | GAGCGGCGCCGCUCGUCGCC |
| 96 | PRS143G | AAGAGUAUGCAGAUAUCGUU |
| 97 | PRS144G | GAGCAGUGCUUGUUCUGCUG |
| 98 | PRS506G | GAUGUAUUCGUACUGAGGCG |
| 99 | PRS507G | AUUGCCUCAAUGUCAACAGG |
| 100 | PRS508G | CACAUAUCUUGGCUCCCUGC |
| 101 | PRS509G | GUGAUGGGCUGGAUAAGAUU |
| 102 | PRS510G | UGGAGGGCGAAAAGAAAGUG |
| 103 | PRS511G | CCUUCUGUAUAUUCAGGCUU |
| 104 | PRS512G | UCCAUUCCUGUUGACAUUGA |
| 105 | PRS513G | UUCUCUUGCAGAUGUUGUUA |
| 106 | PRS514G | CAGUGUUCCAGCGUUUGGCC |
| 107 | PRS515G | ACAGAACUUGUCAUUGGCCA |

ZR2: Transformation and Selection of Regenerated Plants

To produce the first parent MiMe plant, a 2n Hi-II AxB Hybrid mother plant was crossed by pollen from a 2n Fast-Flowering Mini-Maize AT6 (FFMM-AT6) father. 14 days after pollination, the ear was harvested and placed at 4° C. overnight. The next morning, embryos were extracted, plated, and bombarded according to section ZM6 using the Bio-Rad Biolistic® PDS-1000 and 0.6 μm gold particles coated with the guide construct pOGZ1 and the nuclease construct pMEM4 loaded in a 3:1 molar ratio, respectively. Plasmid and gold were mixed in 50 lit sterile water, then a transfection reagent, TransIT®-2020, was added to bind the plasmid to the gold. Each gene gun shot was prepared with 125 ng DNA, 0.1 mg 0.6 μm gold particles, and 0.1 lit TransIT®-2020, and the gold particles were resuspended in 7 μL 100% ethanol and loaded onto macrocarriers, allowed to dry, then fired into the immature zygotic embryos at 450 psi. Gene gun settings were set according to Wang 2020. Plant PED-MN-HetMiMe was selected with a mono-allelic 10 bp frameshift-inducing mutation in osd1-1, bi-allelic frameshift-inducing 13 bp and 17 bp mutations in osd1-2, a 12 bp mutation in rec8 that disrupted a highly conserved coding region, and a mono-allelic 7 bp frameshift-inducing mutation in spo11. The plant was regenerated from tissue culture, grown in the greenhouse, then self-pollinated to produce T1 F2 seed population PED-MN-SPP. This seed was germinated, and a parent MiMe plant PED-MN-MiMe having bi-allelic loss-of-function mutations at osd1-2 and homozygous loss-of-function mutations at rec8 and spo11 was selected. PED-MN-MiMe showed fertility restoration and shed viable pollen. No edits of osd1-1 could be recovered from PED-MN-SPP.

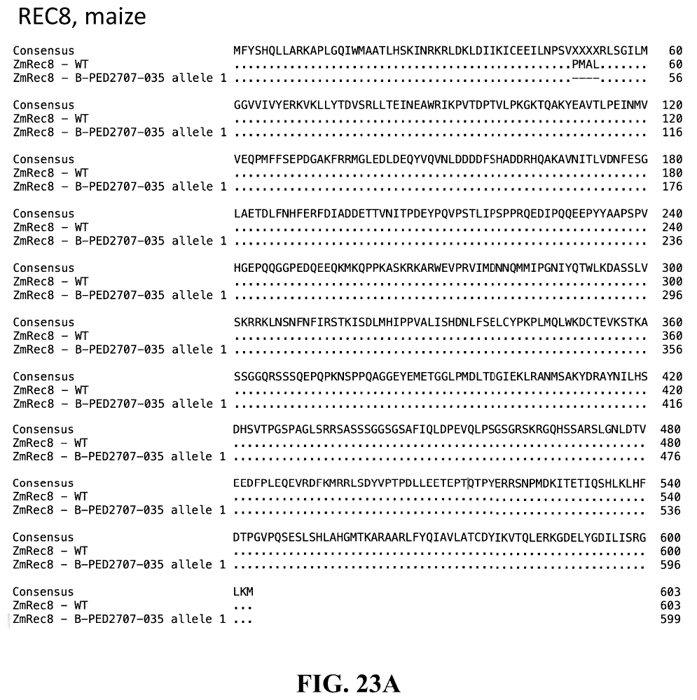

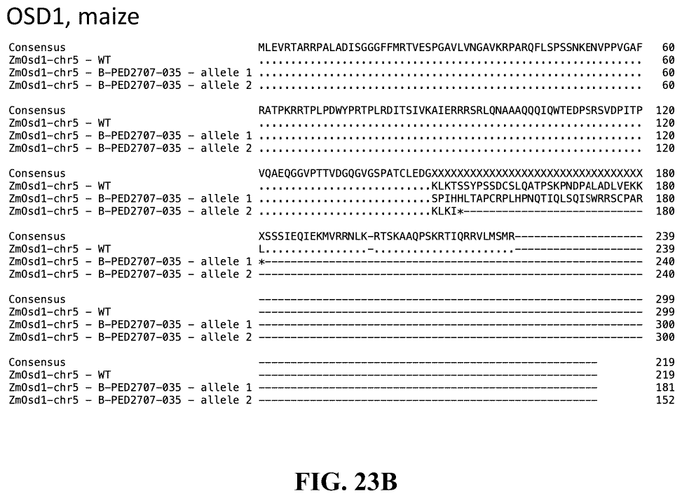

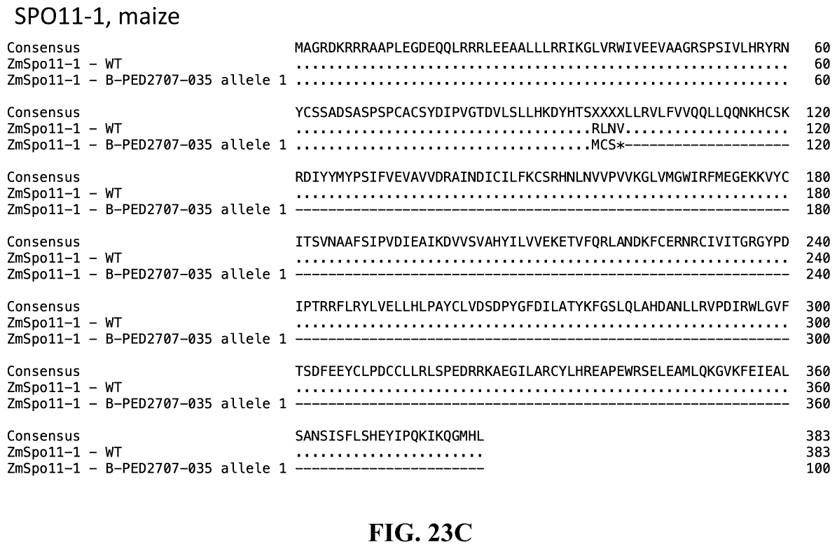

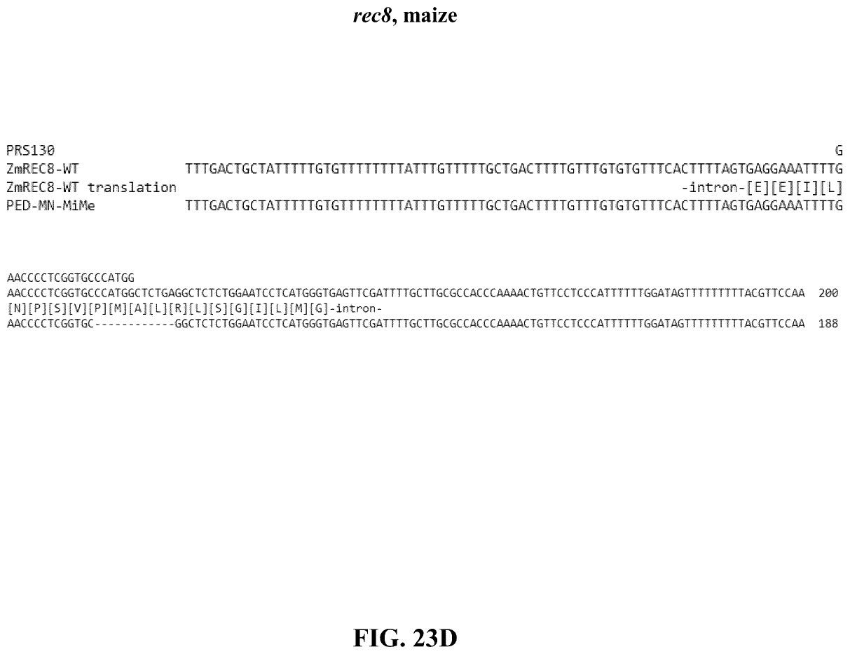

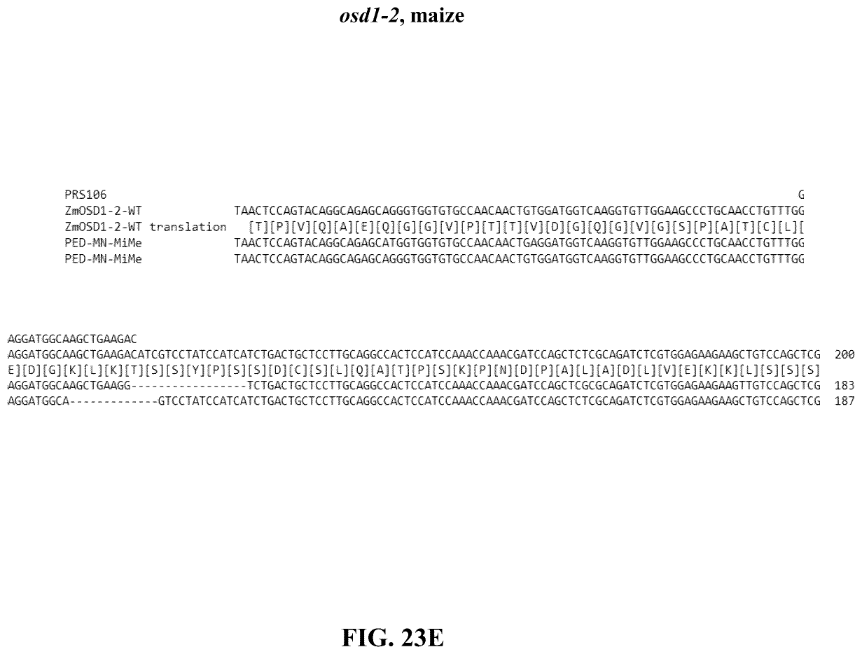

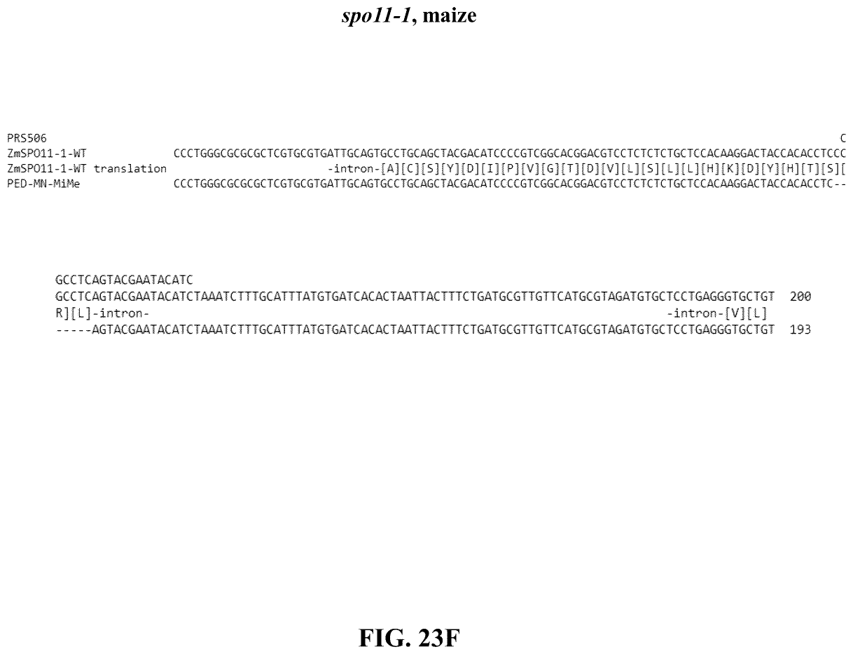

As an example, edits to the parent MiMe plant PED-MN-MiMe (rec8/osd1-2/spo11-1) are illustrated in FIGS. 23A-23C. In that figure, the predicted protein sequences are illustrated with respect to the unedited wildtype protein sequence and a consensus sequence, wherein an “X” in the consensus sequence indicates that there is no consensus for that amino acid position; wherein a dot (“.”) in the parent MiMe plant sequence indicates that the respective amino acid matches the consensus sequence; and wherein a dash (“-”) in the parent MiMe plant sequence indicates that the respective amino acid is present in the consensus sequence but is absent in the parent MiMe plant sample sequence. The deletion in REC8 disrupts a conserved protein sequence motif (FIGS. 23A and 23D). Frameshift inducing deletions in both alleles of osd1-2 (FIGS. 23B and 23E) and spo11-1 (FIGS. 23C and 23F) result in premature stop codons. Premature stop codons are denoted by an asterisk (“*”) in FIGS. 23B-23C.

ZR3: Crossing and Seed Extraction

Pollen from PED-MN-MiMe (osd1-2/rec8/spo11) was collected and crossed to female PED-00-4n plants, where PED-00-4n was an inbred 4n tetraploid LH244, resulting in the Boosted Maize Population (“BMP”). Ears were harvested approximately 21-22 days after pollination, and BMP embryos were rescued to a non-selective MS rooting medium to germinate in the dark at 27° C. Once germinated, the plants were moved to rooting medium in a lighted incubator. Once rooted, the plants were moved to pots with PRO-MIX HP and covered with a humidity dome to acclimate before being transplanted to soil in the greenhouse.

ZR4: Genotypic Evaluation

DNA samples were sequenced by Novogene. Libraries were prepared using the NEBNext® Ultra™ II for DNA Library Prep kit and paired-end 2×150 reads were sequenced on an Illumina NovaSeq 6000 or a NovaSeq X Plus. Markers were established and populations were genotyped as described in GM6.

For the parent MiMe plant PED-MN-MiMe and parent non-MiMe PED-00-4n, DNA samples were sequenced to a depth of 120,987,086,400 bp, and 104,384,408,100 bp, respectively. For the BMP progeny plants, DNA samples were sequenced to an average depth of 96,945,478,675 bp.

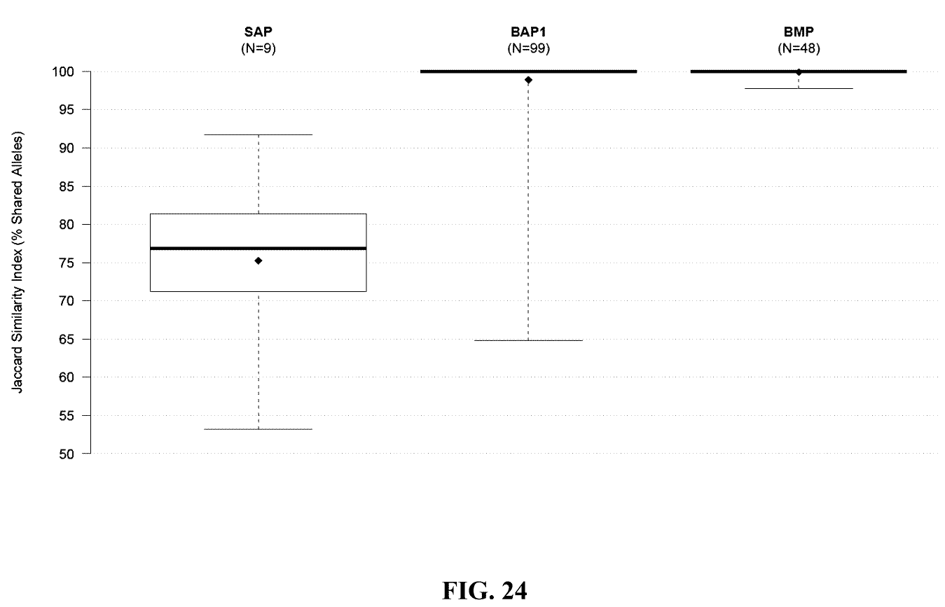

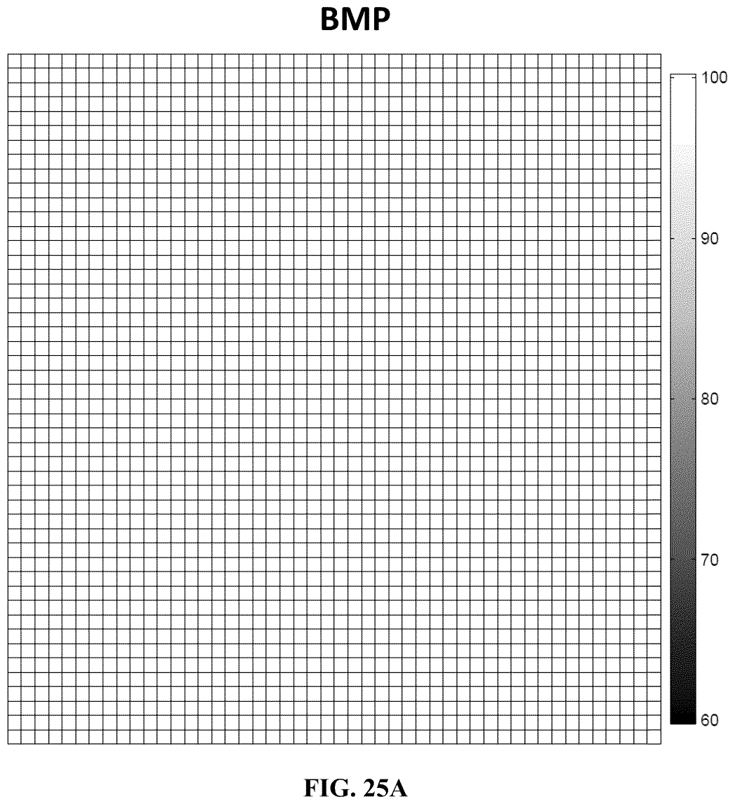

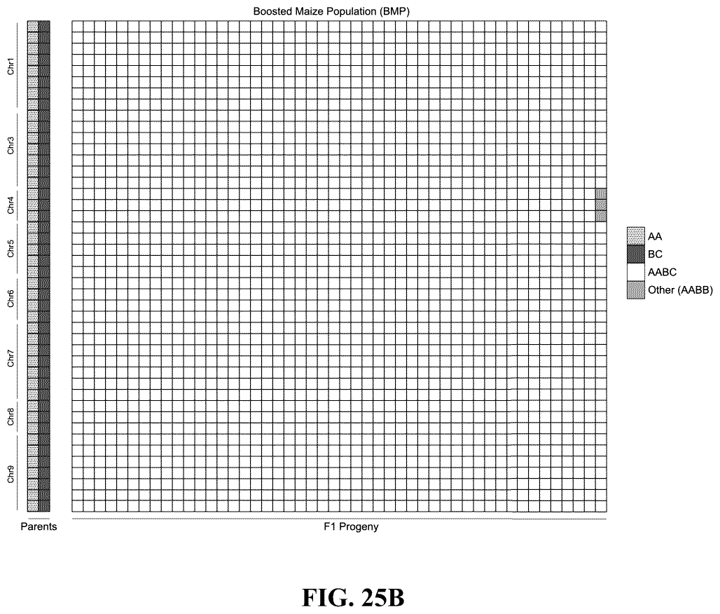

After filtering for high quality markers, a genotype matrix was assembled for BMP (FIG. 25B). Across the BMP population, the average pairwise genetic uniformity as measured by the Jaccard similarity coefficient was 99.9% (visualized as a pairwise matrix in FIG. 25A). This data is illustrated by the population uniformity graph in FIG. 24.

ZR5: Phenotypic Evaluation of MiMe Progeny

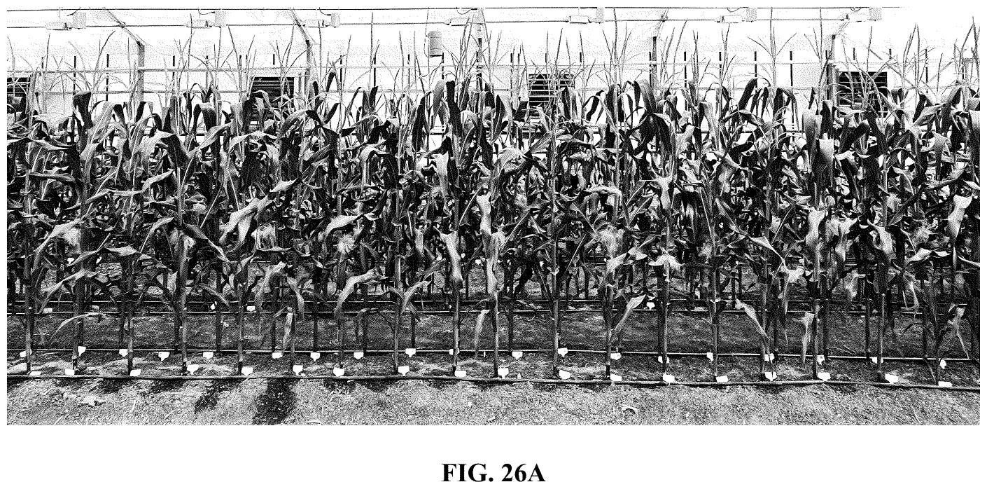

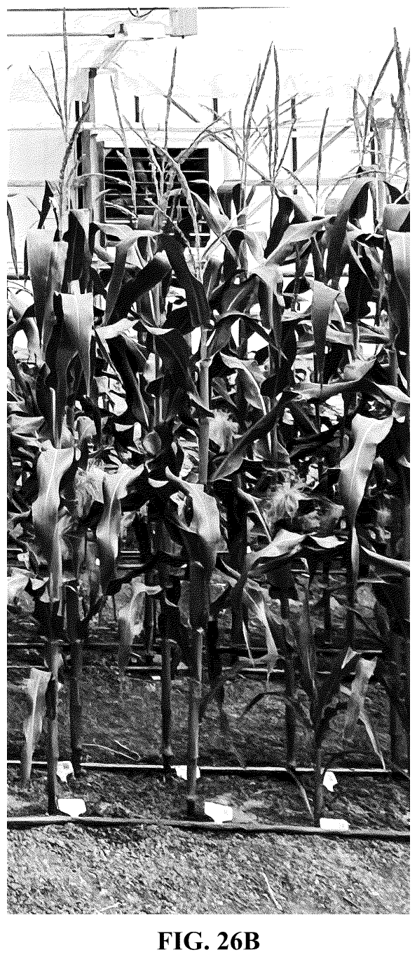

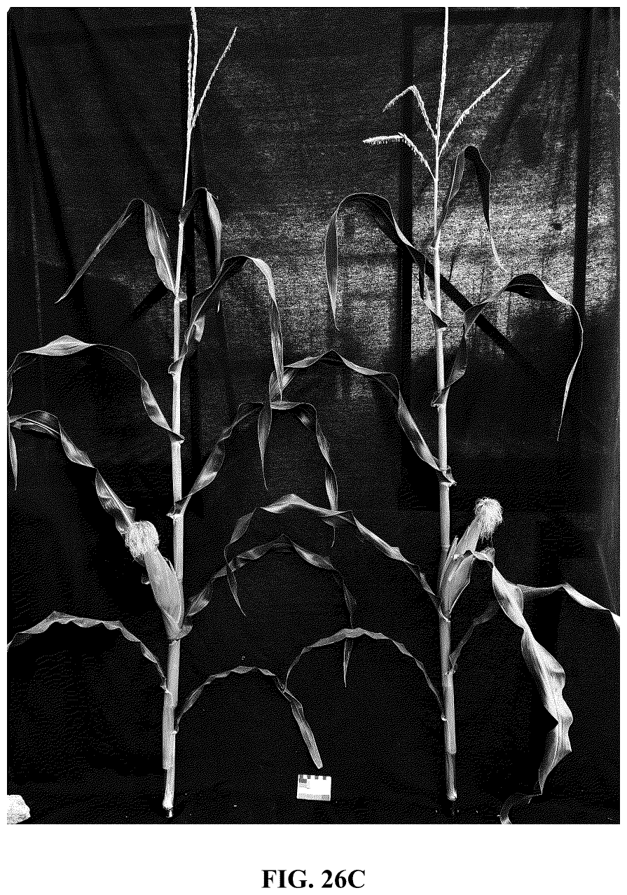

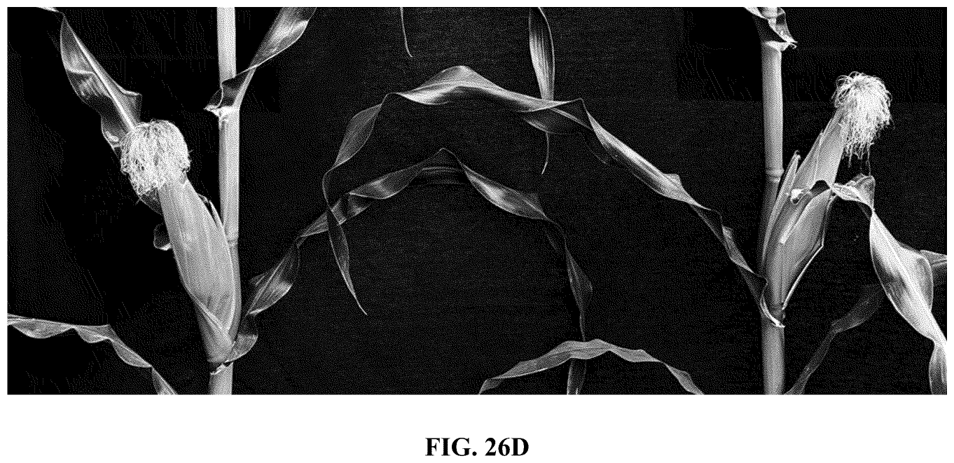

The BMP plants demonstrated a phenotypically uniform size, growth, and appearance, whereas typically the progeny of an F1 hybrid of non-inbred lines would show segregational variance. Maize plants from BMP are shown in FIG. 26A. All plants are 68 days post-embryo rescue and 57 days post-transplant from tissue culture to soil. A close-up of the right-hand side of FIG. 26A is shown in FIG. 26B, showing a close-up view of three BMP plants. FIG. 26C shows healthy BMP plants readily producing fruit, which are further displayed as a close-up view of the ears in FIG. 26D.

### Example 3: Generating Genetically Uniform Tetraploid Hybrid Arabidopsis with Three or Four Haplotypes

AM: Methods

AM1: Plant Materials and Protoplast Isolation

Plants from Arabidopsis accessions that were selected and tested included Landsberg, Shandara, HR-10, Ws-2, Col-0(C5851557-S1), CS851294, Columbia (“Col-0”), Col-0×Shandara F1, Landsberg×Shandara F1, Col-0×Shandara F2, Landsberg×Shandara F2, Col-0(C5851557-S1)×HR-10 F2, Col-0×HR-10 F2, and Shandara×Ws-2 F2.

Before germinating, seeds were surface sterilized using 70% ethanol solution for 5 min. Then, seeds were rinsed with sterile, distilled water five times. Seeds were stratified at 4° C. and then germinated on hormone-free AGM (Arabidopsis Germination Medium) containing half strength Murashige and Skoog basal salts, 10 g/L sucrose, 0.5 g/L MES and 8 g/L Agar (pH 5.8) and grown under 18-hour light/6-hour dark photoperiod (40-60 Imo′ photons m-2 s-1) at 23° C. Approximately 1 gram of leaves from each batch of 10-day old seedlings was removed under aseptic conditions for protoplast extraction.

For protoplast isolation, leaves were sliced into thin sections approximately 1 mm in width and incubated in an enzyme solution composed of 0.5% (weight/volume) Cellulase RS (Duchefa Biochemie, The Netherlands) and 0.5% (weight/volume) Macerozyme R10 (Duchefa Biochemie, The Netherlands) in MMC solution (10 mM MES, 0.47 M Mannitol, 10 mM Calcium Chloride) for a final volume of 10 mL. Sliced leaves in the digest solution were then incubated for around 15 hours at 24° C. in darkness. The protoplasts were then liberated from the leaf tissue with 15-minute shaking at 40 RPM at room temperature.

Protoplasts were then diluted with equal volume of At-W5 buffer (154 mM NaCl, 125 mM CaCl₂, 5 mM KCl, 2 mM MES, pH 5.7) and harvested through 40 μm sterile cell filters into sterile 50 mL conical tubes, then centrifuged at 150×g for 5 minutes. Supernatant was removed and cells were resuspended in 2 mL At-W5 buffer by gently rocking, then slowly layered onto 6 mL of 0.6 M sucrose solution as described in Jeong et al. 2021 (Optimization of protoplast regeneration in the model plant Arabidopsis thaliana. Plant methods 17(1): 1-16) in a new tube. Tubes were centrifuged at 150×g for 8 minutes. After centrifugation a thick dark band of protoplasts appeared at the interface of the two solutions. This band was harvested in one continuous motion using a sterile serological pipette and combined with 30 mL of At-W5 buffer. The samples were centrifuged at 150×g for 5 minutes and resuspended in 10 mL of At-W5 buffer. Cells were quantified using a Bürker hemocytometer and stored at 4° C. in the dark until transfection. A sample was reserved from each isolation to test cell viability using FDA staining as described by Larkin 1976 (Purification and viability determinations of plant protoplasts. Planta. 128(3): 213-216).

AM2: Transfection

Protoplast transfection proceeded in a similar fashion to Yoo et al. 2007 (Arabidopsis mesophyll protoplasts: a versatile cell system for transient gene expression analysis. Nature protocols. 2(7): 1565-1572). Protoplasts were centrifuged at 150×g for 5 minutes and resuspended in a volume of transformation MMG buffer (4 mM MES, pH 5.7, 0.4 M mannitol and 15 mM MgCl₂) sufficient to achieve a cell density of 2×10⁶ protoplasts/mL. For each transfection reaction, 20 lit of freshly prepared RNP solution as described in Example 1's section GM2 was added to the bottom of a 15 mL round bottom tube, and then 100 μL of protoplasts suspended in transformation MMG buffer was added and mixed with the RNP solution. Next, 120 μL of freshly prepared PEG solution (40% PEG4000, 0.2 M mannitol and 100 mM CaCl₂) was added and gently mixed by rotating the tubes. After a 15-minute incubation at room temperature, the transfection mixture was diluted with 5 mL of At-W5 solution and gently mixed to stop the transfection process. The transfected cells were then washed twice following centrifugation at 150×g for 5 minutes using a 0.5 M mannitol solution. Cells with the same transfection reactions were combined to achieve a final protoplast density of 2×10⁶ protoplasts/mL in 0.5 M mannitol solution for alginate encapsulation.

AM3: Encapsulation and Regeneration of Plants

An equal volume of transfected cells was combined with a 2.8% sodium alginate solution and then gently mixed together. The protoplast-alginate mixture was then poured onto a CaCl₂ agar plate and incubated at room temperature for 30 minutes, after which 2 mL of CaCl₂ solution was added to further solidify the hydrogel. The protoplast-alginate gel was then cut into 4 pieces and a quarter of the gel was placed in a Petri dish with Protoplast Induction Medium (PIM), as described in Jeong et al. 2021.

Plant regeneration protocols were based on Jeong et al. 2021 with some modifications summarized as follows. Following encapsulation in thin alginate layers, the protoplasts were incubated in darkness for 4 weeks, and fresh PIM was added weekly. Cell division started after about one week, and when microcalli reached a size of about 500 μm, the PIM was replaced with Callus Induction Medium (CIM). Calli were allowed to grow to about 1-2 mm size in CIM, after which they were plated on solid Shoot Induction Medium (SIM). After 3 weeks, or when the shoots reached a size of approximately 5-10 mm, they were excised from the calli and transferred to Rooting Medium (RM).

Plants were then propagated in vitro and genotyped using the same sequencing methods described in Example 1's section GM3. Plants containing bi-allelic mutations in target MiMe genes were further multiplied and prepared for planting.

AM4: Plant Growth and Crossing Conditions

Once plants had completely regenerated in vitro from protoplasts, rooted plantlets were transferred to 3.5-inch pots filled with wetted PROMIX HP Growing Medium (Premier Tech Horticulture, Quakertown, PA, USA). 18 pots were placed in each tray, and trays were covered with plastic domes for 3 days. Afterwards, lids were slowly removed by opening them more every day over the course of 3 days. Plants were grown under 18-hour light/6-hour dark photoperiod (40-70 viol photons m-2 s-1) at 21-22° C. Plants were watered with distilled water and fertilized with 1× Miracle Grow, indoor concentration (The Scotts Company LLC, Marysville, OH, USA) dissolved in distilled water.

Regenerated plants with confirmed edits in each target gene including REC8, OSDJ, and PAIR1 and/or SPO11 were transplanted from tissue culture into substrate and then acclimatized under cool white lights at 24° C. with approximately 60% relative humidity and 16-hour daylength. When plants produced flowers, pollen was collected, and cross pollination was performed under the same environmental conditions. About 21 days after pollination, siliques were removed and seeds were extracted, dried, and prepared for planting.

AM5: Seed Germination and Genotyping

Following seed extraction, seeds were surface sterilized using 1 mL of 70% ethanol solution for 5 min. Then, seeds were rinsed with 1 mL of sterile, distilled water five times. Sterilized seeds were shown on hormone-free AGM (Arabidopsis Germination Medium) in Petri dishes. After one week, seedlings exhibiting germination were transplanted to soil, where they were allowed to grow for 2 months before leaves were removed for DNA extraction. High quality genomic DNA was extracted from leaves of randomly selected plants from each population used for genotyping using Qiagen Dneasy Plant Mini kits (Cat. No. 69104) (QIAGEN, Germantown, MD, USA). Purity was determined using a spectrophotometer and DNA was quantified with a fluorometer as described in Example 2's section PMS. Samples were adjusted to a final concentration of 20 ng/μL. Markers were established and populations were genotyped as described in GM6.

AM6: Fertility and Parthenocarpy Testing

Fertility was determined by counting the number of seeds per fruit (silique) for 20 unopened, mature fruits per plant. Parthenocarpic fruit development was induced by application of gibberellic acid. Pistils were treated with a 0.11 μmol/μL gibberellic acid solution (“GA3 treated”) or with a buffered control (“mock-treated”) solution as described in Vivian-Smith et al. 1999 (Genetic analysis of growth-regulator-induced parthenocarpy in Arabidopsis. Plant Physiology. 121(2): 437-452) Flowers were not emasculated prior to treatment. Some flowers were left to develop normally (“untreated”). On 8 days post-application, fruit length was measured, and on 12 days post-application, some fruits were removed for clearing according to the protocol outlined in Sprunck et al. 2012 (Egg cell-secreted EC1 triggers sperm cell activation during double fertilization. Science. 338(6110): 1093-1097). Cleared fruits were imaged the following day.

AR: Results

AR1: Guide RNA Screening

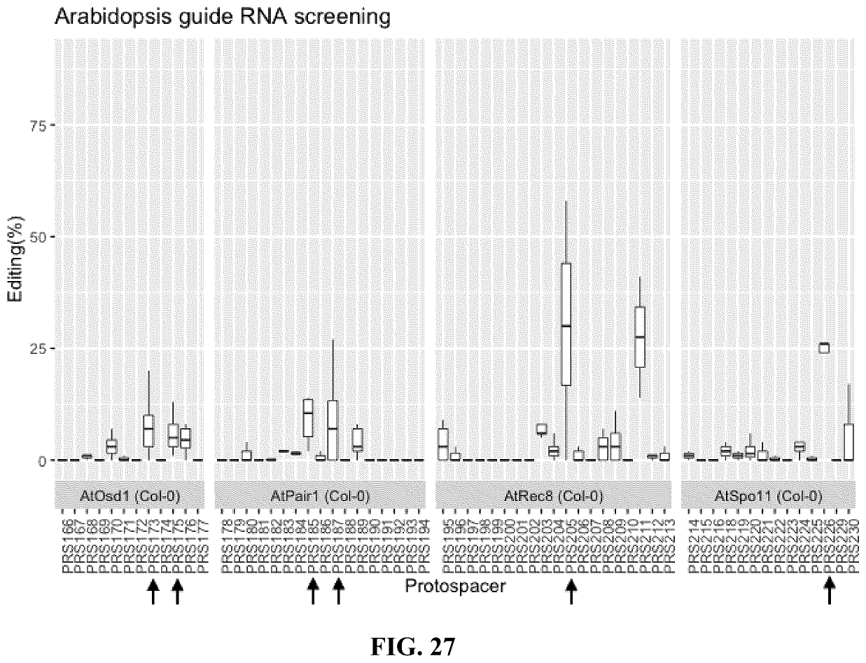

A total of 62 guide RNAs targeting the conserved regions of the OSD1, SPO11-1, REC8 or PAIR1 genes were transfected in triplicate in protoplasts from multiple plants from the Arabidopsis accessions described in section AM1. Editing efficiency was calculated by sequencing as described in Example 1's section GM3, and the resulting data presented in FIG. 27. Based on this data, the guide RNAs indicated with black arrows in FIG. 27 were chosen for editing the OSD1 gene, the PAIR1 gene, the REC8 gene, and the SPO11-1 gene.

AR2: Generation of Parent MiMe Plants

Regenerated plants were screened using the sequencing methods described in Example 1's section GM3. About 1% of the regenerated plants screened (1,903 total plants) had mutations predicted to result in either nonfunctional or non-expressed MiMe alleles, resulting in a MiMe genotype. Such plants were chosen for the crosses described below, which had mutations predicted to result in either nonfunctional or non-expressed rec8, osd1, and pair1 alleles, or nonfunctional or non-expressed rec8, osd1, and spo11-1 alleles.

Two unique plants designated PED-AR-BC and PED-AR-AA were chosen to move forward as parent MiMe plants for partial complementation crosses, within which each parent which had bi-allelic edits resulting in two distinctive complete MiMe genotypes for rec8, osd1, pair1, and rec8, osd1, spo11-1, respectively. Microscopic analysis of microsporogenesis showed these parent MiMe plants also produced dyads instead of tetrads, and also produced viable pollen. The parent MiMe plant PED-AR-BC (rec8/osd1/pair1) was derived from F2 individuals which themselves were derived from a selfed F1 hybrid between Col-0 (CS851557-S1) and diploid inbred HR-10. The parent MiMe plant PED-AR-AA (rec8/osd1/spo11-1) was derived from an inbred individual of Shandara.

Two unique plants designated PED-AR-BC and PED-AR-DE were chosen to move forward as parent MiMe plants for crossing, each of which had bi-allelic edits resulting in a complete MiMe genotype for rec8, osd1 and pair1. Microscopic analysis of microsporogenesis showed that all three parent MiMe plants produced dyads instead of tetrads and produced viable pollen. The parent MiMe plant PED-AR-DE (rec8/osd1/pair1) was derived from an F2 plant which itself derived from a selfed F1 hybrid between diploid inbred Col-0 and diploid inbred HR-10.

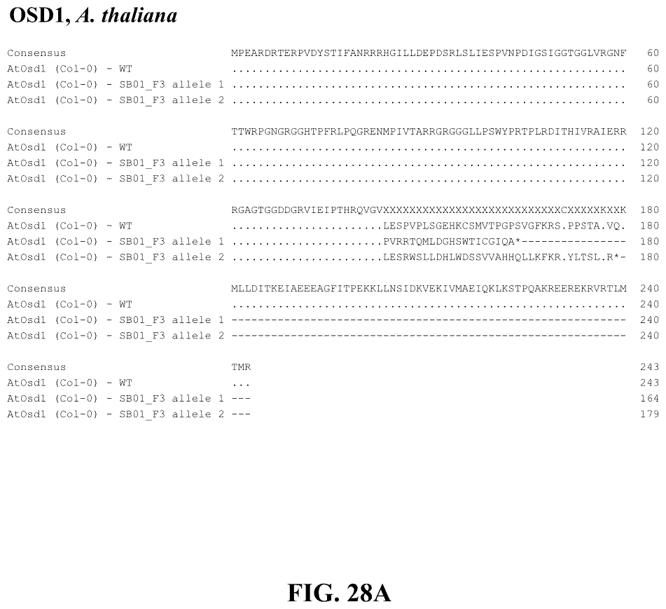

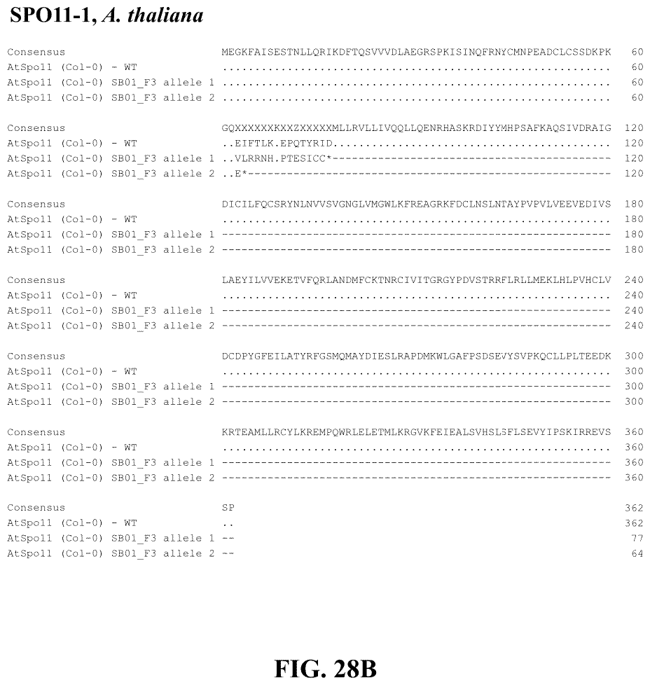

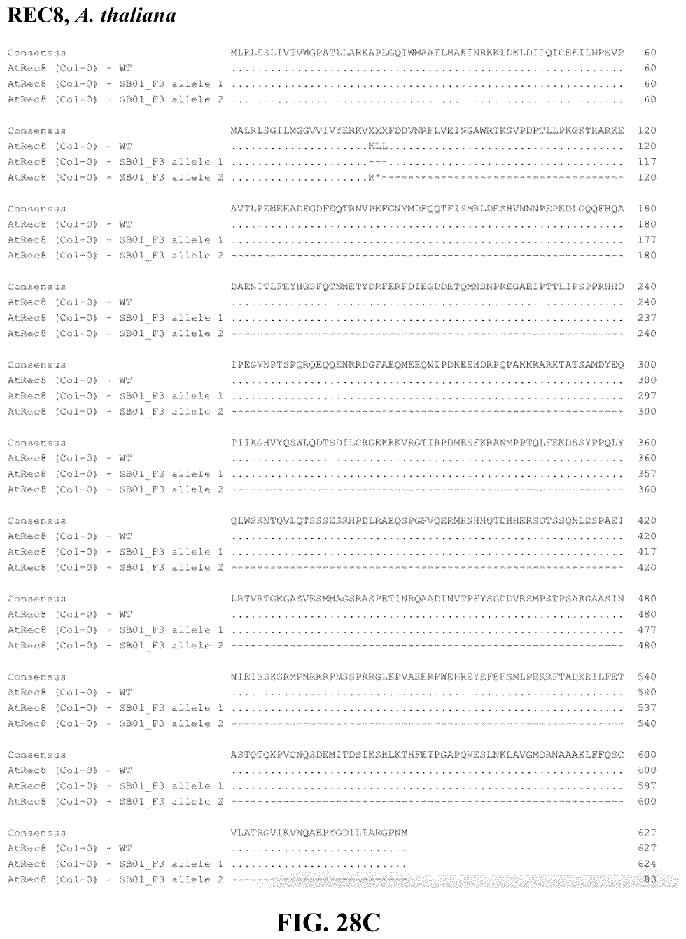

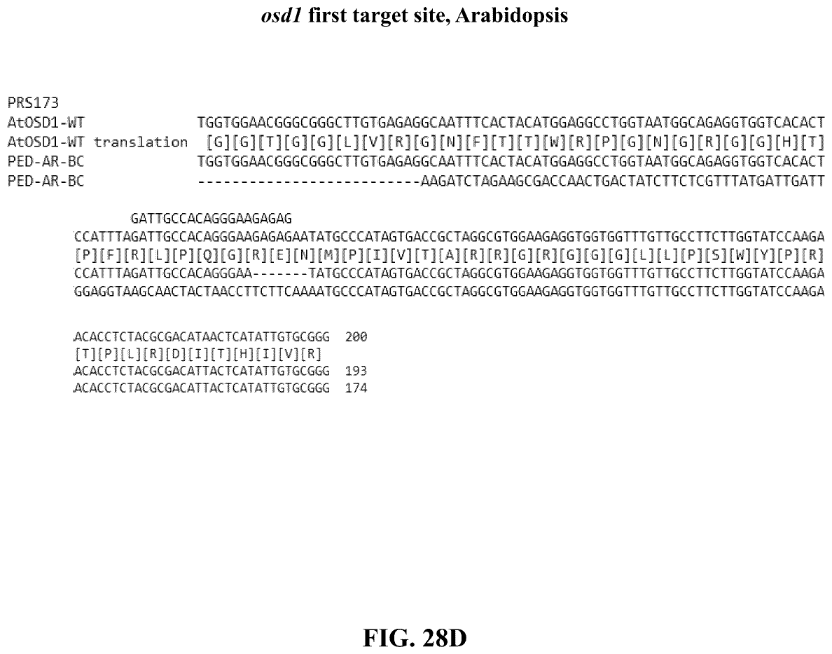

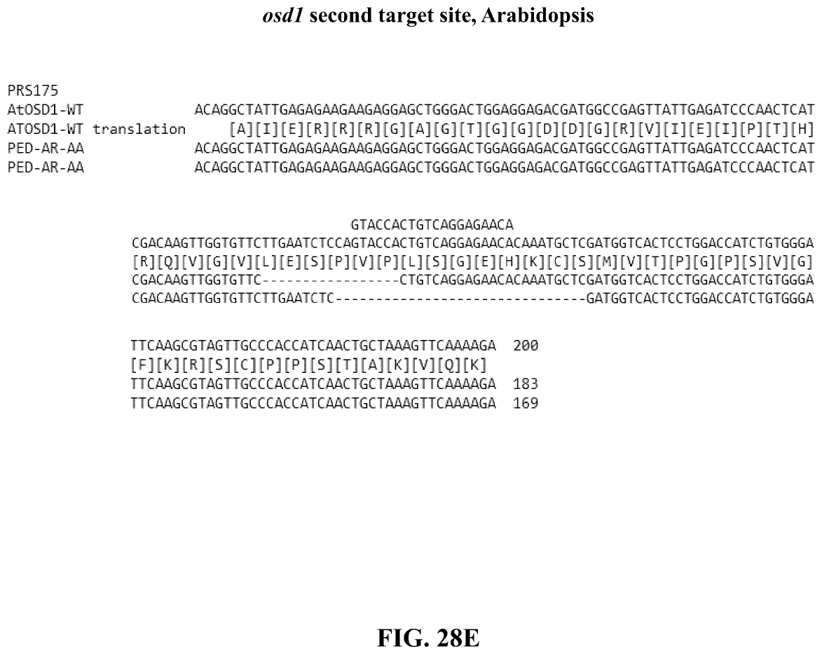

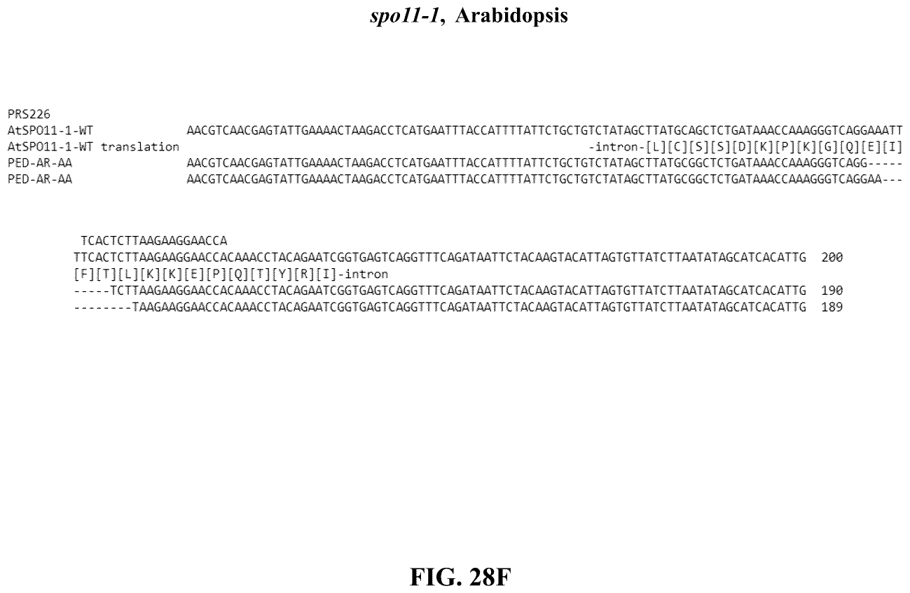

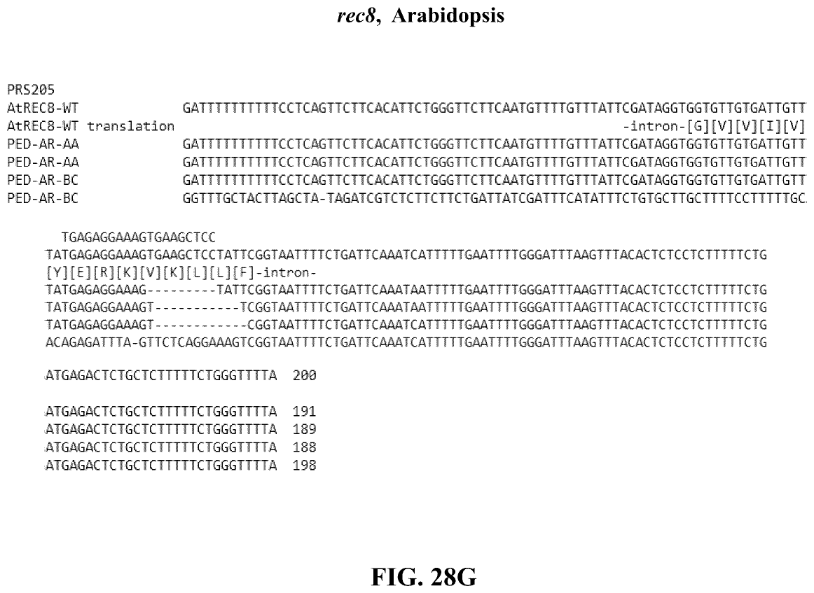

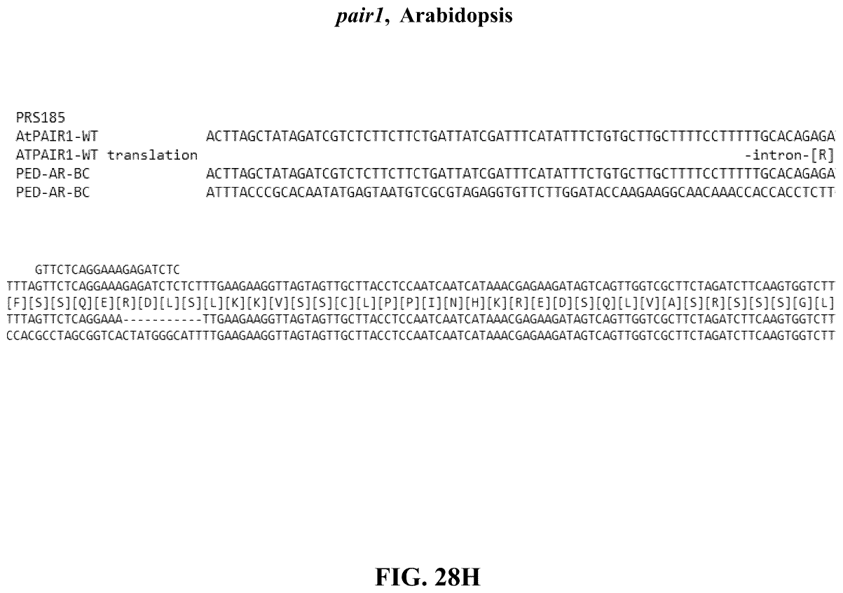

As an example, edits for the parent MiMe plant PED-AR-AA (rec8/osd1/spo11-1) are illustrated in FIGS. 39A-39C. In that figure, the predicted protein sequences are illustrated with respect to the unedited wildtype protein sequence and a consensus sequence, wherein an “X” in the consensus sequence indicates that there is no consensus for that amino acid position; wherein a dot (“·”) in the parent MiMe plant sequence indicates that the respective amino acid matches the consensus sequence; and wherein a dash (“-”) in the parent MiMe plant sequence indicates that the respective amino acid is present in the consensus sequence but is absent in the parent MiMe plant sample sequence. Frameshift inducing deletions in alleles of osd1 (FIGS. 28A, 28D, and 28E), spo11-1 (FIGS. 28B and 28F), one allele of rec8 (FIGS. 28C and 28G), and pair1 (FIG. 2811) result in premature stop codons. Premature stop codons are denoted by an asterisk (“*”) in FIGS. 28A-28C. Another allele of rec8 has a 9 bp deletion resulting in the loss of three (3) amino acid residues in a highly conserved domain of the protein which is expected to disrupt its function (FIGS. 28C and 28G).

AR3: Generation of Genetically Uniform Tetraploid Arabidopsis Seed

Parent MiMe plants were crossed using hand pollination. Seeds resulting from crosses were germinated following the procedures described in section AM1. In total, two genetically uniform populations of tetraploid Arabidopsis seed with either three or four haplotypes were created, from the parent MiMe plants described in section AR2.

The first genetically uniform population of tetraploid Arabidopsis seed referred to herein as Boosted Arabidopsis Population 1 (“BAP1”) consisted tetraploid individuals with three haplotypes, derived from a cross between a female parent MiMe plant PED-AR-BC (rec8/osd1/pair1) and a male parent MiMe plant PED-AR-AA (rec8/osd1/spo11-1).

The second genetically uniform population of tetraploid Arabidopsis seed referred to herein as Boosted Arabidopsis Population 2 (“BAP2”) consisted of tetraploid individuals with four haplotypes derived from a cross between a female parent MiMe plant PED-AR-BC (rec8/osd1/pair1) and a male parent MiMe plant PED-AR-DE (rec8/osd1/pair1).

For each individual, young leaf tissue or inflorescences were chosen for flow cytometry. All MiMe parent plants used for crosses were shown to be diploid having mean peak fluorescences consistent with diploid control plants. All individuals from BAP1 and BAP2 (except 1 individual from BAP1) were shown to be tetraploid with mean peak fluorescence values double that of diploid control plants. One individual from BAP1 had an intermediate mean peak between the tetraploid and diploid peaks, indicative of triploidy.

ARS: Genotypic Evaluation of BAP1, BAP2, and MiMe Parent Plants

Reads for wild type HR-10 were obtained from the Sequence Read Archive SRA (SRR492282). DNA samples were sequenced by Novogene. Libraries were prepared using the NEBNext® Ultra™ II for DNA Library Prep kit, and paired-end 2×150 reads were sequenced on an Illumina NovaSeq 6000 or NovaSeq X plus.

For the parent MiMe plants PED-AR-BC and PED-AR-AA, DNA samples were sequenced to a depth of 6,261,903,000 bp, and 6,856,028,700 bp, respectively. For the BAP1 progeny plants, DNA samples were sequenced to an average depth of 3,735,085,029 bp. For the parent MiMe plant PED-AR-DE, DNA samples were sequenced to a depth of 4,690,109,700 bp. For the BAP2 progeny plants, DNA samples were sequenced to an average depth of 3,938,238,717 bp. Reads were aligned to the TAIR10 reference genome with BWA-MEM (v0.7.17-r1188) (Li, H. 2013) and alignments were sorted and indexed with samtools (v1.15.1).

For BAP1, variants were called and jointly genotyped for parents (PED-AR-AA and PED-AR-BC), wild type HR-10, and all 100 progeny plants with GATK (v4.3.0.0) (McKenna, A et al. (2010) The Genome Analysis Toolkit: a MapReduce framework for analyzing next-generation DNA sequencing data. Genome research, 20(9), pp. 1297-1303). To identify heterozygous blocks in the F2 parent, only SNPs with the following characteristics are considered for analysis: (i) the SNP is either bi-allelic or tri-allelic amongst the three parental haplotypes; (ii) the SNP is homozygous ALT/ALT in HR-10 and not missing in PED-AR-BC; (iii) the SNP has a Mapping Quality (MQ) score of 60 and it is covered by at least 20 reads in PED-AR-BC; and (iv) the SNP is not within 200 bp of any indel. Using these SNPs, larger genotype blocks were defined by binning SNPs into non-overlapping 100 kbp windows. Each bin was assigned the modal genotype of its constituent SNPs. This method partitioned the PED-AR-BC genome into blocks of either homozygous REF/REF (0/0), homozygous ALT/ALT (1/1) or heterozygous REF/ALT (0/1) states. In total, six heterozygous blocks were identified spanning 61,735,056 bp (51.81% of the TAIR10 reference genome). A similar method was used to identify heterozygous blocks in PED-AR-DE. After establishing heterozygous blocks in the hybrid parents, markers were established and populations were genotyped as described in GM6.

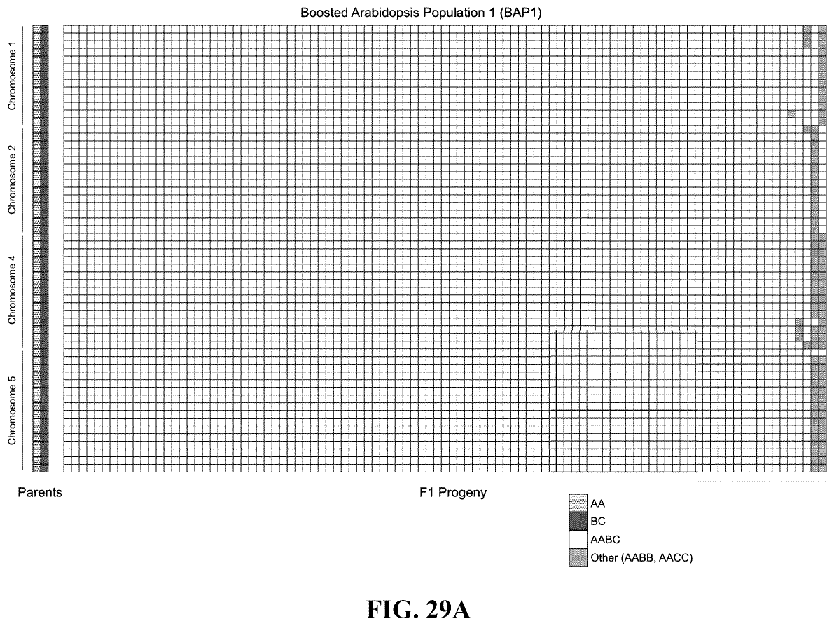

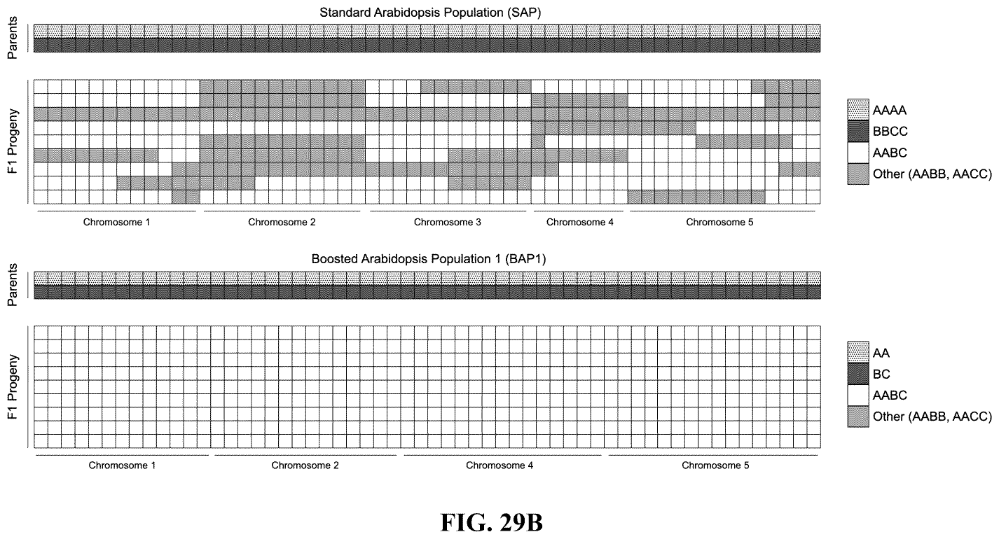

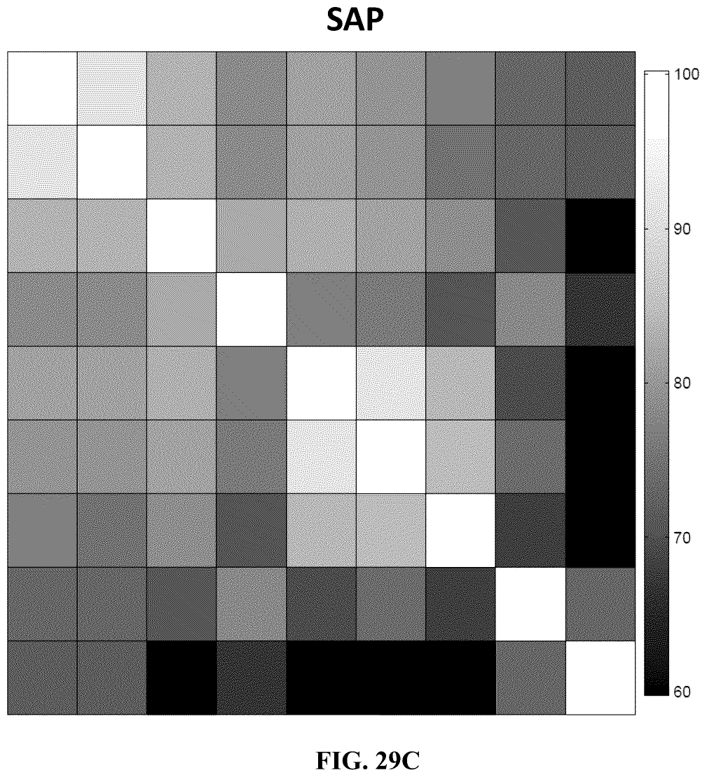

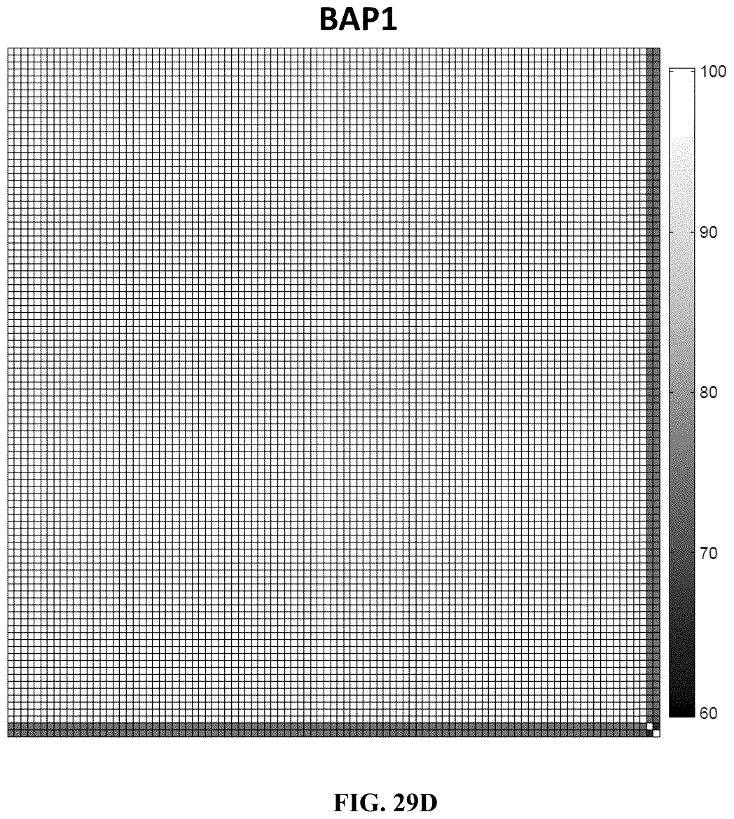

After filtering for high quality markers and excluding the one triploid plant, a genotype matrix was assembled for BAP1 (FIG. 29A). Across the BAP1 population, the average pairwise genetic uniformity as measured by the Jaccard similarity coefficient was 98.9% (visualized as a pairwise similarity matrix in FIG. 29D). These data are illustrated by the population uniformity graph in FIG. 24. The genetic uniformity was similar for the BAP2 population.

AR6: Genotypic Evaluation of Non-MiMe Progeny (SAP)

The extent of genotypic uniformity of a standard population of non-MiMe×non-MiMe tetraploid Arabidopsis seed was also determined. The population of non-MiMe tetraploid Arabidopsis seed referred to herein as the Standard Arabidopsis Population (“SAP”) consisted of nine tetraploid individuals derived from a cross between a female parent non-MiMe tetraploid plant Col-0 (CS851557-S1)×HR-10 (doubled F2 hybrid obtained through protoplast regeneration) and a male parent non-MiMe tetraploid plant Shandara (doubled inbred obtained through protoplast regeneration).

Reads for wild type HR-10 were obtained from the Sequence Read Archive SRA (SRR492282). DNA samples were sequenced by Novogene. Libraries were prepared using the NEBNext® Ultra™ II for DNA Library Prep kit, and paired-end 2×150 reads were sequenced on an Illumina NovaSeq 6000. For the female parent non-MiMe plant (Col-0(C5851557-S1)×HR-10 F2), DNA samples were sequenced to a depth of 5,956,964,100 bp. For SAP plants, DNA samples were sequenced to an average depth of 3,998,928,200 bp. Markers were established and populations were genotyped as described in GM6.

After filtering for high quality markers, a genotype matrix was assembled for SAP (FIG. 29B, top). Across the SAP population, the average pairwise genetic uniformity as measured by the Jaccard similarity coefficient was 75.2% (visualized as a pairwise similarity matrix in FIG. 29C). These data are further illustrated by the population uniformity graph in FIG. 24.

AR7: Phenotypic Evaluation, Parthenocarpy, and Enlarged Seedless Fruit

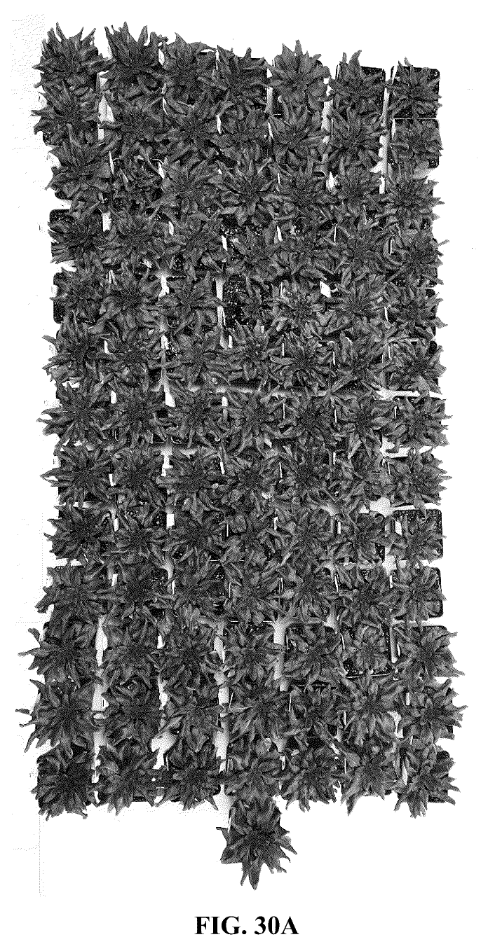

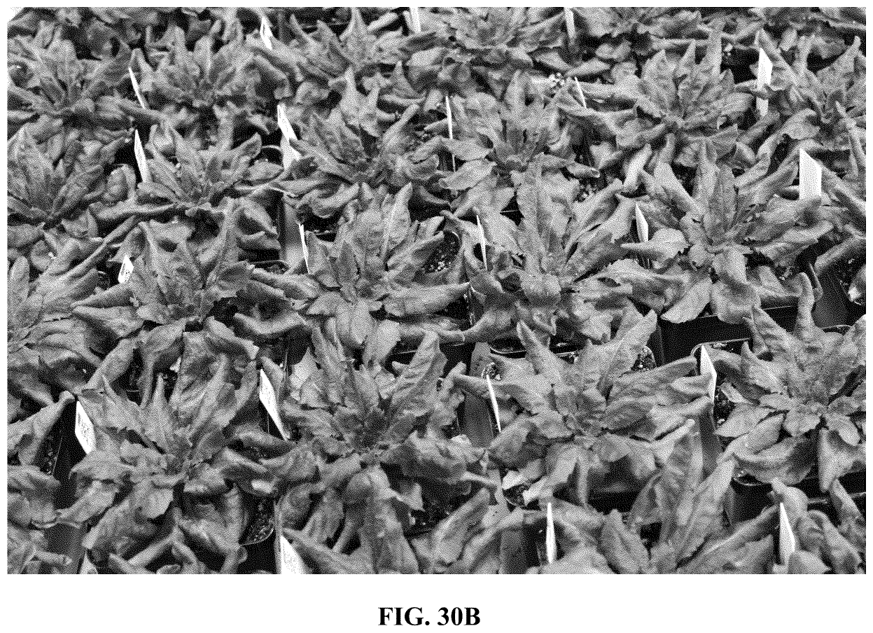

The plants from BAP1 and BAP2 were uniform in size, growth and appearance for their respective population. For example, plants from BAP1 are shown in FIG. 30A at 48 days after planting. A subset of the same plants from BAP1 are shown from a closer distance in FIG. 30B, also at 48 days after planting.

Plants from BAP1 had a partially-complemented spo11-1/pair1 MiMe genotype (rec8/rec8, osd1/osd1, spo11-1/pair1) which did not exhibit a wild-type meiosis phenotype because the MiMe loci of the first MiMe component and the MiMe loci of the third MiMe component of both the first and second parent MiMe plants were rec8 and osd1 respectively at both loci, but which also did not exhibit a MiMe phenotype either due to the complementation of a first MiMe locus (pair1) of the second MiMe component with a second MiMe locus (spoil-1) of the second MiMe component. Instead, plants from BAP1 produced parthenocarpic seedless fruit (siliques)

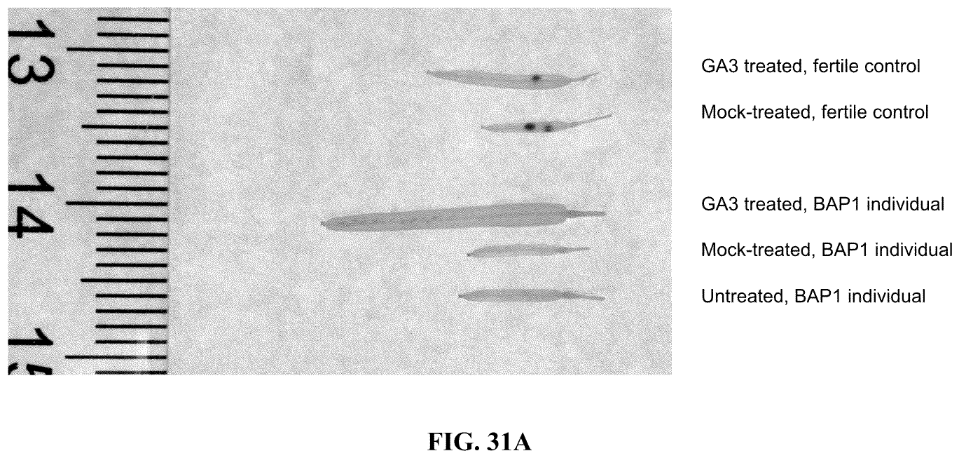

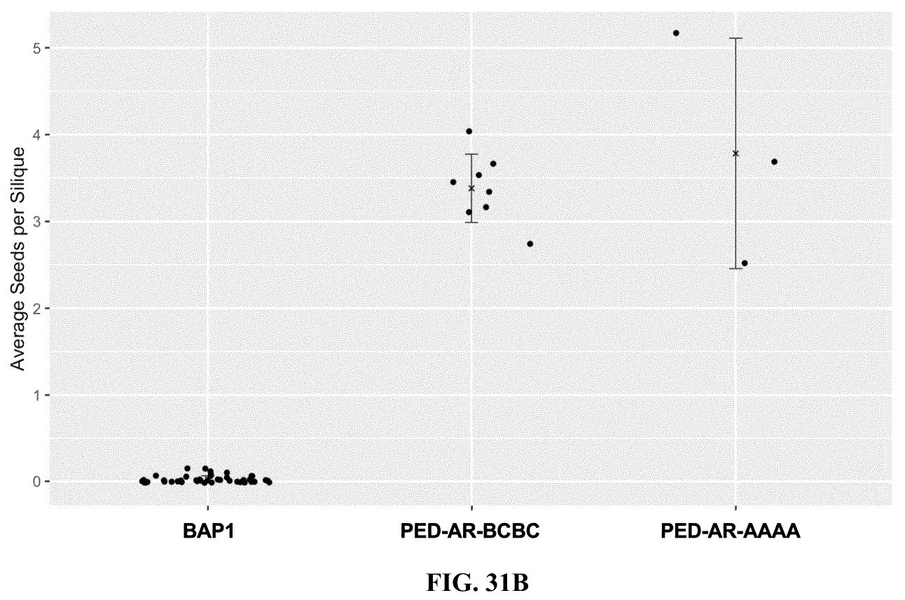

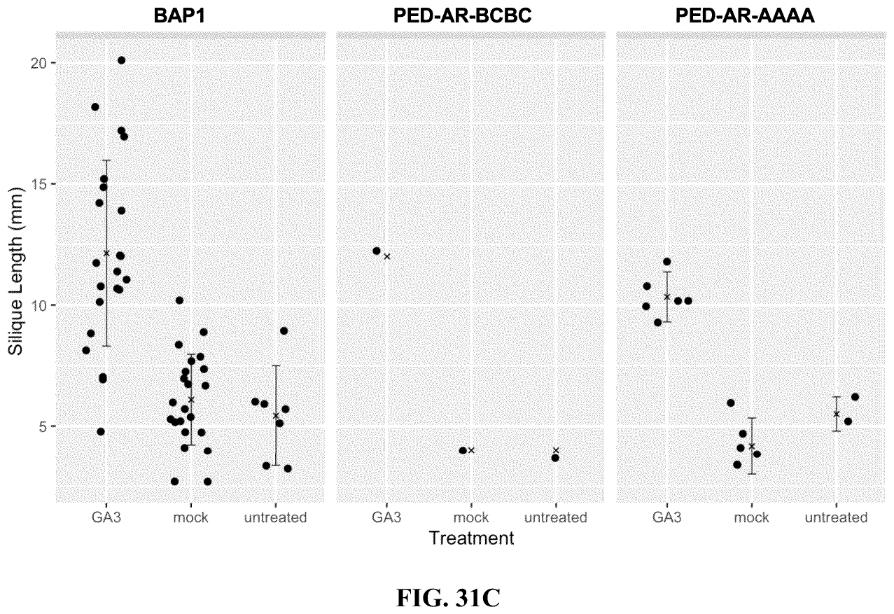

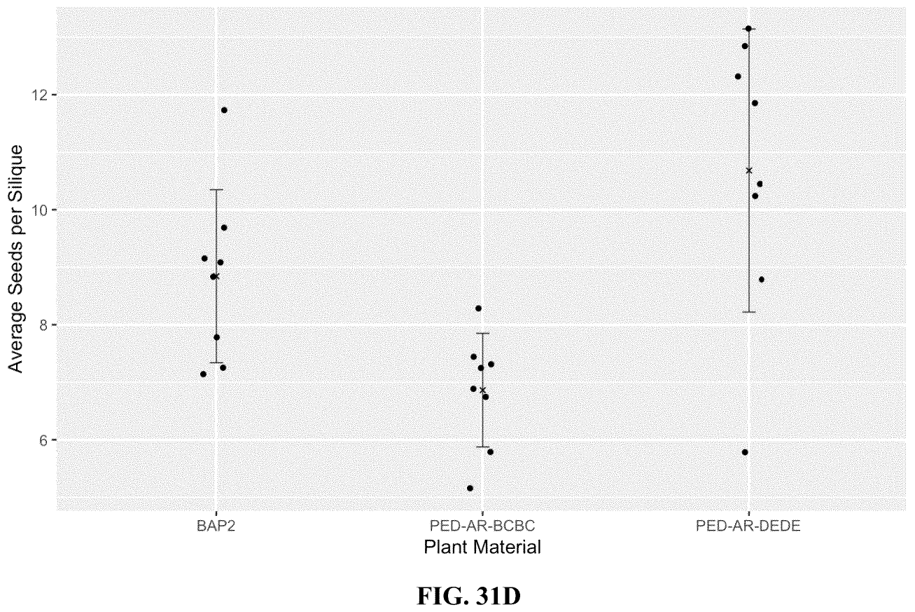

For example, FIG. 31A shows seedless fruit from BAP1 plants treated, untreated or mock-treated with gibberellic acid (GA3) to induce fruit development, compared to fruit from treated or mock-treated fertile Arabidopsis control plants as described in section AM6. When 42 plants from BAP1 were randomly selected and 20 fruits were randomly observed from each selected plant, it was determined that 31 of the 42 BAP1 plants produced no seeds, and the average amount was 0.15 seeds/fruit when considering all 42 plants. The seeds that were collected from the BAP1 plants failed to germinate under standard conditions, and demonstrating that all 42 plants from BAP1 failed to produce viable seed, and can therefore be considered seedless. In contrast, the fertile Arabidopsis control plants produced about 3 to 4 seeds/fruit. (FIG. 31B). The control plants were doubled versions of the BAP1 parent MiMe plants designated as PED-AR-AAAA (doubled version of PED-AR-AA) and PED-AR-BCBC (doubled version of PED-AR-BC).

As illustrated in FIG. 31A, the size of the fruit from GA3-treated BAP1 plants was often enlarged compared to fruit from control plants. The increase in fruit length (mm) was quantified for GA3-treated BAP1 compared to those from controls and illustrated in the graph provided by FIG. 31C. As illustrated in the photograph in FIG. 42A, the thickness and overall size of BAP1 fruit was also enlarged compared to that from a control plant.

On the other hand, plants from BAP2 were fertile with an average of 8.84 seeds/fruit as illustrated in FIG. 31D. The control plants were doubled versions of the BAP2 parent MiMe plants designated as PED-AR-BCBC (doubled version of PED-AR-BC) and PED-AR-DEDE (doubled version of PED-AR-DE). PED-AR-BCBC plants had an average of 6.86 seeds/fruit, whereas PED-AR-DEDE plants had an average of 10.68 seeds/fruit.

Without being held to any one particular theory, we interpret these results to indicate that functional copies of SPO11-1 and PAIR1 complement their respective knockouts and restore the cellular function of DNA double strand breakage, thus having essentially the same phenotype as a rec8 mutant and producing fragmented chromosomes at the end of the first meiotic division. The skipping of a second division becomes irrelevant because the fragmented chromosomes will not produce viable gametes. Because viable gametes are not produced, egg cells cannot be produced to lead to seeds. Similarly, complementing cycal and osd1 will result in similar phenotype due to progression through the second division of meiosis causing random or non-assortment of chromosomes.

### Example 4: Breeding to Combine Edited and/or Natural MiMe Alleles in Grandparent Non-MiMe Plants to Produce Parent MiMe Plants

This Example describes a method of producing a population of polyploid seed by first generating four grandparent non-MiMe plants having partial MiMe genotypes comprising edited and/or natural MiMe alleles, then crossing the grandparent non-MiMe plants to produce two parent MiMe plants having complete MiMe genotypes, and finally crossing the two parent MiMe plants to produce the population of polyploid seeds. Exemplary variations of this method are depicted in FIGS. 14 and 15. This method may be used to produce polyploid hybrid seeds having three or more haplotypes, e.g., if the grandparent non-MiMe plants together comprise three or more haplotypes. The methods are described below for maize, but may be modified to be applied to any polyploid plant for which suitable transformation and gene editing methods are known to one of skill in the art.

A construct for expressing a nuclease (e.g., pMEM4 shown in FIG. 8, or a similar construct) is bombarded into maize line PED495, and events are selected as described in Wang and Frame (2009. Biolistic gun-mediated maize genetic transformation. Transgenic maize: methods and protocols, pp. 29-45). Separately, a construct for expressing a gRNA targeting a MiMe locus (e.g., pMEM6b as shown in FIG. 9 or a similar construct) is bombarded into maize line PED495, and events are selected as described in Wang and Frame 2009. To plants containing the nuclease construct are crossed with To plants containing the gRNA construct to produce T₁F₁ hybrid plants containing both transgenes. The T₁F₁ hybrid plants are screened for editing of the MiMe locus targeted by the gRNA, and any plants with editing are self-pollinated or outcrossed to produce plants heterozygous for knock-out edits of individual MiMe loci. Plants that are heterozygous for edits of single or multiple MiMe loci are crossed together or crossed to natural knockout alleles to produce four plants, each heterozygous for the targeted MiMe genotype, e.g., osd1/−(e.g., osd1-1/−, osd1-2/−, osd1-3/−, or any combination thereof), spo11/−, and spo8/− (i.e., each having a partial MiMe genotype). These plants are called A_{HetMiMe}, B_{HetMiMe}, C_{HetMiMe}, and D_{HetMiMe} and are considered grandparent non-MiMe plants each having a partial MiMe genotype. These plants are crossed in an AxB and CxD fashion to and screened for plants with a homozygous knockout genotype at all 4 MiMe alleles. These plants are called AB_{MiMe} and CD_{MiMe}. These are considered parent MiMe plants and are recovered at a rate of approximately 1/256. These AB_{MiMe} and CD_{MiMe} parent MiMe plants can then be crossed together to produce a population tetraploid ABCD seed which can be grown into a tetraploid ABCD plant.

### Example 5: Editing of Hybrid Maize Lines to Directly Produce Parent MiMe Plants

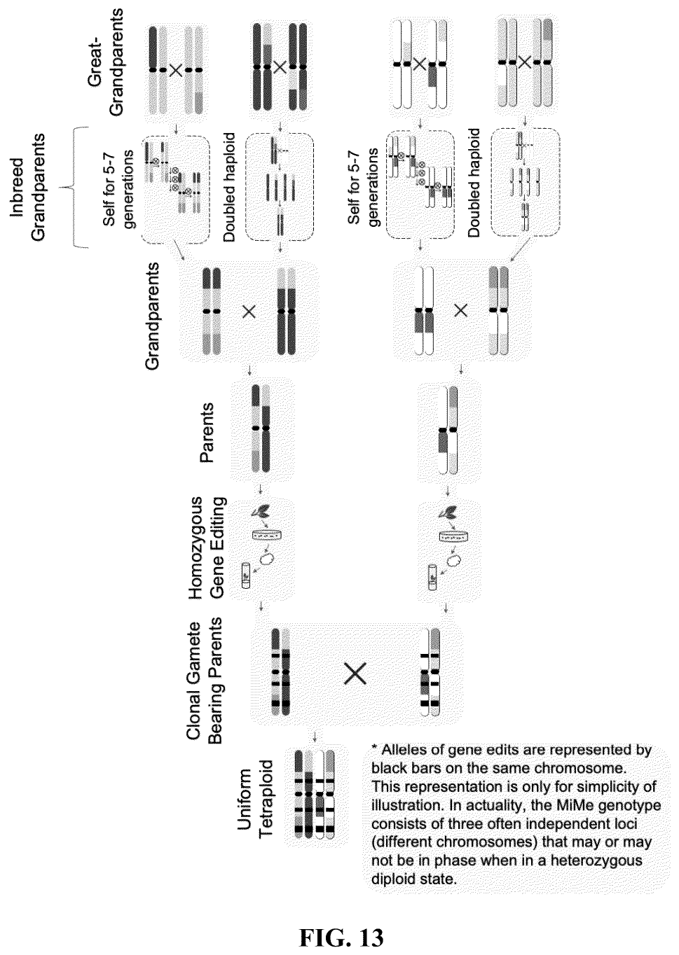

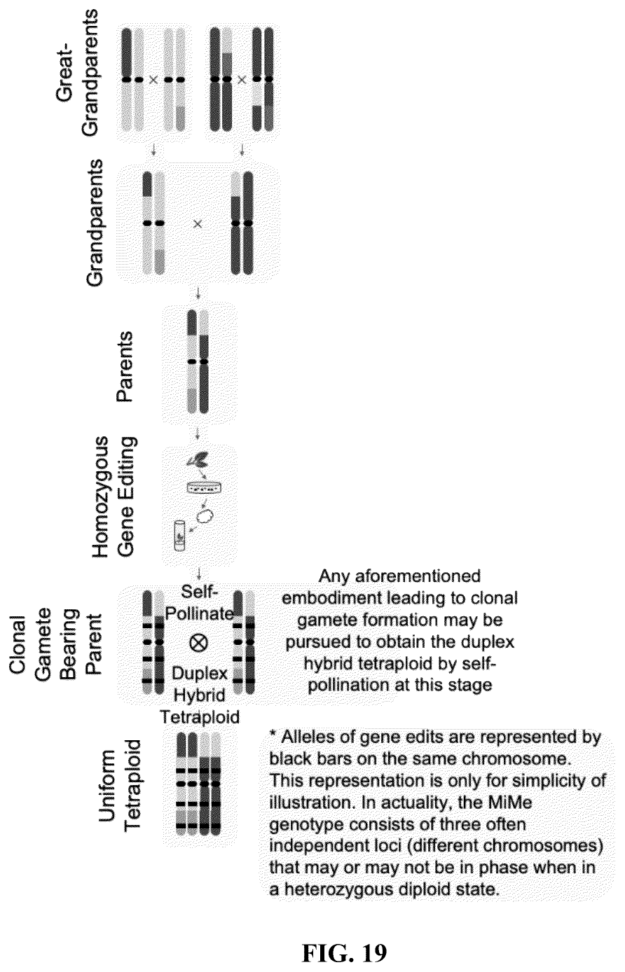

This Example describes an additional method to produce parent MiMe plants by directly editing two hybrid maize lines and screening for plants with a complete MiMe genotype. Exemplary methods of breeding and producing hybrid polyploid seed comprising directly editing two hybrid plant lines to produce parent MiMe plants are depicted in FIGS. 12, 13, and 19. This method may be used to produce polyploid hybrid seeds having three or more haplotypes, e.g., if two hybrid lines together comprising three or more haplotypes are directly edited to produce the parent MiMe plants.

PED071 and hybrid PED495 are crossed together to make hybrid embryos that are co-bombarded with gRNA construct pOGZ2 (FIG. 10), and one of pMEM4 (FIG. 8), or pOGZ4 (FIG. 11) as a nuclease construct as well as a selectable marker for bialaphos resistance as described in Wang and Frame 2009 with modifications (using described bombardment with TransIT-2020). Additionally a hybrid of PED071 and PED195 is transformed with an identical co-bombardment and transgenic plants can be selected per McCaw et al. (2020. Development of a Transformable Fast-Flowering Mini-Maize as a Tool for Maize Gene Editing. Frontiers in Genome Editing. doi: 10.3389/fgeed.2020.622227), with modifications of using bombardment as described (using described bombardment with TransIT-2020) rather than transformation with Agrobacterium. The hybrid transformants are screened for two or more independent events with complete knockout MiMe genotype to act as parent MiMe plants. These parent MiMe plants are crossed to produce a uniform population of tetraploid seed having 3 or more haplotypes, which may be grown into a plant having 3 or more haplotypes. These 3 or more haplotypes may be a combination of PED071, PED195, and an F2 segregant of PED495. They may also be a combination of PED071 and two distinct genotypes that are the result of F2 segregation of PED495.

What is claimed is:

1. A population of polyploid maize seed comprising a subpopulation of genetically uniform polyploid maize seed in an amount of at least 50% of the total number of seeds, the genetically uniform polyploid maize seed comprising three or more haplotypes of the same or related species of maize, wherein the population was obtained from a single maize plant or a set of F1 hybrid maize plants, wherein the subpopulation of genetically uniform polyploid maize seed comprise (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or (B) (2) a germplasm genetic modification means for preventing DNA double strand breakage during meiotic recombination, and (4) a germplasm genetic modification means for preventing progression through the first division of meiosis;
wherein each of the germplasm genetic modifications are (i) a complete set that achieves the inhibition, (ii) a partial set that does not achieve inhibition, or (iii) partially complemented at one or more of the genetic modification means and achieve sterility.

2. The population of claim 1, wherein the subpopulation of genetically uniform polyploid maize seed is triploid, tetraploid, pentaploid, hexaploid, heptaploid, or octoploid.

3. The population of claim 1, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

4. The population of claim 1, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

5. The population of claim 1, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

6. The population of claim 1, wherein the subpopulation of genetically uniform polyploid maize seed has a complete MiMe genotype comprising the complete set of germplasm genetic modification means as follows:
(A)
(1) the germplasm genetic modification means for inhibiting sister chromatid cohesion during the first division of meiosis comprise only MiMe alleles at one or more MiMe loci of a component of sister chromatid cohesion during the first division of meiosis, (2) the germplasm genetic modification means for inhibiting DNA double strand breakage during meiotic recombination comprise only MiMe alleles at one or more MiMe loci of a component of DNA double strand breakage during meiotic recombination, and (3) the germplasm genetic modification means for inhibiting progression through the second division of meiosis comprise only MiMe alleles at one or more Mime loci of progression through the second division of meiosis; or
(B)
(2) the germplasm genetic modification means for inhibiting DNA double strand breakage during meiotic recombination comprise only MiMe alleles at one or more MiMe loci of a component of DNA double strand breakage during meiotic recombination, and (4) the germplasm genetic modification means for inhibiting progression through the first division of meiosis comprise only MiMe alleles at one or more MiMe loci of a component of progression through the first division of meiosis,
wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

7. The population of claim 1, wherein the subpopulation of genetically uniform polyploid maize seed has a partial MiMe genotype comprising the partial set of germplasm genetic modification means as follows:
(A)
(1) the germplasm genetic modification means for inhibiting sister chromatid cohesion during the first division of meiosis comprise at least one MiMe allele and at least one non-MiMe allele at one or more MiMe loci for a component of sister chromatid cohesion during the first division of meiosis comprise, (2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (3) the germplasm genetic modification means for inhibiting progression through the second division of meiosis comprise at least one MiMe allele and at least one non-MiMe allele at one or more MiMe loci for a component of progression through the second division of meiosis; or
(B)
(2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (4) the germplasm genetic modification means for inhibiting progression through the first division of meiosis comprise at least one MiMe allele and at least one non-MiMe allele at one or more MiMe loci for a component of progression through the first division of meiosis,
wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

8. The population of claim 1, wherein the subpopulation of genetically uniform polyploid maize seed has a partially-complemented MiMe genotype comprising the partially complemented set of germplasm genetic modification means as follows:
(A)
(1) the germplasm genetic modification means for inhibiting sister chromatid cohesion during the first division of meiosis comprises at least one MiMe allele at at least a first MiMe locus of a component of sister chromatid cohesion during the first division of meiosis, (2) the germplasm genetic modification means for inhibiting DNA double strand breakage during meiotic recombination comprises at least one MiMe allele at at least a first MiMe locus of a component of DNA double strand breakage during meiotic recombination, and (3) the germplasm genetic modification means for inhibiting progression through the second division of meiosis comprises at least one MiMe allele at at least a first MiMe locus of a component of progression through the second division of meiosis,
wherein at least one of (1), (2), and (3) comprise one or more MiMe alleles and one or more non-MiMe alleles at the first MiMe locus of the component, and one or more MiMe alleles and one or more non-MiMe alleles at a second MiMe locus of the component, and wherein one or both of the others of (1), (2), and (3) comprise only MiMe alleles at at least the first MiMe locus of the component; or
(B)
(2) the germplasm genetic modification means for inhibiting DNA double strand breakage during meiotic recombination comprises at least one MiMe allele at at least a first MiMe locus of a component of DNA double strand breakage during meiotic recombination, and (4) the germplasm genetic modification means for preventing progression through the first division of meiosis comprises at least one MiMe allele at at least a first MiMe locus of a component of progression through the first division of meiosis, wherein one of (2) and (4) comprises one or more MiMe alleles and one or more non-MiMe alleles at the first MiMe locus of the component, and one or more MiMe alleles and one or more non-MiMe alleles at a second MiMe locus of the component, and the other of (2) and (4) comprises only MiMe alleles at at least the first MiMe locus of the component; or
(C)
(2) the germplasm genetic modification means for inhibiting DNA double strand breakage during meiotic recombination comprise at least one MiMe allele at at least a first MiMe locus of a component of DNA double strand breakage during meiotic recombination;
(1) the germplasm genetic modification means for inhibiting sister chromatid cohesion during the first division of meiosis comprises at least one MiMe allele and at least one non-MiMe allele at one or more MiMe loci for a component of sister chromatid cohesion during the first division of meiosis;
(3) the germplasm genetic modification means for inhibiting progression through the second division of meiosis comprises at least one MiMe allele and at least one non-MiMe allele at one or more MiMe loci for a component of progression through the second division of meiosis; and
(4) the germplasm genetic modification means for inhibiting progression through the first division of meiosis comprise at least one MiMe allele and at least one non-MiMe allele at one or more MiMe loci for a component of progression through the first division of meiosis,
wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

9. The population of claim 6, wherein the subpopulation of genetically uniform polyploid maize seed comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(14) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(15) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

10. A method of producing the population of polyploid maize seed of claim 1, the method comprising:
(a) generating a first parent MiMe maize plant and a second parent MiMe maize plant by introducing genetic modifications into germplasms of one or more candidate maize lines to generate the first parent MiMe maize plant, the second parent MiMe maize plant, and/or progenitors thereof, wherein the first parent MiMe maize plant and the second parent MiMe maize plant together comprise three or more haplotypes, and wherein each of the first parent MiMe maize plant, the second parent MiMe maize plant, and/or the progenitors thereof comprises:
(A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
(B) (2) a germplasm genetic modification means for preventing DNA double strand breakage during meiotic recombination, and (4) a germplasm genetic modification means for preventing progression through the first division of meiosis;
wherein each of the germplasm genetic modifications are (i) a complete set that achieves the inhibition, or (ii) a partial set that does not achieve inhibition;
wherein if progenitors are generated, the progenitors are further crossed to generate the first parent MiMe maize plant, the second parent MiMe maize plant, or both, and wherein the generating the first parent MiMe maize plant, the second parent MiMe maize plant, and/or the progenitors thereof comprises selecting for maize plants comprising the germplasm genetic modification means after introducing the germplasm genetic modification means;
(b) providing clonal gametes from the first parent MiMe maize plant and the second parent MiMe maize plant that together comprise the three or more haplotypes; and
(c) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid seed comprising the three or more haplotypes.

11. A method of producing the population of polyploid maize seed of claim 1, the method comprising:
(a) generating a first parent MiMe maize plant by introducing genetic modifications into germplasms of one or more candidate maize lines to generate the first parent MiMe maize plant or progenitors thereof, and wherein each of the first parent MiMe maize plant or the progenitors thereof comprises:
(A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
(B) (2) a germplasm genetic modification means for preventing DNA double strand breakage during meiotic recombination, and (4) a germplasm genetic modification means for preventing progression through the first division of meiosis;
wherein each of the germplasm genetic modifications are (i) a complete set that achieves the inhibition, (ii) a partial set that does not achieve inhibition, or (iii) partially complemented at one or more of the genetic modification means and achieve sterility;
wherein if progenitors are generated, the progenitors are further crossed to generate the first parent MiMe maize plant, and wherein the generating the first parent MiMe maize plant and/or the progenitors thereof comprises selecting for maize plants comprising the germplasm genetic modification means after introducing the germplasm genetic modification means;
(b) providing clonal gametes from the first parent MiMe maize plant;
(c) providing haploid gametes from a homozygous parent non-MiMe maize plant; and
(d) crossing the clonal gametes with the haploid gametes to produce the population of polyploid maize seed,
wherein the clonal gametes and the haploid gametes together comprise three or more haplotypes.

12. A method of breeding a polyploid hybrid maize line, the method comprising:
(a) obtaining a set of maize lines;
(b) breeding the maize lines using traditional plant breeding methods to produce a set of candidate maize lines;
(c) selecting two or more candidate maize lines together comprising three or more haplotypes;
(d) generating a first parent MiMe maize plant and a second parent MiMe maize plant from the two or more candidate maize lines that together comprise the three or more haplotypes;
(e) providing clonal gametes from each of the first and second parent MiMe maize plants;
(f) crossing the clonal gametes to produce a hybrid polyploid maize seed comprising the three or more haplotypes;
(g) growing the hybrid polyploid maize seed to produce a hybrid polyploid maize plant comprising three or more haplotypes; and
(h) evaluating one or more characteristics of the hybrid polyploid maize plant.

13. A method of breeding a polyploid hybrid maize line, the method comprising:
(a) obtaining a set of maize lines;
(b) breeding the maize lines using traditional plant breeding methods to produce a set of candidate maize lines;
(c) selecting two or more candidate maize lines together comprising three or more haplotypes;
(d) generating a first parent MiMe maize plant from one of the two or more candidate maize lines;
(e) providing clonal gametes from the first parent MiMe maize plant;
(f) providing haploid gametes from a homozygous parent non-MiMe maize plant of one of the two or more candidate maize lines;
(g) crossing the clonal gametes with the haploid gametes to produce a hybrid polyploid maize seed;
(h) growing the hybrid polyploid maize seed to produce a hybrid polyploid maize plant; and
(i) evaluating one or more characteristics of the hybrid polyploid maize plant,
wherein the first parent MiMe maize plant and the homozygous parent non-MiMe maize plant together comprise three or more haplotypes, wherein the crossing of step (g) results in the hybrid polyploid maize seed comprising three or more haplotypes, and wherein the growing of step (h) results in the hybrid polyploid maize plant comprising three or more haplotypes.

14. The method of claim 12, wherein the first parent MiMe maize plant, the second parent MiMe maize plant, the parent non-MiMe maize plant, or any combination thereof are diploid, triploid, or tetraploid and the hybrid polyploid maize plant is tetraploid, pentaploid, hexaploid, heptaploid, or octoploid.

15. The method of claim 12, wherein generating the first parent MiMe maize plant, the second parent MiMe maize plant, or both comprises:
(1) introducing a complete MiMe genotype directly into two candidate maize lines to produce the first parent MiMe maize plant, the second parent MiMe maize plant, or both;
(2) introducing a partial MiMe genotype into two candidate maize lines to produce two grandparent non-MiMe maize plants each having a partial MiMe genotype and crossing the grandparent non-MiMe maize plants each having a partial MiMe genotype to produce the first parent MiMe maize plant; or
(3) introducing a partial MiMe genotype into four candidate maize lines to produce four grandparent non-MiMe maize plants each having a partial MiMe genotype, and crossing pairs of said grandparent non-MiMe maize plants each having a partial MiMe genotype to produce the first and second parent MiMe maize plants.

16. The method of claim 12, wherein the first parent MiMe maize plant, the second parent MiMe maize plant, the hybrid polyploid maize plant, or any combination thereof has a complete MiMe genotype comprising:
(A)
MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and
(3) a component of progression through the second division of meiosis, and each of the first MiMe component, the second MiMe component, and the third MiMe component are different MiMe components; or
(B)
MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and
(4) a component of progression through the first division of meiosis, and each of the first MiMe component and the second MiMe component are different MiMe components,
wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

17. The method of claim 12, wherein the hybrid polyploid maize plant has a partial MiMe genotype comprising:
(A)
(a) one or more MiMe alleles conferring decreased expression of one or more MiMe loci of each of a first, second, and third MiMe component; and
(b) one or more non-MiMe alleles at the one or more MiMe loci of each of the first, second, and third MiMe component,
wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
(B)
(a) one or more MiMe alleles conferring decreased expression of one or more MiMe loci of each of the first and second MiMe component; and
(b) one or more non-MiMe alleles at the one or more MiMe loci of each of the first and second MiMe component,
wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components,
wherein:
(1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

18. The method of claim 12, wherein the hybrid polyploid maize plant comprises:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(3) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(5) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(7) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(8) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1; or
(9) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

19. The method of claim 13, wherein the hybrid polyploid maize plant comprises:
(1) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(3) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(5) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1; or
(6) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

20. The method of claim 18, wherein the decreased expression of one or more of the MiMe loci is each independently achieved by introducing into each candidate maize line an insertion, a deletion, one or more nucleotide changes, or an inversion that results in decreased expression of the MiMe locus.

21. The method of claim 20, wherein the insertion, the deletion, the one or more nucleotide changes, or the inversion eliminates expression of the MiMe locus by introducing a premature stop codon present in the 70%, the first 60%, the first 50%, the first 40%, the first 30%, the first 20%, or the first 10% of the nucleotides of the coding sequence of the MiMe locus following the start codon in the 3′ direction.

22. A method of producing a population of polyploid maize seed comprising:
(a) providing clonal gametes from a pair of parent MiMe maize plants together comprising three or more haplotypes that were selected using the method of breeding of claim 12 based upon the polyploid maize plant comprising the three or more haplotypes having one or more desired characteristics; and
(b) crossing the clonal gametes to produce the population of polyploid maize seed;
wherein the population of polyploid maize seed comprises a subpopulation of genetically uniform polyploid maize seed in an amount of at least 50% of the total number of seeds, the subpopulation of genetically uniform polyploid maize seed comprising the three or more haplotypes.

23. A method of producing a population of polyploid maize seed comprising:
(a) selecting three or more haplotypes using the method of breeding of claim 13 based upon the polyploid maize plant comprising said three or more haplotypes having one or more desired characteristics;
(b) providing clonal gametes from a parent MiMe maize plant;
(c) providing haploid gametes from a homozygous parent non-MiMe maize plant;
(d) crossing the clonal gametes with the haploid gametes to produce the population of polyploid maize seed;
wherein the parent MiMe maize plant and the homozygous parent non-MiMe maize plant together comprise the three or more haplotypes selected in step (a), wherein the crossing of step (d) results in a population of polyploid maize seed comprising a subpopulation of genetically uniform polyploid maize seed in an amount of at least 50% of the total number of seeds, the subpopulation of genetically uniform polyploid maize seed comprising the three or more haplotypes.

24. The method of claim 22, wherein the population of polyploid maize seed has an average pairwise genetic uniformity of at least 80%, at least 85%, at least 90%, at least 95%, at least 96%, at least 97%, at least 98%, at least 99%, or 100% as measured by the Jaccard similarity coefficient.

25. The method of claim 22, wherein the population of polyploid maize seed comprises the subpopulation of genetically uniform polyploid maize seed in an amount of at least 70%, at least 90%, at least 95%, at least 96%, at least 97%, at least 98%, or at least 99% of the total number of seeds.

26. The method of claim 22, wherein each pair of seeds in the subpopulation of genetically uniform polyploid maize seed has a pairwise identity of at least 95%, at least 96%, at least 97%, at least 98%, at least 99%, or 100% as measured by the Jaccard similarity coefficient.

27. A genetically modified maize plant, plant part, or plant cell comprising:
(A) three or more haplotypes; and
(B)
(i) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
ii) a partial MiMe genotype comprising:
(a) one or more MiMe alleles conferring decreased expression of one or more MiMe loci of each of a first, second, and third MiMe component; and
(b) one or more non-MiMe alleles at the one or more MiMe loci of each of the first, second, and third MiMe component,
wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
iii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components;
iv) a partial MiMe genotype comprising:
(a) one or more MiMe alleles conferring decreased expression of one or more MiMe loci of each of the first and second MiMe component; and
(b) one or more non-MiMe alleles at the one or more MiMe loci of each of the first and second MiMe component,
wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components;
v) a partially complemented MiMe genotype comprising:
(a) only MiMe alleles at one or more MiMe loci of a first MiMe component;
(b) one or more MiMe alleles and one or more non-MiMe alleles at a first MiMe locus of a second MiMe component, and one or more MiMe alleles and one or more non-MiMe alleles at a second MiMe locus of the second MiMe component; and
(c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
vi) a partially complemented MiMe genotype comprising:
(a) only MiMe alleles at one or more MiMe loci of a first MiMe component; and
(b) one or more MiMe alleles and one or more non-MiMe alleles at a first MiMe locus of a second MiMe component, and one or more MiMe alleles and one or more non-MiMe alleles at a second MiMe locus of the second MiMe component,
wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
vii) a partially complemented MiMe genotype comprising:
(a) only MiMe alleles at one or more MiMe loci of a first MiMe component, wherein the first MiMe component is a component of DNA double strand breakage during meiotic recombination;
(b) one or more MiMe alleles and one or more non-MiMe alleles at one or more MiMe loci of a second MiMe component;
(c) one or more MiMe alleles and one or more non-MiMe alleles at one or more MiMe loci of a third MiMe component; and
(d) one or more MiMe alleles and one or more non-MiMe alleles at one or more MiMe loci of a fourth MiMe component,
wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components,
wherein (1) the one or more MiMe loci of the component of sister chromatid cohesion during the first division of meiosis comprise REC8, SWITCH1/DYAD, or a combination thereof;
(2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
(3) the one or more MiMe loci of the component of progression through the second division of meiosis comprise OSD1, CYCA1, TDM1, PC1, PC2, FC, or any combination thereof; and/or
(4) the one or more MiMe loci of the component of progression through the first division of meiosis comprise PS1, JASON, or a combination thereof.

28. The genetically modified maize plant, plant part, or plant cell of claim 27, comprising:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

29. The genetically modified maize plant, plant part, or plant cell of claim 27, wherein the maize plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.

30. A processed maize product derived from the genetically modified maize plant, plant part, or plant cell of claim 27, wherein the processed maize product comprises a detectable amount of the one or more MiMe alleles of the genetically modified maize plant, plant part, or plant cell.


SEQUENCE LISTING (111 sequences)

SEQ ID NO: 1 - protein, 603 aa, Zea mays
MFYSHQLLARKAPLGQIWMAATLHSKINRKRLDKLDIIKICEEILNPSVPMALRLSGILMGGVVIVYERKVKLLYTDVSRLLTEINEAWRIKPVTDPTVLPKGKTQAKYEAVTLPEINMVVEQPMFFSEPDGAKFRRMGLEDLDEQYVQVNLDDDDFSHADDRHQAKAVNITLVDNFESGLAETDLFNHFERFDIADDETTVNITPDEYPQVPSTLIPSPPRQEDIPQQEEPYYAAPSPVHGEPQQGGPEDQEEQKMKQPPKASKRKARWEVPRVIMDNNQMMIPGNIYQTWLKDASSLVSKRRKLNSNFNFIRSTKISDLMHIPPVALISHDNLFSELCYPKPLMQLWKDCTEVKSTKASSGGQRSSSQEPQPKNSPPQAGGEYEMETGGLPMDLTDGIEKLRANMSAKYDRAYNILHSDHSVTPGSPAGLSRRSASSSGGSGSAFIQLDPEVQLPSGSGRSKRGQHSSARSLGNLDTVEEDFPLEQEVRDFKMRRLSDYVPTPDLLEETEPTQTPYERRSNPMDKITETIQSHLKLHFDTPGVPQSESLSHLAHGMTKARAARLFYQIAVLATCDYIKVTQLERKGDELYGDILISRGLKM

SEQ ID NO: 2 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQQLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSHEYIPQKIKQGMHL

SEQ ID NO: 3 - protein, 562 aa, Zea mays
MKLKINKACDLGSISVLPPRRTGGSGGAGGAGSSAAPVAAGSQQRSQPMSQHSFSQGVVGSGGASSLLHSQSQLSQASLDENLLTLHLASPARDQRFGFHDDLSKKMTSLPVSSASCVREESQLQLAKTSSNPVHRWNPSLPDGRSVGPTEDVERKFQHMASSVHKVGMVLDSVQTDVMQLNRAMKEATLDSGSIQQKIVLLDALLQKNLKGQDDLKALVESNTKSISDQLTVLNSYSNKLDEISSTLSILPKQIETNLKQQQSDTFRIFRKDMEEIVRAVRSLNNKIDAIQMPTDQSCTTNGRPLMNQLPVDRNERPQVNQTPEVTRVSQTPVATMANQRPVAKGRHLVSQTPAANGKPLTNQTSVANGRSLMSQVPAANGKPLTNQIAVPKGRPMMRQKTAEGGRPQMNQIPVADGWPHTNKIHAPEVRHPVPLVCPAAGPRPKVEEGKLRAVPQKLTGCRSRVTPKQEEAATNTKVVVGRAAATEKVVIFIDDDSDDGGSEALASCVILRPAPLGSGAGECDLVKVGAEESQEILRRARKRRRREMQAIVACMPPELGY

SEQ ID NO: 4 - protein, 227 aa, Zea mays
MPESRDGRSEDLADLSGGVGGGGFFIRRVASPGALAVRGVRKPLARRYISPSRNKENLLPVWALRVTPTKRSPLPGWYPRTPLRDITAIAKAIQRSRSRIAAAQQRSQRIEQSSQSVNVTTPAQAEQDAHIAEASHAVASGSGSTEREAVANPATVLADDNLNVSSLAAEGSLNTPPKPMDPALADKKLSGSIEKVEKLVRKNLKRTSRAAQASRRATQRRNLMSMR

SEQ ID NO: 5 - protein, 609 aa, Zea mays
MTGAGMKLKINKACDLSSISVLPPRRTGGSGGAGSSAAAAAVAAGSQQRSQPLSQQSFSQGAVGSGGASSLLHSQSQLSQASLDENLLTLHLASPARDQGFRLHDDSSKKMTSLPVTSASCVREESQLQLAKTSSNPVHRWNPSLPDSRYFIELHSFLFECLTGSGLTGVVPAEDVERKFQHMASSVHKVGMVLDSVQNDTMQLNRAMKEAALDSGSIQQKIVVLDTSMQKNLKGQGDLKVLVESNTKNIADQLAVLNSHSNKLVEISSALSVLPKQIERDLKQQQSDIFRIFRNNMEEIARAVKSLNSKIDAIQMPTEQRCTTNGRPLMNQLPVDRNERPQVNQTPEVTRVSQTTVASLVNQAPAANGRHLMSKTPAANGKSVMRQTPAENGRHLVSQTPAANGKTLKRQTSVTNGISLMSQVPAANGKPVVIQIPAPKGRPMMRQKTAESGRTEMNEIPVASGRPHTNKIPIAEVHPAPLVCPAKQVATDPKPKVDEEKLKALPQKLTSSRSRVAPKQEEAANTKVISRAAATEKVVIFIDDSDDDNDVRASCVILRPSGSGSLGERECDLMKVGAEESQEIPRRARKRRRREMQATVGVACMPLPA

SEQ ID NO: 6 - protein, 224 aa, Zea mays
MRNFKMLEVRTARRPALADISGGGFFMRTVESPGAVLVNGAVKRPARQFLSPSSNKENVPPVGAFRATPKRRTPLPDWYPRTPLRDITSIVKAIERRRSRLQNAAAQQQIQWTEDPSRSVDPITPVQAEQGGVPTTVDGQGVGSPATCLEDGKLKTSSYPSSDCSLQATPSKPNDPALADLVEKKLSSSIEQIEKMVRRNLKRTSKAAQPSKRTIQRRVLMSMR

SEQ ID NO: 7 - protein, 226 aa, Zea mays
MSYTNFSQDYKMPQLRTASRPALASNSAGGFFIRRRVASPGTSQAKGAAKPLARRVRTPAARAKPKRRSPLPDWYPRVPLRDITSIVKALEKRNRLEEDAARQHIQSNEDSSQPVDPTTAEHSNPDSQSTQTQETPGAVASGPSSTSAVANRVTSVAEGKQEATDCSLQVAPSKPNDPSPADLVKKLSGSIEQIEKMVRRHMKETHPKAAQPSKVVVQRRILMSMR

SEQ ID NO: 8 - protein, 453 aa, Zea mays
MRRCAEAIARAVVEFLDAVILAFIRSCFGARPRRGSGLRVRPTPAPPRSPSLSAHLAAVSWSSSFQDALVRGDRAAEVIWDEEGLVRDGRFHEDLADGCGIDEELRREASYLKLCGTISETPAELRNEQSYENNLETTNECDSKLTYGPPANCKLLFEANSSQGQRNPRCEERHSLRSILNSEDAGRHHGAEYVPRSASSEKRLFQNMQHKPLDSGGSPFPTPLVLRDDMQTPGTAYASHRGTSISGKRVRTRKQFIYPVLRSIENRLQQIELTEDSSPLASSDPLKERDLGADSTKDPTQASSTSVVKSGLSETPSYSAPDPNASYGVEESLSKSNSDEKNAALSLSRWLKSSSADAENQDDEDCSFLTEKPVFMVAPDLNLDTSNPTPRLPKAWDGNGIPNTTTRYREDQRVSWHTTPFEQRLLKVLSDEDRCPPRKVVRGKLFHLEETAE

SEQ ID NO: 9 - protein, 432 aa, Zea mays
MMGCLFGCFRASGDGGDAKGAGDRNTSLAPATTTTIQQDGTGRRIRPPSRNALSAVFQREDEGSRAAQAASSWAEQTGESKRMDQELEPQTIIQKNCGALLQTTNNIQSVLRDADSVHQKETHSGCLPVMSDDVHFMEAPKVENCETPPRSHQSSTVPDATSSSKTNDELRTSATSLKNNVEESTNENDTEACVQGEEQQQALDLAEDFEECGVSKEDFFPPEKLEDPKCAKNDHVVSMEISISDECSLFQSSEGSVSPSNKIRGSMNTTSTEKSPKTEATIHATRKKLLKSNTSELELPSLAQWLKPPNPKKAFRDEAVTGDRSHSAKSSDEDRPIIGMVAAHWKDKEPANFTSKWWDGNGIPNSTNKYKEDQKVSWHATPFEERLEKALSEEKSLSERNCSSGKTSQFLGVEGEESDTAGSNRLYATAYA

SEQ ID NO: 10 - protein, 509 aa, Zea mays
MSTIAASRRSSSSSSSATAKRPAIADGPGGPKATAAQAKKRVALGNITNVAARGGRASVGGSLGNVMAPTSSAKLNPTVPLKKPSLATSARSVSSSIRGSADKPASIKPAPPVARHGSATQRHNNVPPPKVPTIADVPSRAPALVSCTGLVSPGRSGDSVSSDETMSTCDSMKSPDFEYVDNQDTSMLASLQRRTSEHLRISEDRDVEENKRKKNAVAPMEIDRICDVDSEYEDPQLCATLASDIYMHLREAETKKRPSTDFMETIQKDVNPSMRAILIDWLVEVAEEYRLVPDTLYLTVNYIDRYLSGNEISRQRLQLLGVACMLIAAKYEEICAPQVEEFCYITDNTYFRDEVLDMEASVLNYLKFEMTAPTAKCFLRRFARAAQACDEDPALHLEFLANYIAELSLLEYSLLSYPPSLIAASAIFLARFILQPTKYPWNSTLAHYTQYKPSKLSECVKALHRLCSVGSGSNLPAIREKYSQHKYKFVAKKQCPPQIPTEFFRDTTC

SEQ ID NO: 11 - protein, 298 aa, Zea mays
MASSKRRFGAAGGADKKDLFHVVHKVPAGDSPYVVAKHLQLVEKQPDVAIVWFWKAINSGDRVDSALKDMAMVMKQQDRSEEAIEAIRSFRHLCSKQAQESLDNLLIDLYKKCGKVEEQIELLKQKLKSIYLGEAFNGKATKKARSHGKKFQVSIQQETSRILGNLGWAYMQQNNFEAAELVYRKAQAIEPDANRACNLGLCLIKQGRHDEARQALEDVRLRRIYGSEDGKVVARAEQLLRELNPLQCVSSPFQVGLSVHEGIMGKPDLVVMNEWTPFRSRRLPVFEEIATFRDQMAC

SEQ ID NO: 12 - protein, 1096 aa, Zea mays
MAAADGDALIAAFAVSKGGIVLKHIFLNAPPPEAMCGSGGREVESDEEDPPVMVGRHPDCHVLVDHPSVSRFHLELRCRRRQSLITVTDLHSVHGTWVSGRRIPPNTPVDLATGDTLRLGASKREYKLLWLSLREAFEMDDLMYMPSLPEEDKEEPYVKEPSSKLLPGHRDSVNMETHQDTSEQIVSEDIAFPAKVAPSAPPLSEFLQPFFVEEHSLSQFHEKRNGVTEEKLVDKNQISESFGSLIIQEMPGTLTNAGKSIQSGEQEDASNKVSKRSKLKSVKSLRVDTGRSSERSSTLSHSFQKGDQNDIVVCSQSCGTECAVCIALFGISEIEKAEEKEELIAEDNVDMNPPASMIMEGNMNERKPDNYIPQDPIGAKLQKKLGLLDSALPLHFKDDVFADKEIPQWNVASVHTESELLSEYLIIPEVKHDDLNHLNLEEGLSKSENINPNKITEGPGNCQLEGTIRGNLFDNLDTDGIEEGEEICPLDKDEITPNGSGNIIMERSNIVLKPTISQQLMDSISPLNLDHGDFSENENSMLNTGDQMKLNEPVSENLNPLIPTDEKYLKSQTEECMPISYLEFKDDILLDRENSVLAPRKYEAMSPVRQENLFSDKENVTPASKVKTVVRGVLGTRMDNSVSAANASNKNKVLGSRVDNSVSTENSSNKKQCELSSKSKKVHTVDFDVFYSDKENLTPISSGGMKARKCFPKDLSVDLDQDQEAFCSDKENLTPLSSAARKTRDMSGNLTRVESAVTKKRVVGRLPFQTLVSNSPLRPASSHDCTCAVARPAGVAAGDLAIKLEDKLNDLSCNGHESGSAGEGMKTWTMVANTDSLLDDESRKAIMLLKGLKGTRLFIPRIVIRELDSMKQREGLFRRSTKATSILQWIEECMARESWWIHVQSSADMFPVAPTPPATPSAQRIDEEIEISSGSFNPMMALFGPRSSAALADMISPRPEDRVLDCALLVSRVRSNEKVVVLSNSVTLKIKAMAEGLPCEGAKEFRESLVDPSSRRFMWAASAPRGSAWSCLDASALAENYYNSRHHAMKRRVLVAARPSESEAAKGLKLILRHNSLYAQATDAVNKTPLVSLAAV

SEQ ID NO: 13 - protein, 502 aa, Zea mays
MSTHAASRRSSSSSAVAKRPAIAEGATKAAGPGPTAAQAKKRTALVNITNVAAPGARVAAVGKVAPPVTGAKLNPATSGAPLKKPSLANARTIRGSAAKSASIKPAPPVSRHDSSSEQKHNVLVPTTVHVPSRAPALVPYSSFVSPGRSRDSVSTDETMSTCDSMKSPDFEYIDNGGCSMLASLQRRADEHLRTSEDRDVEENKWKKNGPAPMEIDSICEVDSNLEDPQLCAALASDIYMHLREAEMKKRPSTDFMKTIQKDVNPSMRAILIDWLVEVAEEYRLAPDTLYLTVNYIDRYLSGNEINRQRLQLLGVACMLIAAKYEEICAPQVEEFCYITDNTYFRDEVLEMEASVLNYLKFEMTAPTAKCFLRRFARSAQACDEDPALHLEFLANYIAELSLLEYSLLSYPPSLIAASAIFLARFVLQPTKYPWNSTLAHYTQYKPSELSECVKTLHRLSSVGPGSNLPAIREKYSQHKYKFVAKKQCPPQIPAEFFRDAAC

SEQ ID NO: 14 - DNA, 20 bp, Zea mays
  source 1..20: mol_type = unassigned DNA
tggtggtgttggtggtggag

SEQ ID NO: 15 - DNA, 20 bp, Zea mays
  source 1..20: mol_type = unassigned DNA
tcaggagggtggcctcgccg

SEQ ID NO: 16 - DNA, 20 bp, Zea mays
  source 1..20: mol_type = unassigned DNA
ggcgttgagggtcacaccga

SEQ ID NO: 17 - DNA, 20 bp, Zea mays
  source 1..20: mol_type = unassigned DNA
tcggtgtgaccctcaacgcc

SEQ ID NO: 18 - DNA, 20 bp, Zea mays
  source 1..20: mol_type = unassigned DNA
caatggccgtaatatcacgg

SEQ ID NO: 19 - DNA, 20 bp, Zea mays
  source 1..20: mol_type = unassigned DNA
atcaggagggtggcctcgcc

SEQ ID NO: 20 - DNA, 20 bp, Zea mays
  source 1..20: mol_type = unassigned DNA
gtcggtgtgaccctcaacgc

SEQ ID NO: 21 - DNA, 20 bp, Zea mays
  source 1..20: mol_type = unassigned DNA
ctgcagagccccgagatgcc

SEQ ID NO: 22 - DNA, 20 bp, Zea mays
  source 1..20: mol_type = unassigned DNA
ctggatggtaccccaggacg

SEQ ID NO: 23 - DNA, 20 bp, Zea mays
  source 1..20: mol_type = unassigned DNA
gcaatggccgtaatatcacg

SEQ ID NO: 24 - DNA, 20 bp, Zea mays
  source 1..20: mol_type = unassigned DNA
gaggatggcaagctgaagac

SEQ ID NO: 25 - DNA, 20 bp, Zea mays
  source 1..20: mol_type = unassigned DNA
acgattgatgtgatgtcacg

SEQ ID NO: 26 - DNA, 20 bp, Zea mays
  source 1..20: mol_type = unassigned DNA
cctgactggtacccgaggac

SEQ ID NO: 27 - DNA, 20 bp, Zea mays
  source 1..20: mol_type = unassigned DNA
aagatgcttgaagtgaggac

SEQ ID NO: 28 - DNA, 20 bp, Zea mays
  source 1..20: mol_type = unassigned DNA
acagcaccattcaccagcac

SEQ ID NO: 29 - DNA, 20 bp, Zea mays
  source 1..20: mol_type = unassigned DNA
ccatcctccaaacaggttgc

SEQ ID NO: 30 - DNA, 20 bp, Zea mays
  source 1..20: mol_type = unassigned DNA
agcttgccatcctccaaaca

SEQ ID NO: 31 - DNA, 20 bp, Zea mays
  source 1..20: mol_type = unassigned DNA
ccgatctgtggatccaataa

SEQ ID NO: 32 - DNA, 20 bp, Zea mays
  source 1..20: mol_type = unassigned DNA
tcctcctctcaattgcctgc

SEQ ID NO: 33 - DNA, 20 bp, Zea mays
  source 1..20: mol_type = unassigned DNA
cggttgatcttcgagtggag

SEQ ID NO: 34 - DNA, 20 bp, Zea mays
  source 1..20: mol_type = unassigned DNA
atgatgtcgagcttgtcgag

SEQ ID NO: 35 - DNA, 20 bp, Zea mays
  source 1..20: mol_type = unassigned DNA
gaacccctcggtgcccatgg

SEQ ID NO: 36 - DNA, 20 bp, Zea mays
  source 1..20: mol_type = unassigned DNA
tcgagccgtttgcggttgat

SEQ ID NO: 37 - DNA, 20 bp, Zea mays
  source 1..20: mol_type = unassigned DNA
ctctcgtacacgatcaccac

SEQ ID NO: 38 - DNA, 20 bp, Zea mays
  source 1..20: mol_type = unassigned DNA
accttcctctcgtacacgat

SEQ ID NO: 39 - DNA, 20 bp, Zea mays
  source 1..20: mol_type = unassigned DNA
ctgtagagaagcttcacctt

SEQ ID NO: 40 - DNA, 20 bp, Zea mays
  source 1..20: mol_type = unassigned DNA
gagtggagcgtcgccgccat

SEQ ID NO: 41 - DNA, 20 bp, Zea mays
  source 1..20: mol_type = unassigned DNA
tcgcgcggaaggcgccgctc

SEQ ID NO: 42 - DNA, 20 bp, Zea mays
  source 1..20: mol_type = unassigned DNA
atcctgcggaggaggagcgc

SEQ ID NO: 43 - DNA, 20 bp, Zea mays
  source 1..20: mol_type = unassigned DNA
ttctgctgcaggagctgctg

SEQ ID NO: 44 - DNA, 20 bp, Zea mays
  source 1..20: mol_type = unassigned DNA
ctccagcctccgccgcaact

SEQ ID NO: 45 - DNA, 20 bp, Zea mays
  source 1..20: mol_type = unassigned DNA
gatcctgcggaggaggagcg

SEQ ID NO: 46 - DNA, 20 bp, Zea mays
  source 1..20: mol_type = unassigned DNA
gctccctgcagaagtagcag

SEQ ID NO: 47 - DNA, 20 bp, Zea mays
  source 1..20: mol_type = unassigned DNA
aagtgcagccggcacaatct

SEQ ID NO: 48 - DNA, 20 bp, Zea mays
  source 1..20: mol_type = unassigned DNA
gagcggcgccgctcgtcgcc

SEQ ID NO: 49 - DNA, 20 bp, Zea mays
  source 1..20: mol_type = unassigned DNA
aagagtatgcagatatcgtt

SEQ ID NO: 50 - DNA, 20 bp, Zea mays
  source 1..20: mol_type = unassigned DNA
gagcagtgcttgttctgctg

SEQ ID NO: 51 - DNA, 20 bp, Zea mays
  source 1..20: mol_type = unassigned DNA
gatgtattcgtactgaggcg

SEQ ID NO: 52 - DNA, 20 bp, Zea mays
  source 1..20: mol_type = unassigned DNA
attgcctcaatgtcaacagg

SEQ ID NO: 53 - DNA, 20 bp, Zea mays
  source 1..20: mol_type = unassigned DNA
cacatatcttggctccctgc

SEQ ID NO: 54 - DNA, 20 bp, Zea mays
  source 1..20: mol_type = unassigned DNA
gtgatgggctggataagatt

SEQ ID NO: 55 - DNA, 20 bp, Zea mays
  source 1..20: mol_type = unassigned DNA
tggagggcgaaaagaaagtg

SEQ ID NO: 56 - DNA, 20 bp, Zea mays
  source 1..20: mol_type = unassigned DNA
ccttctgtatattcaggctt

SEQ ID NO: 57 - DNA, 20 bp, Zea mays
  source 1..20: mol_type = unassigned DNA
tccattcctgttgacattga

SEQ ID NO: 58 - DNA, 20 bp, Zea mays
  source 1..20: mol_type = unassigned DNA
ttctcttgcagatgttgtta

SEQ ID NO: 59 - DNA, 20 bp, Zea mays
  source 1..20: mol_type = unassigned DNA
cagtgttccagcgtttggcc

SEQ ID NO: 60 - DNA, 20 bp, Zea mays
  source 1..20: mol_type = unassigned DNA
acagaacttgtcattggcca

SEQ ID NO: 61 - RNA, 20 nt, Zea mays
  source 1..20: mol_type = unassigned RNA
tggtggtgttggtggtggag

SEQ ID NO: 62 - RNA, 20 nt, Zea mays
  source 1..20: mol_type = unassigned RNA
tcaggagggtggcctcgccg

SEQ ID NO: 63 - RNA, 20 nt, Zea mays
  source 1..20: mol_type = unassigned RNA
ggcgttgagggtcacaccga

SEQ ID NO: 64 - RNA, 20 nt, Zea mays
  source 1..20: mol_type = unassigned RNA
tcggtgtgaccctcaacgcc

SEQ ID NO: 65 - RNA, 20 nt, Zea mays
  source 1..20: mol_type = unassigned RNA
caatggccgtaatatcacgg

SEQ ID NO: 66 - RNA, 20 nt, Zea mays
  source 1..20: mol_type = unassigned RNA
atcaggagggtggcctcgcc

SEQ ID NO: 67 - RNA, 20 nt, Zea mays
  source 1..20: mol_type = unassigned RNA
gtcggtgtgaccctcaacgc

SEQ ID NO: 68 - RNA, 20 nt, Zea mays
  source 1..20: mol_type = unassigned RNA
ctgcagagccccgagatgcc

SEQ ID NO: 69 - RNA, 20 nt, Zea mays
  source 1..20: mol_type = unassigned RNA
ctggatggtaccccaggacg

SEQ ID NO: 70 - RNA, 20 nt, Zea mays
  source 1..20: mol_type = unassigned RNA
gcaatggccgtaatatcacg

SEQ ID NO: 71 - RNA, 20 nt, Zea mays
  source 1..20: mol_type = unassigned RNA
gaggatggcaagctgaagac

SEQ ID NO: 72 - RNA, 20 nt, Zea mays
  source 1..20: mol_type = unassigned RNA
acgattgatgtgatgtcacg

SEQ ID NO: 73 - RNA, 20 nt, Zea mays
  source 1..20: mol_type = unassigned RNA
cctgactggtacccgaggac

SEQ ID NO: 74 - RNA, 20 nt, Zea mays
  source 1..20: mol_type = unassigned RNA
aagatgcttgaagtgaggac

SEQ ID NO: 75 - RNA, 20 nt, Zea mays
  source 1..20: mol_type = unassigned RNA
acagcaccattcaccagcac

SEQ ID NO: 76 - RNA, 20 nt, Zea mays
  source 1..20: mol_type = unassigned RNA
ccatcctccaaacaggttgc

SEQ ID NO: 77 - RNA, 20 nt, Zea mays
  source 1..20: mol_type = unassigned RNA
agcttgccatcctccaaaca

SEQ ID NO: 78 - RNA, 20 nt, Zea mays
  source 1..20: mol_type = unassigned RNA
ccgatctgtggatccaataa

SEQ ID NO: 79 - RNA, 20 nt, Zea mays
  source 1..20: mol_type = unassigned RNA
tcctcctctcaattgcctgc

SEQ ID NO: 80 - RNA, 20 nt, Zea mays
  source 1..20: mol_type = unassigned RNA
cggttgatcttcgagtggag

SEQ ID NO: 81 - RNA, 20 nt, Zea mays
  source 1..20: mol_type = unassigned RNA
atgatgtcgagcttgtcgag

SEQ ID NO: 82 - RNA, 20 nt, Zea mays
  source 1..20: mol_type = unassigned RNA
gaacccctcggtgcccatgg

SEQ ID NO: 83 - RNA, 20 nt, Zea mays
  source 1..20: mol_type = unassigned RNA
tcgagccgtttgcggttgat

SEQ ID NO: 84 - RNA, 20 nt, Zea mays
  source 1..20: mol_type = unassigned RNA
ctctcgtacacgatcaccac

SEQ ID NO: 85 - RNA, 20 nt, Zea mays
  source 1..20: mol_type = unassigned RNA
accttcctctcgtacacgat

SEQ ID NO: 86 - RNA, 20 nt, Zea mays
  source 1..20: mol_type = unassigned RNA
ctgtagagaagcttcacctt

SEQ ID NO: 87 - RNA, 20 nt, Zea mays
  source 1..20: mol_type = unassigned RNA
gagtggagcgtcgccgccat

SEQ ID NO: 88 - RNA, 20 nt, Zea mays
  source 1..20: mol_type = unassigned RNA
tcgcgcggaaggcgccgctc

SEQ ID NO: 89 - RNA, 20 nt, Zea mays
  source 1..20: mol_type = unassigned RNA
atcctgcggaggaggagcgc

SEQ ID NO: 90 - RNA, 20 nt, Zea mays
  source 1..20: mol_type = unassigned RNA
ttctgctgcaggagctgctg

SEQ ID NO: 91 - RNA, 20 nt, Zea mays
  source 1..20: mol_type = unassigned RNA
ctccagcctccgccgcaact

SEQ ID NO: 92 - RNA, 20 nt, Zea mays
  source 1..20: mol_type = unassigned RNA
gatcctgcggaggaggagcg

SEQ ID NO: 93 - RNA, 20 nt, Zea mays
  source 1..20: mol_type = unassigned RNA
gctccctgcagaagtagcag

SEQ ID NO: 94 - RNA, 20 nt, Zea mays
  source 1..20: mol_type = unassigned RNA
aagtgcagccggcacaatct

SEQ ID NO: 95 - RNA, 20 nt, Zea mays
  source 1..20: mol_type = unassigned RNA
gagcggcgccgctcgtcgcc

SEQ ID NO: 96 - RNA, 20 nt, Zea mays
  source 1..20: mol_type = unassigned RNA
aagagtatgcagatatcgtt

SEQ ID NO: 97 - RNA, 20 nt, Zea mays
  source 1..20: mol_type = unassigned RNA
gagcagtgcttgttctgctg

SEQ ID NO: 98 - RNA, 20 nt, Zea mays
  source 1..20: mol_type = unassigned RNA
gatgtattcgtactgaggcg

SEQ ID NO: 99 - RNA, 20 nt, Zea mays
  source 1..20: mol_type = unassigned RNA
attgcctcaatgtcaacagg

SEQ ID NO: 100 - RNA, 20 nt, Zea mays
  source 1..20: mol_type = unassigned RNA
cacatatcttggctccctgc

SEQ ID NO: 101 - RNA, 20 nt, Zea mays
  source 1..20: mol_type = unassigned RNA
gtgatgggctggataagatt

SEQ ID NO: 102 - RNA, 20 nt, Zea mays
  source 1..20: mol_type = unassigned RNA
tggagggcgaaaagaaagtg

SEQ ID NO: 103 - RNA, 20 nt, Zea mays
  source 1..20: mol_type = unassigned RNA
ccttctgtatattcaggctt

SEQ ID NO: 104 - RNA, 20 nt, Zea mays
  source 1..20: mol_type = unassigned RNA
tccattcctgttgacattga

SEQ ID NO: 105 - RNA, 20 nt, Zea mays
  source 1..20: mol_type = unassigned RNA
ttctcttgcagatgttgtta

SEQ ID NO: 106 - RNA, 20 nt, Zea mays
  source 1..20: mol_type = unassigned RNA
cagtgttccagcgtttggcc

SEQ ID NO: 107 - RNA, 20 nt, Zea mays
  source 1..20: mol_type = unassigned RNA
acagaacttgtcattggcca

SEQ ID NO: 108 - DNA, 412 bp, Zea mays
  source 1..412: mol_type = unassigned DNA
tactaactgctagctgcaggcaattgagaggaggagaagtcgtctgcagaatgctgcagctcagcagcagatccagtggacagaagacccttcccgatctgtggatccaataactccagtacaggcagagcagggtggtgtgccaacaactgtggatggtcaaggtgttggaagccctgcaacctgtttggaggatggcagtcctatccatcatctgactgctccttgcaggccactccatccaaaccaaacgatccagctctcgcagatctcgtggagaagaagctgtccagctcgatagagcagatcgagaagatggtgcggcgaaacctgaagagaacttcgaaggccgctcagccttccaagaggaccatccagaggcgcgtcctgatgtccatgcgatgagctgaga

SEQ ID NO: 109 - DNA, 444 bp, Zea mays
  source 1..444: mol_type = unassigned DNA
ctggaaatttgctgatgtactaactgctagttgcaggcaattgagaggaggagaagtcgtctgcagaatgctgcagctcagcagcagatcctgtggacagaagacccttcccgatctgtggatccaataactccagtacaggcagagcatggtggtgtgccaacaactgaggatggtcaaggtgttggaagccctgcaacctgtttggaggatggcaagctgaaggtctgactgctccttgcaggccactccatccaaaccaaacgatccagctcgcgcagatctcgtggagaagaagttgtccagctcgatagagcagatcgagaagatggtgcggcgaaacctgaagagaacttcgaaggccgctcagccttccaagaggaccatccagaggcgcgtcctgatgtccatgcgatgagctgagaaagctatctgctctgccat

SEQ ID NO: 110 - DNA, 400 bp, Zea mays
  source 1..400: mol_type = unassigned DNA
tggcggcgacgctccactcgaagatcaaccgcaaacggctcgacaagctcgacatcatcaaaatctggtggggaaactggtcccagggattcgagttccgcacggttttgactgctatttttgtgttttttttatttgtttttgctgacttttgtttgtgtgtttcacttttagtgaggaaattttgaacccctcggtgcggctctctggaatcctcatgggtgagttcgattttgcttgcgccacccaaaactgttcctcccattttttggatagtttttttttacgttccaatttccatggtcgaaaattcgaaatgcgtgagcaggtggcgtggtgatcgtgtacgagaggaaggtgaagcttctctacagtaagtttcttcctccctacatcctac

SEQ ID NO: 111 - DNA, 400 bp, Zea mays
  source 1..400: mol_type = unassigned DNA
aacattgcacctactgagcacagacctacgaagatggaggggtacatgtagtagatgtccctcttggagcagtgcttgttctgctgcaggagctgctgcaccacgaacagcaccctcaggagcacatctacgcatgaacaacgcatcagaaagtaattagtgtgatcacataaatgcaaagatttagatgtattcgtactgaggtgtggtagtccttgtggagcagagagaggacgtccgtgccgacggggatgtcgtagctgcaggcactgcaatcacgcacgagcgcgcgcccagggtctacggatgtcacacatttgggtgaaaattgttcgagggatgtgacggtgaattggtcgggaaggagatgatcccgggagcgggcgcggaagctaaacca